(19) United States
(12) Patent Application Publication
(10) Pub. No.: US 2023/0405028 A1
(43) Pub. Date: Dec. 21, 2023

(54) COMPOSITIONS FOR REDUCING INFLAMMATION TO IMPROVE OR MAINTAIN MENTAL OR PHYSICAL HEALTH

(71) Applicant: GOODCAP PHARMACEUTICALS LTD., Toronto (CA)

(72) Inventors: Dwight Darryl HUDSON, Raymond (CA); Irie Victoria SELKIRK, Toronto (CA)

(21) Appl. No.: 18/248,626

(22) PCT Filed: Oct. 12, 2021
(86) PCT No.: PCT/IB2021/059301

§ 371 Date: Apr. 11, 2023

Related U.S. Application Data
(60) Provisional application No. 63/090,552, filed on Oct. 12, 2020.

(51) Int. Cl. A61K 31/675; A61K 31/4045; A61K 36/06; A61K 31/48; A61K 31/137; A61K 31/36; A61K 31/085; A61K 31/015; A61K 31/11; A61K 31/12; A61K 9/00; A61P 29/00

(52) U.S. Cl. CPC A61K 31/675; A61K 31/4045; A61K 36/06; A61K 31/48; A61K 31/137; A61K 31/36; A61K 31/085; A61K 31/015; A61K 31/11; A61K 31/12; A61K 9/0053; A61P 29/00

(57) ABSTRACT

The present disclosure provides compositions and methods for reducing inflammation to improve or maintain mental health or physical health in an individual. In some aspects, the compositions include at least one 5HT2A agonist and at least one TRP agonist, wherein the therapeutically effective amount of the 5HT2A agonist is between about 1 μg and about 300 mg and the therapeutically effective amount of the at least one TRP receptor agonist is between about 0.01 mg and about 300 mg. The compositions may be formulated for any suitable ingestion mode, including gastrointestinal, transmucosal and parenteral.

### BACKGROUND OF THE INVENTION

### Field of the Invention

The present disclosure relates to compositions for reducing inflammation to improve or maintain mental or physical health.

### Background

Depression has become an epidemic in the United States and is only projected to get worse. The NIMH lists clinical depression is diagnosed in 7% of U.S. adults and there are growing numbers of children and teenagers in recent years. Depression and mental illness are the leading cause of disability in the world, costing the economy trillions a year in lost productivity, missed days of work and care for the many physical and mental illnesses related to depression, like anxiety, posttraumatic stress disorder, migraines and sleep disorders. Depressive episodes are periods of two weeks or longer experiencing low mood, coupled with other symptoms such as poor self-image, sleep difficulty, loss of appetite, poor concentration, and low energy. Among U.S. adolescents, a disturbingly high rate has reported having experienced either major depression or low-grade depression. According to the World Health Organization, about five percent of the world's population is depressed. Though psychotherapy is available to some individuals, a common strategy for dealing with depression increasingly includes pharmaceuticals and often more than one is prescribed in combination. Pharmaceutical antidepressants work by indirect methods through alter the signaling of stress hormones such as norepinephrine and neurotransmitters serotonin and dopamine that influence mood, energy, focus, and motivation. These drugs were designed based on the assumption that depression is due to biochemical imbalance in the brain. The most common antidepressants are serotonin re-uptake inhibitors (“SSRIs”) which act to prevent re-uptake and subsequent breakdown of 5-hydroxy-tryptamine (“5-HT”), commonly referred to as serotonin, an important neurotransmitter. Increasing the pool of serotonin in the brain through breakdown inhibition improves mood in many individuals after three to four weeks of regularly taking an SSRI medication.

There has been little innovation in the antidepressant field since SSRIs were first introduced. Current medications have limited efficacy in controlling the symptoms of major depressive disorder (“MDD”). SSRIs often take four to eight weeks to show efficacy and are associated with adverse events on long-term use. Side effects of antidepressants are common and include nausea, vomiting or diarrhea, headache, drowsiness, dry mouth, insomnia, nervousness, agitation or restlessness, dizziness, blurred vision, sexual problems, such as reduced sexual desire, difficulty reaching orgasm or inability to maintain an erection (erectile dysfunction), impact on appetite, leading to weight loss or weight gain and increased risk if suicide. With suicides in the U.S. at their highest number in thirty years, there is need for rapid solutions and for more effective solutions.

In addition to diagnosed chronic and clinical depressions, some depression arises for no apparent reason and persists chronically. Such depression sometimes goes undiagnosed. Similarly, situational depression may follow a significant loss or life event, such as the death of a loved one, chronic illness, separation or divorce. To an extent, depression is a normal and necessary adaptive response to the inevitable changes and phases of life. Current medications do little to deal with the chronic depression and the acute depression described in this paragraph in the general population. With depression diagnosis going up and increased prescriptions given for long periods of time, there is need for alternative solutions for dealing with both acute and chronic depression. Furthermore, there is no solution for those who have not been medically diagnosed as “depressed” and/or those who are not depressed (medically diagnosed or not), but are looking to enhance aspects of creativity, mindfulness/awareness, sexual desire and other natural feelings that may enhance the human experience.

### SUMMARY

Compositions disclosed may be used to reduce inflammation for improving or maintaining mental or physical health, including to mitigate, eliminate or otherwise correct depression, anxiety, PTSD, mood disorders, pain and digestive problems, and to also reduce the use of other substances including pharmaceutical and illicit drugs. The compositions include at least one 5HT2A serotonin receptor agonist and one TRP receptor agonist, and may include at least one TRP receptor antagonist. The compositions may be applied to multimodal treatment of inflammation and of conditions associated with inflammation, including depression. The interaction of the compositions with the 5HT2A and TRP receptors results in altered activity of nociceptor cells and may also influence the endocannabinoid signaling system.

A 5HT2A agonist in combination with a TRP agonist provides a multimodal effect and shows improved efficacy over a 5HT2A agonist alone at an equivalent dose. Some examples of 5HT2A agonists used in the compositions provided herein include 4AcO-DMT (psilacetin), psilocybin, serotonin, lysergic acid amide, lysergic acid α-hydroxyethylamide, myristicin and elemicin. These compounds may be found in psilocybin-containing fungi, morning glory seeds, Hawaiian baby woodrose seeds and nutmeg. Some examples of TRP agonists used in the compositions provided herein include capsaicin, carvacrol, cinnamaldehyde, curcumin, eugenol, and piperine, and myristicin. These compounds may be sourced from materials that are generally regarded as safe (“GRAS”), such as cayenne peppers, turmeric, oregano, cloves, cinnamon and nutmeg. Other examples of TRP receptor agonists used in the compositions provided herein include β-caryophyllene, α-terpineol, cannabidiol (“CBD”), cannabidivarin (“CBDV”), cannabigerol (“CBG”), cannabigerolic acid (“CBGA”), delta-9-tetrahydrocannabinol (“THC”), delta-9-tetrahydrocannabivarin (“THCV”), delta-9-tetrahydrocannabivarinic acid (“THCVA”), cannabigevarin (“CBGV”), myrcene, eriodictyol, carvacrol, myrcene, thymol, carvacrol, menthol, 1-8 cineole, piperine, gingerol, allicin, myrhhanol, boswellic acid and derivatives. These compounds may be sourced from materials that are GRAS, such as cannabis, coffee, chocolate, peppermint, thyme, ginger, garlic, onion, myrrh, frankincense and cardamom.

Each of these 5HT2A receptor agonists and TRP receptor agonists may be prepared synthetically, or extracted, purified or otherwise obtained from biomass of plants, fungi or microorganisms, including the GRAS materials described above. In contrast with standard pharmaceutical approaches of increased activity and activation potential at a single receptor target, the compositions provided herein provide therapeutic effects through a multi-modal action at 5HT2A and at least one TRP receptor, which supports dosing at lower concentrations of the 5HT2A agonist than would be observed using only the 5HT2A agonist, and similarly dosing at lower concentrations of TRP receptor agonist that would be observed using only the TRP receptor agonist.

Through targeting the 5HT2A and TRP receptors together, inflammation markers and reactive oxygen species (“ROS”) may be reduced; as such, glutamate metabolism may be regulated to reduce the severity or prevalence of mental health conditions that may be associated with or result from improper sugar signaling in the gut. The compositions may be formulated for use by individuals suffering from conditions including pain, mood disorders (anger/bipolar), inflammation, depression, anxiety, bowel inflammation, peripheral pain, neuropathic pain, traumatic brain injury (“TBI”), headaches, Alzheimer's disease, dementia, concussion, diabetes, arthritis, heart disease and cancer. The compositions may be formulated for use by individuals seeking to maintain or improve mental health, maintain or improve physical health, facilitate relaxation, facilitate focus, facilitate creativity, facilitate digestion, improve euphoria, improve libido and sex drive, and facilitate sleep, including for individuals suffering from pain and inflammation related conditions. Different TRPs influence different genes downstream, allowing the compositions to be adjusted for specific metabolic conditions or benefits.

Through agonism at both the 5HT2A receptor and one or more TRP receptors together, the amount of 5HT2A agonist used may be lowered relative to the amount that would be required to elicit a comparable response without the TRP receptor agonist. This may be advantageous where the 5HT2A receptor is psychoactive or otherwise produces effects that may be uncomfortable and/or inconvenient for an individual taking the 5HT2A agonist. For example, psilocybin is strongly psychoactive. Benefits of psilocybin can be obtained at lower doses of psilocybin when a TRP receptor agonist is taken along with, preceding or shortly after the psilocybin.

Combining multiple 5HT2A agonists with multiple TRP agonists may further enhance the activity or alter the effects of the formulation. Psilocybin mushrooms themselves may have multiple 5HT2A agonists present. The seeds of Hawaiian Baby woodrose and Morning glory also contain multiple 5HT2As. Spices or essential oils from medical plants often contain multiple TRP agonists. Furthermore, the addition of various ingredients from different sources allowed for a reduced dose of each individual ingredient while still achieving medically relevant results.

Some TRP receptor agonists, such as myristicin present in nutmeg, may themselves be 5HT2A agonists and may enhance the effects of another 5HT2A agonist. Compositions including psilocybin and nutmeg show greater psychoactive effects resulting from psilocybin than would be expected at the doses being applied, because myristicin may influence both 5HT2A and GABA receptors, and is thought to be processing into MMDA. As a result, including nutmeg in the compositions may allow for a reduced dose of psilocybin or other 5HT2A agonist. For example, about 0.5 grams of nutmeg taken with 0.5 grams of psilocybin produced a more extensive level of euphoria and other psychoactive effects than psilocybin alone, indicating synergy between psilocybin and nutmeg. Bergamot, which includes myrcene, eriodictyol, carvacrol, linalool and other compounds, also intensifies the psychoactive effects of psilocybin, especially in compositions that also include nutmeg.

Combining cayenne with psilocybin improved painkilling and anti-inflammatory effects of psilocybin, providing pain relief with about 0.2 g of dried fruiting bodies that include psilocybin and 0.2 g of crushed cayenne peppers. Cayenne may decrease the onset time of psilocybin, but may also result in greater anxiety being experienced that with psilocybin alone.

Combining turmeric with psilocybin may prolong the effects of psilocybin and increase antidepressant qualities by improving mood without appreciable negative side effects at the doses assessed. Turmeric may also increase analgesic effects from psilocybin, and may calm the anxiety that may result from consuming psilocybin, or from a combination of cayenne and psilocybin. Turmeric allowed for reduced amounts of both cayenne and psilocybin in the compositions while maintaining efficacy. Reduction of the amount of psilocybin and cayenne in the compositions may facilitated mitigation of stomach cramps, intestinal indigestion and anxiety that may result from consumption of psilocybin and cayenne.

Combining clove with psilocybin may reduce inflammation, as well as improve mood and digestion, and may mitigate anxiety associated with psilocybin use (including in combination with cayenne). Black Pepper had a similar effect, particularly the mental effects around mood and anxiety, but an equivalent amount of clove may be more palatable and may mix better with other ingredients of the composition.

Combining cinnamon with psilocybin may result in improved positive impacts on mood from the psilocybin and may also help with diet, reducing sugar cravings. The calm that results from consuming cinnamon may reduce impulses such as fidgeting and may support increased focus.

Combining bergamot with psilocybin may enhance the effects of psilocybin and in some cases resulted in what was described as a “glow” or “light” feeling. Pure limonene or extract of orange flower did not result in the same effects as bergamot, indicating specific molecules in bergamot were essential to achieve this effect.

Combining chocolate or cocoa with psilocybin provides a TRP agonist that is synergistic with psilocybin the TRP3 receptor agonist (−)-epicatechin, a flavonol present in cocoa. Cacao may also provide stimulating effects through theophylline and other molecules present in chocolate. Chocolate may help mask strong flavours of other ingredients when the composition is formulated as a chew or other edible product.

Green tea also contains (−)-epicatechin and may be used as a means of administration of the formulations. (Uchida, 2018) Caffeine in green tea also does enhances the stimulant properties of psilocybin, mitigating side effects of psilocybin including yawning and drowsiness.

Combining coffee with psilocybin did not appear to be synergistic in terms of TRP receptor agonism or reduced dosage of psilocybin. Caffeine in coffee enhances the stimulant properties of psilocybin, mitigating side effects of psilocybin including yawning and drowsiness.

Combining peppermint or other plants high in menthol with psilocybin may improve mood enhancement and uplifting effects of psilocybin, increasing motivation and activity. Enhanced energy and concentration may also be reported. Peppermint also helped with stomach pain and digestion issues, particularly those resulting from psilocybin, and particularly stomach pain that may result from capsaicin.

Combining ginger which has gingerol, as well as garlic or onion which contain allicin with psilocybin may support correction of digestion issues and though there did not appear to be any effect with respect to mental health related issues such as mood, there does appear to be a synergistic effect with psilocybin with respect to inflammation which facilitates benefits at a lower dose of psilocybin.

Combining myrrh with psilocybin may result in stronger psychoactive effects from the psilocybin, and also enhance benefits in terms of neuroplasticity and neuroregeneration (Premkumar, 2014). Myrrh is a resin from the species of the genus Commiphora. The active ingredient in myrrh and similar resins is incensole, which has psychoactive properties and is a potent activator of TRPV3 (Moussaieff, 2008).

Combining frankincense with psilocybin may result in stronger psychoactive effects from the psilocybin, and also enhance benefits in terms of neuroplasticity and neuroregeneration. Frankincense is a resin obtained from the frankincense tree (Boswellia thurifera), which belongs to the family Buseraceae. The active ingredient is boswellic acid (Premkumar, 2014). Formulations including myrrh or frankincense may provide benefits to individuals suffering from post-traumatic stress disorder (“PTSD”) and traumatic brain injury (“TBI”), and may help with libido.

Combining cardamom with psilocybin may amplify anti-inflammatory properties especially with respect to the digestive tracts. Cardamom may be phototoxic and may also carry contraindications for some medications.

Combining oregano with psilocybin may amplify anti-inflammatory properties especially with respect to the digestive tract issues.

Cannabis may also be included in the compositions, and may have effects on both the body and the mind depending on the dose and variety consumed. The variety of phytocannabinoids, terpenoids, flavonoids, phenylpropanoids and other secondary metabolites in Cannabis sativa complicate work with cannabis as an active ingredient. In addition, the consumption of two strongly psychoactive substances—psilocybin and delta-9-tetrahydrocannabinol (“THC”)—may result in much stronger psychoactive effects. Other phytocannabinoids, such as cannabigerol (“CBG”), cannabidiol (“CBD”), and degradation products such as cannabinol (“CBN”) may enhance specific compositions, including those directed to neuroprotection for CBD and sleep for CBN. In addition to broad-spectrum cannabis extracts or other preparations, individual phytocannabinoids may modulate the effect of specific formulations.

Combining psilocybin with TRP receptor agonists from preparations of cayenne pepper, turmeric, clove, cinnamon and nutmeg, and optionally chocolate, may provide effective enhancement of the 5HT2A activity resulting from psilocybin. In addition, optional additives such as bergamot, oregano, myrrh and frankincense further extend the duration of perceived effects resulting from the TRP receptor agonists. Addition of bergamot to compositions that also include cayenne pepper, turmeric, clove, cinnamon and nutmeg may improve focus of the individual taking a formulation of the composition, but may also result in some psychoactive effects remaining between 12 and 24 hours after the dose was taken. As a result, bergamot may be avoided in compositions that are being formulated for use cases where a long return to baseline is unacceptable, and where there is a strong potential for overconsumption.

In a first aspect, herein disclosed is a composition for reducing inflammation to improve or maintain mental health or physical health in an individual. The composition includes at least on 5HT2A agonist and at least one TRP agonist. The 5HT2A agonist may include a tryptamine, ergoline, phenethylamine, phenylpropanoid or other 5HT2A agonist. The TRP agonist may include an agonist for one or more of the TRPV1, TRPA1, TRPM3, TRPM8, TRPV3 and TRPV4 receptors. The TRP agonist may include capsaicin, eugenol, curcumin, β-caryophyllene, myristicin or other TRP agonists. The agonist may include extracts from psilocybin-containing fungi, morning glory seeds, Hawaiian baby woodrose seeds or other fungi and plants. In some aspects, the agonist has a purity of at least about 99%. The TRP agonist may include extracts from cayenne pepper, clove, turmeric, nutmeg and other plants. In some aspects, the TRP agonist has a purity of at least about 99%. The compositions may be formulated for any suitable ingestion mode, including gastrointestinal, transmucosal and parenteral.

In a further aspect, herein disclosed is a composition for reducing inflammation to improve or maintain mental health or physical health in an individual comprising an effective amount of a 5HT2A agonist compound and an effective amount of a TRP agonist compound, wherein the therapeutically effective amount of the 5HT2A agonist is between about 1 μg and about 300 mg; and the therapeutically effective amount of the at least one TRP receptor agonist is between about 0.01 mg and about 300 mg. In some aspects, the therapeutically effective amount is per dose. In some aspects, the dose is administered 1 to 10 times per day.

In some aspects and embodiments, the therapeutically effective amount of the agonist is between about 10 μg and about 195 mg, about 50 μg and about 190 mg, about 100 μg and about 185 mg, about 200 μg and about 180 mg, about 300 μg and about 175 mg, about 400 μg and about 170 mg, about 500 μg and about 165 mg, about 600 μg and about 160 mg, about 700 μg and about 155 mg, about 800 μg and about 150 mg, about 900 μg and about 145 mg, about 1 mg and about 140 mg, about 5 mg and about 135 mg, about 10 mg and about 130 mg, about 15 mg and about 125 mg, about 20 mg and about 120 mg, about 25 mg and about 115 mg, about 30 mg and about 110 mg, about 35 mg and about 105 mg, about 40 mg and about 100 mg, about 45 mg and about mg, about 50 mg and about 90 mg, about 55 mg and about 85 mg, about 60 mg and about 80 mg, or about 65 mg and about 75 mg. In some aspects, the therapeutically effective amount is per dose. In some aspects, the dose is administered 1 to 10 times per day.

In some aspects and embodiments, the therapeutically effective amount of the at least one TRP receptor agonist is between about 0.1 mg and about 24 mg, about 0.5 mg and about 23 mg, about 1 mg and about 22 mg, about 2 mg and about 21 mg, about 3 mg and about 20 mg, about 4 mg and about 19 mg, about 5 mg and about 18 mg, about 6 mg and about 17 mg, about 7 mg and about 16 mg, about 8 mg and about 15 mg, about 9 mg and about 14 mg, about 10 mg and about 13 mg, or about 11 mg and about 12 mg. In some aspects, the therapeutically effective amount is per dose. In some aspects, the dose is administered 1 to 10 times per day.

In some aspects and embodiments, the therapeutically effective amount of the agonist is between about 10 μg and about 195 mg, about 50 μg and about 190 mg, about 100 μg and about 185 mg, about 200 μg and about 180 mg, about 300 μg and about 175 mg, about 400 μg and about 170 mg, about 500 μg and about 165 mg, about 600 μg and about 160 mg, about 700 μg and about 155 mg, about 800 μg and about 150 mg, about 900 μg and about 145 mg, about 1 mg and about 140 mg, about 5 mg and about 135 mg, about 10 mg and about 130 mg, about 15 mg and about 125 mg, about 20 mg and about 120 mg, about 25 mg and about 115 mg, about 30 mg and about 110 mg, about 35 mg and about 105 mg, about 40 mg and about 100 mg, about 45 mg and about mg, about 50 mg and about 90 mg, about 55 mg and about 85 mg, about 60 mg and about 80 mg, or about 65 mg and about 75 mg, and wherein the therapeutically effective amount of the at least one TRP receptor agonist is between about 0.1 mg and about 24 mg, about 0.5 mg and about 23 mg, about 1 mg and about 22 mg, about 2 mg and about 21 mg, about 3 mg and about 20 mg, about 4 mg and about 19 mg, about 5 mg and about 18 mg, about 6 mg and about 17 mg, about 7 mg and about 16 mg, about 8 mg and about mg, about 9 mg and about 14 mg, about 10 mg and about 13 mg, or about 11 mg and about 12 mg. In some aspects, the therapeutically effective amount is per dose. In some aspects, the dose is administered 1 to 10 times per day.

In some aspects and embodiments, the 5HT2A agonist comprises a tryptamine. In some aspects and embodiments, the tryptamine comprises a 4-substituted tryptamine. In some aspects and embodiments, the 4-substituted tryptamine comprises a 4-substituted DMT compound. In some aspects and embodiments, the 4-substituted DMT compound comprises a compound selected from the group consisting of 3-[2-(dimethylamino)ethyl]-4-phosphoryloxyindole (psilocybin), 3-[2-(dimethylamino)ethyl]-4-hydroxyindole (psilocin), 3-[2-(dimethylamino)ethyl]-4-acetoxyindole (4-acetyl-DMT; also known as 4-ACO-DMT)) and any suitable salt of any of the foregoing. In some aspects and embodiments, the effective amount of the 4-substituted DMT compound comprises between 0.001 mg/kg and 0.30 mg/kg, with reference to the body weight of the individual. Average adult body weight is about 70 kg. In some aspects and embodiments, the 4-substituted tryptamine comprises a compound selected from the group consisting of 3-[2-(trimethylamino)ethyl]-4-phosphoryloxyindole (aeruginascin), 3-[2-(methylamino)ethyl]-4-phosphoryloxyindole (baeocystin), 3-[2-(methylamino)ethyl]-4-hydroxyindole, 3-[2-(amino)ethyl]-4-hydroxyindole (norpsilocin), 3-[2-(amino)ethyl]-4-phosphoryloxyindole (called norbaeocystin) and any suitable salt of any of the foregoing. In some aspects and embodiments, the 4-substituted tryptamine comprises a 4-substituted tryptamine sourced from fungi that biosynthesize the 4-substituted tryptamine. In some aspects and embodiments, the fungi includes a species selected from the group consisting of Conocybe species including C. cyanopus, C. siligineoides and C. kuehneriana; Copelandia species including C. affinis, C. anomala, C. bispora, C. cambodginiensis, C. chlorocystis, C. cyanescens, C. lentisporus, C. tirunelveliensis, C. tropica, C. tropicalis and C. westii; Galerina species including G. steglichii; Gymnopilus species including G. thiersii, G. aeruginosus, G. braendlei, G. cyanopalmicola, G. intermedius, G. junonius, G. lateritius, G. liquiritiae, G. luteofolius, G. luteoviridis, G. luteus, G. purpuratus, G. subpurpuratus, G. validipes and G. viridans; Inocybe species including I. aeruginascens, I. aeruginascens, I. coelestium, I. corydalina, I. corydalina var. corydalina, I. corydalina var. erinaceomorpha, I. haemacta and I. tricolor; Panaeolus species including P. cinctulus, P. affinis, P. africanus, P. bisporus, P. cambodginiensis, P. castaneifolius, P. chlorocystis, P. cinctulus, P. cyanescens, P. fimicola, P. lentisporus, P. microsporus, P. moellerianus, P. olivaceus, P. rubricaulis, P. tirunelveliensis, P. tropicalis and P. venezolanus; Pholiotina species including P. cyanopus and P. smithii; Pluteus species including P. americanus, P. albostipitatus, P. americanus, P. cyanopus, P. glaucus, P. glaucotinctus, P. nigroviridis, P. phaeocyanopus, P. salicinus, P. saupei and P. villosus; and Psilocybe species including P. tampanensis, P. acutipilea, P. allenii, P. angustipleurocystidiata, P. antioquiensis, P. atlantis, P. aquamarina, P. armandii, P. aucklandii, P. atlantis, P. aztecorum, P. aztecorum var. aztecorum, P. aztecorum var. bonetii, P. azurescens, P. baeocystis, P. banderillensis, P. bispora, P. brasiliensis, P. brunneocystidiata, P. cubensis, P. caeruleoannulata, P. caerulescens, P. caerulescens var. caerulescens, P. caerulescens var. ombrophila, P. caerulipes, P. callosa, P. carbonaria, P. caribaea, P. chuxiongensis, P. collybioides, P. columbiana, P. cordispora, P. cubensis, P. cyanescens, P. cyanofibrillosa, P. dumontii, P. egonii, P. fagicola, P. fagicola var. fagicola, P. fagicola var. mesocystidiata, P. farinacea, P. fimetaria, P. fuliginosa, P. furtadoana, P. tampanensis, P. galindoi, P. gallaeciae, P. graveolens, P. guatapensis, P. guilartensis, P. heimii Guzman, P. herrerae Guzman, P. hispanica Guzman, P. hoogshagenii, P. hoogshagenii var. hoogshagenii, P. hoogshagenii var. convexa, P. inconspicua, P. indica, P. isabelae, P. jacobsii, P. jaliscana, P. kumaenorum, P. laurae, P. lazoi, P. liniformans, P. liniformans var. liniformans, P. liniformans var. americana, P. mexicana, P. mairei, P. makarorae, P. mammillata, P. medullosa, P. meridensis, P. meridionalis, P. mescaleroensis, P. mexicana, P. moseri, P. muliercula, P. naematoliformis, P. natalensis, P. natarajanii, P. neorhombispora, P. neoxalapensis, P. ovoideocystidiata, P. ovoideocystidiata, P. papuana, P. paulensis, P. pelliculosa, P. pintonii, P. pleurocystidiosa, P. plutonia, P. portoricensis, P. pseudoaztecorum, P. puberula, P. quebecensis, P. ricki, P. rostrata, P. rzedowskii, P. samuiensis, P. schultesii, P. semilanceata, P. septentrionalis, P. serbica, P. sierrae, P. silvatica, P. singeri, P. squamosa, P. strictipes, P. stuntzii, P. subacutipilea, P. subaeruginascens, P. subaeruginosa, P. subbrunneocystidiata, P. subcaerulipes, P. subcubensis, P. subpsilocybioides, P. subtropicalis, P. tampanensis, P. tampanensis, P. thaicordispora, P. thaiaerugineomaculans, P. thaiduplicatocystidiata, P. uruguayensis, P. uxpanapensis, P. venenata, P. villarrealiae, P. weraroa, P. wassoniorum, P. weilii, P. weldenii, P. weraroa, P. wrightii, P. xalapensis, P. yungensis, P. zapotecorum, P. zapotecoantillarum, P. zapotecocaribaea and P. zapotecorum. In some aspects and embodiments, the composition includes dried fungal matter selected from the group consisting of fruiting bodies, mycelia, sclerotia and hyphae. In some aspects and embodiments, the composition is formulated into a dosage form for oral ingestion wherein the TRP agonist compound comprises capsaicin, and wherein the composition includes between 0.1 and 20 mg of the 4-substituted DMT compound and between 0.1 mg and 1 mg of capsaicin in the dosage form. In some aspects and embodiments, the composition is formulated into a dosage form for oral ingestion wherein the TRP agonist compound comprises capsaicin, and wherein the composition includes a ratio (w/w) of between 22:1 and 270,000:1 of the 4-substituted DMT compound:capsaicin in the dosage form. In some aspects and embodiments, the composition is formulated into a dosage form for oral ingestion wherein the TRP agonist compound comprises eugenol, and wherein the composition includes between 0.1 and 20 mg of the 4-substituted DMT compound and between 1 mg and 300 mg of eugenol in the dosage form. In some aspects and embodiments, the composition is formulated into a dosage form for oral ingestion wherein the TRP agonist compound comprises eugenol, and wherein the composition includes a ratio (w/w) of between 0.6:1 and 270,000:1 of the 4-substituted DMT compound:eugenol in the dosage form. In some aspects and embodiments, the composition is formulated into a dosage form for oral ingestion wherein the TRP agonist compound comprises curcumin, and the composition includes between 0.5 and 20 mg of the 4-substituted DMT compound and between 1.00 mg and 15 mg of curcumin in the dosage form. In some aspects and embodiments, the composition is formulated into a dosage form for oral ingestion wherein the TRP agonist compound comprises curcumin, and wherein the composition includes a ratio (w/w) of between 0.04:1 and 10:1 of the 4-substituted DMT compound:curcumin in the dosage form. In some aspects and embodiments, the composition is formulated into a dosage form for oral ingestion wherein the TRP agonist compound comprises β-caryophyllene, and the composition includes between 0.5 and 20 mg of the 4-substituted DMT compound and between 0.25 and 1.50 mg of β-caryophyllene in the dosage form. In some aspects and embodiments, the composition is formulated into a dosage form for oral ingestion wherein the TRP agonist compound comprises β-caryophyllene, and wherein the composition includes a ratio (w/w) of between 0.33:1 and 36:1 of the 4-substituted DMT compound:0-caryophyllene in the dosage form. In some aspects and embodiments, the composition is formulated into a dosage form for oral ingestion wherein the TRP agonist compound comprises cinnamaldehyde, and the composition includes between 0.5 and 20 mg of the 4-substituted DMT compound and between 0.25 and 1.0 mg of cinnamaldehyde in the dosage form. In some aspects and embodiments, the composition is formulated into a dosage form for oral ingestion wherein the TRP agonist compound comprises cinnamaldehyde, and wherein the composition includes a ratio (w/w) of between 0.5:1 and 36:1 of the 4-substituted DMT compound:cinnamaldehyde in the dosage form. In some aspects and embodiments, the composition is formulated into a dosage form for oral ingestion wherein the TRP agonist compound comprises myristicin, and the composition includes between 0.5 and 20 mg of the 4-substituted DMT compound and between 0.50 and 3.0 mg of myristicin in the dosage form. In some aspects and embodiments, the composition is formulated into a dosage form for oral ingestion wherein the TRP agonist compound comprises myristicin, and wherein the composition includes a ratio (w/w) of and 20:1 of the 4-substituted DMT compound: myristicin in the dosage form. In some aspects and embodiments, the tryptamine comprises a 5-substituted tryptamine. In some aspects and embodiments, the 5-substituted tryptamine comprises a compound selected from the group consisting of 5-methoxy-DMT (bufotenin), N-acetyl-5-methoxy tryptamine (melatonin), 5-hydroxy tryptamine (serotonin), 5-hydroxy-tryptophan (5-HTP) and any suitable salt of any of the foregoing. In some aspects and embodiments, the agonist compound comprises an ergoline. In some aspects and embodiments, the ergoline comprises a compound selected from the group consisting of D-lysergic acid ethylamide (“LAE”), D-lysergic acid beta-propanolamide, also called ergometrine or ergonovine, D-lysergic acid 2-butyl amide (“LSB”), D-lysergic acid 1-butanolamide, also called methylergometrine or methylergonovine, 1-methyl-D-lysergic acid butanolamide, also called methysergide, D-lysergic acid 3-pentyl amide (“LSP”), D-N-morpholinyllysergamide (“LSM-775”), D-N-pyrrolidyllysergamide (“LPD-824”), (8(3)-6-methyl-8-(piperidin-1-ylcarbonyl)-9,10-didehydroergoline (“LSD-Pip”), N,N-dimethyllysergamide (“DAM”), D-lysergic acid methylisopropyl amide (“LAMIDE”) also called methylisopropyllysergamide (“MIPLA”), D-lysergic acid 2,4-dimethylazetidide (“LSZ”), LSD, D-1-acetyl-lysergic acid diethylamide (“ALD-52”), D-1-propionyl-lysergic acid diethylamide (“1P-LSD”), D-N1-butyryl-lysergic acid diethylamide (“1B-LSD”), D-N1-(cyclopropylmethanoyl)-lysergic acid diethylamide (“1 cP-LSD”), D-N1-methyl-lysergic acid diethylamide (“MLD”), D-6-ethyl-6-nor-lysergic acid diethylamide (“ETH-LAD”), D-1-propionyl-6-ethyl-6-nor-lysergic acid diethyamide (“IP-ETH-LAD”), D-6-allyl-6-nor-lysergic acid diethylamide (“AL-LAD”), D-6-propyl-6-nor-lysergic acid diethylamide (“PRO-LAD”), D-6-isopropyl-6-nor-lysergic acid diethylamide (“IP-LAD”), D-6-propynyl-6-nor-lysergic acid diethylamide (“PARGY-LAD”), D-6-butyl-6-nor-lysergic acid diethylamide (“BU-LAD”), N,N-diallyllysergamide (“DAL”) and D-N-ethyl-N-cyclopropyllysergamide (“ECPLA”). In some aspects and embodiments, the ergoline comprises an ergoline sourced from a fungus or plant that biosynthesizes the ergoline. In some aspects and embodiments, the fungi or plant includes a species selected from the group consisting of Claviceps purpurea, other species of Claviceps, Rivea corymbosa, Ipomoea violacea, I. tricolor, I. purpurae, I. alba, Periglandula spp. other species of morning glory, Argeyreia nervosa or other species of Hawaiian baby woodrose. In some aspects and embodiments, the 5HT2A agonist compound comprises a phenethylamine. In some aspects and embodiments, the phenethylamine comprises a compound selected from the group consisting of 3,4,5-trimethoxyphenethylamine (mescaline), trimethoxyamphetamine (“TMA”), 4-bromo-2,5-dimethoxybenzeneethanamine (“2C-B”), 4-bromo-2,5-dimethoxyamphetamine (“DOB”), 4-methyl-2,5-dimethoxyamphetamine (“DOM”), 4-methyl-2,5-dimethoxybenzeneethanamine (“2C-D”), 3,4-methylenedioxyamphetamine (“MDA”), N-methyl-3,4-methylenedioxyamphetamine (“MDMA”). In some aspects and embodiments, the phenethylamine comprises a phenethylamine sourced from a plant that biosynthesizes the phenethylamine. In some aspects and embodiments, the plant includes a species selected from the group consisting of Lophophora williamsii, other Lophophora species, Trichocereus pachanoi, and other Trichocereus species. In some aspects and embodiments, the 5HT2A agonist compound comprises a phenylpropanoid. In some aspects and embodiments, the phenylpropanoid comprises a compound selected from the group consisting of 5-methoxy-3,4-methylenedioxy-allylbenzene (myristicin) and 1,2,3-timethoxy-5-(prop-2-en-1-yl)benzene (elemicin). In some aspects and embodiments, the phenylpropanoid comprises a phenylpropanoid sourced from a plant that biosynthesizes the phenylpropanoid. In some aspects and embodiments, the plant includes a species selected from the group consisting of Myristica fragrans or other species in the Myristicaceae family. In some aspects and embodiments, the TRP agonist compound comprises a TRPV1 agonist compound. In some aspects and embodiments, the TRPV1 agonist compound comprises a capsiate. In some aspects and embodiments, the TRPV1 agonist compound comprises eugenol. In some aspects and embodiments, the TRPV1 agonist compound comprises a compound selected from the group consisting of capsaicin, eugenol, myristicin, elemicin, CBD, CBDA, CBDV, CBG, CBGA, CBGV, THCV, THCVA, myrcene, piperine and gingerol. In some aspects and embodiments, the TRPV1 agonist compound is sourced from biomass of a plant that biosynthesizes the TRPV1 agonist compound. In some aspects and embodiments, the plant includes one or more species selected from the group consisting of cayenne pepper, turmeric, clove, cinnamon, nutmeg, pepper, cannabis, bergamot and ginger. In some aspects and embodiments, the TRP agonist compound comprises a TRPA1 agonist compound. In some aspects and embodiments, the TRP agonist compound comprises a curcuminoid. In some aspects and embodiments, the curcuminoid comprises curcumin. In some aspects and embodiments, the TRPA1 agonist compound comprises a compound selected from the group consisting of curcumin, cinnamaldehyde, alpha terpineol, CBD, CBDA, CBDV, CBG, CBGA, CBGV, THCV, THCVA, thymol, piperine and allicin. In some aspects and embodiments, the TRPA1 agonist compound is sourced from biomass of a plant that biosynthesizes the TRPA1 agonist compound. In some aspects and embodiments, the plant includes one or more species selected from the group consisting of curcumin, cinnamon, turmeric, nutmeg, cannabis, thyme, pepper, garlic and onion. In some aspects and embodiments, the TRP agonist compound comprises a TRPM8 agonist compound. In some aspects and embodimentsects, the TRPM8 agonist compound comprises a compound selected from the group consisting of eugenol, cinnamaldehyde, CBD, CBDA, CBDV, CBG, CBGA, CBGV, THC, THCA, THCV, THCVA, carvacrol, thymol, menthol and 1-8 cineole. In some aspects and embodiments, the TRPM8 agonist compound is sourced from biomass of a plant that biosynthesizes the TRPM8 agonist compound. In some aspects and embodiments, the plant includes one or more species selected from the group consisting of turmeric, clove, cinnamon, pepper, nutmeg, cannabis, bergamot, oregano, thyme, cardamom, peppermint and eucalyptus. In some aspects and embodiments, the TRP agonist compound comprises a TRPV3 agonist compound. In some aspects and embodiments, the TRPV3 agonist compound comprises β-caryophyllene. In some aspects and embodiments, the TRPV3 compound comprises a compound selected from the group consisting of eugenol, β-caryophyllene, (−)-epicatechin, CBD, CBDA, CBGA, CBGV, THCV, THCVA, eriodictyol, cinnamaldehyde, incensole, boswellic acid, thymol. In some aspects and embodiments, the TRPM8 agonist compound is sourced from biomass of a plant that biosynthesizes the TRPM8 agonist compound. In some aspects and embodiments, the plant includes one or more species selected from the group consisting of turmeric, clove, cinnamon, pepper, nutmeg, cannabis, bergamot, oregano, thyme, cardamom, peppermint and eucalyptus. In some aspects and embodiments, the composition is formulated into an oral formulation for ingestion. In some aspects and embodiments, the composition comprises a pharmaceutically acceptable excipient, diluent or filler material.

In a further aspect, herein disclosed is a method of treating a health condition comprising administering a composition as described herein to an individual suffering from the condition. In some aspects and embodiments, the condition comprises a condition selected from the group consisting of cancer, neurological disorders, diabetic complications, mental health disorders, bone, muscular and skeletal disease, metabolic disorders, chronic or acute inflammatory disorders and cardiovascular disease. In some aspects and embodiments, the 5HT2A agonist compound comprises a 4-substituted DMT compound, and the effective amount of the 4-substituted DMT compound comprises between 0.015 mg/kg and 0.30 mg/kg, with reference to the body weight of the individual. In some aspects and embodiments, the TRP agonist compound comprises capsaicin, and the effective amount of capsaicin comprises between 0.0005 μg/kg and 1.0 μg/kg per day, with reference to the body weight of the individual. In some aspects and embodiments, the TRP agonist compound comprises eugenol, and the effective amount of eugenol comprises between 0.005 μg/kg and 400 μg/kg per day, with reference to the body weight of the individual. In some aspects and embodiments, the TRP agonist compound comprises curcumin, and the effective amount of curcumin comprises between 0.014 mg/kg and 0.55 mg/kg per day, with reference to the body weight of the individual. In some aspects and embodiments, the TRP agonist compound comprises β-caryophyllene, and the effective amount of β-caryophyllene comprises between 0.004 mg/kg and 0.064 mg/kg per day, with reference to the body weight of the individual. In some aspects and embodiments, the TRP agonist compound comprises myristicin, and the effective amount of myristicin comprises between 0.007 mg/kg and 0.130 mg/kg per day, with reference to the body weight of the individual.

In a further aspect, herein disclosed is use of a composition as described herein in the treatment of an individual suffering from a mental illness condition or physically debilitating condition. In some aspects and embodiments, the condition comprises a condition selected from the group consisting of cancer, neurological disorders, diabetic complications, mental health disorders, bone, muscular and skeletal disease, metabolic disorders, chronic inflammatory disorders and cardiovascular disease, and post-traumatic stress disorder.

In further aspects and embodiments, herein disclosed is a composition for reducing inflammation to improve or maintain mental health or physical health in an individual comprising an effective amount of a 5HT2A agonist compound, an effective amount of a capsaicinoid, an effective amount of a curcuminoid and an effective amount of eugenol. In some aspects and embodiments, the 5HT2A agonist compound comprises a compound as described herein. In some aspects and embodiments, the capsaicinoid comprises capsaicin. In some aspects and embodiments, the curcuminoid comprises curcumin.

In one aspect and embodiment, a composition comprises a therapeutic combination of a 5HT2A agonist compound and at least one TRP agonist compound, wherein the therapeutically effective amount of the 5HT2A agonist is between about 1 μg and about 200 mg; and the therapeutically effective amount of the at least one TRP receptor agonist is between about 0.1 mg and about 25 mg.

In some aspects and embodiments, the 5HT2A agonist compound is selected from the group consisting of a tryptamine, an ergoline, a phenethylamine, and a phenylpropanoid.

In some aspects and embodiments, the tryptamine is a 4-substituted tryptamine.

In some aspects and embodiments, the 4-substituted tryptamine is a 4-substituted DMT compound.

In some aspects and embodiments, the 4-substituted DMT compound is selected from the group consisting of 3-[2-(dimethylamino)ethyl]-4-phosphoryloxyindole (psilocybin), 3-[2-(dimethylamino)ethyl]-4-hydroxyindole (psilocin), 3-[2-(dimethylamino)ethyl]-4-acetoxyindole (psilacetin), and any suitable salt of any of the foregoing.

In some aspects and embodiments, the 4-substituted tryptamine is selected from the group consisting of 3-[2(trimethylamino)ethyl]-4-phosphoryloxyindole (aeruginascin), 3-[2-(methylamino)ethyl]-4-phosphoryloxyindole (baeocystin), 3-[2-(methylamino)ethyl]-4-hydroxyindole, 3-[2-(amino)ethyl]-4-hydroxyindole (norpsilocin), 3-[2-(amino)ethyl]-4-phosphoryloxyindole (norbaeocystin), and any suitable salt of any of the foregoing.

In some aspects and embodiments, the 4-substituted tryptamine is derived from fungi.

In some aspects and embodiments, the fungi is a species of a genus selected from the group consisting of Gymnopilus, Inocybe, Panaeolus, Pholiotina, Pluteus, and Psilocybe.

In some aspects and embodiments, the fungi is a species of a genus selected from the group consisting of Gymnopilus, Inocybe, Panaeolus, Pholiotina, Pluteus, and Psilocybe.

In some aspects and embodiments, the fungi is selected from the group consisting of C. cyanopus, C. siligineoides and C. kuehneriana; Copelandia species including C. affinis, C. anomala, C. bispora, C. cambodginiensis, C. chlorocystis, C. cyanescens, C. lentisporus, C. tirunelveliensis, C. tropica, C. tropicalis and C. westii; G. steglichii; G. thiersii, G. aeruginosus, G. braendlei, G. cyanopalmicola, G. intermedius, G. junonius, G. lateritius, G. liquiritiae, G. luteofolius, G. luteoviridis, G. luteus, G. purpuratus, G. subpurpuratus, G. validipes and G. viridans; I. aeruginascens, I. aeruginascens, I. coelestium, I. corydalina, I. corydalina var. corydalina, I. corydalina var. erinaceomorpha, I. haemacta and I. tricolor; P. cinctulus, P. affinis, P. africanus, P. bisporus, P. cambodginiensis, P. castaneifolius, P. chlorocystis, P. cinctulus, P. cyanescens, P. fimicola, P. lentisporus, P. microsporus, P. moellerianus, P. olivaceus, P. rubricaulis, P. tirunelveliensis, P. tropicalis and P. venezolanus; P. cyanopus and P. smithii; P. americanus, P. albostipitatus, P. americanus, P. cyanopus, P. glaucus, P. glaucotinctus, P. nigroviridis, P. phaeocyanopus, P. salicinus, P. saupei and P. villosus; P. tampanensis, P. acutipilea, P. allenii, P. angustipleurocystidiata, P. antioquiensis, P. atlantis, P. aquamarina, P. armandii, P. aucklandii, P. atlantis, P. aztecorum, P. aztecorum var. aztecorum, P. aztecorum var. bonetii, P. azurescens, P. baeocystis, P. banderillensis, P. bispora, P. brasiliensis, P. brunneocystidiata, P. cubensis, P. caeruleoannulata, P. caerulescens, P. caerulescens var. caerulescens, P. caerulescens var. ombrophila, P. caerulipes, P. callosa, P. carbonaria, P. caribaea, P. chuxiongensis, P. collybioides, P. columbiana, P. cordispora, P. cubensis, P. cyanescens, P. cyanofibrillosa, P. dumontii, P. egonii, P. fagicola, P. fagicola var. fagicola, P. fagicola var. mesocystidiata, P. farinacea, P. fimetaria, P. fuliginosa, P. furtadoana, P. tampanensis, P. galindoi, P. gallaeciae, P. graveolens, P. guatapensis, P. guilartensis, P. heimii Guzman, P. herrerae Guzman, P. hispanica Guzman, P. hoogshagenii, P. hoogshagenii var. hoogshagenii, P. hoogshagenii var. convexa, P. inconspicua, P. indica, P. isabelae, P. jacobsii, P. jaliscana, P. kumaenorum, P. laurae, P. lazoi, P. liniformans, P. liniformans var. liniformans, P. liniformans var. americana, P. mexicana, P. mairei, P. makarorae, P. mammillata, P. medullosa, P. meridensis, P. meridionalis, P. mescaleroensis, P. mexicana, P. moseri, P. muliercula, P. naematoliformis, P. natalensis, P. natarajanii, P. neorhombispora, P. neoxalapensis, P. ovoideocystidiata, P. ovoideocystidiata, P. papuana, P. paulensis, P. pelliculosa, P. pintonii, P. pleurocystidiosa, P. plutonia, P. portoricensis, P. pseudoaztecorum, P. puberula, P. quebecensis, P. ricki, P. rostrata, P. rzedowskii, P. samuiensis, P. schultesii, P. semilanceata, P. septentrionalis, P. serbica, P. sierrae, P. silvatica, P. singeri, P. squamosa, P. strictipes, P. stuntzii, P. subacutipilea, P. subaeruginascens, P. subaeruginosa, P. subbrunneocystidiata, P. subcaerulipes, P. subcubensis, P. subpsilocybioides, P. subtropicalis, P. tampanensis, P. tampanensis, P. thaicordispora, P. thaiaerugineomaculans, P. thaiduplicatocystidiata, P. uruguayensis, P. uxpanapensis, P. venenata, P. villarrealiae, P. weraroa, P. wassoniorum, P. weihi, P. weldenii, P. weraroa, P. wrightii, P. xalapensis, P. yungensis, P. zapotecorum, P. zapotecoantillarum, P. zapotecocaribaea, and P. zapotecorum.

In some aspects and embodiments, the composition further comprises dried matter of the fungi, wherein the dried matter is selected from the group consisting of fruiting bodies, mycelia, sclerotia, and hyphae, or combinations thereof.

In some aspects and embodiments, the tryptamine is a 5-substituted tryptamine.

In some aspects and embodiments, the 5-substituted tryptamine is selected from the group consisting of 5-methoxy-DMT (bufotenin), N-acetyl-5-methoxy tryptamine (melatonin), 5-hydroxy tryptamine (serotonin), 5-hydroxy-tryptophan (5-HTP), and any suitable salt of any of the foregoing.

In some aspects and embodiments, the 5HT2A agonist compound is an ergoline.

In some aspects and embodiments, the ergoline is selected from the group consisting of D-lysergic acid ethylamide (“LAE”),D-lysergic acid beta-propanolamide, D-lysergic acid 2-butyl amide (“LSB”), D-lysergic acid 1-butanolamide, 1-methyl-D-lysergic acid butanolamide, D-lysergic acid 3-pentyl amide (“LSP”), D-N-morpholinyllysergamide (“LSM-775”), D-N-pyrrolidyllysergamide (“LPD-824”), (8(3)-6-methyl-8-(piperidin-1-ylcarbonyl)-9,10-didehydroergoline (“LSD-Pip”), N,N-dimethyllysergamide (“DAM”), D-lysergic acid methylisopropyl amide (“LAMIDE”), D-lysergic acid 2,4-dimethylazetidide (“LSZ”), LSD, D-1-acetyl-lysergic acid diethylamide (“ALD-52”), D-1-propionyl-lysergic acid diethylamide (“1P-LSD”), D-N1-butyryl-lysergic acid diethylamide (“1B-LSD”), D-N1-(cyclopropylmethanoyl)-lysergic acid diethylamide (“1 cP-LSD”), D-N1-methyl-lysergic acid diethylamide (“MLD”), D-6-ethyl-6-nor-lysergic acid diethylamide (“ETH-LAD”), D-1-propionyl-6-ethyl-6-nor-lysergic acid diethyamide (“1P-ETH-LAD”), D-6-allyl-6-nor-lysergic acid diethylamide (“AL-LAD”), D-6-propyl-6-nor-lysergic acid diethylamide (“PRO-LAD”), D-6-isopropyl-6-nor-lysergic acid diethylamide (“IP-LAD”), D-6-propynyl-6-nor-lysergic acid diethylamide (“PARGY-LAD”), D-6-butyl-6-norlysergic acid diethylamide (“BU-LAD”), N,N-diallyllysergamide (“DAL”) and D-N-ethyl-N-cyclopropyllysergamide (“ECPLA”).

In some aspects and embodiments, the ergoline is derived from fungi or a plant. In some aspects and embodiments, the fungi or plant is a species selected from the group consisting of Claviceps purpurea, Rivea corymbosa, Ipomoea violacea, I. tricolor, I. purpurae, I. alba, Argeyreia nervosa, and a Periglandula species.

In some aspects and embodiments, the 5HT2A agonist compound is a phenethylamine.

In some aspects and embodiments, the phenethylamine is selected from the group consisting of 3,4,5-trimethoxyphenethylamine (mescaline), trimethoxyamphetamine (“TMA”), 4-bromo-2,5-dimethoxybenzeneethanamine (“2C-B”), 4-bromo-2,5-dimethoxyamphetamine (“DOB”), 4-methyl-2,5-dimethoxyamphetamine (“DOM”), 4-methyl-2,5-dimethoxybenzeneethanamine (“2C-D”), 3,4-methylenedioxyamphetamine (“MDA”), N-methyl-3,4-methylenedioxyamphetamine (“MDMA”).

In some aspects and embodiments, the phenethylamine is plant-derived.

In some aspects and embodiments, the plant includes a species selected from the group consisting of Lophophora wilhamsii, Trichocereus pachanoi, Echinopsis pachanoi, Trichocereus peruvianus, Echinopsis peruviana, Trichocereus bridgesii, Echinopsis lageniformis, and Trichocereus/Echinopsis scopulicola.

In some aspects and embodiments, the 5HT2A agonist compound is a phenylpropanoid.

In some aspects and embodiments, the phenylpropanoid is 1,2,3-timethoxy-5-(prop-2-en-1-yl)benzene (elemicin).

In some aspects and embodiments, the phenylpropanoid is plant-derived.

In some aspects and embodiments, the plant is a species in the Myristicaceae family.

In some aspects and embodiments, the TRP agonist compound is selected from the group consisting of a capsiate, eugenol, elemicin, myrcene, piperine and gingerol.

In some aspects and embodiments, the capsiate is capsaicin.

In some aspects and embodiments, the TRP agonist compound is plant-derived.

In some aspects and embodiments, the plant includes one or more species selected from the group consisting of cayenne pepper, turmeric, clove, cinnamon, nutmeg, pepper, cannabis, bergamot and ginger.

In some aspects and embodiments, the TRP agonist compound is selected from the group consisting of a curcuminoid, cinnamaldehyde, alpha terpineol, thymol, piperine and allicin.

In some aspects and embodiments, the curcuminoid is curcumin.

In some aspects and embodiments, the plant includes one or more species selected from the group consisting of curcumin, cinnamon, turmeric, nutmeg, cannabis, thyme, pepper, garlic, and onion.

In some aspects and embodiments, the TRP agonist compound is selected from the group consisting of eugenol, cinnamaldehyde, carvacrol, thymol, menthol, and 1-8 cineole.

In some aspects and embodiments, the plant includes one or more species selected from the group consisting of turmeric, clove, cinnamon, pepper, nutmeg, cannabis, bergamot, oregano, thyme, cardamom, peppermint, and eucalyptus.

In some aspects and embodiments, the TRP agonist compound is selected from the group consisting of eugenol, β-caryophyllene, (−)-epicatechin, CBD, CBDA, CBGA, CBGV, THCV, THCVA, eriodictyol, cinnamaldehyde, incensole, eucalyptol, and thymol.

In aspects and embodiments, the plant includes one or more species selected from the group consisting of turmeric, clove, cinnamon, pepper, nutmeg, cannabis, bergamot, oregano, thyme, cardamom, peppermint, and eucalyptus.

In some aspects and embodiments, the 5HT2A agonist compound is psilocybin, and wherein the therapeutically effective amount of psilocybin is between about 100 mg and about 300 mg, or between about 0.5 mg and about 20 mg.

In some aspects and embodiments, the 5HT2A agonist is psilocybin, in an amount of between about 110 mg and about 290 mg, about 120 mg and about 280 mg, about 130 mg and about 270 mg, about 140 mg and about 260 mg, about 150 mg and about 250 mg, about 160 mg and about 240 mg, about 170 mg and about 230 mg, about 180 mg and about 220 mg, about 190 mg and about 210 mg, or about 195 mg and about 205 mg.

In some aspects and embodiments, the at least one TRP agonist compound is capsaicin in an amount of about 0.1 mg and about 1 mg, about 0.2 mg and about 0.9 mg, about 0.3 mg and about 0.8 mg, about 0.4 and about 0.7 mg, or about 0.5 mg and about 0.6 mg

In some aspects and embodiments, the at least one TRP agonist compound is capsaicin, and wherein the composition comprises a ratio (w/w) of between about 22:1 and about 270,000:1 of the 5HT2A agonist to capsaicin, about 50:1 and about 200,000:1 of the 5HT2A agonist to capsaicin, about 100:1 and about 150,000:1 of the 5HT2A agonist to capsaicin, about 500:1 and about 100,000:1 of the 5HT2A agonist to capsaicin, about 1,000:1 and about 50,000:1 of the 5HT2A agonist to capsaicin, about 5,000:1 and about 40,000:1 of the 5HT2A agonist to capsaicin, about 10,000:1 and about 30,000:1 of the 5HT2A agonist to capsaicin, or about 15,000:1 and about 25,000:1 of the 5HT2A agonist to capsaicin.

In some aspects and embodiments, the at least one TRP agonist compound is eugenol in an amount of about 1 mg and about 300 mg, about 5 mg and about 290 mg, about 10 mg and about 280 mg, about 15 mg and about 270 mg, about 20 mg and about 260 mg, about 25 mg and about 250 mg, about 30 mg and about 240 mg, about 35 mg and about 230 mg, about 40 mg and about 220 mg, about 40 mg and about 210 mg, about 50 mg and about 210 mg, about 55 mg and about 200 mg, about 60 mg and about 190 mg, about 65 mg and about 180 mg, about 70 mg and about 170 mg, about 75 mg and about 160 mg, about 80 mg and about 150 mg, about 85 mg and about 140 mg, about mg and about 130 mg, about 95 mg and about 120 mg, or about 100 mg and about 110 mg.

In some aspects and embodiments, the at least one TRP agonist compound is eugenol, and wherein the composition comprises a ratio (w/w) of between about 0.6:1 and about 270,000:1 of the 5HT2A agonist to eugenol, about 1:1 and about 250,000:1 of the agonist to eugenol, about 5:1 and about 225,000:1 of the 5HT2A agonist to eugenol, about 10:1 and about 200,000:1 of the 5HT2A agonist to eugenol, about 50:1 and about 175,000:1 of the 5HT2A agonist to eugenol, about 100:1 and about 150,000:1 of the 5HT2A agonist to eugenol, about 150:1 and about 125,000:1 of the 5HT2A agonist to eugenol, about 300:1 and about 100,000:1 of the 5HT2A agonist to eugenol, about 500:1 and about 75,000:1 of the 5HT2A agonist to eugenol, about 1,000:1 and about of the 5HT2A agonist to eugenol, about 5,000:1 and about 45,000:1 of the agonist to eugenol, about 10,000:1 and about 40,000:1 of the 5HT2A agonist to eugenol, about 15,000:1 and about 35,000:1 of the 5HT2A agonist to eugenol, or about and about 30,000:1 of the 5HT2A agonist to eugenol.

In some aspects and embodiments, the at least one TRP agonist compound is curcumin in an amount of about 0.1 mg to about 10 mg, about 0.5 mg to about 9 mg, about 1 mg to about 8 mg, about 2 mg to about 7 mg, about 3 mg to about 6 mg, or about 4 mg to about 5 mg.

In some aspects and embodiments, the at least one TRP agonist compound is curcumin, and wherein the composition comprises a ratio (w/w) of between about 0.04:1 and about 10:1 of the 5HT2A agonist to curcumin, about 0.1:1 and about 9.5:1 of the agonist to curcumin, about 0.5:1 and about 9:1 of the 5HT2A agonist to curcumin, about 1:1 and about 8.5:1 of the 5HT2A agonist to curcumin, about 1.5:1 and about 8:1 of the 5HT2A agonist to curcumin, about 2:1 and about 7.5:1 of the 5HT2A agonist to curcumin, about 2.5:1 and about 7:1 of the 5HT2A agonist to curcumin, about 3:1 and about 6.5:1 of the 5HT2A agonist to curcumin, about 3.5:1 and about 6:1 of the agonist to curcumin, about 4:1 and about 5.5:1 of the 5HT2A agonist to curcumin, or about 4.5:1 and about 5:1 of the 5HT2A agonist to curcumin.

In some aspects and embodiments, the at least one TRP agonist compound is (3-caryophyllene, and wherein the composition comprises a ratio (w/w) of between about 0.33:1 and about 36:1 of the 5HT2A agonist to β-caryophyllene, about 1:1 and about 33:1 of the 5HT2A agonist to β-caryophyllene, about 3:1 and about 30:1 of the 5HT2A agonist to β-caryophyllene, about 5:1 and about 27:1 of the 5HT2A agonist to β-caryophyllene, about 7:1 and about 25:1 of the 5HT2A agonist to β-caryophyllene, about 10:1 and about 22:1 of the 5HT2A agonist to β-caryophyllene, about 15:1 and about 20:1 of the 5HT2A agonist to β-caryophyllene, or about 17:1 and about 18:1 of the 5HT2A agonist to (3-caryophyllene.

In some aspects and embodiments, the at least one TRP agonist compound is cinnamaldehyde in an amount of between about 0.1 mg and about 10 mg, about 0.5 mg and about 9.5 mg, about 1 mg and about 9 mg, about 1.5 mg and about 8.5 mg, about 2 mg and about 8 mg, about 2.5 mg and about 7.5 mg, about 3 mg and about 7 mg, about 3.5 mg and about 6.5 mg, about 4 mg and about 6 mg, or about 4.5 mg and about 5.5 mg.

In some aspects and embodiments, the at least one TRP agonist compound is cinnamaldehyde, and wherein the composition comprises a ratio (w/w) of between about 0.5:1 and about 36:1 of the 5HT2A agonist to cinnamaldehyde, about 1:1 and about 33:1 of the 5HT2A agonist to cinnamaldehyde, about 3:1 and about 30:1 of the 5HT2A agonist to cinnamaldehyde, about 5:1 and about 27:1 of the 5HT2A agonist to cinnamaldehyde, about 7:1 and about 25:1 of the 5HT2A agonist to cinnamaldehyde, about 10:1 and about 22:1 of the 5HT2A agonist to cinnamaldehyde, about 15:1 and about 20:1 of the 5HT2A agonist to cinnamaldehyde, or about 17:1 and about 18:1 of the 5HT2A agonist to cinnamaldehyde.

In some aspects and embodiments, the composition is formulated for oral administration.

In some aspects and embodiments, the composition further comprises at least one pharmaceutically acceptable excipient, diluent, or filler.

In some aspects and embodiments, the composition is selected from the group consisting of a tablet, capsule, sachets, granules, sublingual film, buccal film, and a suspension.

In another aspect and embodiments, a method for reducing inflammation in a subject, comprising administering any of the compositions described herein to the subject.

In some aspects and embodiments, the inflammation is acute or chronic.

In some aspects and embodiments, the method comprises administering the composition from 1-10 times per day.

In some aspects and embodiments, the reduction in inflammation is measured by a 40-60% or a 50% reduction of at least one biomarker selected from the group consisting of COX-2, interferon-y, interleukin 1, interleukin-2, interleukin-6, interleukin-8, interleukin-1β, tumor necrosis factor (TNF), and reactive oxygen species (ROS) when measured via densitometry.

In some aspects and embodiments, the ROS is inducible nitric oxide synthase (iNOS).

In some aspects and embodiments, the subject is suffering from a condition selected from the group consisting of cancer, neurological disorder, diabetic complications, mental health disorder (MHD), bone, muscular and skeletal disease, metabolic disorder, chronic inflammatory disorder and cardiovascular disease.

In some aspects and embodiments, the MHD is selected from depression, anxiety, post-traumatic stress disorder, schizophrenia, bipolar disorder, ADD, ADHD, borderline personality disorder, seasonal affective disorder, and premenstrual dysphoric disorder.

In some aspects and embodiments, the MHD is depression, and the reduction in inflammation is accompanied by a reduction in at least one symptom of depression.

In some aspects and embodiments, the MHD is anxiety, and the reduction in inflammation is accompanied by a reduction in at least one symptom of anxiety.

In another aspect and embodiment, a method for reducing at least one biomarker in a mammalian cell, wherein the biomarker is selected from the group consisting of COX-2, interferon-γ, interleukin 1, interleukin-2, interleukin-6, interleukin-8, interleukin-10, tumor necrosis factor (TNF), and reactive oxygen species (ROS), comprises administering the composition of any one of claims X to X to a subject, wherein administering the composition reduces the biomarker in the mammalian cell between about X % and about Y %.

In some aspects and embodiments, the 5HT2A agonist is psilocybin in an amount of about 100 mg to about 300 mg, and wherein the TRP agonist is eugenol in an amount of about 100 mg to about 300 mg. In some aspects, the amount of psilocybin and eugenol is per dose. In some aspects, the dose is administered 1 to 10 times per day.

In some aspect and embodiments, the 5HT2A agonist is psilocybin in an amount of about 100 mg to about 300 mg, about 110 mg to about 290 mg, about 120 mg to about 280 mg, about 130 mg to about 270 mg, about 140 mg to about 260 mg, about 150 mg to about 250 mg, about 160 mg to about 240 mg, about 170 mg to about 230 mg, about 180 mg to about 220 mg, about 190 mg to about 210 mg, or about 195 mg to about 205 mg, and wherein TRP agonist is eugenol in an amount of about 100 mg to about 300 mg, about 110 mg to about 290 mg, about 120 mg to about 280 mg, about 130 mg to about 270 mg, about 140 mg to about 260 mg, about 150 mg to about 250 mg, about 160 mg to about 240 mg, about 170 mg to about 230 mg, about 180 mg to about 220 mg, about 190 mg to about 210 mg, or about 195 mg to about 205 mg. In some aspects, the amount of psilocybin is per dose. In some aspects, the dose is administered 1 to 10 times per day.

In some aspects and embodiments, administering the composition reduces IL-6 in the mammalian cell by about an additional 20% relative to administering the therapeutically effective amount of psilocybin alone.

In some aspects and embodiments, administering the composition reduces IL-6 in the mammalian cell by about an additional 25% relative to administering the therapeutically effective amount of psilocybin alone.

Other aspects and features of the present disclosure will become apparent to those ordinarily skilled in the art upon review of the following description of specific aspects and embodiments in conjunction with the accompanying figures.

### BRIEF DESCRIPTION OF THE DRAWINGS

Embodiments of the present disclosure will now be described, by way of example only, with reference to the attached figures.

FIG. 1 shows a schematic of a generalized single-molecule approach, as is used in most traditional pharmaceutical therapeutics.

FIG. 2 shows a schematic of a generalized multi-modal approach, providing a therapeutic that accounts for multiple portions of a metabolic pathway.

FIG. 3 shows a schematic illustrating the relationship between energy, sugar metabolism, homeostasis and depression.

FIG. 4 shows interactions between the gut and the brain related to sugar signaling.

FIG. 5 shows a schematic for the mechanism of action and effects of the compositions on 5HT2A receptors and TRP receptors together for reducing inflammation and improving mood.

FIG. 6 shows data used to establish MED50 with psilocybin alone and with a formulation of the 07 Base. A complete composition is described in Table 8 with psilocybin.

FIG. 7 shows data used to establish MED50 with morning glory seeds alone and with a formulation of the 07 Base. A complete composition is described in Table 8 with morning glory seeds.

FIG. 8 shows data used to establish MED50 with Hawaiian baby woodrose seeds alone and with a formulation of the 07 Base. A complete composition is described in Table 8 with Hawaiian baby woodrose seeds.

FIG. 9 shows data used to establish a timepoint post-TNF-α/IFN-γ treatment to evaluate the anti-inflammatory effects of the compositions described herein on human primary small intestinal epithelial cells (HSIEC).

FIG. 10 shows the structures of TRP agonists eugenol, capsaicin, and curcumin.

FIG. 11 shows the effects of escalating doses of eugenol on COX-2 in the HSIEC cells treated with TNF-α/IFN-γ.

FIG. 12 shows the effects of escalating doses of capsaicin on COX-2 in the HSIEC cells treated with TNF-α/IFN-γ.

FIG. 13 shows the effects of escalating doses of curcumin on COX-2 in the HSIEC cells treated with TNF-α/IFN-γ.

FIG. 14 shows the structures of 5HT2A agonists psilocybin, 4-ACO-DMT, psilocin, and serotonin and 5HT2A antagonist ketanserin.

FIG. 15 shows the effects of escalating doses of psilocybin on COX-2 in the HSIEC cells treated with TNF-α/IFN-γ.

FIG. 16 shows the effects of escalating doses of 4-ACO-DMT on COX-2 in the HSIEC cells treated with TNF-α/IFN-γ.

FIG. 17 shows the effects of escalating doses of ketanserin on COX-2 in the HSIEC cells treated with TNF-α/IFN-γ.

FIG. 18 shows the separate and combined effects of psilocybin and eugenol on COX-2 in the HSIEC cells treated with TNF-α/IFN-γ.

FIG. 19 shows the separate and combined effects of psilocybin and eugenol on IL-6 in the HSIEC cells treated with TNF-α/IFN-γ.

FIG. 20 shows the separate and combined effects of psilocybin and eugenol on IL-8 in the HSIEC cells treated with TNF-α/IFN-γ.

FIG. 21 shows the separate and combined effects of psilocybin and eugenol on TNF receptor 2 (TNF-R2) in the HSIEC cells treated with TNF-α/IFN-γ.

FIG. 22 shows the separate and combined effects of 4-ACO-DMT and eugenol on COX-2 in the HSIEC cells treated with TNF-α/IFN-γ.

FIG. 23 shows the separate and combined effects of 4-ACO-DMT and eugenol on IL-6 in the HSIEC cells treated with TNF-α/IFN-γ.

FIG. 24 shows the separate and combined effects of ketanserin and eugenol on COX-2 in the HSIEC cells treated with TNF-α/IFN-γ.

FIG. 25 shows the separate and combined effects of psilocybin and capsaicin on COX-2 in the HSIEC cells treated with TNF-α/IFN-γ.

FIG. 26 shows the separate and combined effects of psilocybin and capsaicin on IL-6 in the HSIEC cells treated with TNF-α/IFN-γ.

FIG. 27 shows the separate and combined effects of 4-ACO-DMT and capsaicin on IL-6 in the HSIEC cells treated with TNF-α/IFN-γ.

FIG. 28 shows the separate and combined effects of 4-ACO-DMT and capsaicin on COX-2 in the HSIEC cells treated with TNF-α/IFN-γ.

FIGS. 29A and 29B show the separate and combined effects of ketanserin and capsaicin on COX-2 and IL-6 in the HSIEC cells treated with TNF-α/IFN-γ.

FIG. 30 shows the separate and combined effects of psilocybin and curcumin on COX-2 in the HSIEC cells treated with TNF-α/IFN-γ.

FIG. 31 shows the separate and combined effects of psilocybin and curcumin on IL-6 in the HSIEC cells treated with TNF-α/IFN-γ.

FIG. 32 shows the separate and combined effects of 4-ACO-DMT and curcumin on IL-6 in the HSIEC cells treated with TNF-α/IFN-γ.

FIGS. 33A and 33B show the separate and combined effects of ketanserin and curcumin on COX-2 and IL-6 in the HSIEC cells treated with TNF-α/IFN-γ.

FIG. 34 shows the effects of psilocybin on iNOS in the HSIEC cells treated with TNF-α/IFN-γ.

FIG. 35 shows the separate and combined effects of psilocybin and eugenol on iNOS in the HSIEC cells treated with TNF-α/IFN-γ.

FIG. 36 shows the effects of 4-ACO-DMT on iNOS in the HSIEC cells treated with TNF-α/IFN-γ.

FIG. 37 shows the separate and combined effects of 4-ACO-DMT and eugenol on iNOS in the HSIEC cells treated with TNF-α/IFN-γ.

FIG. 38 shows the separate and combined effects of ketanserin and eugenol on COX-2 in the HSIEC cells treated with TNF-α/IFN-γ.

FIG. 39 shows the structures of TRP agonists carvacrol, piperine, and cinnemaldehyde.

FIG. 40 shows the effects of carvacrol on COX-2 in the HSIEC cells treated with TNF-α/IFN-γ.

FIG. 41 shows the separate and combined effects of psilocybin and carvacrol on COX-2 in the HSIEC cells treated with TNF-α/IFN-γ.

FIGS. 42A and 42B respectively show the effects of psilocybin on IL-6 and IL-8 in the HSIEC cells treated with TNF-α/IFN-γ.

FIGS. 43A and 43B respectively show the effects of carvacrol on IL-6 and IL-8 in the HSIEC cells treated with TNF-α/IFN-γ.

FIG. 44 shows the separate and combined effects of psilocybin and carvacrol on IL-6 in the HSIEC cells treated with TNF-α/IFN-γ.

FIG. 45 shows the separate and combined effects of psilocybin and carvacrol on IL-6 in the HSIEC cells treated with TNF-α/IFN-γ.

FIG. 46 shows the effects of piperine on COX-2 in the HSIEC cells treated with TNF-α/IFN-γ.

FIG. 47 shows the separate and combined effects of psilocybin and piperine on COX-2 in the HSIEC cells treated with TNF-α/IFN-γ.

FIGS. 48A and 48B respectively show the effects of piperine on IL-6 and IL-8 in the HSIEC cells treated with TNF-α/IFN-γ.

FIG. 49 shows the separate and combined effects of psilocybin and piperine on IL-6 in the HSIEC cells treated with TNF-α/IFN-γ.

FIG. 50 shows the separate and combined effects of psilocybin and piperine on IL-8 in the HSIEC cells treated with TNF-α/IFN-γ.

FIG. 51 shows the effects of cinnemaldehyde on IL-6 in the HSIEC cells treated with TNF-α/IFN-γ.

FIG. 52 shows the separate and combined effects of psilocybin and cinnemaldehyde on IL-6 in the HSIEC cells treated with TNF-α/IFN-γ.

FIG. 53 shows data used to establish a timepoint post-TNF-α/IFN-γ treatment to evaluate the anti-inflammatory effects of the compositions described herein on in vitro 3D tissue models.

FIG. 54 shows the separate and combined effects of psilocybin and capsaicin on COX-2 in the 3D tissue models treated with TNF-α/IFN-γ.

FIG. 55 shows the separate and combined effects of psilocybin and capsaicin on IL-6 in the 3D tissue models treated with TNF-α/IFN-γ.

FIGS. 56A and 56B show the separate effects of psilocybin and eugenol on GABA in A-172 cells.

FIGS. 57A and 57B show the separate effects of psilocybin and eugenol on BDNF in the A-172 cells.

FIG. 58A shows the separate and combined effects of psilocybin and eugenol on COX-2 in the A-172 cells.

FIG. 58B shows the separate and combined effects of psilocybin and eugenol on GABA in the A-172 cells.

FIG. 58C shows the separate and combined effects of psilocybin and eugenol on IL-6 in the A-172 cells.

FIG. 58D shows the separate and combined effects of psilocybin and eugenol on BDNF in the A-172 cells.

### DETAILED DESCRIPTION

Generally, the present disclosure provides compositions useful for reducing inflammation through agonism of both 5HT2A signalling receptors and transient receptor potential (“TRP”) nociceptors, thereby promoting and/or maintaining health and wellness in an individual. The compositions disclosed herein include at least two active compounds. A first compound is a serotonin 5HT2A receptor agonist. A second compound is an agonist for one or more TRP receptors. The second compound or additional compounds may also be an antagonist for one or more TRP receptors. The combination of molecules in the compositions shows enhanced effects over 5HT2A agonists alone for combined multimodal action providing benefits at lower doses of both the 5HT2A agonist and the TRP agonist. The interaction of the compositions with the 5HT2A and TRP receptors results in altered activity of nociceptors and may also influence the endocannabinoid signaling system (the “ECS”) and/or glucose metabolism. The compositions may be prepared from medicinal plant and fungi, pure compounds or combinations of both.

The compositions disclosed herein may promote mental health and wellness in healthy individuals, and have applications as a therapeutic for promoting mental health and wellness in individuals suffering from addiction, depression or other mental health conditions. The compositions may reduce the need for other drugs, including pharmaceuticals taken at the direction of a health care practitioner (“HCP”), substances take under self-directed care and abused substances. The compositions may have application in broad harm reduction programs. The compositions may also mitigate inflammation issues unrelated to mental health, including inflammation of gut, inflammation of the bowels and pain associated with inflammation. The composition may also promote the enhancement of cognition, creativity, focus, concentration, sex drive and/or other functions in those considered to be part of the ‘healthy normal’ population.

### Serotonin Receptors

5-hydroxy-tryptamine (“5-HT”), commonly referred to as serotonin and its biosynthetic precursor tryptophan, play an important role in regulating many biological processes. Tryptophan is converted to serotonin in both the intestine and the brain. Serotonin is released by the cell to trigger receptors and cause signaling cascades. Serotonin is typically reabsorbed and broken down by monoamine oxygenase (“MAO”) enzyme. Serotonin affects sleep, appetite, temperature, sexual behavior, and pain sensation. While serotonin an important neurotransmitter, the majority of serotonin is produced the intestine. Brain-gut abnormalities in serotonin signaling have recently been implicated to play a role in several disease processes. Serotonin is primarily synthesized in the intestinal epithelium by enterochromaffin (“EC”) cells from tryptophan. This production then regulates aspects of gut-brain communication. Lower levels of serotonin in the brain elicit sugar craving, while ingestion of sugar rich diet generally improves mood and alleviates anxiety in the short term. However, high sugar diets also disrupt glucose metabolism and feed microbial populations that may produce toxic byproducts, resulting in inflammation. Neuromodulators that influence nociceptor activity are released in the periphery during the inflammation (Loyd, 2012). Serotonin is one such proinflammatory mediator that has been shown to acts as an inflammatory mediator that contributes to inflammatory pain. The presence of multiple 5-HT subtype receptors on peripheral and central nociceptors is relevant to the role of 5-HT in sugar metabolism, mood, pain and inflammation.

The 5HT2A Receptor is a G protein-coupled receptor (“GPCR”). 5HT2A is the main excitatory serotonin GPCR subtype. Stimulation of 5HT serotonin receptors, and specifically the 5HT2A receptor, by molecules has been shown to be primarily responsible for the psychedelic response in humans including illusions, hallucinations, delusions and altered states of perception, often at relatively low concentrations of micrograms to milligrams. 5HT2A receptor agonists may include tryptamines, ergolines, phenethylamines, amphetamines, phenylpropanoids or other families of compounds.

TRP Receptors The TRP receptor superfamily is divided into seven subfamilies, including the five group 1 TRPs (TRPC, TRPV, TRPM, TRPN and TRPA) and two group 2 subfamilies (TRPP and TRPML). Many TRP receptors are activated by a variety of stimuli, providing the function of a signal integrator. TRP receptors are ion channel receptors and are a family of non-selective cation channels that play important roles in cellular signaling. TRP receptors have six transmembrane segments, varying degrees of sequence homology and permeability to various cations.

Transient receptor potential subfamily V member 1 (“TRPV1”) has six members divided into two groups. A first group includes TRPV1, TRPV2, TRPV3, and TRPV4. TRPV1, TRPV2, TRPV3 and TRPV4 include thermo-TRPs that are activated by heat. TRPV1 is a non-selective cation channel involved primarily in in pain sensation. TRPV1 may affect mood and neuroplasticity in the brain, and there is a clear molecular link between TRPV1 activity and stress responses. TRPV3 mediates a weakly Ca²⁺-selective cationic conductance in response to non-noxious heat, camphor and other molecules. TRPV3 is widely expressed in humans, with an important role in thermosensation. TRPV4 is a constitutively active Ca²⁺-permeable cation channel displaying a response to moderate heating, hypotonic challenge, or the phorbol ester 4a-PDD. TRPV4 is involved in pressure and osmotic sensitivity, thermal selection and hearing. A second group of TRPV receptors includes TRPV5 and TRPV6, which are selective Ca²⁺ transporters but do not respond to heat.

Transient receptor potential canonical (“TRPC”) channels represent a group of receptor-operated calcium-permeable nonselective cation channels of the TRP superfamily. Seven mammalian TRPC members can be divided into four subgroups (TRPC1, TRPC2, TRPC4/5, and TRPC3/6/7) are involved in a range of cellular and physiological functions. TRPC channels have been implicated in calcium release activated channels in many cell types and have been suspected to be involved in Alzheimer's disease and various cardiomyopathies.

Transient receptor potential subfamily ‘A’ ion channels (“TRPA”) is a family of receptors made up of 7 subfamilies: TRPA1, TRPA- or TRPA1-like, TRPA5, painless, pyrexia, waterwitch, and HsTRPA. These channels have been identified as mechanical stress, temperature, and chemical sensors. TRPA1 was initially described as a cold sensitive nonselective cation channel, there is evidence that it functions as a ligand-gated channel in expression systems and sensory neurons. TRPA1 appears to be regulated by PLC-coupled receptors, exhibiting many functional characteristics of other TRP channels. TRPA1 also has been tentatively identified as a hearing transduction channel, while the multiple ankyrin repeats in TRPA1 have been suggested to function as a mechanical spring, linking TRPA1 to cytoskeletal proteins.

Transient receptor potential subfamily M (M is shorthand for melastatin) subfamily comprises eight members divided into three groups. A first group includes TRPM1, TRPM2 and TRPM3. A second group includes TRPM4, TRPM5 and TRPM8. A third group includes TPRM6 and TRPM7. TRPM receptors have been implicated in various processes including regulation of calcium, modulation of insulin secretion and cold and heat sensation, inflammatory pain, magnesium reabsorption and regulation of cell adhesion.

TRPM3 has alternate functional splice variants. Mouse TRPM3α1 is monovalent-selective, while TRPM3α2 is divalent selective, suggesting that in vivo TRPM3 function may depend on the relative abundance of these variants. TRPM3α1 and TRPM3α2 both display constitutively active rectifying currents which are blocked by intracellular Mg²⁺. TRPM3 has been implicated in microglial and choroid plexus functions.

TRPM8 has been mostly described as a cold- and menthol-activated nonselective cation channel displaying voltage dependent gating properties. TRPM8 is expressed in sensory neurons, potentially acting as a cold thermosensor. Channel agonists such as cold or menthol may shift the voltage dependence of TRPM8 to more negative potentials.

TRPN is a member of the transient receptor potential channel family of ion channels. The TRPN gene was named no mechanoreceptor potential C (nompC) when first discovered in a Drosophila species. TRPN receptors are mechanoreceptors. Studies of TRPN indicate that null mutants in fruit flies have difficulty moving, suggesting a role in proprioception. Studies in worms have shown null mutants to have various locomotion defects while electrophysiological studies of single channels in worms have shown that TRPN responds to mechanical stimuli and has a preference for sodium ions.

TRPP (transient receptor potential polycystic) is a family of transient receptor potential ion channels named for mutations that can cause polycystic kidney disease.

TRPML (transient receptor potential cation channel, mucolipin subfamily) represent a group of three related proteins. The three proteins TRPML1, TRPML2 and TRPML3 are encoded by the mucolipin-1 (MCOLN1), mucolipin-2 (MCOLN2) and mucolipin-3 (MCOLN3) genes. The three members of the TRPML sub-family are not well characterized, while TRPML1 is known to be localized in late endosomes. This subunit also contains a lipase domain between its S1 and S2 segments, with the domain hypothesized to be involved in channel regulation. Studies have described TRPML1 channels as proton leak channels in lysosomes responsible for preventing these organelles from becoming too acidic, with deficiencies leading to enlarged vesicles.

### Multi-Modal Treatment of Health Conditions

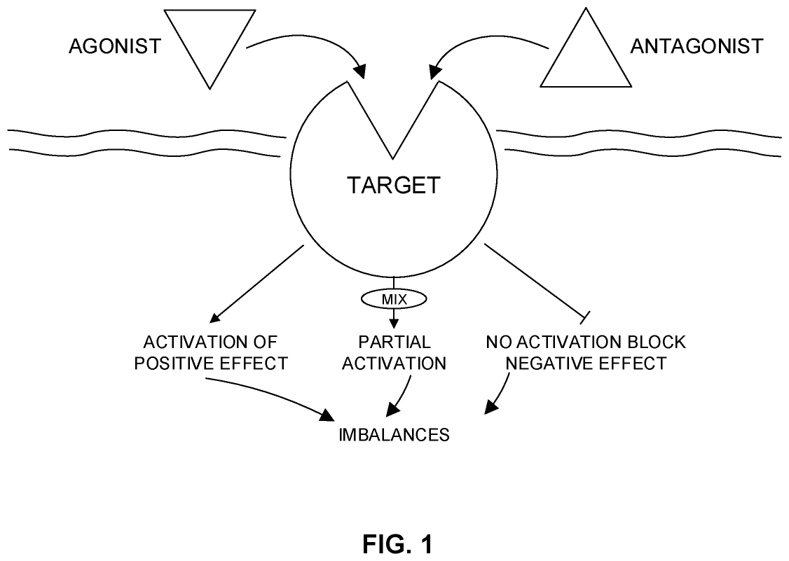

FIG. 1 shows a schematic of a generalized single-molecule approach, as is used in most traditional pharmaceutical therapeutics. Traditional pharmaceuticals target a single receptor with a single molecule to produce a predicted result of complete or partial activation or deactivation. Most commonly a receptor agonist or antagonist may be applied at a specific concentration to reduce the symptoms of a disease for a period of time. The single-molecule approach, particularly with strong agonists and strong antagonists, rarely restores balance to a system or cures a disease. This linear approach does not account for or otherwise leverage the complicated feedback mechanisms that may upregulate or downregulate the process itself. Furthermore, single molecule approaches have often focused on molecules with increased potency or pharmacodynamic properties under the assumption that increased activation is more efficacious in treating disease. As a result, these approaches often result in imbalanced biological systems rather than restoration of balance to cellular processes and other biological systems. The imbalances that often result from single-molecule pharmaceutical medications often lead to side effects that are commonly dealt with by a second medication, which may result in further imbalance.

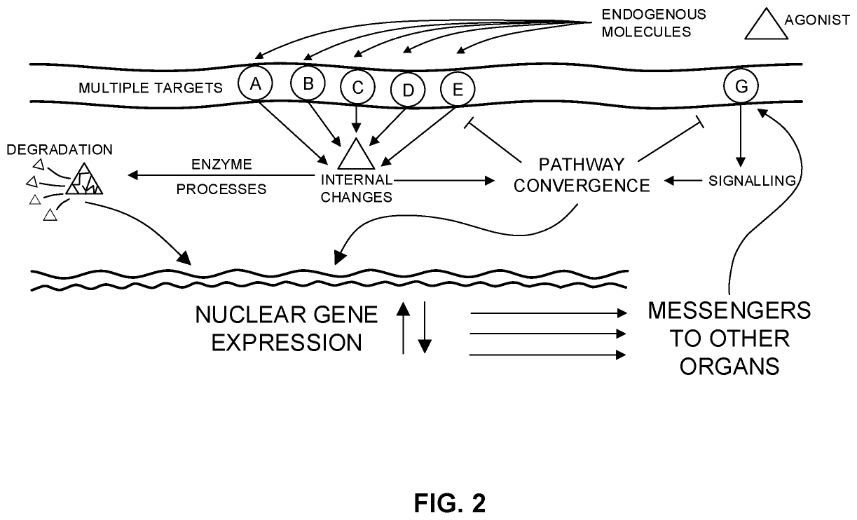

FIG. 2 shows a schematic of a generalized multi-modal approach, providing a therapeutic that accounts for multiple portions of a metabolic pathway, accounting for stimulation and feedback loops that may result in imbalance or toxicity. A drug may have a single target or multiple receptor targets, and interaction with the targets of the drug will be affected by other features of the system. Endogenous molecules may interact with the same receptor and compete for receptor sites. Introduction of strong agonists or strong antagonists may amplify, reduce, prevent or otherwise perturb signaling within endogenous pathways. The extent and distribution of expression of receptors in the body may be heterogeneous. Interaction with any given receptor may have downstream changes in gene expression and signaling to other parts of the body. Multiple signaling pathways may converge within a cell to regulate one another and maintain homeostasis between many processes. Interaction of a drug with a given receptor may influence homeostasis through downstream effects, in some cases drastically. Degradation of the drug may have result in byproducts that have further signaling consequences and potential side effects, including by modulating metabolites or other byproducts of endogenous processes. Natural molecules often have clear metabolic breakdown pathways and may a lack some of toxic bi-products resulting from synthetic drugs. As seen through traditional medicine approaches (Chinese medicine, Aruvedic formulas, etc), combinations of multiple natural molecules shows potential for treating disease with little to no side effects.

Restoring homeostasis to metabolic pathways requires attention to the production, reception, signaling and breakdown of each neurotransmitter or other signaling molecule entering the system, present in the system and exiting the system. Biological and molecular pathways are cycles with multiple inputs, convergence points and branch points resulting in different products or effects. Multiple enzymes are involved in most biological processes, including those that signal events in the nucleus and trigger gene expression. Such signals may result in positive or negative feedback at different parts of a metabolic pathway. If a metabolic system has been downregulated, stimulation at multiple points may facilitate restoration of homeostasis. If a metabolic system has been upregulated, attenuation at multiple points may facilitate restoration of homeostasis. Molecular pathways may be downregulated in one part and upregulated in another through interaction with one or multiple chemicals. Blocking or overstimulating any one part of the cycle may create imbalances resulting in side effects. For example, while serotonin does not cause intoxication, psilocin and other 5HT2A agonists may overstimulate the 5HT2A receptor causing hallucinations and other psychoactive effects. The compositions include ingredients in addition to a 5HT2A agonist that provide a multimodal molecular approach, combining both agonists and antagonists for other receptors that converge with 5HT2A signaling to provide benefits while mitigating psychoactive effects of 5HT2A agonists.

### Mental Health and Gut Health

Many components found in food show antidepressant effects. Many of these same ingredients have also been associated with a reduction in inflammation and play a role in sugar metabolism. Relatively low doses of certain natural molecules including a 5HT2A agonist and a TRP receptor agonist may facilitate balance to inflammatory mediators and use of compositions including a 5HT2A agonist and a TRP receptor agonist may present an option for treatment and prevention of inflammatory disease, mental illness and sugar metabolism issues.

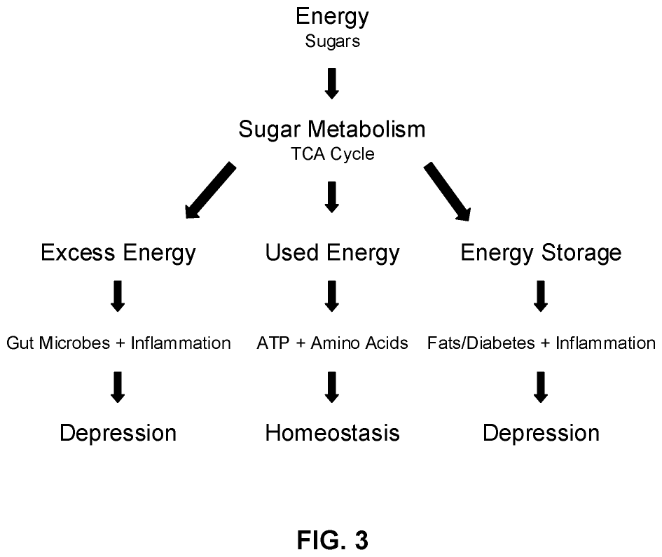

FIG. 3 shows a schematic illustrating the relationship between energy, sugar metabolism, homeostasis and depression. Inflammation may result from an energetic imbalance. Where energy enters a cell or other system and is not converted by the system to something useful, and subsequently removed from the system, inflammation ensues. Inflammation, if left unchecked, will lead to a multitude of negative consequences that can be additive and exacerbate one another, compounding into much larger events. Correlative links between consumption of excess sugar and inflammation have long been suspected (Corte, 2018).

In humans, energy is primarily provided to the body as sugars (e.g. glucose, fructose, sucrose, etc.). Glucose is converted into energy in cells. Excess sugars, particularly those from high-fructose corn syrup or other processed sources, have been shown to influence inflammatory markers and result in inflammation. Inflammation associated with excess sugar intake may partially be a result of microbial metabolites from flora feeding on sugars in the intestine. Inflammation associated with excess sugar intake may also be due to excess energy that the body has no means to deal with and therefore results in the energy being stored as fats, subsequently altering glucose metabolism and leading to conditions including diabetes.

MDD has not been specifically defined or characterized as an inflammatory disease. However, there is evidence is to show crosstalk between depression and inflammation (Patel, 2013; Krishnadas, 2012; Lee, 2019; Slavich, 2014). Many metabolic pathways in the body may be influenced simultaneously in disease conditions and may also be influenced simultaneously by therapies. Individuals with inflammatory diseases are more likely to show increased rates of depression and individuals with depression are more likely to show increased rates of inflammatory diseases. Over 30% of people with MDD show elevated peripheral inflammatory biomarkers even in the absence of a diagnosed illness associated with inflammation (Slavich, 2014). Inflammation plays a role in the pathogenesis of a number of immune system, neurological and behavioral disorders, including depression, cognitive impairment, metabolic and autoimmune diseases. With respect to depression and inflammation, neither appears to be absolutely necessary or sufficient for the other. Each may occur in the absence of the other, but depression and inflammation are often comorbid. In a significant subset of individuals, inflammation may precipitate or prolong depression or may significantly contribute to the inflammatory response, the course and the outcome of a comorbid disease. Biofeedback mechanisms between these inflammation and depression may exacerbate each condition.

Metabolism of sugar is vitally important for the body the primary source of energy. Glucose is normally broken down to pyruvate in the cytosol of cells. Pyruvate enters mitochondria to create adenosine triphosphate and other energy useful to the body. Pyruvate is also a direct metabolic precursor to alanine, to aspartate and asparagine through oxaloacetate, and to glutamate and glutamine through a-ketoglutarate. Glucose that is not used in this manner may enter other biosynthetic process or can be stored.

Glutamine is the most abundant amino acid in the human body and is involved in many metabolic processes, including glucose metabolism and biosynthesis of other amino acids. Some cells can directly transform glutamine into glucose through a series of chemical reactions. When glucose is then released into the blood, blood sugar level rises causing a cascade of changes throughout the body.

In addition to regulation of primary metabolism, glutamine and glutamate signaling appear to be intrinsically linked to brain activity and inflammation. Inflammatory mediators have been found to alter glutamate and monoamine neurotransmission, glucocorticoid receptor resistance and hippocampal neurogenesis. Inflammation can alter brain signaling patterns, altering cognition and contributing to the production of a pattern of symptoms, closely related to depression. Inflammation may exacerbate the complexity and severity of many illnesses, and influence treatment response. As such, inflammatory responses may lead to depression and depression may lead to inflammation in a way that each exacerbate each other.

The connection between consumption of processed sugar and diabetes is well known. There appears to also be direct correlations between excess consumption of processed sugar in a population and prevalence of mental health issues in the population. Specifically, high fructose corn syrup and artificial sweeteners can interfere with intestinal sugar signaling and brain signaling functions (de Sousa, 2017). A significant number of depression and mood disorder diagnoses may be a result of intestinal inflammation, which may be correlated to high sugar diets. Correction or mitigation of the intestinal inflammation may correct or mitigate the depression connected with the intestinal inflammation. Compositions that include both a 5HT2A receptor agonist and a TRP receptor agonist may mitigate or correct the intestinal inflammation.

As individuals consume and process food, communication between the enteric nervous system (the “ENS”) and the central nervous systems (the “CNS”) is vital for maintaining homeostasis, particularly around sugars. The ENS is a portion of the peripheral nervous system (the “PNS”). The pathway between the ENS and the CNS is known as the gut-brain axis.

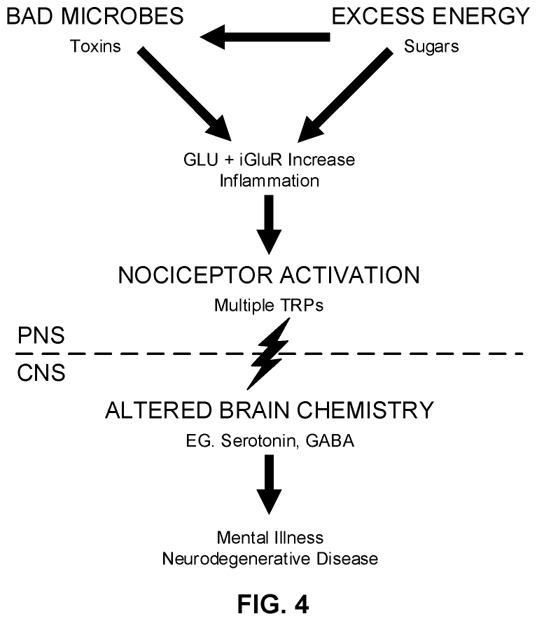

FIG. 4 shows interactions between the gut and the brain and sugar signaling progressing from the gut to the brain. Glutamate is key in sugar and energy signaling, but is also an important excitatory neurotransmitter in the nervous system. Glutamate plays an important role in nociception by transmission of signals from the PNR to the CNS following stimulation of specialized sensory TRP nociceptors. As such, glutamate is directly involved in the sensitization of inflammation pain and neuropathic pain.

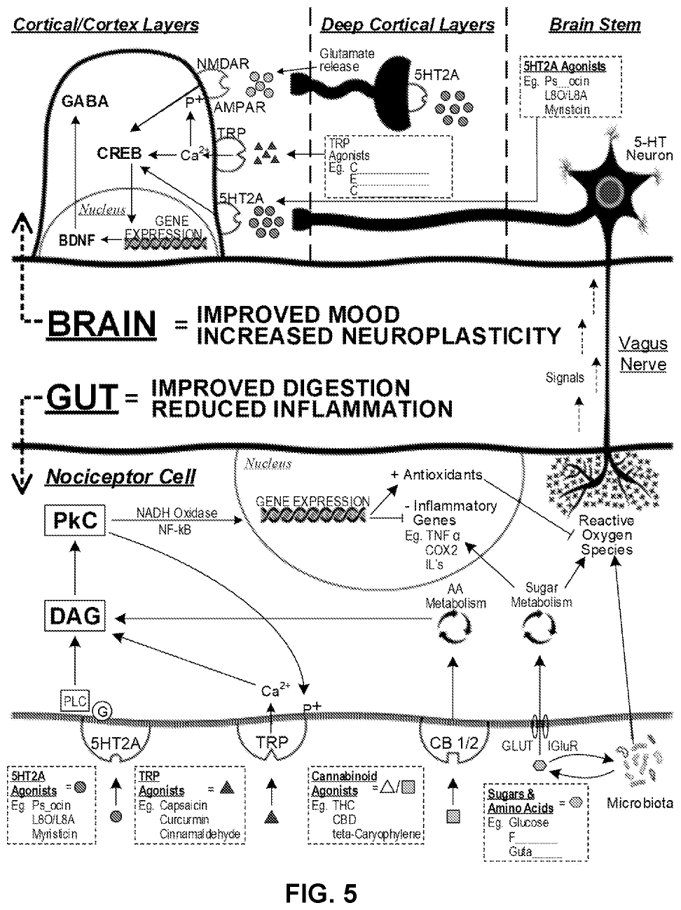

Glutamate levels elevate and expression of ionotropic glutamate receptors (“iGluRs”) is upregulated during cutaneous inflammation and during deep tissue inflammation. Peripheral inflammation increases the proportions of both unmyelinated and myelinated nerves expressing iGluRs. Nociceptor cells are found in any area of the body that can sense stimuli and are prevalent in the digestive tract. The peripheral terminal of a mature nociceptor is where the stimuli are detected and transduced into electrical energy. When the electrical energy reaches a threshold value, an action potential is induced and driven towards the CNS, where specific changes in metabolic activity result in production of neurotransmitters. Glutamate signaling in the brain and TRP signaling from the gut alters brain chemistry in at least the manner described in this paragraph, and shown in FIGS. 4 and 5.

Nociceptor neuron sensitivity is modulated by a large variety of mediators in the extracellular space (Woolf, 2007). Peripheral sensitization represents a form of functional plasticity of the nociceptor. The nociceptor can change from being simply a noxious stimulus detector to a detector of non-noxious stimuli. The result is that low intensity stimuli from regular activity, initiates a painful sensation. Inflammation results in the sensitization of nociceptors. Normally problems cease when inflammation goes down; however, sometimes genetic defect or repeated injury, including from chronic exposure to excess energy from processed sugars, can result in aberrant issues and lead to chronic issues in this pathway.

Glutamate plays an important role in transmitting the nociceptive signals from the PNS to the spinal cord. Glutamate injections provoked nociceptive responses mediated by neuropeptides (substance P) released from C fibers by activation of glutamate receptors that stimulate the production of a variety of intracellular secondary messengers. These include nitric oxide, pro-inflammatory cytokines, such as tumor necrosis factor alpha (“TNF-α”) and interleukins such as interleukin-1β (“IL-1β”), which act synergistically in the excitation of the neurons (Goldstein, 2009).

Several studies have pointed to the presence of increased inflammatory cytokines in individuals with MDD. Inflammatory cytokines are cell-signaling protein molecules that are released during inflammation and launch signaling cascades able to activate the immune system. Type 1 cytokines include TNF-α, interferon-γ, interleukin-1β. Type 1 cytokines enhance cellular immune responses. Pro-inflammatory cytokines may be produced in the brain itself or reach the brain from the PNS through active transport or “leaky” regions across the blood-brain barrier. Cytokines may signal the brain through the afferent vagal pathway or via the entry of activated monocytes into the brain from periphery. The compositions with both 5HT2A and TRP agonists reduce gut inflammation markers, which results in improved mental health.

Meta-analyses have compared individuals suffering from MDD to a control group, and shown differences in proinflammatory cytokines such as TNF-α (Liu, 2012, Ma, 2016). The meta-analyses showed that individuals with MDD had significantly higher concentrations of TNF-α compared with controls. Interleukin 6 (“IL-6”) has also been linked to depression especially when comorbid with physical disorders. Both animal and clinical studies demonstrate increased peripheral or central cytokine interleukin-6 levels play an important role in stress reactions and depressive disorders. Ting, E. et al., “Role of Interleukin-6 in Depressive Disorder,” Int. J. Mol. Sci. 21(6):2194 (March 2020). The majority of researches indicate that individuals suffering from depression have shown elevations in the proinflammatory cytokines.

Cyclooxygenase (“COX”), officially known as prostaglandin-endoperoxide synthase (“PTGS”) is a protein that is responsible for the synthesis of prostaglandins. Prostaglandins are bioactive lipids that have potent actions in inflammation, fever and pain as well as provide protection of gastric mucosa and platelet aggregation. COX2 specifically is another proinflammatory regulator of inflammation and has been linked depression. COX-2 inhibition directly effects the CNS and some components of the inflammatory system, kynurenine-metabolism and glutamatergic neurotransmission. COX-2 inhibitors have been tested in animal models of depression and in preliminary clinical trials, the latter showing favorable effects compared to placebo, both, in schizophrenia and in MDD. The compositions are effective at reducing COX2.

Subsets of vagal afferent nerves have activation properties indicative of specialization to detect potentially harmful stimuli. A cascade of events occurs in the brain including regulation of inflammation markers and the perception of pain. When a high threshold is reached by either chemical, thermal, or mechanical environments, the nociceptors are triggered under normal conditions. TRP receptors are responsible for whether and how specific nerve endings respond to stimuli, providing for a variety of potential responses. Inclusion of different TRP agonists that may interact with different TRP receptors can differentially influence downstream gene expression as well as the over-perception of the effects of combined 5HT2A formulation.

Studies investigating the gut-brain axis demonstrate that the gut microbiota is an important regulator of interactions relevant to brain development, behavior and the immune system. Microbes influence the activation of peripheral immune cells, which regulates responses to neuroinflammation, brain injury, autoimmunity and neurogenesis. Both the gut microbiota and immune system are implicated in the etiopathogenesis or manifestation of neurodevelopmental, psychiatric and neurodegenerative diseases. Compositions with both 5HT2A and TRP agonists may influence gut microbiota in various ways.

Stress causes changes in neurotransmission in the brain and influences stress-induced behaviors. However, it is unclear how neurotransmission systems orchestrate stress responses at the molecular and cellular levels. Although the extent to which inflammation contributes to depression onset and relapse is unknown, studies have shown that elevated serum cytokine levels of inflammatory markers often precede, and therefore potentially cause depressive symptoms (Karlovie, 2012; Krishnadas 2012) Many illnesses show difference in inflammation markers including diabetes, metabolic syndrome, rheumatoid arthritis, asthma, multiple sclerosis, cardiovascular disease, chronic pain, and psoriasis, which are also characterized by increased risk for depression (Gan, 2004; Stenvinkel 2002). For instance, nearly 20% of individuals with cardiovascular disease experience MDD (O'Neil, 2013), individuals suffering from diabetes are twice as likely to develop depression (Mezuk, 2008) and up to 70% of individuals suffering from autoimmune diseases, such as systemic lupus erythematosus (Palagini, 2013) or rheumatoid arthritis (Dickens, 2002) all experience higher rates of depression. Compositions with both 5HT2A and TRP agonists may also help with disease states found to be comorbid with depression due to treatment of the underlying inflammation.

Conditions associated with chronic inflammation are listed in Table 1.

TABLE 1
Conditions Associated with Chronic Inflammation

| Disease | Type | Signs and Symptoms |
|---|---|---|
| Arthritis | Bone, | Joint pain, tenderness, stiffness, inflammation in and |
| | Muscular, | around the joints, restricted movements of the joints, |
| | Skeletal | warm red skin over the affected joint, weakness and |
| | | muscle wasting. |
| Osteoporosis | Bone, | Back pain (caused by a fractured or collapsed vertebra), |
| | Muscular, | loss of height over time, stooped posture, a bone that |
| | Skeletal | breaks much more easily than expected. |
| Osteoarthritis | Bone, | Pain and stiffness in joints, joint tenderness, increased |
| | Muscular, | pain and stiffness when immobile for long periods, |
| | Skeletal | joints swelling, grating/crackling sound or sensation in |
| | | joints, limited range of movement in joints, weakness |
| | | and muscle wasting (loss of muscle bulk). |
| Degenerative | Bone, | Range from mild to severe pain, neck and lower back |
| Disc Disease | Muscular, | pain, pain extending to the arms and hands, pain extends |
| (DDD) | Skeletal | through buttocks and thighs, pain amplifies when |
| | | sitting/bending/lifting/twisting, weakness in leg muscles |
| | | or foot drop |
| Muscular | Bone, | MD can include the following types; duchenne MD, |
| Dystrophy | Muscular, | myotonic dystrophy, facioscapulohumeral MD, becker |
| | Skeletal | MD, limb-girdle MD, oculopharyngeal MD and emery- |
| | | dreifuss MD. All types of MD cause muscle weakness, |
| | | but the areas affected and the severity of the symptoms |
| | | differ by type. |
| Breast Cancer | Cancer | A change in the size or shape of one or both breasts, |
| | | discharge from either of your nipples, which may be |
| | | streaked with blood, a lump or swelling in either of your |
| | | armpits, dimpling on the skin of your breasts, a rash on |
| | | or around your nipple and a change in the appearance of |
| | | your nipple (such as becoming sunken into your breast). |
| Colon Cancer | Cancer | Persistent change in bowel habit - pooing more often, |
| | | with looser, runnier poos and sometimes tummy |
| | | (abdominal) pain, blood in the poo without other |
| | | symptoms of piles (haemorrhoids) - this makes it |
| | | unlikely the cause is haemorrhoids, abdominal pain, |
| | | discomfort or bloating always brought on by eating - |
| | | sometimes resulting in a reduction in the amount of food |
| | | eaten and weight loss. |
| Lung Cancer | Cancer | A cough that doesn't go away after 2 or 3 weeks, a long- |
| | | standing cough that gets worse, chest infections that |
| | | keep coming back, coughing up blood, an ache or pain |
| | | when breathing or coughing, persistent breathlessness, |
| | | persistent tiredness or lack of energy, loss of appetite or |
| | | unexplained weight loss. |
| Kidney Cancer | Cancer | blood in your pee - you may notice your pee is darker |
| | | than usual or reddish in colour, a persistent pain in your |
| | | lower back or side, just below your ribs, a lump or |
| | | swelling in your side (although kidney cancer is often |
| | | too small to feel), extreme tiredness, loss of appetite and |
| | | unintentional weight loss, persistent high blood pressure |
| | | (hypertension), a high temperature, night sweats, |
| | | swelling of the veins in the testicles (for men), swollen |
| | | glands in your neck, bone pain and coughing up blood. |
| Gastric Cancer | Cancer | heartburn or acid reflux, having problems swallowing |
| | | (dysphagia), feeling or being sick, symptoms of |
| | | indigestion, such as burping a lot |
| | | feeling full very quickly when eating, loss of appetite or |
| | | losing weight without trying to, a lump at the top of |
| | | your tummy, pain at the top of your tummy and feeling |
| | | tired or having no energy. |
| Pancreatic | Cancer | The whites of your eyes or your skin turn yellow |
| Cancer | | (jaundice), you may also have itchy skin, darker pee and |
| | | paler poo than usual, loss of appetite or losing weight |
| | | without trying to, feeling tired or having no energy, a |
| | | high temperature, or feeling hot or shivery. |
| Lymphoma | Cancer | The two most common types of lymphoma are Hodgkin |
| | | and non-Hodgkin lymphoma. The most common |
| | | symptom of Hodgkin lymphoma is a swelling in the |
| | | neck, armpit or groin. The swelling is usually painless, |
| | | although some people find that it aches. The most |
| | | common symptom of non-Hodgkin lymphoma is a |
| | | painless swelling in a lymph node, usually in the neck, |
| | | armpit or groin. Lymph nodes, also known as lymph |
| | | glands, are pea-sized lumps of tissue found throughout |
| | | the body. |
| Atherosclerosis | Cardiovascular + | Chest pain, numbness, weakness in arms/legs, difficulty |
| | Diabetic | speaking, slurred speech, temporary loss of vision in one |
| | | eye, drooping facial muscles, claudication, high blood |
| | | pressure and kidney failure. |
| Cardiomyopathy | Cardiovascular + | Shortness of breath, on exertion or at rest, recurrent |
| | Diabetic | chest infections, coughing up sputum or blood, |
| | | palpitations, and an awareness of your heart beating |
| | | faster or in an irregular way, chest pains, and/or angina |
| | | pain, abnormal heart rhythms - called arrhythmias, |
| | | fainting or near fainting, dizziness, swelling to the face, |
| | | abdomen or extremities, undue tiredness and reduced |
| | | exercise tolerance. |
| Cerebrovascular | Cardiovascular | There are a number of different types of cerebrovascular |
| disorder | Disease | disease. The four most common types are; Stroke - a |
| | | serious medical condition where one part of the brain is |
| | | damaged by a lack of blood supply or bleeding into the |
| | | brain from a burst blood vessel, Transient Ischaemic |
| | | Attack (TIA) - a temporary fall in the blood supply to |
| | | one part of the brain, resulting in brief symptoms similar |
| | | to stroke, Subarachnoid Haemorrhage - a type of stroke |
| | | where blood leaks out of the brain's blood vessels on to |
| | | the surface of the brain, Vascular Dementia - persistent |
| | | impairment in mental ability resulting from stroke or |
| | | other problems with blood circulation to the brain. |
| Heart Failure | Cardiovascular | Breathlessness - this may occur after activity or at rest; |
| | Disease | it may be worse when you're lying down, and you may |
| | | wake up at night needing to catch your breath, Fatigue - |
| | | you may feel tired most of the time and find exercise |
| | | exhausting, Swollen ankles and legs - this is caused by |
| | | a build-up of fluid (oedema); it may be better in the |
| | | morning and get worse later in the day. |
| Stroke | Cardiovascular | The main stroke symptoms can be remembered with the |
| | Disease | word FAST; |
| | | Face - the face may have dropped on 1 side, the person |
| | | may not be able to smile, or their mouth or eye may |
| | | have drooped. |
| | | Arms - the person may not be able to lift both arms and |
| | | keep them there because of weakness or numbness in 1 |
| | | arm. |
| | | Speech - their speech may be slurred or garbled, or the |
| | | person may not be able to talk at all despite appearing to |
| | | be awake; they may also have problems understanding |
| | | what you're saying to them. |
| | | Time - it's time to dial 999 immediately if you notice |
| | | any of these signs or symptoms. |
| Leaky Gut | Chronic | Proponents of “leaky gut syndrome” claim that many |
| | Inflammatory | symptoms and conditions are caused by the immune |
| | Disorder | system reacting to germs, toxins or other substances that |
| | | have been absorbed into the bloodstream via a porous |
| | | (“leaky”) bowel. |
| Ulcerative | Chronic | The main symptoms of UC are: recurring diarrhoea, |
| Colitis | Inflammatory | which may contain blood, mucus or pus, tummy pain, |
| | Disorder | needing to empty your bowels frequently. You may also |
| | | experience extreme fatigue, loss of appetite and weight |
| | | loss. |
| Irritable Bowel | Chronic | The main symptoms of IBS are: |
| Syndrome (IBD) | Inflammatory | stomach pain or cramps - usually worse after eating and |
| | Disorder | better after doing a poo |
| | | bloating - your tummy may feel uncomfortably full and |
| | | swollen |
| | | diarrhoea - you may have watery poo and sometimes |
| | | need to poo suddenly |
| | | constipation - you may strain when pooing and feel like |
| | | you cannot empty your bowels fully |
| Chronic | Chronic | ongoing cough (wet/dry), shortness of breath, wheezing, |
| Obstructive | Inflammatory | whistling/squeaky sound when breathing, chest |
| Pulmonary | Disorder | tightness, hard time catching your breath, |
| Disease (COPD) | | lips/fingernails turn blue or gray, lack of mental |
| | | alertness, very fast heartbeat. |
| Rheumatoid | Chronic | Pain or aching in more than one joint, stiffness in more |
| Arthritis (RA) | Inflammatory | than one joint, tenderness and swelling in more than one |
| | Disorder | joint, the same symptoms on both sides of the body |
| | | (such as in both hands or both knees), weight loss, fever, |
| | | fatigue, tiredness, weakness. |
| Psoriasis | Chronic | Symptoms are dry red skin lesions, known as plaques, |
| | Inflammatory | covered in silver scales. They normally appear on your |
| | Disorder | elbows, knees, scalp and lower back, but can appear |
| | | anywhere on your body. The plaques can be itchy or |
| | | sore, or both. In severe cases, the skin around your |
| | | joints may crack and bleed. |
| Chronic | Chronic | Chronic pancreatitis is a condition where the pancreas |
| Pancreatitis | Inflammatory | has become permanently damaged from inflammation |
| | Disorder | and stops working properly. The most common |
| | | symptom of chronic pancreatitis is repeated episodes of |
| | | severe pain in your tummy (abdomen). The pain usually |
| | | develops in the middle or left side of your tummy and |
| | | can move along your back. It's been described as a |
| | | burning or shooting pain that comes and goes, but may |
| | | last for several hours or days. Although the pain |
| | | sometimes comes on after eating a meal, there's often no |
| | | trigger. Some people might feel sick and vomit. |
| Chronic | Chronic | Tingling in arms and legs, gradual weakening of arms |
| Inflammatory | Inflammatory | and legs, loss of reflexes, loss of balance and your |
| Demyelinating | Disorder | ability to walk, loss of feeling in your arms and legs, |
| Polyradiculoneuropathy | | which often starts with your inability to feel a pin prick. |
| (CIDP) | | |
| Chronic | Chronic | More than 200 different types of connective tissue |
| Inflammatory | Inflammatory | diseases including rheumatoid arthritis (RA), |
| Connective | Disorder | scleroderma, granulomatosis with polyangiitis (GPA), |
| Tissue Diseases | | churf-strauss syndrome, systemic lupus erythematosus |
| (CICTD) | | (SLE), microscopic polyangiitis (MPA), |
| | | polymyositis/dermatomyositis, mixed connective tissue |
| | | disease (MCTD) and undifferentiated connective tissue |
| | | diseases. |
| Gingivitis | Dental | Swollen gums, dark red gums, gums that bleed easily, |
| | | bad breath, receding gum line, tender/sensitive gums. |
| Retinopathy | Diabetic | Symptoms include gradually worsening vision, sudden |
| | Complications | vision loss, shapes floating in your field of vision |
| | | (floaters), blurred or patchy vision and eye pain or |
| | | redness. |
| Sepsis | Diabetic | Sepsis can be hard to spot. There are lots of possible |
| | Complications | symptoms. Symptoms can be vague. They can be like |
| | | symptoms of other conditions, including flu or a chest |
| | | infection. |
| Neuropathy | Diabetic | The main symptoms of peripheral neuropathy can |
| | Complications | include; numbness and tingling in the feet or hands, |
| | | burning, stabbing or shooting pain in affected areas, loss |
| | | of balance and co-ordination and muscle weakness, |
| | | especially in the feet. |
| Chronic Kidney | Metabolic | Symptoms include tiredness, swollen ankles, feet or |
| Disease (CKD) | Disorder | hands (due to water retention), shortness of breath, |
| | | nausea and blood in the urine. |
| Fatty Liver | Metabolic | There aren't usually any prevalent symptoms in the early |
| Disease | Disorder | stages. Some symptoms in more serious cases may |
| | | include; a dull or aching pain in the top right of the |
| | | tummy (over the lower right side of the ribs), fatigue |
| | | (extreme tiredness), unexplained weight loss and |
| | | weakness. |
| Heart Disease | Metabolic | Symptoms of a heart attack can include; |
| | Disorder | pain in other parts of the body - it can feel as if the |
| | | pain is travelling from your chest to your arms, jaw, |
| | | neck, back or stomach |
| | | light-headedness |
| | | sweating |
| | | nausea |
| | | breathlessness |
| Sleep Apnea | Metabolic | Symptoms of sleep apnea during sleep: breathing |
| | Disorder | stopping and starting, making gasping, snorting or |
| | | choking noises, waking up frequently, loud snoring. |
| | | Symptoms of sleep apnea during the day: feel very |
| | | tired, find it hard to concentrate, have mood swings, |
| | | have a headache when you wake up. |
| Type 2 Diabetes | Metabolic | Typical symptoms include: |
| | Disorder | feeling very thirsty |
| | | passing urine more often than usual, particularly at |
| | | night |
| | | feeling very tired |
| | | weight loss and loss of muscle bulk |
| | | slow to heal cuts or ulcers |
| | | frequent vaginal or penile thrush |
| | | blurred vision |
| Metabolic | Metabolic | High blood sugar, hypertension, high triglycerides, low |
| Syndrome | Disorders | HDL-cholesterol, Visceral Obesity |
| Autoimmune | Metabolic | Despite the varying types of autoimmune disease, many |
| Diseases | Disorders | of them share similar symptoms. Common symptoms of |
| | | autoimmune disease include: |
| | | Fatigue |
| | | Joint pain and swelling |
| | | Skin problems |
| | | Abdominal pain or digestive issues |
| | | Recurring fever |
| | | Swollen glands |
| Amyotrophic | Neurological | Early symptoms can include: |
| Lateral Sclerosis | | weakness in your ankle or leg - you might trip, or find |
| (ALS) | | it harder to climb stairs |
| | | slurred speech, which may develop into difficulty |
| | | swallowing some foods |
| | | a weak grip - you might drop things, or find it hard to |
| | | open jars or do up buttons |
| | | muscle cramps and twitches |
| | | weight loss - your arms or leg muscles may have |
| | | become thinner over time |
| | | difficulty stopping yourself from crying or laughing in |
| | | inappropriate situations |
| Alzheimer's | Neurological | The symptoms of Alzheimer's disease progress slowly |
| | | over several years. Sometimes these symptoms are |
| | | confused with other conditions and may initially be put |
| | | down to old age. |
| | | The rate at which the symptoms progress is different for |
| | | each individual. |
| | | In some cases, other conditions can be responsible for |
| | | symptoms getting worse. |
| | | These conditions include: |
| | | infections |
| | | stroke |
| | | delirium |
| Anxiety | Neurological | Symptoms can include: |
| | | restlessness |
| | | a sense of dread |
| | | feeling constantly “on edge” |
| | | difficulty concentrating |
| | | irritability |
| Dementia | Neurological | Some common early symptoms that may appear some |
| | | time before a diagnosis of dementia. These include: |
| | | memory loss |
| | | difficulty concentrating |
| | | finding it hard to carry out familiar daily tasks, such as |
| | | getting confused over the correct change when shopping |
| | | struggling to follow a conversation or find the right |
| | | word |
| | | being confused about time and place |
| | | mood changes |
| Depression | Neurological | The symptoms of depression can be complex and vary |
| | | widely between people. If you're depressed, you may |
| | | feel sad, hopeless and lose interest in things you used to |
| | | enjoy. |
| Stress | Neurological | Physical symptoms: |
| | | headaches or dizziness |
| | | muscle tension or pain |
| | | stomach problems |
| | | chest pain or a faster heartbeat |
| | | sexual problems |
| | | Mental symptoms: |
| | | difficulty concentrating |
| | | struggling to make decisions |
| | | feeling overwhelmed |
| | | constantly worrying |
| | | being forgetful |
| Asthma | Respiratory | The most common symptoms of asthma are: |
| Allergies | | wheezing (a whistling sound when breathing) |
| | | breathlessness |
| | | a tight chest - it may feel like a band is tightening |
| | | around it |
| | | coughing |

### Improving Mental Health by Mitigating Gut Inflammation

Mental health issues may be treated in many individuals by reducing inflammation in the intestines, specifically nociception of the enterochromaffin cells where the majority of serotonin is produced in the gut. Natural sources from plant and fungal medicines may be utilized to reduce gut inflammation through targeting specific receptors that regulate pain sensitization and inflammation thereby transmitting signals to the brain.

While serotonin is well known for its central nervous system regulation of mood, appetite, and vasoconstriction, it is becoming increasingly clear that 5-HT also plays a modulatory role in various acute and persistent pain states (Stiedl, 2015; (Loyd 2013; Sommer, 2004). The vast majority of 5-HT in the mammalian body is located in peripheral tissues with significant production in the intestinal enterochromaffin cells where 5-HT is actively taken up by the gut and released with other chemical messengers to interact with the PNS at other parts of the body. For example, serotonin acts as a neuromodulator of nociception by altering TRPV1 activity during inflammation (Loyd 2013). In sensory neurons 5-HT increases excitability to thermal stimuli and enhances capsaicin and heat evoked currents (Sugivar, 2004; Ohta 2006). Depleting 5-HT attenuates visceral pain and reduces TRPV1 activation. 5-HT receptors regulate the stimulus threshold for sensory neurons by changing the properties of TRP receptor channels (Loyd, 2013).

FIG. 5 illustrates the mechanism of action of the compositions on 5HT2A receptors and TRP receptors together for reducing inflammation and improving mental health. Crossover with cannabinoid signaling is also illustrated. Simultaneous activation of these signaling pathways result in communication between the nociceptor cells of the gut (lower) and neurons in the brain (upper). The arrows demonstrate where the 5HT2A receptor agonists, the TRP agonists and CB receptor agonists included in the compositions bind with the 5HT2A, TPR and CB receptors. The arrows also show convergence of signaling pathways including sugars and on microbiomes that flourish in the presence of sugars, and that result in inflammation and ROS.

Coactivation of 5HT2A and TRP receptors reduces the intensity of negative signals resulting from sugars and from microbiome changes resulting from altered sugar signaling. One approach to treating depression is to increase serotonin signaling while also allowing serotonin breakdown in turn balances output signaling molecules such as diacylglycerol (“DAG”). DAG acts as a secondary messenger that is a physiological activator of protein kinase C (“PKC”). DAG facilitates translocation of PKC from the cytosol to the plasma membrane and is linked to many processes such as sugar metabolism. Phosphorylation of TRP channels, involving Protein Kinase C (PKC) appears to be the predominant mechanism for channel sensitization. For example, Ca′ influx through TRPV1 activates PKC and diacylglycerol kinase (“DAGK”) enzymes. DAGK limits formation of DAG, providing a feedback mechanism to maintain homeostasis by inhibiting Ca′ influx. Convergence of 5HT2A and TRP signaling pathways and the involvement of DAG and PKC results in an enhanced effect on the expression of inflammatory mediators and ROS.

The change in brain signaling may be affected through both altered signals from the gut and direct activity of the 5HT2A and TRP agonists in the brain. 5HT2A and TRP agonist activity in the brain, including glutamate signaling resulting from 5HT2A agonist activity, activates cAMP-response element binding protein (“CREB”). CREB increases expression of brain derived neurotrophic factor (“BDNF”) expression, which in turn increases GABA production and signaling. Taken together, this results in increased neuroplasticity and improved mood.

Endocannabinoid receptor signaling also converges on this pathway involving PKC through arachidonic acid (“AA”) metabolism and may further enhance the anti-inflammatory effects. While some cannabinoids may have direct psychoactive effects in the brain, some are thought to directly interact with TRP receptors as well. The corresponding reduction in inflammatory markers and ROS in the gut due to simultaneous signaling from 5HT2A and TRP in turn alters signaling cascades from the gut and subsequently improves brain chemistry.

Stimulation of the 5HT2A receptor with stimulation of one or more TRP receptors modulate key molecular signaling pathways that converge to reduce inflammation signaling from the gut to the brain and the developed compositions for regulating these signals show enhanced activity. Nociceptor cells have TRP channels that respond to a wide variety of spices due to the presence of specific agonist molecules they contain. The compositions include combinations of substances, including natural substances from plants and fungi or extracted from plants and fungi, in ratios that show synergistic effects, which may be due to multimodal activity at numerous receptor sites, specifically 5HT2A and at least one TRP receptor. Synergy between the 5HT2A agonist and the TRP receptor agonist allows a reduced effective amount of each compound when the composition is used, while still achieving a medicinal, therapeutic or other positive effect. The lowered amount of the 5HT2A agonist and of the TRP receptor agonist mitigates side effects of either agonist. A multimodal approach of mixed TRP agonists and TRP antagonists allow for modulation of these pathways to treat a variety of medicinal diagnosis resulting from inflammation. Inflammation associated medical conditions that the compositions may be used to treat include cancer, neurological disorders (e.g. Alzheimer's, Huntington's chorea, dementia, Parkinson's, neurodevelopmental disorders, ALS, multiple sclerosis, etc.), diabetic complications (e.g. cardiovascular disease, neuropathy, nephropathy, sepsis, hypertension, retinopathy, atherosclerosis, etc.), mental health disorders (clinical depression, post-traumatic stress disorder, bipolar disorder, schizophrenia, etc.), bone, muscular and skeletal disease (e.g. osteoporosis, osteoarthritis, muscular dystrophy, rheumatoid arthritis, osteopenia, etc.), metabolic disorders (e.g. fatty liver disease, heart disease, diabetes, metabolic syndrome, chronic fatigue syndrome, renal failure, etc.), chronic inflammatory disorders (e.g. irritable bowel disease, chronic obstructive pulmonary disease, pancreatitis, psoriasis, rheumatoid arthritis, colitis, lupus, etc.), cardiovascular disease (e.g. stroke, heart failure, congenital heart disease, atherosclerosis, cardiomyopathy, etc.).

TNF-α and COX2 are common markers for inflammatory disease and have been the targets of many drugs. TNF-α mediated inflammation was utilized in tissue cultures treated with ingredients from many of the compositions to assess anti-inflammatory potential. Many of the ingredients of the compositions reduce the prevalence of COX2 and interleukins, and may also reduce ROS, restoring homeostasis in terms of these biological signaling messengers. For each composition, there is a reduction in inflammation markers, with synergy demonstrated between the 5HT2A receptor agonist and the TRP receptor agonists.

Inflammatory cytokines COX2 and TNF-α, and ROS (e.g., inducible nitric oxide synthase (“iNOS”) are increased within the cell in response to inflammation. The inflammatory state alters sensitization of the TRP channels and results in a lower threshold signal. Inflammatory markers are key in the process also play a role in cancer development. Intracellular Ca′ buffering capacity by extracellular Ca′ entering through TRP receptor channels and being released from intracellular stores, along with subsequent activation of calcium-dependent proteases is vital to maintenance of homeostasis. Dysregulation of this process results in disease progression.

TRPV1 and other TRP proteins regulate cell-environment crosstalk, thereby influencing cell behavior. Importantly, inflammatory sensitization leads to dramatically reduced activation thresholds of TRP channels (TRPV1, TRP ankyrin type 1 (TRPA1), and TRP melastatin type 8 (TRPM8). Reduced activation thresholds in turn contribute to establishing a systemic response of energy expenditure, energy allocation, and water retention which directly modulates the immune system in chronic inflammatory diseases (Straub, 2014).

5HT2A and TRPV1 are expressed in nociceptors cells of both the ENS and the CNS. Cell bodies of sensory neurons for peripheral nociceptors are located in dorsal root ganglia alongside the spinal cord and in the trigeminal (V) ganglion for cranial nociceptors. These sensory neurons show a high expression of 5-HT receptors, including Many of these same neurons co-express TRPV1. As such, compositions were developed to specifically activate the 5HT2A and TRP signaling pathways to reduce inflammation signaling. Reducing inflammation signaling may improve mental health and other conditions.

Inflammation and sugar signaling can lead to activation of TRP receptors at reduced threshold potential, leading to an influx of Ca′ and upregulation of proinflammatory cytokines. High glutamate signaling suppresses serotonin signaling. receptor agonists including psilocin bind to 5HT2A receptors present in deep cortical layers, increasing extracellular glutamate levels in the prefrontal cortex.

The glutamate release activates AMPA and N-methyl-D-aspartate acid (“NMDA”) receptors, leading to increased expression BDNF. BDNF acts on neurons of the CNS and helps to support the survival of existing neurons and to stimulate growth and differentiation of new neurons and synapses. CREB is one of the major regulators of neurotrophin responses since phosphorylated CREB binds to a specific sequence in the promoter of BDNF and regulates its transcription. Coactivation of 5HT2A and TRP receptor channels leads to a synergistic effect from increased expression of BDNF and reactivation of pathways modulated by BDNF including an increase in GABA signaling. As a result, some balance is restored to this system, inflammation markers are downregulated, and significant mental health effects are observed.

Inflammatory cytokines can change brain function and structure through mechanisms including effects on neurotransmission. Proinflammatory cytokines increase the activity of serotonin transporter (“SERT”) proteins, resulting in an increase of serotonin reuptake and a reduction of extracellular serotonin. Moreover, proinflammatory cytokines are able to up-regulate enzymes such as tryptophan 2,3-dioxygenase (“TDO”) and indoleamine 2,3-dioxygenase (“IDO”), with a resulting decrease in tryptophan availability for serotonin synthesis, an increase in glutamate induced neurotoxicity and effects on the hypothalamic-pituitary-adrenal axis (the “HPA”) or on hippocampal neurogenesis have been observed (Lu, 2018). The HPA is an interactive neuroendocrine unit comprising of the hypothalamus, the pituitary gland, and the adrenal glands. The HPA plays a key roles in basal homeostasis and in the body's response to stress. This overall reduction in serotonin signaling also has downstream effects on gamma-aminobutyric acid, (“GABA”) signaling, which is the chief inhibitory neurotransmitter in the developmentally mature mammalian CNS. Co-Activation of TRP and 5HT2A regulates inflammation signaling pathways, leading to manipulation of these process and reduced symptoms of many diseases based on the ingredients included and which TRP channels are being influenced.

The combination of a 5HT2A agonist with a TRP agonist provides a multi-modal composition for reducing inflammation in the digestive tracts and simultaneously stimulate positive brain signalling processes. The reduced inflammation achieved through this mechanism may be useful for improving or maintaining mental health. Improving or maintaining mental health maybe useful for healthy individuals and for individuals suffering from a mental health condition or other conditions that result from inflammation. The combination a 5HT2A agonist with a TRP agonist is effective for reducing inflammation markers, which may provide benefits including a positive impact on certain mental health conditions, at lower doses (i.e. lower relative concentrations) of the 5HT2A agonist compound than would be expected through administration of the 5HT2A agonist without the TRP agonist.

Serotonin breakdown has also been a focus of treatment for depression. Monoamine oxidases (“MAO”) play an important role in the central and peripheral nervous system (CNS and PNS) by modulating the levels of monoamine neurotransmitters MAOs exist as two isoforms, MAOA and MAOB where MAOA predominantly oxidizes norepinephrine and serotonin and MAOB predominantly oxidizes dopamine. MAO inhibitors mitigate symptoms of depression through blocking breakdown of serotonin. Some ingredients have been shown to have effects on MAO, though these could be indirect though TRP signaling. MAO inhibitors are known to increase psychedelic effects. Inclusion of ingredients that may influence the activity of MAO is likely to further alter the effects of the formulations.

### Minimal Effective Dose

A therapeutically effective amount of the 5HT2A agonist may be lower in the presence of the TRP receptor agonist. By co-administering separate formulations or simultaneously administering the 5HT2A receptor agonist with the TRP receptor agonist in a single formulation, the therapeutically effective amount of the 5HT2A agonist required to achieve a therapeutic result may be lowered. Similarly, in the absence of a specific therapeutic indication but to maintain or improve wellness, co-administering the receptor agonist with the TRP receptor agonist, the amount of the 5HT2A receptor agonist needed to achieve the result is lowered. In the case of either a therapeutic indication or maintenance of wellness, a “minimum effective dose” or “MED” of the 5HT2A receptor agonist may be used when the 5HT2A receptor agonist is co-administered with a TRP receptor agonist. MED is defined as the least amount of a substance required to produce a given result, which in this case is perceptible effects and/or benefits from consumption of the 5HT2A agonist. With strongly psychoactive receptor agonists, such as some 4-substituted tryptamines, some ergolines and some phenethylamines, a therapeutic effect may be observed without inducing profound psychoactive effects in the user.

Use of a 5HT2A receptor agonist in combination with a TRP receptor agonist facilitates use of a lowered dose of the 5HT2A agonist for a defined benefit or other effect than would be necessary using the 5HT2A agonist alone. Many 5HT2A receptor agonists may be used in the compositions described here, including as detailed below. A low-cost, safe and simple 5HT2A agonist is psilocybin, which is found in fungi that biosynthesize psilocybin. Fresh sclerotia including psilocybin are available in a regulated specialty foods market in the Netherlands. Dried fruiting bodies of fungi including psilocybin are readily available in unregulated and illicit markets worldwide. Psilocybin is listed in Schedule I of the Convention on Psychotropic Substances, 21 Feb. 1971, 1019 UNTS 14956 (entered into force 8 Aug. 1975). As a result, possession of psilocybin is prohibited in many jurisdictions. However, in Jamaica and a few other jurisdictions, psilocybin is simply not scheduled in domestic controlled substance legislation and as a consequence is unregulated.

With access to Jamaica and to dried fruiting bodies that contain psilocybin, experiments with dried fruiting bodies combined with TRP receptor agonists were undertaken to qualitatively measure any change in the effects of psilocybin observed from relatively low doses of the fruiting bodies. A standardized batch of a single genetic isolate of P. cubensis was selected. Because psilocybin is psychoactive and ingestion of larger amounts of the mushroom resulted in psychoactivity, the effects were discernable in a qualitative way that supported initial experiments to identify promising TRP receptor agonists.

Microdosing psilocybin by eating dried fruiting bodies is typically done with approximately 0.3 g of dried fruiting bodies, with a general range of between 0.1 and 1.0 g, 0.2 and 0.9 g, 0.3 and 0.8 g, 0.4 and 0.7 g, 0.5 and 0.5 g of dried fruiting bodies depending on the individual and the active metabolite profile of mushroom being consumed. The effects that were observed using the selected dried psilocybin mushroom combined with one or more of the TRP receptor agonists used in the compositions. At least one published study suggests subjective effects that are less pronounced at about 0.3 g of dried fruiting bodies than through using 0.3 g of dried fruiting bodies along with at least one TRP receptor agonist (Polito, 2019).

A baseline amount of psilocybin for a MED was selected as being 0.3 g of dried fruiting bodies of the fungal species used in the formulations. This dose was consumed regularly to establish and familiarize individuals with baseline psilocybin effects resulting from consuming 0.3 g of dried fruiting bodies. For each of the initial experiments using one or more additional herbal medicinal ingredients, the same batch of ground fruiting bodies was used as a source of psilocybin.

Additional herbal medicinal ingredient were added at a 1:1 ratio of dried fruiting bodies to herbal ingredient to assess the potential for any enhancement of the medical effect of the psilocybin. Each ingredient was selected due to its reported medicinal benefits and agonist activity at least one TRP receptor. When a potential synergy was observed at 0.3 grams, the dose of both psilocybin and the additional herbal medicinal ingredient was reduced incrementally by 0.1 g, to 0.05 g from 0.1 g, of each ingredient. Incremental reductions of both ingredients were made to a point at which the effects were no longer felt. When no synergy was observed, the TRP agonist was increased incrementally in amounts of 0.3, 0.6, 0.9, 1.2 and 1.5, up to 5× the amount of psilocybin to determine whether the synergy at higher doses of TRP agonist or at different ratios of 5HT2A agonist to TRP agonist.

Combination of the 5HT2A receptor agonist (psilocybin) with additional herbal medicinal ingredients, including those with only mild effects, enhanced and altered the effects of psilocybin. The TRP agonists assessed produced synergistic effects with the psilocybin, resulting in a noticeable change in the effect of the psilocybin. In each case, the MED of psilocybin was reduced by inclusion of a TRP agonist, and the particular effects observed of a given formulation were variable depending on which TRP agonists were included with the psilocybin. The TRP agonists with the greatest effect, and particularly those resulting in a prolonged effect or in reduced side effects were considered for further compositions. The side effects that were reduced by lowering the MED were psychoactive effects from psilocybin and stomach issues from psilocybin.

An equivalent dose of amount of dried fruiting bodies and the additional herbal medicinal ingredient was taken to determine the medicinal effect or synergistic effect. A formulation including a composition of 0.3 gram of fruiting bodies and 0.3 gram of the additional herbal medicinal ingredient was consumed typically in gel capsules to reduce the difficulty of ingesting hundreds of milligrams of the additional herbal medicinal ingredients. For example, directly consuming cayenne pepper is difficult and painful. Alternatively, for oil extracts such as bergamot, peppermint and oregano, the recommended internal dose recommended on the package was consumed, which ranged from a few drops to about 1 ml depending on the liquid's concentration and the formulation.

The MED was defined as the dose required for 100% of individuals to report feeling a psychoactive effect of psilocybin, as defined subjectively by the individual. MED50 was set at the dosage where 50% of individuals report feeling the effects. Formulations of the composition were prepared based on the lowest MED50 value observed and not the average. A microdose is considered a subperceptual dose, and since MED was pegged at the lowest perceptual dose, MED50 was selected for medical benefits. For psilocybin, or for a combination of psilocybin and other 4-substituted tryptamines, between 0.1 g and 1.0 g of dried fruiting bodies is between about 1 mg and mg of psilocybin, or of psilocybin and other 4-substituted tryptamines.

Psilocybin was taken at doses of 0.0 to 1.0 g dried fruiting body, estimated to be equivalent to between 1 and 10 mg of psilocybin, suggesting a dose of 1 to 10 mg of psilocybin as 5HT2A agonist, with some of those doses being well above the microdosing range and well above the MED. For psilocybin alone, the MED was generally found to be over 0.3 g of dried fruiting bodies. For the compositions including the additional herbal medicinal ingredient, the MED was generally found to be between 0.90 and 0.12 g of dried fruiting bodies for a 70 kg individual.

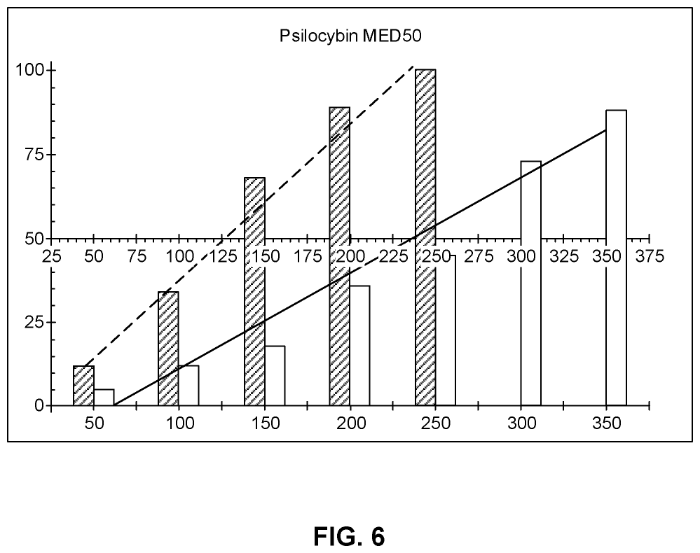

FIG. 6 shows results of MED50 determination in individuals consuming a capsule including psilocybin formulated in the 07 Base—Complete Composition (diagonal lines in bar graphs and dashed regression line) compared with capsules including psilocybin alone (white bar graphs and solid regression line). MED50 is the Dose at which 50% of participants report feeling the effect of psilocybin. Data from microdosing different amounts of psilocybin compared to the complete recipe demonstrates a reduced amount of psilocybin required to achieve an effective perceptual dose in 50% of individuals. The MED50 was about 0.240 g of dried fruiting bodies alone but about 0.125 g of dried fruiting bodies with the 07 Base—Complete Composition. Most compositions used about g of dried fruiting bodies, representing approximately 1 mg of psilocybin.

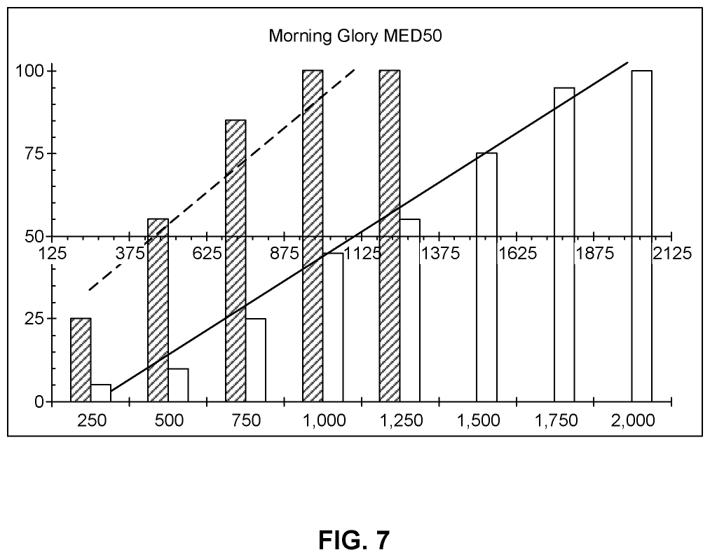

FIG. 7 shows results of MED50 determination in individuals consuming a capsule including morning glory seeds formulated in the 07 Base—Complete Composition (diagonal lines in bar graphs and dashed regression line) compared with capsules including morning glory seeds alone (white bar graphs and solid regression line) MED50 is the Dose at which 50% of participants report feeling the effect of the LSA, LSH and other ergolines present in morning glory. Data from microdosing different amounts of morning glory seeds compared to the complete recipe demonstrates a reduced amount of morning glory seeds required to achieve an effective perceptual dose in 50% of individuals. The MED50 was about 1.1 g of morning glory seeds alone but about 0.5 g of dried fruiting bodies with the 07 Base—Complete Composition. Most compositions used about 0.4 g of dried morning glory seeds, representing between approximately 260 μg and 300 μg LSA per dosage unit and between 130 μg and 525 μg LSH.

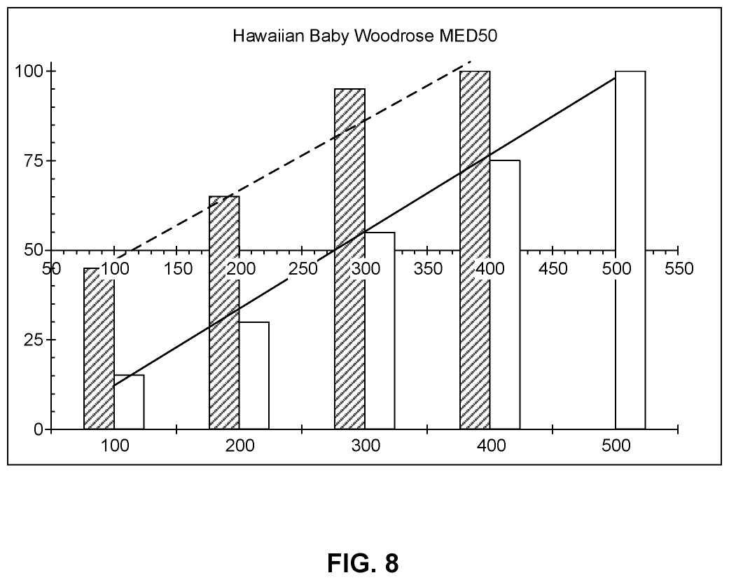

FIG. 8 shows results of MED50 determination in individuals consuming a capsule including Hawaiian baby woodrose seeds formulated in the 07 Base—Complete Composition (diagonal lines in bar graphs and dashed regression line) compared with capsules including Hawaiian baby woodrose seeds alone (white bar graphs and solid regression line) MED50 is the Dose at which 50% of participants report feeling the effect of the LSA, LSH and other ergolines present in Hawaiian baby woodrose. Data from microdosing different amounts of Hawaiian baby woodrose seeds compared to the complete recipe demonstrates a reduced amount of Hawaiian baby woodrose seeds required to achieve an effective perceptual dose in 50% of individuals. The MED50 was about 0.280 g of Hawaiian baby woodrose seeds alone but about 0.125 g of Hawaiian baby woodrose seeds with the 07 Base—Complete Composition. Most compositions used about 0.15 g of dried Hawaiian baby woodrose seeds, representing approximately 220 μg LSA, 290 μg iso-LSA, 60 μg LSH and 40 μg iso-LSH.

As detailed below in the Examples, a positive effect on mental health observed rapidly after taking the compositions. Adjustment of inflammation signalling alters signals to the brain to reduce depressive effects. Reduction in TNF-α pro-inflammatory cytokine signalling, COX2 pro-inflammatory cytokine signalling, and reduction in ROS as well as increased BDNF may all result in this positive effect on mental health.

### Receptor Agonist

### Tryptamines

The at least one 5HT2A agonist may include a tryptamine that binds the 5HT2A receptor. N,N-dimethyltryptamine or (3-[(2-dimethylamino) ethyl]indole) (“DMT”) is a hallucinogenic tryptamine drug that occurs naturally in many plants and animals. DMT is produced in the pineal gland of rats (Dean, 2019). When used orally in the absence of a monoamine oxidase inhibitor, DMT is rapidly metabolized and inactivated in the blood of most individuals. 4-substituted DMT compounds are not metabolized and inactivated in the blood to the same extent as DMT. Orally-active 4-substituted DMT compounds display psychoactive effects associated with binding at 5HT2A receptors in the human brain.

A large number of 4-substituted DMT compounds show 5HT2A agonist activity. 4-substituted DMT compounds are potent 5HT2A agonists. Many 4-substituted DMT compounds show strong psychoactive effects at doses ranging between 20 mg and above. Microdoses of 4-substituted DMT compounds may be in the range of between 5 and 50% of a flood dose of the 4-substituted DMT compounds. Using 3-[2-(dimethylamino)ethyl]-4-phosphoryloxyindole, also called psilocybin, the best known 4-substituted DMT compounds, as a reference point, a typical flood dose may be between 20 mg and 50 mg. A typical microdose of psilocybin may be between 1 and 10 mg.

The at least one 5HT2A agonist may include a 4-substituted DMT compound or where applicable, suitable salt thereof. Examples of orally-active 4-substituted DMT compounds include 3-[2-(dimethylamino)ethyl]-4-hydroxyindole, also called psilocin, any suitable salt of psilocin, psilocybin, 3-[2-(dimethylamino)ethyl]-4-acetoxyindole, also called 4-acetyl-DMT and any suitable salt of 4-acteyl-DMT.

The at least one 5HT2A agonist may include 4-substituted tryptamines that are not dimethyltryptamines. Examples of orally active 4-substituted tryptamines that are not dimethylated include the trimethyltryptamine 3-[2-(trimethylamino)ethyl]-4-phosphoryloxyindole, also called aeruginascin, the monomethyltryptamine 3-[2-(methylamino)ethyl]-4-phosphoryloxyindole, also called baeocystin, 3-[2-(methylamino)ethyl]-4-hydroxyindole, and the unmethylated tryptamines 3-[2-(amino)ethyl]-4-hydroxyindole, also called norpsilocin, and 3-[2-(amino)ethyl]-4-phosphoryloxyindole, also called norbaeocystin. Tri-methylated, monomethylated, or unmethylated tryptamines alone shows each show some 5HT2A agonism and may be preferable to psilocybin, psilocin or 4-substituted DMT compounds for use minors, those with mental health contraindications (e.g. schizophrenia, etc.) or those averse to strongly psychoactive effects.

The compositions may be formulated from fungal biomass, extracts from fungal biomass, plant biomass, plant extracts from biomass, extracts from yeast or bacterial culture systems, synthetic compounds or combinations thereof. For example, any of the 5HT2A agonists (e.g. any of the 4-substituted tryptamines) or TRP agonists described herein may be synthesized in bacterial culture systems such as E. coli. Such biomass may be sourced from fruiting bodies, mycelia, sclerotia or other biomass of fungi. Psilocin and psilocybin are 4-substituted DMT compounds found in nature. Psilocybin is more chemically stable than psilocin and is a prodrug of psilocin.

Species of fungi containing psilocybin and psilocin have been studied extensively. Psilocybin is a name based on the use of “psilocybienne” as described by the French mycologist, Roger Heim (Heim, 1958). Psilocybin shows promise for treating mental health disorder, and high dose therapies using flood doses of psilocybin have been granted breakthrough status by the FDA. Despite being regulated in many countries, consumption of psilocybin is generally considered safe in that there is little if any evidence long term negative physical consequences from ingestion of large amounts of this substance. Some studies have shown psilocybin to be one of the least damaging psychoactive substances in terms of harm to society, far behind tobacco and alcohol (Nutt, 2007). Psilocin and psilocybin are found in many genera and species of fungi, as listed below.

Psilocin and psilocybin may be found in Conocybe species including C. cyanopus, C. siligineoides and C. kuehneriana.

Psilocin and psilocybin may be found in Copelandia species including C. affinis, C. anomala, C. bispora, C. cambodginiensis, C. chlorocystis, C. cyanescens, C. lentisporus, C. tirunelveliensis, C. tropica, C. tropicalis and C. westii.

Psilocin and psilocybin may be found in Galerina species including G. steglichii.

Psilocin and psilocybin may be found in Gymnopilus species including G. thiersii, G. aeruginosus, G. braendlei, G. cyanopalmicola, G. intermedius, G. junonius, G. lateritius, G. liquiritiae, G. luteofolius, G. luteoviridis, G. luteus, G. purpuratus, G. subpurpuratus, G. validipes and G. viridans.

Psilocin and psilocybin may be found in Inocybe species including I. aeruginascens, I. aeruginascens, I. coelestium, I. corydalina, I. corydalina var. corydalina, I. corydalina var. erinaceomorpha, I. haemacta and I. tricolor.

Psilocin and psilocybin may be found in Panaeolus species including P. cinctulus, P. affinis, P. africanus, P. bisporus, P. cambodginiensis, P. castaneifolius, P. chlorocystis, P. cinctulus, P. cyanescens, P. fimicola, P. lentisporus, P. microsporus, P. moellerianus, P. olivaceus, P. rubricaulis, P. tirunelveliensis, P. tropicalis and P. venezolanus.

Psilocin and psilocybin may be found in Pholiotina species including P. cyanopus and P. smithii.

Psilocin and psilocybin may be found in Pluteus species including P. americanus, P. albostipitatus, P. americanus, P. cyanopus, P. glaucus, P. glaucotinctus, P. nigroviridis, P. phaeocyanopus, P. salicinus, P. saupei and P. villosus.

Psilocin and psilocybin may be found in Psilocybe species including P. tampanensis, P. acutipilea, P. allenii, P. angustipleurocystidiata, P. antioquiensis, P. atlantis, P. aquamarina, P. armandii, P. aucklandii, P. atlantis, P. aztecorum, P. aztecorum var. aztecorum, P. aztecorum var. bonetii, P. azurescens, P. baeocystis, P. banderillensis, P. bispora, P. brasiliensis, P. brunneocystidiata, P. cubensis, P. caeruleoannulata, P. caerulescens, P. caerulescens var. caerulescens, P. caerulescens var. ombrophila, P. caerulipes, P. callosa, P. carbonaria, P. caribaea, P. chuxiongensis, P. collybioides, P. columbiana, P. cordispora, P. cubensis, P. cyanescens, P. cyanofibrillosa, P. dumontii, P. egonii, P. fagicola, P. fagicola var. fagicola, P. fagicola var. mesocystidiata, P. farinacea, P. fimetaria, P. fuliginosa, P. furtadoana, P. tampanensis, P. galindoi, P. gallaeciae, P. graveolens, P. guatapensis, P. guilartensis, P. heimii Guzman, P. herrerae Guzman, P. hispanica Guzman, P. hoogshagenii, P. hoogshagenii var. hoogshagenii, P. hoogshagenii var. convexa, P. inconspicua, P. indica, P. isabelae, P. jacobsii, P. jaliscana, P. kumaenorum, P. laurae, P. lazoi, P. liniformans, P. liniformans var. liniformans, P. liniformans var. americana, P. mexicana, P. mairei, P. makarorae, P. mammillata, P. medullosa, P. meridensis, P. meridionalis, P. mescaleroensis, P. mexicana, P. moseri, P. muliercula, P. naematoliformis, P. natalensis, P. natarajanii, P. neorhombispora, P. neoxalapensis, P. ovoideocystidiata, P. ovoideocystidiata, P. papuana, P. paulensis, P. pelliculosa, P. pintonii, P. pleurocystidiosa, P. plutonia, P. portoricensis, P. pseudoaztecorum, P. puberula, P. quebecensis, P. ricki, P. rostrata, P. rzedowskii, P. samuiensis, P. schultesii, P. semilanceata, P. septentrionalis, P. serbica, P. sierrae, P. silvatica, P. singeri, P. squamosa, P. strictipes, P. stuntzii, P. subacutipilea, P. subaeruginascens, P. subaeruginosa, P. subbrunneocystidiata, P. subcaerulipes, P. subcubensis, P. subpsilocybioides, P. subtropicalis, P. tampanensis, P. tampanensis, P. thaicordispora, P. thaiaerugineomaculans, P. thaiduplicatocystidiata, P. uruguayensis, P. uxpanapensis, P. venenata, P. villarrealiae, P. weraroa, P. wassoniorum, P. weilii, P. weldenii, P. weraroa, P. wrightii, P. xalapensis, P. yungensis, P. zapotecorum, P. zapotecoantillarum, P. zapotecocaribaea and P. zapotecorum.

The at least one 5HT2A agonist may include 5-substituted tryptamines. Examples of 5-substituted tryptamines include 5-methoxy-DMT, also called bufotenin, N-acetyl-5-methoxy tryptamine, also called melatonin, 5-hydroxy tryptamine, also called serotonin, also called 5-HTP, 5-hydroxyl-DMT, 3-[2-(dimethylamino)ethyl]-3-[2-(dimethylamino)ethyl]-5-hydroxyindole, 3-[2-(dimethylamino)ethyl]-5-acetoxyindole, also called 5-acetyl-DMT, 3-[2-(trimethylamino)ethyl]-5-phosphoryloxyindole, 3-[2-(methylamino)ethyl]-5-phosphoryloxyindole, 3-[2-(methylamino)ethyl]-5-hydroxyindole, 3-[2-(amino)ethyl]-5-hydroxyindole and 3-[2-(amino)ethyl]-5-phosphoryloxyindole. Serotonin, melatonin and alone each some efficacy and may be preferable to bufotenine, other 5-substituted tryptamines, psilocybin, psilocin, 4-substituted-DMT compounds or other 4-substituted tryptamine compounds for use minors, those with mental health contraindications (e.g. schizophrenia, etc.) or those averse to strongly psychoactive effects.

### Ergolines

The at least one 5HT2A agonist may include ergolines. Examples of ergolines that are 5HT2A receptor agonists include lysergamides.

D-lysergic acid amide (“LSA”), also called d-lysergamide or ergine and its epimer isoergine (“iso-LSA”), is a 5HT2A agonist. A large number of lysergamides and other analogs of LSA show potent 5HT2A agonist activity. Many lysergamides show strong psychoactive effects at doses ranging between 50 μg and 5 mg, 100 μg and 4.5 mg, 200 μg and 4 mg, 300 μg and 3.5 mg, 400 μg and 3 mg, 500 μg and 2.5 mg, 600 μg and 2 mg, 700 μg and 1.5 mg, and 800 μg and 1 mg. Microdoses of lysergamides may be in the range of between 5 and 25%, 7.5 and 22.5%, 10 and 20%, and 12.5 and 17.5% of a flood dose of the lysergamide. Using D-lysergic acid diethylamide (“LSD”), the best known lysergamide, as a reference point, a typical flood dose may be between 100 μg and 350 μg, 150 μg and 300 μg, and 200 μg and 250 μg. A typical microdose of LSD may be between 10 and 25 μg, 12.5 and 22.5 μg, and 15 and 20 μg.

Lysergamides that are 5TH2A agonists with single substitutions at the amide group include D-lysergic acid ethylamide (“LAE”), lysergic acid a-hydroxyethylamide (“LSH”) and its epimer iso lysergic acid a-hydroxyethylamide (“iso-LSH”), D-lysergic acid beta-propanolamide, also called ergometrine or ergonovine, D-lysergic acid 2-butyl amide (“LSB”), D-lysergic acid 1-butanolamide, also called methylergometrine or methylergonovine, 1-methyl-D-lysergic acid butanolamide, also called methysergide, D-lysergic acid 3-pentyl amide (“LSP”), D-N-morpholinyllysergamide (“LSM-775”), D-N-pyrrolidyllysergamide (“LPD-824”), (8(3)-6-methyl-8-(piperidin-1-ylcarbonyl)-9,10-didehydroergoline (“LSD-Pip”). Lysergamides that are 5TH2A agonists with single substitutions at the amide group include N,N-dimethyllysergamide (“DAM”).

Lysergamides that are 5TH2A agonists with double substitutions at the amide group include D-lysergic acid methylisopropyl amide (“LAMIDE”), also called methylisopropyllysergamide (“MIPLA”), D-lysergic acid 2,4-dimethylazetidide (“LSZ”), LSD, D-1-acetyl-lysergic acid diethylamide (“ALD-52”), D-1-propionyl-lysergic acid diethylamide (“1P-LSD”), D-N1-butyryl-lysergic acid diethylamide (“1B-LSD”), D-N1-(cyclopropylmethanoyl)-lysergic acid diethylamide (“1 cP-LSD”), D-N1-methyl-lysergic acid diethylamide (“MLD”), D-6-ethyl-6-nor-lysergic acid diethylamide (“ETH-LAD”), D-1-propionyl-6-ethyl-6-nor-lysergic acid diethyamide (“1P-ETH-LAD”), D-6-allyl-6-nor-lysergic acid diethylamide (“AL-LAD”), D-6-propyl-6-nor-lysergic acid diethylamide (“PRO-LAD”), D-6-isopropyl-6-nor-lysergic acid diethylamide (“IP-LAD”), D-6-propynyl-6-nor-lysergic acid diethylamide (“PARGY-LAD”), D-6-butyl-6-nor-lysergic acid diethylamide (“BU-LAD”), N,N-diallyllysergamide (“DAL”) and D-N-ethyl-N-cyclopropyllysergamide (“ECPLA”).

The compositions may be formulated from fungal biomass, extracts from fungal biomass, plant biomass, microorganism biomass, extracts from biomass, synthetic compounds or combinations thereof. Such biomass may be sourced from organisms including Lysergamides or other ergolines, such as Claviceps purpurea, other species of Claviceps, some species of morning glory, including Rivea corymbosa, Ipomoea violacea, I. tricolor, I. purpurae, I. alba, Hawaiian baby woodrose species (also called elephant creeper), including Argeyreia nervosa, and Periglandula species.

### Phenethylamines

The at least one 5HT2A agonist may include phenethylamines, including amphetamines. Examples of phenethylamines that are 5HT2A receptor agonists include 3,4,5-trimethoxyphenethylamine, also known as mescaline, trimethoxyamphetamine (“TMA”), 4-bromo-2,5-dimethoxybenzeneethanamine (“2C-B”), 4-bromo-2,5-dimethoxyamphetamine (“DOB”), 4-methyl-2,5-dimethoxyamphetamine (“DOM”), 4-methyl-2,5-dimethoxybenzeneethanamine (“2C-D”), 3,4-methylenedioxyamphetamine (“MDA”), N-methyl-3,4-methylenedioxyamphetamine (“MDMA”).

The compositions may be formulated from fungal biomass, extracts from fungal biomass, plant biomass, microorganism biomass, extracts from biomass, synthetic compounds or combinations thereof. Such biomass may be sourced from organisms including mescaline and other phenethylamines, such as Lophophora williamsii, other Lophophora species, Trichocereus pachanoi, Trichocereus peruvianus, Trichocereus bridgesii, and other Trichocereus species, Echinopsis pachanoi, Echinopsis peruviana, and Trichocereus/Echinopsis scopulicola.

### Phenylpropanoids

The at least one 5HT2A agonist may include phenylpropanoids. Examples of phenylpropenes and other phenylpropanoids that are 5HT2A agonists include 5-methoxy-3,4-methylenedioxy-allylbenzene, also called myristicin, 1,2,3-timethoxy-5-(prop-2-en-1-yl)benzene, also called elemicin.

The compositions may be formulated from fungal biomass, extracts from fungal biomass, plant biomass, microorganism biomass, extracts from biomass, synthetic compounds or combinations thereof. Such biomass may be sourced from organisms including myristicin, elemicin or other phenylpropanoids, including Myristica fragrans or other species in the Myristicaceae family.

### TRP Receptor Agonist

The at least one TRP agonist compound is an agonist for a TRP receptor, and may also be an antagonist for a TRP receptor. Agonists for various TRP receptors may be applied in the compositions. TRP receptor expression is variable in different neurons as well, and the effects of 5HT2A agonists in the presence of TRP receptor agonists, including at different dosage ranges and in different ratios, allows the compositions to be targeted to various specific indications that are caused by inflammation. The at least one TRP agonist compound may include a compound that is generally regarded as safe (“GRAS”). The at least one TRP agonist compound may be sourced from cayenne, turmeric, clove, cinnamon or nutmeg. The at least one TRP agonist compound may include capsaicin or other capsiates, curcumin or other curcuminoids, eugenol, β-caryophyllene, cinnamaldehyde, myristicin, elemicin, α-terpineol or 8-O-4′-neolignans.

The at least one TRP agonist compound may include a compound that is GRAS. The at least one TRP agonist compound may be sourced from cannabis, bergamot, oregano, thyme, cardamom, peppermint, eucalyptus, pepper, ginger, garlic or onion. The at least one TRP agonist compound may include β-caryophyllene, α-terpineol, cannabidiol (“CBD”), cannabidivarin (“CBDV”), cannabigerol (“CBG”), cannabigerolic acid (“CBGA”), delta-9-tetrahydrocannabivarin (“THCV”), delta-9-tetrahydrocannabivarinic acid (“THCVA”), cannabigevarin (“CBGV”), myrcene, eriodictyol, carvacrol, myrcene, thymol, carvacrol, menthol, 1-8 cineole, piperine, gingerol, allicin, thymol and combinations thereof. Cacao also seemed to show some effect both with onset and duration though not as pronounced.

The specific compounds sourced from cayenne, turmeric, clove, cinnamon, nutmeg, cannabis, bergamot, oregano, thyme, cardamom, peppermint, eucalyptus, pepper, ginger, garlic and onion listed above have activity at the TRPV1, TRPA1, TRPM8 and TRPV3 receptors as illustrated in Table 2.

TABLE 2
Agonism and antagonism of TRPV1, TRPA1, TRPM8 and TRPV3

| | GRAS | | | | |
|---|---|---|---|---|---|
| Active | Substance | TRPV1 | TRPA1 | TRPM8 | TRPV3 |
| capsaicin | Cayenne | +++ | n/a | n/a | n/a |
| | Pepper | | | | |
| curcumin | Turmeric | (−) | ++ | n/a | n/a |
| eugenol | Turmeric | + | n/a | ++ | ++ |
| | Clove | | | | |
| | Cinnamon | | | | |
| | Pepper | | | | |
| β-caryophyllene | Turmeric | n/a | n/a | n/a | + |
| | Clove | | | | |
| | Cinnamon | | | | |
| | Cannabis | | | | |
| | Pepper | | | | |
| cinnamaldehyde | Cinnamon | n/a | ++ | + | n/a |
| myristicin | Nutmeg | + | n/a | n/a | n/a |
| elemicin | Nutmeg | + | n/a | n/a | n/a |
| alpha terpineol | Turmeric | n/a | + | n/a | n/a |
| | Nutmeg | | | | |
| | Cannabis | | | | |
| 8-O-4′-neolignans | Nutmeg | n/a | n/a | + | n/a |
| (−)-epicatechin | Cacao | n/a | n/a | n/a | + |
| | Green Tea | | | | |
| CBD | Cannabis | + | + | + | + |
| CBDA | Cannabis | + | + | + | + |
| CBDV | Cannabis | + | + | + | n/a |
| CBG | Cannabis | + | + | + | n/a |
| CBGA | Cannabis | + | + | + | + |
| CBGV | Cannabis | + | + | + | + |
| THC | Cannabis | n/a | n/a | + | n/a |
| THCA | Cannabis | n/a | n/a | + | n/a |
| THCV | Cannabis | + | + | + | + |
| THCVA | Cannabis | + | + | + | + |
| myrcene | Cannabis | + | n/a | n/a | n/a |
| | Bergamot | | | | |
| | Others | | | | |
| eriodictyol | Bergamot | n/a | n/a | n/a | + |
| carvacrol | Bergamot | n/a | n/a | + | n/a |
| | Oregano | | | | |
| thymol | Oregano | n/a | + | + | + |
| | Thyme | | | | |
| linalool | Bergamot | n/a | n/a | + | n/a |
| | Cardamom | | | | |
| | Cannabis | | | | |
| menthol | Peppermint | n/a | n/a | +++ | n/a |
| 1-8 cineole | Peppermint | n/a | (−) | + | n/a |
| | Eucalyptus | | | | |
| | Cardamom | | | | |
| piperine | Pepper | + | + | n/a | n/a |
| gingerol | Ginger | + | n/a | n/a | n/a |
| allicin | Garlic | n/a | + | n/a | n/a |
| | Onion | | | | |
| incensole | Myrrh | n/a | n/a | n/a | +++ |
| incensole acetate | Frankincense | n/a | n/a | n/a | + |

As shown in Table 2, the at least one TRP agonist compound may include a TRPV1 receptor agonist. TRPV1 agonists may include capsaicin, eugenol, myristicin, elemicin, CBD, CBDA, CBDV, CBG, CBGA, CBGV, THCV, THCVA, myrcene, piperine and gingerol. TRPV1 agonists reduce inflammation by competing for the TRP receptor site and sending altered signals that reduce expression of inflammation markers and generation of ROS. Inclusion of a TRPV1 agonist lowers the amount of 5HT2A agonist required for a given effect, including the MED of 5HT2A agonist. Inclusion of a TRPV1 agonist alters the effects of the formulation by reducing inflammation, improving digestion and improving symptoms of depression.

As shown in Table 2, the at least one TRP agonist compound may include a TRPA1 receptor agonist. TRPA1 agonists may include curcumin, cinnamaldehyde, alpha terpineol, CBD, CBDA, CBDV, CBG, CBGA, CBGV, THCV, THCVA, thymol, piperine, allicin. Some of these molecules may also act as antagonists for other TRP receptors by reducing signalling associated with other endogenous compounds associated with inflammation. For example, curcumin has shown to antagonize the effects of capsaicin at TRPV1 (Zhi, 2013). Inclusion of these molecules allowed for a reduced dose of the 5HT2A agonist and may alter the effects of the formulation reducing inflammation, improving digestion and improving mental health.

As shown in Table 2, the at least one TRP agonist compound may include a TRPM8 receptor agonist. TRPM8 agonists may include eugenol, cinnamaldehyde, CBD, CBDA, CBDV, CBG, CBGA, CBGV, THC, THCA, THCV, THCVA, carvacrol, thymol, menthol and 1-8 cineole. Activation of TRPM8 typically results from sensitization from cold and inclusion of molecules with activity at this receptor may alter the effects of the formulations, in some cases improving alertness, ability, and enhancing sexual experiences, including by increased libido.

As shown in Table 2, the at least one TRP agonist compound may include a TRPV3 receptor agonist. TRPV3 agonists may include eugenol, β-caryophyllene, (−)-epicatechin, eriodictyol, cinnamaldehyde, incensole, thymol and cannabinoids. Activation of TRPV3 typically results from sensitization to warm temperature and inclusion of molecules with activity at this receptor alters the effects of the formulations mood.

### Capsaicin

Capsaicin is a well-known and potent potentiator of the TRPV1 receptor. Capsaicin has been shown to have healing properties as a topical for inflammation and pain. A product including capsaicin as an active pharmaceutical ingredient has been approved by the United States Food and Drug Administration (the “FDA”) for topical pain relief.

A combination of capsaicin and a 5HT2A agonist improves the anti-inflammatory properties and the psychological benefits of both compounds. A combination of capsaicin and psilocybin decreased the onset time of the psilocybin, with perceived effects occurring within 15 to 30 minutes after ingestion. The peak effects of the psilocybin had a longer duration but the overall duration of the effects appeared unchanged. Capsaicin with psilocybin improved pain and inflammation in individuals with acute symptoms and improved mood often within one dose. Some individuals reported lasting positive benefits on pain levels and mental health for days following a single dose, and long after psychoactive effects of psilocybin had ended. The combination of capsaicin with psilocybin allowed a reduction in the dose of psilocybin required for perceived effects to reduce inflammation and peripheral pain, resulting in a lower amount of psilocybin at MED50. Increased alertness was also reported relative to psilocybin alone or relative to baseline.

The dose of capsaicinoids in dosage forms of the composition was calculated at between 0 and 2.5 mg based on inclusion of between 0 and 1 g of dried cayenne pepper. However, the dose of capsaicinoids in dosage forms of the composition can also be between 0.5 and 2 mg, and 1 and 1.5 mg. Cayenne pepper, or red chili pepper, typically includes about 300 μg capsaicinoids of gram dry weight reported in ground cayenne pepper (Al Othman, 2011) but has a reported range up to 2.5% w/w.

The spicy flavor of capsaicin may upset stomach and result in pain in some individuals. Capsaicin also caused intestinal issues at large doses. Various peppers were utilized as a source of capsaicin, with cayenne pepper being the most effective without side effects. While a similar amount of capsaicin per gram would be expected from many species, though there may be variations in minor capsiates. Many species caused serious stomach pains or gastrointestinal pains in some people, particularly people who do not typically consume spicy food. Heartburn was also occasionally reported. However, no negative effects were reported from the cayenne pepper utilized at the doses listed. While the spicy flavor is still notable in chocolate formulations that include the compositions, it was often masked to some extent by the other ingredients in the composition and was tolerable to most people at the ratio included. For some, capsules were preferred over chocolates due to the spicy flavor of cayenne.

Species from the Solanaceae family that contain capsaicin are summarized in the below Table 3.

TABLE 3
Capsaicinoids in commonly used peppers

| | | | Capsaicinoids |
|---|---|---|---|
| Species | Common Names | Part | (ppm) |
| Capsicum | Cherry Pepper, Sweet | Resin, exudate, sap | 634,000 |
| annuum | Pepper, Bell Pepper, | Fruit | 4,000 |
| | Paprika, Green Pepper, | Tissue culture | 590 |
| | Cone Pepper | | |
| Capsicum | Hot Pepper, Tabasco, | Fruit | 17,900 |
| frutescens | Cayenne, Chili, Red | Seed | 5 |
| | Chili, Spur Pepper | | |

### Curcumin

Curcuminoids, are found in the spice turmeric from the ginger family Zingiberacea. (1E,6E)-1,7-bis(4-hydroxy-3-methoxyphenyl) hepta-1,6-diene-3,5-dione, also called curcumin, is a linear diarylheptanoid and curcuminoid. Curcumin is a phytopolylphenol pigment isolated from Curcuma longa. Curcumin is used in a variety of products including herbal supplements, food coloring, food flavoring, and cosmetics. Curcumin belongs to a group of compounds known as curcuminoids. Curcumin is a tautomeric compound, stable in both enolic form in an organic solvent and in a keto form when in water. Curcumin is a TRPA1 receptor agonist and antagonize the effects of TRPV1 receptor agonists when combined (Yeon et al, 2010).

In addition to curcumin, species of turmeric also contain various amounts of antioxidant molecules sybisabolone-9-one, 4-methyllene-5-hydroxybisabola-2,10-diene-9-one, turmeronol B, 5-hydroxy-1,7-bis(4-hydroxy-3-methoxyphenyl)-1-hepten-3-one, 3-hydroxy-1,7-bis(4-hydroxyphenyl)-6-hepten-1,5-dione, cyclobisdemethoxycurcumin, bisdemethoxycurcumin and demethoxycurcumin (Akter, 2019). Anti-inflammatory and antidepressant activity of turmeric and extracts from turmeric may be due to biologically active molecule other than curcumin, such as these antioxidants or other molecules present in the turmeric, in addition to curcumin.

Curcumin has been investigated in numerous clinical and laboratory trials. Curcumin has been challenging to develop as an API in a drug because it is unstable, reactive and has limited bioavailability (Nelson, 2017). Nonetheless, curcumin has shown promise as a therapeutic agent for its activity as an antioxidant (Sreejayan, 1994), anti-inflammatory (Brouet, 1995), anticarcinogenic (Rao, 1995), antimicrobial (Limtrakul, 1997) hypoglycemic (Arun, 2002) and anti-depressive effects (Kulkarni, 2009). Curcumin has antidepressant effects in animal models. Chronic, but not acute, administration of 150 mg/kg curcumin significantly raised anandamide levels in a variety of brain regions (Smalheiser, 2019).

Curcumin shows agonist activity at TRPA1 and antagonist activity at TRPV1. Curcumin is also listed as an MAO inhibitor (Kulkarni, 2008) and while it does not appear to cross the blood-brain barrier and the active form is not detected to a significant extent in the blood, yet curcumin has been shown to increase levels of BDNF and reduce depression. Studies suggest that curcumin can normalize depressive-like behaviors, which may be independent of concurrent analgesic action. Correction of depressive behavior could potentially be mediated by the supraspinal serotonergic system and downstream GABA receptor (Zhao, 2014).

Curcumin has been extensively studied as a medicine, with nearly 6,000 published citations, most of which have appeared within the past 20 years. Wikipedia indicates that “Curcumin, which shows positive results in most drug discovery assays, is regarded as a false lead that medicinal chemists include among “pan-assay interference compounds”. This attracts undue experimental attention while failing to advance as viable therapeutic or drug leads. [3][11][12]. Factors that limit the bioactivity of curcumin or its analogs include chemical instability, water insolubility, absence of potent and selective target activity, low bioavailability, limited tissue distribution, and extensive metabolism.[3] Very little curcumin escapes the GI tract and most is excreted in feces unchanged” This interpretation shows a failure to understand that gut biology was also important for brain health, and that signals could be sent to the brain without the molecule itself ever leaving the digestive tract or entering the blood stream. The lack of attention on curcumin specifically demonstrates how a traditional pharmaceutical approach to depression that targets the brain has neglected and ignored useful compounds in favor of compounds that cross the blood brain barrier and target a specific receptor with high affinity. Put otherwise, due to the complexity of the system and lack of molecular evidence for a mechanism of action on these compounds in the brain, they were ignored in favor of compounds with measurable activity at a neurotransmitter receptor in the brain.

A combination of curcumin and a 5HT2A agonist improves the anti-inflammatory properties and the psychological benefits of both compounds. Curcumins presence in the compositions increased the length of the subjective effects of psilocybin reported by the user and also improved mental health benefits. A combination of curcumin and psilocybin decreased the dosage of psilocybin at MED and prolonged the effects of the psilocybin at the dosage. Doses of curcumin were based on curcumin accounting for about 6% of the dry weight of turmeric. Turmeric was provided in approximately a 1:1 ratio of weight compared with dried fruiting bodies.

A combination of psilocybin and curcumin may antagonize or provide competition for binding at the TRPV receptor when present with capsaicin or other TRPV agonists, resulting in a longer duration of effects and a greater impact on mental health. Rather than a quick onset and rapid drop off with effects stopping within two hours, this combination resulted in a sustained effect for 4 to 6 hours. Combining curcumin with capsaicin and psilocybin also lead to greater anti-inflammatory activity than capsaicin and psilocybin alone. Combining curcumin with capsaicin and psilocybin also allowed the MED of capsaicin to be reduced by 50% while still achieving a similar effect. For example, a 2:2:1 ratio of dried psilocybin fruiting bodies:turmeric:cayenne pepper provided an effective MED at 160 mg:160 mg:80 mg. A combination of psilocybin with turmeric synergistically improved inflammation and depression without increasing anxiety, in contrast with a combination of capsaicin and psilocybin, or psilocybin alone.

Species from the Curcuma genus that may be applied to the compositions disclosed herein include C. aeruginosa (pink and blue ginger), C. albicoma, C. albi ora, C. alismatifolia (summer tulip), C. amada (mango ginger), C. amarissima, C. Americana, C. angustifolia (tall hidden ginger), C. aromatica, C. attenuata, C. aurantiaca (rainbow curcuma), C. aurantiflora, C. australasica (Cape York turmeric), C. bakeriana, C. bicolor (candy corn), C. brog, C. burttii, C. caesia, C. cannanorensis, C. cannanorensis var. Lutea, C. caulina, C. careyana, C. certothecca, C. chuanezhe, C. chuanhuangjiang, C. chuanyujin, C. cochinchinensis, C. codonantha, C. coerulea, C. colorata, C. comosa, C. cordata (jewel of Thailand), C. cordifolia, C. coriacea, C. decipiens, C. domestica (Emperor variegated), C. ecalcarata, C. ecomata, C. elata (giant plume), C. erubescens, C. euchroma, C. euclroma, C. exigua, C. ferruginea, C. flaviflora (red fireball ginger), C. glans, C. glaucophylla, C. gracillima (chocolate zebra), C. grahamiana, C. grandiflora, C. haritha, C. harmandii (emerald pagoda ginger), C. heyneana, C. inodora (pink ginger), C. karnatakensis, C. kudagensis, C. Ifwangsiensis, C. Icwangsiensis varaffinis, C. Ifwangsiensis var. puberula, C. lanceolata, C. latiflora, C. latifolia, C. leopoldi, C. leucorhiza, C. loerzingii, C. lillicina (pink cloud), C. longa (turmeric), C. longiflora, C. longi spica, C. lutea, C. malabarica, C. mangga, C. meraukensis, C. montana, C. musacea, C. mutabilis, C. neilgherrensis, C. nilamburensis, C. ochrorhiza, C. ofcinalis, C. olena, C. oligantha (white turmeric), C. oligantha var. lutea, C. ornate (ornate plume ginger), C. pallida, C. parviflora (white angel), C. parvula, C. peethapushpa, C. petiolata (Temu Puteri in Java), C. phaeocaulis, C. pierreana (sleeping princess), C. plicata, C. porphyrotaenia, C. prakasha, C. pseudomontana, C. purpurascens, C. purpurea, C. raktakanta, C. ranadei, C. reclinata, C. rhabdota, C. rhomba, C. roscoeana (pride of Burma), C. rotunda, C. rubescens (wine red plume), C. rubricaulis, C. rubrobracteata (fire plug), C. sessilis, C. siamensis, C. sichuanensis, C. singularis (Easan white), C. soloensis, C. sparganifolia, C. speciosa, C. spicata, C. stenochila, C. strobilifera, C. sulcata, C. sumatrana (Sumatra ginger), C. sylvatica, C. sylvestris, C. thalakaveriensis, C. thorelii (Chiang Mai Snow), C. trichosantha, C. vamana, C. vellanikkarensis, C. viridi ora, C. wenchowensis, C. wenyujin, C. xanthorrhiza, C. yunnanensis (Yunnan plume ginger), C. zanthorrhiza (temulawak), C. zedoaria (zedoary white turmeric) and C. zerumbet.

### Eugenol

In addition to curcumin, turmeric also contains eugenol. Eugenol can act as a local analgesic and is an agonist for TRPV1 and a strong agonist for TRPV3 (Xu, 2006). Turmeric may include about 0.3% essential oil, of which about 8% may be eugenol (Stanojević, 2015), supporting an estimate of about 240 μg eugenol per gram of turmeric.

In addition to turmeric, eugenol is also found in clove from the Caryophyllus aromaticus tree. Eugenol is used in dentistry as a topical analgesic (Chung, 2013). Eugenol is found in concentrations of ranging from 9,400 mg to 14,600 mg per 100 g of fresh plant material (Cortes-Rojas, 2014). Clove represents one of the major vegetal sources of phenolic compounds as flavonoids, hydroxybenzoic acids, hydroxycinnamic acids and hydroxyphenyl propenes. Clove has historic use for toothache, joint pain and antispasmodic. The voltage dependent effects of eugenol in sodium and calcium channels and in receptors expressed in the trigeminal ganglion may be primarily responsible for the analgesic effect of clove (Wang, 2015).

Eugenol was selected for its potential to bind TRP receptors and its potential to interfere with several other cell-signaling pathways, specifically the nuclear factor kappa B (“NF-κB”). This factor is activated by free radicals and results in the expression of genes that suppress apoptosis and induce cellular transformation, proliferation, invasion, metastasis and other phenomena associated with cancer progression (Hoesel, 2013). Inclusion of the eugenol reduces inflammatory markers and ROS signaling from the gut to the brain.

Inclusion of ingredients including significant amounts of eugenol resulted in reports of a calming effect compared with psilocybin alone, with capsaicin and psilocybin or with capsaicin, curcumin and psilocybin. Eugenol is included in the compositions disclosed herein primarily through clove, turmeric and black pepper. The dose of eugenol was calculated at between 0.1 and 1.6 g based on 17-20% maximum amount in the ingredients, with between 0.1 and 5 grams of turmeric and clove daily, and in some cases also the addition of black pepper. In some aspects, other doses of eugenol could be between 0.2 and 1.5 g, 0.3 and 1.4 g, 0.4 and 1.3 g, 0.5 and 1.2 g, 0.6 and 1.1 g, 0.7 and 1.0 g, and 0.8 and 0.9 g. Peripheral antinociceptive activity of eugenol has been reported as showing significant activity at doses of 50, 75 and 100 mg/kg (Daniel, 2009).

Other herbal sources of eugenol are summarized in Table 4. The concentrations for some of the herbal ingredients listed in Table 4 are cited from (Khalil, 2017). In addition to the sources of eugenol listed in Table 4, ginger, oregano, basil, mace, pepper and marjoram also include eugenol.

TABLE 4
Eugenol in commonly used herbal ingredients

| Common Names | Part | Eugenol (mg/g) |
|---|---|---|
| Clove | Flowers and buds | 180 |
| Clover | Fruit | 36 |
| Betel Pepper | Leaves | 18 |
| Cinnamon | Bark | 3.5 |
| Tulsi | Leaves | 4 to 5 |
| Bay | Leaves | 1.3 |
| Turmeric | Leaves and Essential Oil | 2.1 |
| Nutmeg | Seeds | 0.3 |
| Thyme | Shoots | 0.02 |

### β-Caryophyllene

Clove and turmeric contains significant amounts of (β-caryophyllene (“BCP”), which is thought to be an agonist of the CB1 receptor and also a TRPV1 receptor agonist (Sharma, 2015). Clove may include about 2% BCP. Turmeric may also include BCP. Agonism at both CB1 and TRP receptors highlights the indirect linkage and cross talk between the TRP and ECS pathways. Numerous molecules are agonists on both sets of receptors. As shown in FIG. 5, arachidonic acid signaling interacts with TRP receptor signaling pathways. ECS stimulation results in arachidonic acid metabolism.

BCP was approved by the FDA and the European Food Safety Authority (EFSA) for human consumption. BCP is also used as flavor enhancer and in cosmetics (Sköld, 2016). Neuroinflammation or inflammation of the brain a process leading to nervous system degeneration, characterized by the activation of monocytes, macrophages, mast cells, lymphocytes, and the production of inflammation mediators, such as nitric oxide (“NO”), various cytokines (IL-1β, interleukin-6 (“IL-6”), interleukin-8 (“IL-8”) and TNF-α), NF-κB and prostaglandins. While curcuminoids do not pass the blood-brain barrier, BCP does pass the blood-brain barrier and has anti-inflammatory effects directly on the brain.

Even at the low doses being administered, BCP being included in the compositions in addition to psilocybin alone improved mood in those with both depression and anxiety issues. Recipes including clove or clove oil contain both significant eugenol and some BCP. The isolated molecules have been tested in vitro and found to both possess anti-inflammatory properties.

Herbal sources of BCP are summarized in Table 5.

TABLE 5
Herbal Sources of BCP

| Species | Family | Common Names | Part | BCP (mg/g) |
|---|---|---|---|---|
| Cannabis sativa | Cannabaceae | Marijuana | flower | 3.8-37.5* |
| Carum nigrum | Ranunculacea | Black caraway | seeds | 7.8* |
| Syzygium aromaticum | Myrtaceae | Clove | fruit, | 1.7-19.5* |
| | | | stems, | |
| | | | buds | |
| Humulus lupulus | Cannabaceae | Hops | fruits | 5.1-14.5* |
| Ocimum gratissimum | Lamiaceae | Clove basil | leaves | 5.3-10.5* |
| Ocimum micranthum | Lamiaceae | Wild basil | leaves | 4-19.8* |
| Origanum vulgare | Lamiaceae | Oregano | leaves | 4.9-15.7* |
| Piper nigrum | Piperaceae | Black pepper | fruit | 7.29* |
| Piper aleyreanum | Piperaceae | Black pepper | fruit | 15.9 |
| Lavandula angustifolia | Lamiaceae | Lavendar | Flowers, | 4.62-7.55* |
| | | | leaves | |
| Rosmarinus officinalis | Lamiaceae | Rosemary | Flowers, | 0.1-8.3* |
| | | | leaves | |
| Cinnamomum | Lauraceae | True cinnamon | Seeds, | 6.9-11.1* |
| zeylanicum | | | bark, | |
| | | | leaves | |
| Cinnamomum tamala | Lauraceae | Tamala | Leaves | 25* |
| Cananga odorata | Annonaceae | Cananga | Leaves, | 3.1-10.7* |
| | | | flowers | |
| Copaifera langsdorffii | Fabaceae | Diesel tree | Fruit | |
| Orthosiphon stamineus | Lamiaceae | | | 24-35* |
| Knema kunstleri | Myristicaceae | | | 23* |
| Croton glandulosus | Euphorbiaceae | | | 53.2* |
| Pterodon emarginatus | Fabaceae | Sucupira | fruits | 20.3 |
| Cymbopogon olivieri | | | | 14.4 |
| Pachira aquatica | Malvaceae | Money tree | | 11.5 |
| Staudtia gabonensis | Myristicaceae | | Seeds | 12.2 |
| | | | and bark | |
| Gnetum africanum | Gnetaceae | | leaves | 18.1 |
| Lallemantia peltata | Lamiaceae | | flowers | 20 |
| Piper cyrtopodon | Piperaceae | | Aerial | 34.6 |
| | | | parts | |
| Mentha aquatica | lamiaceae | | Aerial | 12.8 |
| | | | parts | |
| Renealmia alpinia | Zingiberacea | Honey bract | Leaves | 22.9 |
| Persea americana | Lauraceae | Avocado | leaves | 43.9 |
| Vitex negundo | lamiaceae | Chinese | fruits | 36 |
| | | chaste tree | | |
| Oyedaea verbesinoides | Asteraceae | | | 27.1 |
| Vitex doniana | Verbenaceae | Sweet | leaves | 12.6 |
| Salvia leucantha | lamiaceae | | leaves | 10.7 |
| Phlomis cancellata | Labiatae | | Aerial | 17 |
| | | | parts | |
| Chromolaena odorata | Asteraceae | Christmas bush | leaves | 25.2 |
| Cnidium silaifolium | Apiaceae | | Aerial | 8.2 |
| | | | parts | |
| Zosimia absinthifolia | Umbelliferae | | | 22.2 |
| Lantana canescens | Verbenaceae | | | 16.3 |
| Pimpinella kotschyana | Apiaceae | | Full | 40 |
| | | | plant | |
| Acalypha fruticosa | Euphorbiaceae | | leaves | 42 |
| Petitia domingensis | lamiaceae | | flowers | 35.7 |
| Scutellaria havanensis | lamiaceae | | leaves | 75.6 |
| Garcinia mangostana | Clusiaceae | Mangosteen | Leaves | 17-21 |
| | | | bark | |
| Clerodendrum | lamiaceae | Bagflower | leaf | 28.9 |
| polycephalum | | | | |
| Centaurea imperialis | Asteraceae | Centaury | | 14.1 |
| Lantana camara | Verbenaceae | | | 10.1 |

*percentage BCP in essential oil from that part

Other sources of BCP include thyme (Thymus vulgaris), sage (Salvia officinalis), mint (Mentha piperita) and ginger (Zingiber officinale),

### Cinnamaldehyde

Cinnamon zeylanicum (cinnamon) is widely used in traditional system of medicine to treat diabetes in India. Cinnamon contains both eugenol and BCP. In addition, Cinnamon has a significant amount of the biologically active molecule (2E)-3-phenylprop-2-enal, also known as cinnamaldehyde, which is a phenylpropanoid that gives cinnamon its flavor and odor. Cinnamaldehyde is found in the bark of cinnamon trees and other species of the genus Cinnamomum. Cinnamaldehyde has a range of uses including as a flavoring, an agrichemical and as a corrosion inhibitor. Cinnamaldehyde has also been shown to possess antibacterial (Doyle, 2019), antiviral (Feng, 2020), antifungal, anticancer (Tian, 2017), antipyretic (Sui, 2010), and anti-obesity (Jiang, 2017) properties. Though some of these may be attributed to the activity of BCP and eugenol, cinnamaldehyde has been shown to activate TRPA1, which may provide an additional synergistic effect (Bandell, 2004).

Cinnamaldehyde was found to cause nociceptive behavior in mice when administered via intraplantar injections (Bandell, 2004). Iwasaki et al. (2008) demonstrated that cinnamaldehyde was capable of increasing adrenaline secretions in rats (Iwasaki, 2008; Anderson 2013).

The dose of cinnamaldehyde was based on a content of 0.5% to 3.0% in cinnamon species and the inclusion of between 0 and 5 grams of ingredients containing cinnamaldehyde per dose. In some aspects, doses of cinnamaldehyde could be between 0.6% to 2.9%, 0.7% to 2.8%, 0.8% to 2.7%, 0.9% to 2.6%, 1.0% to 2.5%, 1.1% to 2.4%, 1.2% to 2.3%, 1.3% to 2.2%, 1.4% to 2.1%, 1.5% to 2.0%, 1.6% to 1.9%, and 1.7% to 1.8%. The dosing of cinnamaldehyde was estimated based on up to 3.0% cinnamaldehyde in cinnamon.

Addition of cinnamon allowed a reduction in the amount of clove in the recipe. Reducing clove and adding cinnamon may also improve flavor in formulations of the composition. Addition of cinnamon also appeared to reduce sugar cravings. Addition of cinnamon also may provide appetite suppression, weight loss and mitigation of uric acid crystals, which may be associated with gout. Cinnamon also helped mitigate aggression in men, potentially due to testosterone reduction in cases with elevated testosterone.

### Endocannabinoids and Phytocannabinoid Agonists

Endocannabinoids (“ECs”) and phytocannabinoids both at least partially modulate biological process via cannabinoid receptor types 1 (“CB1”) and cannabinoid receptor type 2 (“CB2”). ECs and phytocannabinoids have also been shown to interact with the TRP receptors. The TRP receptors and the CB receptors are linked through the activity of endogenous ECs, such as AA and arachidonyl ethanolamide/anandamide (“AEA”). AA has been shown to be an agonist at TRPA1, TRPM5, TRPV3 and TRPM2. AA is also an antagonist at TRPM8. AEA is an agonist of TRPV1 and TRPA1.

The differencing activity of ECs at the various TRP receptors indicated that phytocannabinoids also influence TRP channels and modulated the effects of the composition. Indeed, TRPA1 is also activated by THC and cannabinol (“CBN”). THC is the major psychoactive cannabinoid in Cannabis sativa. THC and cannabinol activate TRPA1 in TRPA1 overexpressing CHO cells and in trigeminal neurons. When consumed by humans, THC produces a wide range of biological effects, such as an increase in pulse rate, decreased blood-pressure, muscle weakening, increased appetite, and euphoria, followed by drowsiness (Ciardo, 2017). CBD, CBG, CBN and other cannabinoids in contract may produce markedly different effects without significant psychoactive effects.

Extracts or inclusion of either clove or cannabis were each found to have synergistic effects with low doses of psilocybin. Each such composition includes molecules active at TRP receptors and molecules active at CB receptors.

Similar metabolic pathways in individuals suffering from either post-traumatic stress disorder (“PTSD”) and traumatic brain injury (“TBI”). Specifically, lower endocannabinoid signaling coupled with blocked calcium signaling downstream of TRP receptors CBD and other brain stimulation and neuroprotective molecules were included in compositions directed to improvement of mental health or treatment of the effects of brain injury.

A demographic of regular psilocybin users are often regular cannabis consumers as well. Cannabis is typically consumed as an unknown varietal and smoked in most instances where the actual amount has not been measured. However, low doses of cannabis in combination with psilocybin have not been reported or studied. In addition to the inclusion of a TRP agonist, the compositions may optionally include cannabis or ingredients from cannabis at doses below those required to cause significant psychoactive effects. Cannabis contains many phytocannabinoids that interact with the CB1 and CB2 receptors. THC and CBD are the most common phytocannabinoids. The precursor to each of THC and CBD is CBG. Cannabinol is a degradation product of THC that may be present in cannabis. THC converts to CBN under heat or catalysis. Additional rare phytocannabinoids such as CBDV have also shown to bind with TRP receptors.

Inclusion of phytocannabinoids extracted from select varieties of cannabis administered at low doses in the compositions facilitated reduced anxiety and increased relaxation, and also showed increased libido in some individuals. Cannabis also includes many terpenoids, some of which have been shown to influence the TRP receptors. A dominant terpene in many cannabis varieties is myrcene. The compositions may also be prepared with an isolate of a phytocannabinoids to mitigate potential synergy, antagonism or contra-indications that may have occurred form the addition of unknown phytocannabinoids or terpenoids. In some cases CBD isolate was used to avoid the psychoactive properties that may result from the presence of other cannabinoids.

In many jurisdictions, cannabis is prohibited, and while an adult use market for cannabis is regulated in Canada, psilocybin remains prohibited in 2020 and Canadian regulations do not allow for 5HT2A agonists to be added to cannabis products in significant quantities. Clove was chosen as the preferred ingredient to include in most compositions to stimulate both a TRP receptor and potentially the CB receptors. Including cannabis or clove provided a more relaxing effect and promoted deeper sleep after the dose and again further reduced inflammation.

### Myristicin

Nutmeg contains myristicin, which is also found in carrot, basil, cinnamon, and parsley. Myristicin, or methoxysafrole, is a benzodioxole that is active at the 5HT2A receptors in the brain with slight MAO inhibiting properties. Myristicin has hypotensive, sedative, anti-depressant, anesthetic, hallucinogenic, and serotonergic properties. Elemicin is another molecule has similar structure and shows similar effects. Both myristicin and elemicin appear to act as a significant potentiator of GABA. Large doses may cause hyper-excitability and may cause CNS depression among other negative effects. Nutmeg may include between 20 and 30 mg per gram, and the amount of myristicin in the compositions formulated with nutmeg was estimated to be up to 3% w/w.

Nutmeg also contains alpha terpineol and 8-O-4′-neolignans, which activate the TRPA1 and TRPM8 receptors respectively. Agonism at TRPA1 and TRPM8 may amplify antidepressant effects of the compositions and potentially support cessation of SSRIs. Many individuals who were taking SSRIs required a higher dose of the compositions in order to feel the effects of the compositions. With psilocybin and nutmeg, in some cases individuals were able to ween from (through sequentially reducing the dose) and/or replace their SSRIs within a matter of weeks without significant issues.

Nutmeg appeared to help with the and improve the overall antidepressant qualities and allow for a reduction in the amount of psilocybin (to about 0.1 g of dried fruiting bodies) while still achieving an improvement in mood, reduced anxiety, reduced depression or other therapeutic effects. This translated to improved happiness and elevation of positive thoughts associated with the compositions. Including nutmeg and reducing the amount of psilocybin also improved the therapeutic effects and allow for a better sleep. However, including nutmeg with psilocybin amplified the psychoactive effects of the psilocybin, which increase in psychoactivity may be offset by the reduced amount of psilocybin used when nutmeg is included in the composition.

Formulations that included nutmeg produced a more uplifting and euphoric effect and were often included stronger aphrodisiac properties, especially when combined with frankincense and myrrh.

Nutmeg was included in compositions for depression but not for stimulant compositions designed to treat ADD/ADHD. Individuals using stimulant medication often have difficulties sleeping. Nutmeg helped many individuals stop using stimulants and have an easier time falling asleep, as well as a more restful sleep, particularly when combined with phytocannabinoids.

### 1-8 Cineole

Bergamot citrus fruit flavonoids, including 1-8 cineole, are potent and selective blockers of TRPM3. Eucalyptus also includes 1-8 cineole, in addition to pinene and eucalyptol. The dose of 1-8 cineole vas variable based on inclusion of ingredients with or without it. Bergamot resulted in uplifting and extended effects but also amplified the psychoactive effects of the psilocybin, allowing a lowered dose of psilocybin relative to formulations that lacked bergamot.

Bergamot was found to support increased focus and concentration, and prolong the effects of the compositions. Some reported feeling a lasting benefit 12 to 24 hours after ingestion of a single dose. TRPM3 is expressed in nociceptive sensory neurons in dorsal root and trigeminal ganglia, similar to TRPV1. Similarly to TRPV1, activation of TRPM3 has been linked to thermal pain as well.

The physiological functions of TRPM3 and the antagonists known to interact with TRPM3 are different than TRVP1, although they both respond to thermal nociceptive stimuli, an effect that is maintained after induction of inflammatory hyperalgesia. The ability of molecules in bergamot such as eriodictyol to block the capsaicin-induced activation of rat TRPV1 but still show activity on TRPM3 indicate a different mode of action, and it has been shown to be involved in signaling from the eyes (Janda, 2016).

Some individuals described the psychoactive effects of formulations including bergamot in some cases as “a light was turned on inside the brain” or “a glow”. This may be connected to TRP receptors having been implicated in light sensitivity for both mammals and insects. Bergamot essential oil has phototoxic effects in humans when consumed in high doses, and transduction by TRP channels is associated with better information transfer in bright light (Katz, 2018). A relatively low amount of bergamot oil was sufficient to result in this effect and was notably different than formulations which lacked the bergamot. A dose of about was 0.012% (w/w) of the composition. Enhanced focus, concentration and energy were observed and reported from the compositions including citrus terpenoids in the bergamot.

### TRPM8 Agonists

Menthol, commonly known for its cooling sensation when eaten, inhaled or applied to the skin through agonism of TRPM8. Menthol is a natural monoterpenoid synthesized in plants from the Mentha genus (Salehi, 2018). Peppermint menthol, a compound obtained from the oil of peppermint (Mentha piperita), popularly known for its cooling effect, activates heat-activated TRPV3 (Oz, 2017). At warm temperatures menthol might be interpreted as warm based on its sensitizing effect on TRPV3, while at cooler temperatures, its activation of TRPM8 dominates its sensory quality (Oz, 2017). Eugenol also shows activity at TRPM8.

Other monoterpenes that activate TRPM8 include eucalyptol or 1-8 cineole (present in essential oils from Eucalyptus polybractea), menthone (the precursor of menthol in monoterpene biosynthesis), geraniol (found in lemon-grass and aromatic herb oils), linalool (found in floral scents of Onagraceae species), menthyl lactate (from peppermint oil), trans- and cis-p-menthane-3,8-diol (from E. citriodora), L-carvone (from spearmint or Kuromoji oil), isopulegol (from M. pulegium or Lilium ledebourri) and hydroxyl-citronellal (from citronella oils, volatile oils such as lemon, lemongrass or melissa oils) (Bharate, 2012).

### TRPA1 Agonists

TRPA1 is activated by pungent chemicals as allyl isothiocyanate (mustard oil), allicin (from garlic), cinnamaldehyde (from cinnamon), methylsalicylate (winter-green), eugenol (cloves) and gingerol (ginger).

Garlic and onion also contain allium. Including garlic and onion in the compositions may be practical in capsule formulation but was not typically included in food-based formulations as the combination with other spices may be off-putting. People with gastrointestinal issues often took garlic and ginger tablets, pills or capsules separately to mitigate bowel symptoms. The dose of allium was included for compositions directed to facilitating digestion. Garlic and onion may also improve weight loss, and may improve gastrointestinal issues along.

### Other TRP Agonists

Black pepper is high in BCP and contains myristicin. In addition, black pepper, includes another potent TRPV1 agonist—piperine (McNamara, 2005). Black pepper also includes guineensine, an anandamide reuptake inhibitor (Nicolussi, 2014) which further improve the effects, especially with cannabis present in the composition.

Ginger contains gingerol, a bioactive compound with demonstrated anti-inflammatory and antioxidant effects. (Wang, 2014.)

Carvacrol, the major ingredient of oregano (Origanum majoranal O. vulgare), and thymol, a lesser component of oregano but an important constituent of thyme (Thymus vulgaris) are both known to evoke a sense of warmth and sensitize skin (Can, 2008). Enhanced digestion through oregano oil and thyme may be due to carvacrol agonist binding at TRPV3.

### Additional Ingredients

Additional molecules and carriers may influence the effects. These were consciously chosen so as to either prevent any additional effect or to enhance the effect and flavor, making the medication more enjoyable.

Chocolate cacao and its derivatives cocoa and chocolate contain N-linoleoylethanolamide and N-oleoylethanolamide, compounds which inhibit anandamide breakdown, as well as variable amounts of anandamide. Inclusion of at least 70% dark cacao not only was used for flavor, but also increased the positive and euphoric affects associated with the formula (Smalheiser, 2019).

Vegan recipes were used to reduce any symptoms associated with cacao, which has been associated with an increase blood pressure and therefore removed from anxiety medications or for those with dietary issues. Vegan Carib chips maybe used to replace cacao and milk substitutes such as almond, soy or oat milk may be used to replace dairy milk as the liquid matrix.

Coffee was added to some compositions for stimulation where stimulation is consistent with the intended effect of the composition. It was also common for individuals to decide to take the edible chew formulation dissolved in coffee.

The compositions may include ingredients to suppress appetite as well and reduce sugar craving, which ultimately helps reduce inflammation in the body. Significant weight loss has been reported in many case studies, though theses cannot be attributed to the drug alone. Reduced sugar cravings and better adjusted appetite may have been a result of the experience during the psychoactive effects of the psilocybin and not due to neurochemical effects per se of the drug. In some cases, individuals using the compositions simply make decision to exercise more often and eat differently. Due to improved cognitive function and reduced depression, most individuals show drastic lifestyle changes that can also attribute to this. This includes an increased connection with nature, an increased connection with the environment and often drastic alterations in diet. Specific recipes were made to deal with dietary issues such as bowel inflammation.

### Formulations

The compositions may be formulated in dry form, as extracts or solubilized. The compositions may be formulated with acceptable carrier, excipient or diluent for oral administration and absorption through the gut or oral mucosa (e.g. sublingual, gingival, etc.), for dermal application or suppository. The compositions may be prepared from purified or synthesized compounds, from extracts with broad spectrum ingredients from source biomass, from raw biomass or from other preparations of raw biomass (e.g. dried, ground, sifted or otherwise processed without extraction, etc.). In any such examples, the 5HT2A agonist(s) and/or the TRP agonist(s) in the compositions may be at least about 99% pure. Pharmaceutically acceptable forms of the compounds in the compositions include salts, solvates, esters, carbamates, and phosphate esters.

Formulations including the compositions may be used for promotion and maintenance of mental health in health individuals. The formulations could be prepared as edible chews, capsules such as gel caps or soft gels, tinctures, tablets, dissolvable strips (e.g., sublingual films or buccal films), sachets, granules, suspensions, beverages, as foods or any other suitable formulation. In some aspects, the formulations may be administered according to any suitable dosing regimen, such as from 1-10 times per day. In other aspects, the formulations may be administered from 1-9 times per day, 1-8 times per day, 1-7 times per day, 1-6 times per day, 1-5 times per day, 1-4 times per day, 1-3 times per day, 1-2 times per day, or once per day. In some aspects, the formulations may be administered 10, 9, 8, 7, 6, 5, 4, 3, 2, or 1 times per day.

The formulations may be used in a therapeutic product (e.g. a drug product, a natural health product, a nutraceutical, etc.) for the treatment of inflammation related to bowel conditions including IBS, Crohn's disease, colitis, leaky gut syndrome, as well a mental illness conditions including ADD, ADHD, situational depression, MDD, minor depression, bipolar disorder, borderline personality disorder, seasonal affective disorder, postpartum depression, premenstrual dysphoric disorder, any treatment resistant depressions, post-traumatic stress disorder (“PTSD”), any of which may be co-morbid with other conditions listed above, with other psychological conditions, or with physical conditions such as peripheral pain, neurological pain or other forms of pain.

A “therapeutically effective amount” of a drug is an amount effective to demonstrate a selected activity of the drug in an individual receiving the drug. A “therapeutically effective amount” may also be referred to as an “effective dose range.” Preferred doses, effective dose ranges, recommended maximum doses, and/or recommended daily intake amounts for TRP agonists and 5HT2A agonists are listed below in Tables 6 and 7.

TABLE 6
Preferred, and Total Daily Intake Amounts for Selected TRP Agonists

| Substance | Molecule | Preferred Doses | Total Daily Amount | |
|---|---|---|---|---|
| Cayenne pepper | Capsiacin/Capsiates | 0.1-1 mg, 0.2-0.9 mg, 0.3-0.8 mg, | 0.1-50 | mg |
| | | 0.4-0.7 mg, or 0.5-0.6 mg | | |
| Tumeric | Curciminoids | 0.1-10 mg, 1-9 mg, 2-8 mg, 3-7 | 1-500 | mg |
| | | mg, or 4-6 mg | | |
| Clove | Eugenol | 0.1-25 mg, 1-24 mg, 2-23 mg, 3- | 1-500 | mg |
| | | 22 mg, 4-21 mg, 5-20 mg, 6-19 mg, | | |
| | | 7-18 mg, 8-17 mg, 9-16 mg, 10- | | |
| | | 15 mg, 11-14 mg, or 12-13 mg | | |
| | BetaCaryophyllene | 0.1-10 mg, 1-9 mg, 2-8 mg, 3-7 | 1-200 | mg |
| | | mg, or 4-6 mg | | |
| Cinnamon | Cinnamaldehye | 0.1-10 mg, 1-9 mg, 2-8 mg, 3-7 | 1-100 | mg |
| | | mg, or 4-6 mg | | |
| Nutmeg*** | Elemicin | 0.1-5 mg, 0.5-4.5 mg, 1-4 mg, 1.5- | 1-200 | mg |
| | | 3.5 mg, or 2-3 mg | | |
| | Myristicin | 0.1-5 mg, 0.5-4.5 mg, 1-4 mg, 1.5- | 1-200 | mg |
| | | 3.5 mg, or 2-3 mg | | |
| Oregano | Carvacrol | 0.1-10 mg, 1-9 mg, 2-8 mg, 3-7 | 1-100 | mg |
| | | mg, or 4-6 mg | | |
| Thyme | Thymol | 0.1-10 mg, 1-9 mg, 2-8 mg, 3-7 | 1-200 | mg |
| | | mg, or 4-6 mg | | |
| Pepper | Piperine | 0.1-10 mg, 1-9 mg, 2-8 mg, 3-7 | 1-150 | mg |
| | | mg, or 4-6 mg | | |
| Peppermint | Menthol | 0.1-10 mg, 1-9 mg, 2-8 mg, 3-7 | 0.5-150 | mg |
| | | mg, or 4-6 mg | | |
| Eucalyptus/ | Eucalyptol | 0.1-10 mg, 1-9 mg, 2-8 mg, 3-7 | 1-150 | mg |
| Cardamom | (1-8 cineole) | mg, or 4-6 mg | | |
| Onion/Garlic | Allicin | 0.5-10 mg, 1-9 mg, 2-8 mg, 3-7 mg, | 0.1-100 | mg |
| | | or 4-6 mg | | |
| Bergamot | Eriodictyol | 0.01-1 mg, 0.05-0.9 mg, 0.1-0.8 mg, | 0.1-20 | mg |
| | | 0.2-0.7 mg, 0.3-0.6 mg, or 0.4- | | |
| | | 0.5 mg | | |
| | Myrcene | 0.01-1 mg, 0.05-0.9 mg, 0.1-0.8 mg, | 0.1-20 | mg |
| | | 0.2-0.7 mg, 0.3-0.6 mg, or 0.4- | | |
| | | 0.5 mg | | |
| Cannabis | THC | 0.01-0.5 mg, 0.05-0.45 mg, 0.1-0.4 | 0.1-10 | mg |
| | | mg, 0.15-3.5 mg, or 0.2-0.3 mg | | |
| | CBD | 0.1-10 mg, 1-9 mg, 2-8 mg, 3-7 | 1-100 | mg |
| | | mg, or 4-6 mg | | |
| | CBG | 0.1-10 mg, 1-9 mg, 2-8 mg, 3-7 | 1-100 | mg |
| | | mg, or 4-6 mg | | |
| | CBN | 0.1-10 mg, 1-9 mg, 2-8 mg, 3-7 | 1-100 | mg |
| | | mg, or 4-6 mg | | |
| | THCV | 0.01-0.5 mg, 0.05-0.45 mg, 0.1-0.4 | 0.1-10 | mg |
| | | mg, 0.15-3.5 mg, or 0.2-0.3 mg | | |
| | CBDV | 0.01-0.5 mg, 0.05-0.45 mg, 0.1-0.4 | 0.1-10 | mg |
| | | mg, 0.15-3.5 mg, or 0.2-0.3 mg | | |
| | Minor Cannabinoids | 0.01-0.5 mg, 0.05-0.45 mg, 0.1-0.4 | 0.1-10 | mg |
| | | mg, 0.15-3.5 mg, or 0.2-0.3 mg | | |

***also a 5HT2A

TABLE 7
Preferred, Average, and Recommended Maximum Intake Amounts for Selected 5HT2A Agonists

| | | | | | Recommended | |
|---|---|---|---|---|---|---|
| Molecule | Chemical Name | Preferred Doses | Average Dose | | Maximum Dose | |
| Tryptamines | | 0.1-20 mg, 1- | | | | |
| | | 19 mg, 2-18 mg, 3- | | | | |
| | | 17 mg, 4-16 mg, 5- | | | | |
| | | 15 mg, 6-14 mg, 7- | | | | |
| | | 13 mg, 8-12 mg, or | | | | |
| | | 9-11 mg | | | | |
| Psilocybin | 3-[2- | 0.1-20 mg, 1- | 15-25 | mg | 40 | mg |
| | (dimethylamino)ethyl]-4- | 19 mg, 2-18 mg, 3- | | | | |
| | phosphoryloxyindole | 17 mg, 4-16 mg, 5- | | | | |
| | | 15 mg, 6-14 mg, 7- | | | | |
| | | 13 mg, 8-12 mg, or | | | | |
| | | 9-11 mg | | | | |
| Psilocin | 3-[2- | 0.1-20 mg, 1- | 15-25 | mg | 40 | mg |
| | (dimethylamino)ethyl]- | 19 mg, 2-18 mg, 3- | | | | |
| | 4-hydroxyindole | 17 mg, 4-16 mg, 5- | | | | |
| | | 15 mg, 6-14 mg, 7- | | | | |
| | | 13 mg, 8-12 mg, or | | | | |
| | | 9-11 mg | | | | |
| Psilacetin | 3-[2- | 0.1-20 mg, 1- | 15-30 | mg | 60 | mg |
| (4AcO-DMT) | (dimethylamino)ethyl]-4- | 19 mg, 2-18 mg, 3- | | | | |
| | acetoxyindole | 17 mg, 4-16 mg, 5- | | | | |
| | | 15 mg, 6-14 mg, 7- | | | | |
| | | 13 mg, 8-12 mg, or | | | | |
| | | 9-11 mg | | | | |
| Aeruginascin | 3- | 0.1-20 mg, 1- | No tox studies found | | | |
| | [2(trimethylamino)ethyl]- | 19 mg, 2-18 mg, 3- | | | | |
| | 4-phosphoryloxyindole | 17 mg, 4-16 mg, 5- | | | | |
| | | 15 mg, 6-14 mg, 7- | | | | |
| | | 13 mg, 8-12 mg, or | | | | |
| | | 9-11 mg | | | | |
| Baeocystin | 3-[2- | 0.1-20 mg, 1- | No tox studies found | | | |
| | (methylamino)ethyl]-4- | 19 mg, 2-18 mg, 3- | | | | |
| | phosphoryloxyindole | 17 mg, 4-16 mg, 5- | | | | |
| | | 15 mg, 6-14 mg, 7- | | | | |
| | | 13 mg, 8-12 mg, or | | | | |
| | | 9-11 mg | | | | |
| Norpsilocin | 3-[2- | 0.1-20 mg, 1- | No tox studies found | | | |
| | (methylamino)ethyl]-4- | 19 mg, 2-18 mg, 3- | | | | |
| | hydroxyindole, 3-[2- | 17 mg, 4-16 mg, 5- | | | | |
| | (amino)ethyl]-4- | 15 mg, 6-14 mg, 7- | | | | |
| | hydroxyindole | 13 mg, 8-12 mg, or | | | | |
| | | 9-11 mg | | | | |
| Norbaeocystin | 3-[2-(amino)ethyl]-4- | 0.1-20 mg, 1- | No tox studies found | | | |
| | phosphoryloxyindole | 19 mg, 2-18 mg, 3- | | | | |
| | | 17 mg, 4-16 mg, 5- | | | | |
| | | 15 mg, 6-14 mg, 7- | | | | |
| | | 13 mg, 8-12 mg, or | | | | |
| | | 9-11 mg | | | | |
| Bufotenin | 5-methoxy-DMT | 1-20 mg, 1- | 20-40 | mg | 60 | mg |
| | | 19 mg, 2-18 mg, 3- | | | | |
| | | 17 mg, 4-16 mg, 5- | | | | |
| | | 15 mg, 6-14 mg, 7- | | | | |
| | | 13 mg, 8-12 mg, or | | | | |
| | | 9-11 mg | | | | |
| DMT | N,N- | 1-10 mg, 2-9 mg, 3- | 20-40 | | 40-60 | |
| | Dimethyltryptamine | 8 mg, 4-7 mg, or 5- | | | | |
| | | 6 mg | | | | |
| Endogenous | | 0.1-400 mg, 1- | | | | |
| molecules | | 375 mg, 10- | | | | |
| | | 350 mg, 50- | | | | |
| | | 325 mg, 75- | | | | |
| | | 300 mg, 100- | | | | |
| | | 275 mg, 125- | | | | |
| | | 250 mg, 150- | | | | |
| | | 225 mg, or 175- | | | | |
| | | 200 mg | | | | |
| Melatonin | N-acetyl-5-methoxy | 0.25-1 mg, 0.3- | 1-3 | mg | 6 | mg |
| | tryptamine | 0.9 mg, 0.4-0.8 mg, | | | | |
| | | or 0.5-0.7 mg | | | | |
| Serotonin | 5-hydroxy tryptamine | 1-400 mg, 25- | | | | |
| | | 375 mg, 50-350 mg, | | | | |
| | | 75-325 mg, 100- | | | | |
| | | 300 mg, 125- | | | | |
| | | 275 mg, 150- | | | | |
| | | 250 mg, or 175- | | | | |
| | | 225 mg | | | | |
| 5-HTP | 5-hydroxy-tryptophan | 50-200 mg, 60- | 100-300 | mg | 300-400 | mg |
| | | 190 mg, 70-180 mg, | | | | |
| | | 80-170 mg, 90- | | | | |
| | | 160 mg, 100- | | | | |
| | | 150 mg, 110- | | | | |
| | | 140 mg, or 120- | | | | |
| | | 130 mg | | | | |
| Ergolines | | 10-500 μg, 50-450 | | | | |
| | | μg, 100-400 μg, | | | | |
| | | 150-350 μg, or | | | | |
| | | 200-300 μg | | | | |
| LAE | D-lysergic acid | 0.1-0.5 mg, 0.15- | 0.5-1.5 | mg | | |
| | ethylamide | 0.45 mg, 0.2-4 mg, | | | | |
| | | or 0.25-0.35 mg | | | | |
| D-lysergic acid | | | 0.2-0.4 | mg | | |
| beta- | | | | | | |
| propanolamide | | | | | | |
| LSA (ergine) | Lysergic acid amide | 1-500 μg, 50-450 | | | | |
| | | μg, 100-400 μg, | | | | |
| | | 150-350 μg, or | | | | |
| | | 200-300 μg | | | | |
| LSB | D-lysergic acid 2-butyl | 0.03-0.06 mg | 0.07-0.12 | mg | 0.24 | mg |
| | amide | | | | | |
| D-lysergic acid | | 0.1-0.2 mg | 0.2 | mg | 0.8 | mg |
| 1-butanolamide | | | | | | |
| 1-methyl-D- | | 1-2 mg, 1.1- | 2 | mg | 8 | mg |
| lysergic acid | | 1.9 mg, 1.2-1.8 mg, | | | | |
| butanolamide | | 1.3-1.7 mg, or 1.4- | | | | |
| | | 1.6 mg | | | | |
| LSP | D-lysergic acid 3-pentyl | | 75% potency | | | |
| | amide | | of LSD? | | | |
| LSM-775 | D-N- | 500-750 μg, 525- | 750-1250 | μg | 1.5 | mg |
| | morpholinyllysergamide | 725 μg, 550-700 | | | | |
| | | μg, 575-675 μg, | | | | |
| | | or 600-650 μg | | | | |
| LPD-824 | D-N- | | 800 | μg | | |
| | pyrrolidyllysergamide | | | | | |
| LSD-Pip | (8β)-6-methyl-8- | More potent than LPD-824 and LSM-775, but | | | | |
| | (piperidin-1-ylcarbonyl)- | still several times less potent than LSD as a | | | | |
| | 9,10-didehydroergoline | 5HT2A agonist | | | | |
| DAM | N,N- | | 1 | mg | | |
| | dimethyllysergamide | | | | | |
| LAMIDE | D-lysergic acid | 50-200 μg, 60- | 180-300 | μg | | |
| | methylisopropyl amide | 190 μg, 70-180 | | | | |
| | | μg, 80-170 μg, | | | | |
| | | 90-160 μg, 100- | | | | |
| | | 150 μg, 110-140 | | | | |
| | | μg, or 120-130 | | | | |
| | | μg | | | | |
| LSZ | D-lysergic acid 2,4- | 10-150 μg, 20- | 100-300 | μg | 200 | μg |
| | dimethylazetidide | 140 μg, 30-130 | | | | |
| | | μg, 40-120 μg, | | | | |
| | | 50-110 μg, 60- | | | | |
| | | 100 μg, or 70-90 | | | | |
| | | μg | | | | |
| LSD | Lysergic acid | 10-75 μg, 20-65 | 50-150 | μg | 400 | μg |
| | diethylamimde | μg, 30-55 μg, or | | | | |
| | | 40-55 μg | | | | |
| ALD-52 | D-1-acetyl-lysergic acid | 30-125 μg, 40- | 100-175 | μg | 325 | μg |
| | diethylamide | 115 μg, 50-105 | | | | |
| | | μg, 60-95 μg, or | | | | |
| | | 70-85 μg | | | | |
| 1P-LSD | D-1-propionyl-lysergic | 10-50 μg, 15-45 | 50-150 | μg | 200-300 | μg |
| | acid diethylamide | μg, 20-40 μg, or | | | | |
| | | 25-35 μg | | | | |
| 1B-LSD | D-N1-butyryl-lysergic | 15-75 μg, 20-70 | 75-150 | μg | 150-300 | μg |
| | acid diethylamide | μg, 25-65 μg, 30- | | | | |
| | | 60 μg, 35-55 | | | | |
| | | μg, or 40-50 μg | | | | |
| 1cP-LSD | D-N1- | 15-75 μg, 20-70 | 75-150 | μg | 150-300 | μg |
| | (cyclopropylmethanoyl)- | μg, 25-65 μg, 30- | | | | |
| | lysergic acid | 60 μg, 35-55 | | | | |
| | diethylamide | μg, or 40-50 μg | | | | |
| LSH | D-Lysergic acid α- | 1-500 μg, 25- | | | | |
| | hydroxyethylamide | 475 μg, 50-450 μg, | | | | |
| | | 75-425 μg, 100- | | | | |
| | | 400 μg, 125- | | | | |
| | | 375 μg, 150- | | | | |
| | | 350 μg, 175- | | | | |
| | | 325 μg, 200- | | | | |
| | | 300 μg, or 225- | | | | |
| | | 275 μg | | | | |
| MLD | D-N1-methyl-lysergic | | 100-300 | μg | | |
| | acid diethylamide | | | | | |
| ETH-LAD | D-6-ethyl-6-nor-lysergic | 15-60 μg, 20-55 | 60-150 | μg | 225 | μg |
| | acid diethylamide | μg, 25-50 μg, 30- | | | | |
| | | 45 μg, or 35-40 | | | | |
| | | μg | | | | |
| 1P-ETH-LAD | D-1-propionyl-6-ethyl-6- | 25-60 μg, 30-55 | 60-100 | μg | 100-200 | μg |
| | nor-lysergic acid | μg, 35-50 μg, or | | | | |
| | diethyamide | 40-45 μg | | | | |
| AL-LAD | D-6-allyl-6-nor-lysergic | 50-100 μg, 55- | 100-200 | μg | 200-300 | μg |
| | acid diethylamide | 95 μg, 60-90 μg, | | | | |
| | | 65-85 μg, or 70- | | | | |
| | | 80 μg | | | | |
| PRO-LAD | D-6-propyl-6-nor- | 10-75 μg, 15-70 | 100-200 | μg | 200-300 | μg |
| | lysergic acid | μg, 20-65 μg, 25- | | | | |
| | diethylamide | 60 μg, 30-55 | | | | |
| | | μg, 35-50 μg, or | | | | |
| | | 40-45 μg | | | | |
| IP-LAD | D-6-isopropyl-6-nor- | 10-75 μg, 15-70 | 100-200 | μg | 200-300 | μg |
| | lysergic acid | μg, 20-65 μg, 25- | | | | |
| | diethylamide | 60 μg, 30-55 | | | | |
| | | μg, 35-50 μg, or | | | | |
| | | 40-45 μg | | | | |
| PARGY-LAD | D-6-propynyl-6-nor- | 50-275 μg, 75- | 275-650 | μg | 700 | μg |
| | lysergic acid | 250 μg, 100-225 | | | | |
| | diethylamide | μg, 125-200 μg, | | | | |
| | | or 150-175 μg | | | | |
| BU-LAD | D-6-butyl-6-norlysergic | 100-500 μg, 125- | | | | |
| | acid diethylamide | 475 μg, 150- | | | | |
| | | 450 μg, 175-425 | | | | |
| | | μg, 200-400 μg, | | | | |
| | | 225-375 μg, 250- | | | | |
| | | 350 μg, or 275- | | | | |
| | | 325 μg | | | | |
| DAL | N,N-diallyllysergamide | | No tox studies found | | | |
| ECPLA | D-N-ethyl-N- | | 40% potency | | | |
| | cyclopropyllysergamide | | of LSD? | | | |
| Phenethylamines | | 0.1-200 mg, 1- | | | | |
| | | 190 mg, 10- | | | | |
| | | 180 mg, 20- | | | | |
| | | 170 mg, 30- | | | | |
| | | 160 mg, 40- | | | | |
| | | 150 mg, 50- | | | | |
| | | 140 mg, 60- | | | | |
| | | 130 mg, 70- | | | | |
| | | 120 mg, 80- | | | | |
| | | 110 mg, or 90- | | | | |
| | | 100 mg | | | | |
| Mescaline | 3,4,5- | 10-200 mg, 20- | 200-300 | mg | 700 | mg |
| | trimethoxyphenethylamine | 190 mg, 30-180 mg, | | | | |
| | | 40-170 mg, 50- | | | | |
| | | 160 mg, 60-150 mg, | | | | |
| | | 70-140 mg, 80- | | | | |
| | | 130 mg, 90-120 mg, | | | | |
| | | or 100-110 mg | | | | |
| TMA | trimethoxyamphetamine | 20-100 mg, 25- | 100-250 | mg | Shulgin | |
| | | 95 mg, 30-90 mg, | | | Research | |
| | | 35-85 mg, 40- | | | | |
| | | 80 mg, 45-75 mg, | | | | |
| | | 50-70 mg, or 55- | | | | |
| | | 65 mg | | | | |
| 2C-B | 4-bromo-2,5- | 2-15 mg, 3-14 | 15-25 | mg | 50 | mg |
| | dimethoxybenzeneethanamine | mg, 4-13 mg, 5-12 | | | | |
| | | mg, 6-11 mg, 7-10 | | | | |
| | | mg, or 8-9 mg | | | | |
| DOB | 4-bromo-2,5- | 0.2-0.75 mg, | 0.75-1.75 | mg | 3.5 | mg |
| | dimethoxyamphetamine | 0.25-0.7 mg, 0.3- | | | | |
| | | 0.65 mg, 0.35-0.6 | | | | |
| | | mg, 0.4-0.55 mg, | | | | |
| | | or 0.45-0.5 mg | | | | |
| DOM | 4-methyl-2,5- | 0.5-2 mg, 0.6- | 2-6 | mg | 12 | mg |
| | dimethoxyamphetamine | 1.9 mg, 0.7-1.8 mg, | | | | |
| | | 0.8-1.7 mg, 0.9- | | | | |
| | | 1.6 mg, 1-1.5 mg, | | | | |
| | | 1.1-1.4 mg, or 1.2- | | | | |
| | | 1.3 mg | | | | |
| 2C-D | 4-methyl-2,5- | 3-15 mg, 4-14 | 20-50 | mg | 100 | mg |
| | dimethoxybenzeneethanamine | mg, 5-13 mg, 6-12 | | | | |
| | | mg, 7-11 mg, or 8- | | | | |
| | | 10 mg | | | | |
| MDA | 3,4- | 1-100 mg, 5-95 mg, | 60-150 | mg | 200 | mg |
| | methylenedioxyamphetamine | 10-90 mg, 15- | | | | |
| | | 85 mg, 20-80 mg, | | | | |
| | | 25-75 mg, 30- | | | | |
| | | 70 mg, 35-65 mg, | | | | |
| | | 40-60 mg, or 45- | | | | |
| | | 55 mg | | | | |
| MDMA | N-methyl-3,4- | 1-100 mg, 5-95 mg, | 60-150 | mg | 200 | mg |
| | methylenedioxyamphetamine | 10-90 mg, 15- | | | | |
| | | 85 mg, 20-80 mg, | | | | |
| | | 25-75 mg, 30- | | | | |
| | | 70 mg, 35-65 mg, | | | | |
| | | 40-60 mg, or 45- | | | | |
| | | 55 mg | | | | |
| Isoroscaline | 4-isopropoxy-3,5- | 1-50 mg, 5-45 mg, | 50-200 | mg | | |
| | dimethoxyphenethylamine | 10-40 mg, 15- | | | | |
| | | 35 mg, or 20-30 mg | | | | |
| Proscaline | 4-propoxy-3,5-DMPEA | 1-50 mg, 5-45 mg, | 20-40 | mg | 60 | mg |
| | | 10-40 mg, 15- | | | | |
| | | 35 mg, or 20-30 mg | | | | |
| allylescaline | | 1-50 mg, 5-45 mg, | | | | |
| | | 10-40 mg, 15- | | | | |
| | | 35 mg, or 20-30 mg | | | | |
| Escaline | | 1-50 mg, 5-45 mg, | | | | |
| | | 10-40 mg, 15- | | | | |
| | | 35 mg, or 20-30 mg | | | | |
| Phenylpropanoids/ | | 0.1-200 mg, 1- | | | | |
| Aromatic | | 190 mg, 10- | | | | |
| Ethers | | 180 mg, 20- | | | | |
| | | 170 mg, 30- | | | | |
| | | 160 mg, 40- | | | | |
| | | 150 mg, 50- | | | | |
| | | 140 mg, 60- | | | | |
| | | 130 mg, 70- | | | | |
| | | 120 mg, 80- | | | | |
| | | 110 mg, or 90- | | | | |
| | | 100 mg | | | | |
| Elemicin | 1,2,3-timethoxy-5-(prop- | 50-200 mg, 55- | 200-500 | mg | 500-800 | mg |
| | 2-en-1-yl)benzene | 195 mg, 60- | | | | |
| | | 190 mg, 65-185 mg, | | | | |
| | | 70-180 mg, 75- | | | | |
| | | 175 mg, 80-170 mg, | | | | |
| | | 85-165 mg, 90- | | | | |
| | | 160 mg, 95-155 mg, | | | | |
| | | 100-150 mg, 105- | | | | |
| | | 145 mg, 110- | | | | |
| | | 140 mg, 115- | | | | |
| | | 135 mg, or 120- | | | | |
| | | 130 mg | | | | |

| | |
|---|---|
| Myristicin | 50-200 mg, 55- |
| | 195 mg, 60- |
| | 190 mg, 65-185 mg, |
| | 70-180 mg, 75- |
| | 175 mg, 80-170 mg, |
| | 85-165 mg, 90- |
| | 160 mg, 95-155 mg, |
| | 100-150 mg, 105- |
| | 145 mg, 110- |
| | 140 mg, 115- |
| | 135 mg, or 120- |
| | 130 mg |

A “combination therapy” is a treatment with a certain substance or composition in which an individual is treated with, or given, one or more other compositions or drugs for the disorder or condition in conjunction with the first therapy or in conjunction with one or more other therapies. A combination therapy may be sequential therapy wherein an individual is treated first with one treatment modality (e.g. drug, psychotherapy, etc.) and then the other (e.g. drug, psychotherapy, etc.) or one more drugs, one or more therapies, or one or more drugs and one or more therapies, can be administered simultaneously. In either case, these drugs or therapies are said to be “coadministered”. It is to be understood that “coadministered” does not necessarily mean that the drugs or therapies are administered in a combined form. The drugs or therapies may be administered separately or together to the same or different sites at the same or different times.

### EXAMPLES

### Example 1

Eighteen initial experimental compositions including combinations of TRP receptor agonists were prepared are named based on their intended use. Each of these compositions could be formulated, for example into edible chews or gel caps as described in specific examples provided herein.

Each of the initial eighteen compositions utilized may be prepared including any suitable 5HT2A agonist obtained from a natural source with no special chemistry or extractions required. Each of these compositions was prepared using food ingredients or simple extracts from these food ingredients, as sources of the TRP receptor agonists described herein, including at Table 1 and Table 2. Any suitable source of the active ingredients present in these food ingredients as described in Table 1 and in Table 2 could be used to prepare the compositions described herein.

Seven base compositions were prepared, as shown in Table 8.

TABLE 8
Base Compositions

| Composition | Cayenne | Clove | Turmeric | Cinnamon | Nutmeg |
|---|---|---|---|---|---|
| Base 1 - Analgesia | Yes | Yes | No | No | No |
| Base 2 - Mood Support | No | Yes | Yes | No | No |
| Base 3 - Anti-Inflammatory | Yes | No | Yes | No | No |
| Base 4 - General | Yes | Yes | Yes | No | No |
| Base 5 - Anti-Depression | Yes | Yes | Yes | No | Yes |
| Base 6 - Anti-Anxiety | Yes | Yes | Yes | Yes | No |
| Base 7 - Complete | Yes | Yes | Yes | Yes | Yes |

From the above seven base compositions listed in Table 8, eleven additional example compositions were prepared and named based on their intended use, as shown in Table 9.

TABLE 9
Specific Compositions

| No/Base | Composition | Additional ingredients | Optional Ingredients |
|---|---|---|---|
| 8; 2 | Sleep | cinnamon, nutmeg, | cannabis, passion flower |
| | | serotonin, melatonin | |
| 9; 5 | Anti- | (none), cacao | cinnamon, cannabis, kratom, |
| | depression | | frankincense, myrrh, kava, |
| | | | pepper |
| 10; 6 | Anti-anxiety | (none) | cannabis, kratom |
| 11; 7 | Relaxation | coffee, cannabis, bergamot | peppercorns, cacao |
| 12; 7 | Focus | gingko biloba | cannabis, cacao, peppermint, |
| | | | peppercorns |
| 13; 7 | Creativity | coffee, pepper, peppermint | cannabis, cacao, gingko |
| | | | biloba, bergamot, myrrh |
| 14; 6 | Anti- | garlic, ginger, cardamom, | cannabis, frankincense, |
| | inflammatory | onion, thyme | oregano, cacao, pepper |
| | for bowel | | |
| 15; 2 | Digestion | cinnamon, garlic, ginger, | cannabis, frankincense, |
| | | peppermint, cardamom | oregano, cacao, pepper |
| 16; 6 | Analgesia | pepper, peppermint, thyme, | cannabis, kratom, cacao, |
| | | oregano | pepper |
| 17; 7 | TBI Treatment | cacao, gingko biloba, | Cannabis, pepper |
| | | myrrh, frankincense | |
| 18; 7 | Aphrodisiac | peppermint, gingko biloba, | peppercorns, ginseng, |
| | | cardamom, myrrh, | cannabis, kratom |
| | | frankincense, kava, | |

### Example 2

Thirty experimental formulations were prepared including many of the example compositions described herein. The formulations were prepared as capsules and as cocoa-based chews. Fourteen of the above compositions were prepared for each of these two formulations. Specifically, example formulations are provided for the compositions Base 1—Analgesia, Base 2—Mood Support, Base 3—Anti-Inflammatory, Base 6—Anti-Anxiety, Sleep, Anti-depression, Relaxation, Focus, Creativity, Anti-inflammatory for bowel, Digestion, Analgesia, TBI Treatment and Aphrodisiac.

Each of these formulations included psilocybin as a 5HT2A agonist, but could be prepared with any suitable 5HT2A agonist. The psilocybin used in each of these formulations was sourced from dried fruiting bodies of P. cubensis, but the compositions could be prepared with fruiting bodies, sclerotia, mycelia, cell culture or any suitable species of psilocybin containing fungi or other source of psilocybin. Since dried fruiting bodies were used as a source of psilocybin, it is likely that some amounts of psilocin, baeocystin, aeruginascin, norpsilocybin, norpsilocin, norbaeocystin or other tryptamines that are 5HT2A agonists were also present in the formulations.

The 5HT2A agonist used in each of these formulations included psilocybin from dried fruiting bodies. In sleep, the 5HT2A agonist also included serotonin and melatonin. In all formulations other than Base 1—Analgesia, Base 2—Mood Support, Base 3—Anti-Inflammatory, Base 6—Anti-Anxiety, Sleep, Anti-inflammatory for bowel, Digestion and Analgesia, the 5HT2A agonist also included myristicin and elemicin.

All ingredients in the capsule formulation were dried and ground other than as indicated in the below Tables with even numbers from among Tables 10 to 37.

The “liquid matrix” in the edible chew formulation may be any suitable and palatable liquid for mixing with the cacao (e.g. dairy milk, almond milk, hemp milk, soy milk, oat milk, etc.). All ingredients in the edible chew formulation were dried and ground other than ingredients that were not dried and ground in the corresponding capsule formulation, the dark chocolate and the liquid matrix.

When formulated as an edible chew, the dark chocolate is heated with sufficient liquid matrix to melt the dark chocolate without burning. The liquid matrix is further added as additional dried powdered ingredients are added. Finely ground dried fruiting bodies were added last without heating and stirred thoroughly to homogenize. Once the material had mixed sufficiently and begun to cool below flow temperature, then the material was put into moulds and cooled to allow solidifying into individual dosage units.

In formulations that included the ingredients, capsaicin was present at between 0.05 and 2.5 mg per dosage unit, curcumin was present at between 1.00 and 15 mg per dosage unit, eugenol was included at between 0.5 and 15 mg per dosage unit, BCP was included at between 0.25 and 5 mg per dosage unit, cinnamaldehyde was included at between 0.25 and 3 mg per dosage unit and myristicin and elemicin was present at between 0.50 and 3 mg per dosage unit.

### Example Formulation 01—Base Analgesia

Table 10 shows the ingredients for Base Analgesia formulated as material to be included in a capsule dosage form.

TABLE 10
Ingredients for Base Analgesia formulated for capsules

| Ingredient | Weight (g) | Ratio |
|---|---|---|
| P. cubensis dried fruiting bodies | 20 | 0.50 |
| Cayenne pepper fruit and seeds | 10 | 0.25 |
| Clove fruit, stems and buds | 10 | 0.25 |

The ingredients in Table 10 provide a total mass of 40 g. The weight per dosage unit is 0.3 g, including about 0.15 g of dried fruiting bodies of the P. cubensis. This provides approximately 133 dosage units from the ingredients in Table 10. Through a Controlled Substance Dealers license obtained through the Office of Controlled Substances with Health Canada, we were able to legally propagate and analyze the exact strain of mushrooms being utilized in the formulations referred to as P. cubensis. HPLC analysis on Psilocybin/psilocin content in the fungal biomass was determined using methanol mushroom extracts (100 mg of dry homogenized mushroom biomass+5 mL of 100% methanol incubated at 60° C. for 1 h with vortexing and then filtered through a 0.2 um filter) were separated on an Agilent 1200 Series HPLC system using the following parameters:

Column: iHILIC-Fusion, PEEK, P/N: 100.152.0310.

Column temperature: 40° C.

Mobile Phase: 80:20 (v/v) acetonitrile—ammonium formate (10 mM, pH 3.5).

Flow Rate: 0.3 mL/min.

Results indicate a range between approximately 0.5-2.25% active metabolites by weight at various stages of growth with an average of around 1% in the freshly harvested and dried fruiting bodies. As such, we calculate the amount of psilocybin to be approximately 1-1.5 mg in most of the recipes and formulations tested depending on the amount of dried fruiting bodies included. As the dried fruiting bodies are about 1.0% psilocybin in this formulation, this provides about 1.5 mg psilocybin per dosage unit.

Table 11. HPLC results from cultivated Psilocybin cubensis mushrooms including many results from various harvests of the mushroom as well as some more common strains for comparison. This specific strain of mushroom also has a relative psilocybin to psilocin content of 10:1 compared to other varieties found to be closer to 1:1 or 2:1 ratios.

TABLE 11
HPLC results from cultivated Psilocybin cubensis mushrooms including results
from various harvests of the mushroom and more common strains for comparison.
HPLC of isolated P. cubensis variety

| | | | | | |
|---|---|---|---|---|---|
| | | | | Combined | Ratio |
| | | Psilocin | Psilocybin | Total | Psilocybin/ |
| Experiment | Sample Description | (% w/w) | (% w/w) | (% w/w) | Psilocin |

Anatomical analysis

| | | | | | |
|---|---|---|---|---|---|
| P. cubensis Batch 16 | Primordial stage | 0.05 | 0.65 | 0.70 | 13.13 |
| P. cubensis Batch 16 | Small Immature mushroom | 0.21 | 2.01 | 2.23 | 9.55 |
| P. cubensis Batch 16 | Medium Immature | 0.19 | 1.22 | 1.41 | 6.54 |
| | mushroom | | | | |
| P. cubensis Batch 16 | Mature Stem (interior) | 0.08 | 0.53 | 0.60 | 6.85 |
| P. cubensis Batch 16 | Mature Stem (exterior) | 0.09 | 0.94 | 1.02 | 10.89 |
| P. cubensis Batch 16 | Mature Blended stems | 0.07 | 1.00 | 1.07 | 14.29 |
| P. cubensis Batch 16 | Mature Gills | 0.09 | 1.50 | 1.59 | 17.60 |
| P. cubensis Batch 16 | Mature Cap | 0.10 | 0.74 | 0.85 | 7.11 |
| P. cubensis Batch 16 | Blended Harvests | 0.08 | 0.84 | 0.92 | 10.04 |
| P. cubensis Batch 16 | Spores | ND | ND | NA | NA |
| | Average | 0.11 | 1.05 | 1.15 | 10.67 |

Sequential Harvests

| | | | | | |
|---|---|---|---|---|---|
| P. cubensis Batch 42 | First Harvest (Flush) | 0.16 | 0.87 | 1.03 | 5.44 |
| P. cubensis Batch 42 | Second Harvest | 0.11 | 0.96 | 1.07 | 9.16 |
| P. cubensis Batch 42 | Third Harvest | 0.08 | 0.84 | 0.92 | 10.54 |
| P. cubensis Batch 42 | Forth Harvest | 0.06 | 0.87 | 0.93 | 13.85 |
| | Average | 0.10 | 0.89 | 0.99 | 9.75 |

Degradation analysis

| | | | | | |
|---|---|---|---|---|---|
| P. cubensis Batch 2 | ~6 months post harvest | 0.06 | 0.51 | 0.57 | 8.00 |
| P. cubensis Batch 9 | ~5 months post harvest | 0.06 | 0.55 | 0.61 | 9.50 |
| P. cubensis Batch 23 | ~3 months post harvest | 0.06 | 0.51 | 0.57 | 8.09 |
| P. cubensis Batch 25 | ~3 months post harvest | 0.07 | 0.57 | 0.64 | 8.34 |
| P. cubensis Batch 26 | ~3 months post harvest | 0.05 | 0.47 | 0.52 | 8.68 |
| P. cubensis Batch 42 | ~2 months post harvest | 0.06 | 0.74 | 0.79 | 12.81 |
| | Average | 0.06 | 0.56 | 0.62 | 9.24 |

Strain Comparison

| | | | | |
|---|---|---|---|---|
| P. cubensis strain | 0.25 | 0.48 | 0.73 | 1.89 |
| ‘Alacabenzi’ | | | | |
| P. cubensis strain | 0.25 | 0.45 | 0.70 | 1.82 |
| ‘Avery's Albino’ | | | | |
| P. cubensis strain ‘B+’ | 1.64 | 1.86 | 3.50 | 1.13 |

Table 12 shows the ingredients for Base Analgesia formulated as an edible chew dosage form.

TABLE 12
Ingredients for Base Analgesia formulated as edible chews

| Ingredient | Volume | | Weight (g) | Ratio |
|---|---|---|---|---|
| Dark chocolate (70% cacao) | n/a | | 550 | 0.821 |
| Liquid matrix | 80 | ml | 80 | 0.119 |
| Dried fruiting bodies | n/a | | 20 | 0.030 |
| Clove fruit, stems and buds | 2 | tbsp | 10 | 0.015 |
| Cayenne pepper fruit and seeds | 2 | tbsp | 10 | 0.015 |

The ingredients in Table 12 provide a total mass of 670. The weight per dosage unit is 5 g, including about 0.15 g of dried fruiting bodies. This provides 133 dosage units from the ingredients in Table 12. The dried fruiting bodies are about 1.0% psilocybin, providing about 1.5 mg psilocybin per dosage unit.

### Example Formulation 02—Base Mood Support

Table 13 shows the ingredients for Base Mood Support formulated as material to be included in a capsule dosage form.

TABLE 13
Ingredients for Base Mood Support formulated for capsules

| Ingredient | Weight (g) | Ratio |
|---|---|---|
| Dried fruiting bodies | 20 | 0.40 |
| Turmeric root | 20 | 0.40 |
| Clove fruit, stems and buds | 10 | 0.20 |

The ingredients in Table 13 provide a total mass of 50 g. The weight per dosage unit is 0.3 g, including about 0.12 g of dried fruiting bodies. This provides 133 dosage units from the ingredients in Table 13. The dried fruiting bodies are about 1.0% psilocybin, providing about 1.2 mg psilocybin per dosage unit.

Table 14 shows the ingredients for Base Mood Support formulated as an edible chew dosage form.

TABLE 14
Ingredients for Base Mood Support formulated as edible chews

| Ingredient | Volume | | Weight (g) | Ratio |
|---|---|---|---|---|
| Dark chocolate (70% cacao) | n/a | | 550 | 0.821 |
| Liquid matrix | 80 | ml | 80 | 0.119 |
| Turmeric root | 2 | tbsp | 20 | 0.030 |
| Dried fruiting bodies | n/a | | 15 | 0.022 |
| Clove fruit, stems and buds | 2 | tsp | 5 | 0.007 |

The ingredients in Table 14 provide a total mass of 670 g. The weight per dosage unit is 5 g, including about 0.15 g of dried fruiting bodies. This provides 133 dosage units from the ingredients in Table 14. The dried fruiting bodies are about 1.0% psilocybin, providing about 0.11 mg psilocybin per dosage unit.

### Example Formulation 03—Base Anti-Inflammatory

Table 15 shows the ingredients for Base Anti-Inflammatory formulated as material to be included in a capsule dosage form.

TABLE 15
Ingredients for Base Anti-Inflammatory formulated for capsules

| Ingredient | Weight (g) | Ratio |
|---|---|---|
| Dried fruiting bodies | 25 | 0.50 |
| Turmeric root | 20 | 0.40 |
| Cayenne pepper fruit and seeds | 5 | 0.10 |

The ingredients in Table 15 provide a total mass of 50 g. The weight per dosage unit is 0.3 g, including about 0.15 g of dried fruiting bodies. This provides 166 dosage units from the ingredients in Table 15. The dried fruiting bodies are about 1.0% psilocybin, providing about 1.5 mg psilocybin per dosage unit.

Table 16 shows the ingredients for Base Anti-Inflammatory formulated as an edible chew dosage form.

TABLE 16
Ingredients for Base Anti-Inflammatory formulated as edible chews

| Ingredient | Volume | | Weight (g) | Ratio |
|---|---|---|---|---|
| Dark chocolate (70% cacao) | n/a | | 600 | 0.828 |
| Liquid matrix | 80 | ml | 80 | 0.110 |
| Dried fruiting bodies | n/a | | 20 | 0.028 |
| Turmeric root | 2 | tbsp | 20 | 0.028 |
| Cayenne pepper fruit and seeds | 1 | tbsp | 5 | 0.007 |

The ingredients in Table 16 provide a total mass of 725 g. The weight per dosage unit is 5 g, including about 0.14 g of dried fruiting bodies. This provides 145 dosage units from the ingredients in Table 16. The dried fruiting bodies are about 1.0% psilocybin, providing about 1.4 mg psilocybin per dosage unit.

### Example Formulation 06—Base Anti-Anxiety

Table 17 shows the ingredients for Base Anti-Anxiety formulated as material to be included in a capsule dosage form.

TABLE 17
Ingredients for Base Anti-Anxiety formulated for capsules

| Ingredient | Weight (g) | Ratio |
|---|---|---|
| Dried fruiting bodies | 25 | 0.42 |
| Turmeric root | 20 | 0.33 |
| Cayenne pepper fruit and seeds | 5 | 0.08 |
| Cinnamon stalk | 5 | 0.08 |
| Clove fruit, stems and buds | 5 | 0.08 |

The ingredients in Table 17 provide a total mass of 70 g. The weight per dosage unit is 0.3 g, including about 0.11 g of dried fruiting bodies. This provides 233 dosage units from the ingredients in Table 17. The dried fruiting bodies are about 1.0% psilocybin, providing about 1.1 mg psilocybin per dosage unit.

Table 18 shows the ingredients for Base Anti-Anxiety formulated as an edible chew dosage form.

TABLE 18
Ingredients for Base Anti-Anxiety formulated as edible chews

| Ingredient | Volume | | Weight (g) | Ratio |
|---|---|---|---|---|
| Vegan carob chips | n/a | | 600 | 0.821 |
| Liquid matrix | 80 | ml | 80 | 0.110 |
| Turmeric root | 2 | tbsp | 20 | 0.027 |
| Dried fruiting bodies | n/a | | 15 | 0.021 |
| Cayenne pepper fruit and seeds | 1 | tbsp | 5 | 0.007 |
| Cinnamon stalk | 2 | tsp | 5 | 0.007 |
| Clove fruit, stems and buds | 2 | tsp | 5 | 0.007 |

The ingredients in Table 18 provide a total mass of 730 g. The weight per dosage unit is 5 g, including about 0.10 g of dried fruiting bodies. This provides 146 dosage units from the ingredients in Table 18. The dried fruiting bodies are about 1.0% psilocybin, providing about 1.0 mg psilocybin per dosage unit.

### Example Formulation 08—Sleep

Table 19 shows the ingredients for Sleep formulated as material to be included in a capsule dosage form.

TABLE 19
Ingredients for Sleep formulated for capsules

| Ingredient | Weight (g) | Ratio |
|---|---|---|
| Dried fruiting bodies | 15 | 0.23 |
| Turmeric root | 10 | 0.15 |
| Nutmeg seed | 10 | 0.15 |
| Cinnamon stalk | 5 | 0.08 |
| Clove fruit, stems and buds | 5 | 0.08 |
| 70% cacao dark chocolate | 5 | 0.08 |
| Cannabis flower with about 10% CBD | 5 | 0.08 |
| and about 0.5% CBN | | |
| Serotonin formulated into 400 mg | 5 | 0.08 |
| capsules including 50 mg serotonin | | |
| Melatonin formulated into 250 mg | 5 | 0.08 |
| capsules including 5 mg melatonin | | |

The ingredients in Table 19 provide a total mass of 65 g. The weight per dosage unit is 0.3 g, including about 0.07 g of dried fruiting bodies. This provides 217 dosage units from the ingredients in Table 19. The dried fruiting bodies are about 1.0% psilocybin, providing about 0.7 mg psilocybin per dosage unit.

Table 20 shows the ingredients for Sleep formulated as an edible chew dosage form.

TABLE 20
Ingredients for Sleep formulated as edible chews

| Ingredient | Volume | | Weight (g) | Ratio |
|---|---|---|---|---|
| Dark chocolate (70% cacao) | n/a | | 600 | 0.816 |
| Liquid matrix | 80 | ml | 80 | 0.110 |
| Dried fruiting bodies | n/a | | 10 | 0.013 |
| Turmeric root | 1 | tbsp | 10 | 0.013 |
| Nutmeg seed | 1½ | tbsp | 10 | 0.013 |
| Cinnamon stalk | 2 | tsp | 5 | 0.07 |
| Clove fruit, stems and buds | 2 | tsp | 5 | 0.07 |
| cannabis extract with over 50% CBD | 1 | tsp | 5 | 0.07 |
| and over 2% CBN content | | | | |

| | | | |
|---|---|---|---|
| Serotonin formulated into 400 mg | n/a | 5 | 0.07 |
| capsules including 50 mg serotonin | | | |
| Melatonin formulated into 250 mg | n/a | 5 | 0.07 |
| capsules including 5 mg melatonin | | | |

The ingredients in Table 20 provide a total mass of 735 g. The weight per dosage unit is 5 g, including about 0.07 g of dried fruiting bodies. This provides 147 dosage units from the ingredients in Table 20. The dried fruiting bodies are about 1.0% psilocybin, providing about 0.7 mg psilocybin per dosage unit.

### Example Formulation 09—Anti-Depression

Table 21 shows the ingredients for Anti-Depression formulated as material to be included in a capsule dosage form.

TABLE 21
Ingredients for Anti-Depression formulated for capsules

| Ingredient | Weight (g) | Ratio |
|---|---|---|
| Dried fruiting bodies | 25 | 0.33 |
| Turmeric root | 20 | 0.27 |
| 70% cacao dark chocolate | 10 | 0.13 |
| Nutmeg seed | 5 | 0.07 |
| Cayenne pepper fruit and seeds | 5 | 0.07 |
| Cinnamon stalk | 5 | 0.07 |
| Clove fruit, stems and buds | 5 | 0.07 |

The ingredients in Table 21 provide a total mass of 75 g. The weight per dosage unit is 0.3 g, including about 0.10 g of dried fruiting bodies. This provides 250 dosage units from the ingredients in Table 21. The dried fruiting bodies are about 1.0% psilocybin, providing about 1.0 mg psilocybin per dosage unit.

Table 22 shows the ingredients for Anti-Depression formulated as an edible chew dosage form.

TABLE 22
Ingredients for Anti-Depression formulated as edible chews

| Ingredient | Volume | | Weight (g) | Ratio |
|---|---|---|---|---|
| Dark chocolate (70% cacao) | n/a | | 550 | 0.797 |
| Liquid matrix | 80 | ml | 80 | 0.116 |
| Turmeric root | 2 | tbsp | 20 | 0.029 |
| Dried fruiting bodies | n/a | | 15 | 0.022 |
| Nutmeg seed | 1½ | tbsp | 10 | 0.014 |
| Cayenne pepper fruit and seeds | 1 | tbsp | 5 | 0.007 |
| Cinnamon stalk | 2 | tsp | 5 | 0.007 |
| Clove fruit, stems and buds | 2 | tsp | 5 | 0.007 |

The ingredients in Table 22 provide a total mass of 690 g. The weight per dosage unit is 5 g, including about 0.11 g of dried fruiting bodies. This provides 138 dosage units from the ingredients in Table 22. The dried fruiting bodies are about 1.0% psilocybin, providing about 1.1 mg psilocybin per dosage unit.

### Example Formulation 11—Relax

Table 23 shows the ingredients for Relax formulated as material to be included in a capsule dosage form.

TABLE 23
Ingredients for Relax formulated for capsules

| Ingredient | Weight (g) | Ratio |
|---|---|---|
| Dried fruiting bodies | 30 | 0.33 |
| Turmeric root | 20 | 0.22 |
| Nutmeg seed | 10 | 0.11 |
| Cayenne pepper fruit and seeds | 5 | 0.05 |
| Cinnamon stalk | 5 | 0.05 |
| Clove fruit, stems and buds | 5 | 0.05 |
| 70% cacao dark chocolate | 5 | 0.05 |
| Ground coffee beans | 5 | 0.05 |
| Bergamot flower | 3 | 0.03 |
| Peppercorns | 3 | 0.03 |

The ingredients in Table 23 provide a total mass of 88 g. The weight per dosage unit is 0.3 g, including about 0.09 g of dried fruiting bodies. This provides 293 dosage units from the ingredients in Table 23. The dried fruiting bodies are about 1.0% psilocybin, providing about 0.9 mg psilocybin per dosage unit.

Table 24 shows the ingredients for Relax formulated as an edible chew dosage form.

TABLE 24
Ingredients for Relax formulated as edible chews

| Ingredient | Volume | | Weight (g) | Ratio |
|---|---|---|---|---|
| Dark chocolate (70% cacao) | n/a | | 600 | 0.801 |
| Liquid matrix | 80 | ml | 80 | 0.107 |
| Turmeric root | 2 | tbsp | 20 | 0.027 |
| Dried fruiting bodies | n/a | | 15 | 0.020 |
| Nutmeg seed | 1½ | tbsp | 10 | 0.013 |
| Cayenne pepper fruit and seeds | 1 | tbsp | 5 | 0.007 |
| Cinnamon stalk | 2 | tsp | 5 | 0.007 |
| Clove fruit, stems and buds | 2 | tsp | 5 | 0.007 |
| Coffee beans | 1 | tbsp | 5 | 0.007 |
| Peppercorns | 1¼ | tsp | 3 | 0.004 |
| Cannabis extract with 60% w/w THC | n/a | | 1 | 0.001 |
| (not present in capsule formulation) | | | | |
| Bergamot 100% essential oil | 0.15 | ml | 0.15 | <0.001 |

The ingredients in Table 24 provide a total mass of 748 g. The weight per dosage unit is 5 g, including about 0.10 g of dried fruiting bodies. This provides 149 dosage units from the ingredients in Table 24. The dried fruiting bodies are about 1.0% psilocybin, providing about 1.0 mg psilocybin per dosage unit.

### Example Formulation 12—Focus

Table 25 shows the ingredients for Focus formulated as material to be included in a capsule dosage form.

TABLE 25
Ingredients for Focus formulated for capsules

| Ingredient | Weight (g) | Ratio |
|---|---|---|
| Dried fruiting bodies | 25 | 0.27 |
| Turmeric root | 20 | 0.22 |
| Nutmeg seed | 10 | 0.11 |
| Cayenne pepper fruit and seeds | 5 | 0.05 |
| Cinnamon stalk | 5 | 0.05 |
| Clove fruit, stems and buds | 5 | 0.05 |
| Coffee beans | 5 | 0.05 |
| 70% cacao dark chocolate | 5 | 0.05 |
| Peppermint leaves | 5 | 0.05 |
| Ginkgo biloba leaves | 3 | 0.03 |
| Peppercorns | 3 | 0.03 |

The ingredients in Table 25 provide a total mass of 91 g. The weight per dosage unit is 0.3 g, including about 0.08 g of dried fruiting bodies. This provides 303 dosage units from the ingredients in Table 25. The dried fruiting bodies are about 1.0% psilocybin, providing about 0.8 mg psilocybin per dosage unit.

Table 26 shows the ingredients for Focus formulated as an edible chew dosage form.

TABLE 26
Ingredients for Focus formulated as edible chews

| Ingredient | Volume | | Weight (g) | Ratio |
|---|---|---|---|---|
| Dark chocolate (70% cacao) | n/a | | 500 | 0.772 |
| Liquid matrix | 80 | ml | 80 | 0.123 |
| Turmeric root | 2 | tbsp | 20 | 0.031 |
| Dried fruiting bodies | n/a | | 15 | 0.023 |
| Nutmeg seed | ¾ | tbsp | 5 | 0.008 |
| Cayenne pepper fruit and seeds | 1 | tbsp | 5 | 0.008 |
| Cinnamon stalk | 2 | tsp | 5 | 0.008 |
| Clove fruit, stems and buds | 2 | tsp | 5 | 0.008 |
| Coffee beans | 1 | tbsp | 5 | 0.008 |
| Peppercorns | 2 | tsp | 5 | 0.008 |
| Ginkgo biloba leaves | n/a | | 2 | 0.003 |
| Peppermint extract | 1 | ml | 1 | 0.002 |

The ingredients in Table 26 provide a total mass of 648 g. The weight per dosage unit is 5 g, including about 0.12 g of dried fruiting bodies. This provides 129 dosage units from the ingredients in Table 26. The dried fruiting bodies are about 1.0% psilocybin, providing about 1.2 mg psilocybin per dosage unit.

### Example Formulation 13—Creative

Table 27 shows the ingredients for Creative formulated as material to be included in a capsule dosage form.

TABLE 27
Ingredients for Creative formulated for capsules

| Ingredient | Weight (g) | Ratio |
|---|---|---|
| Dried fruiting bodies | 30 | 0.31 |
| Turmeric root | 20 | 0.21 |
| Nutmeg seed | 10 | 0.10 |
| Cayenne pepper fruit and seeds | 5 | 0.05 |
| Cinnamon stalk | 5 | 0.05 |
| Clove fruit, stems and buds | 5 | 0.05 |
| 70% cacao dark chocolate | 5 | 0.05 |
| Coffee beans | 5 | 0.05 |
| Peppermint leaves | 5 | 0.05 |
| Peppercorns | 3 | 0.03 |
| Bergamot flower | 3 | 0.03 |

The ingredients in Table 27 provide a total mass of 96 g. The weight per dosage unit is 0.3 g, including about 0.09 g of dried fruiting bodies. This provides 320 dosage units from the ingredients in Table 27. The dried fruiting bodies are about 1.0% psilocybin, providing about 0.9 mg psilocybin per dosage unit.

Table 28 shows the ingredients for Creative formulated as an edible chew dosage form.

TABLE 28
Ingredients for Creative formulated as edible chews

| Ingredient | Volume | | Weight (g) | Ratio |
|---|---|---|---|---|
| Dark chocolate (70% cacao) | n/a | | 500 | 0.768 |
| Liquid matrix | 80 | ml | 80 | 0.123 |
| Dried fruiting bodies | n/a | | 20 | 0.031 |
| Turmeric root | 2 | tbsp | 20 | 0.031 |
| Nutmeg seed | 1½ | tbsp | 10 | 0.015 |
| Cayenne pepper fruit and seeds | 1 | tbsp | 5 | 0.008 |
| Cinnamon stalk | 2 | tsp | 5 | 0.008 |
| Clove fruit, stems and buds | 2 | tsp | 5 | 0.008 |
| Coffee beans | 1 | tbsp | 5 | 0.008 |
| Peppermint extract | 1 | ml | 1 | 0.002 |
| Bergamot extract | 0.15 | ml | 0.15 | <0.001 |

The ingredients in Table 28 provide a total mass of 665 g. The weight per dosage unit is 5 g, including about 0.15 g of dried fruiting bodies. This provides 133 dosage units from the ingredients in Table 28. The dried fruiting bodies are about 1.0% psilocybin, providing about 1.5 mg psilocybin per dosage unit.

### Example Formulation 14—Anti-Inflammatory for Bowel

Table 29 shows the ingredients for Anti-Inflammatory for Bowel formulated as material to be included in a capsule dosage form.

TABLE 29
Ingredients for Anti-Inflammatory
for Bowel formulated for capsules

| Ingredient | Weight (g) | Ratio |
|---|---|---|
| Dried fruiting bodies | 30 | 0.385 |
| Turmeric root | 10 | 0.128 |
| Cayenne pepper fruit and seeds | 5 | 0.064 |
| Cinnamon stalk | 5 | 0.064 |
| Clove fruit, stems and buds | 5 | 0.064 |
| Cannabis flower (not present in edible | 5 | 0.064 |
| chew formulation) with between 10 and | | |
| 20% combined THC and CBD | | |
| 70% cacao dark chocolate | 5 | 0.064 |
| Ginger | 3 | 0.038 |
| Garlic | 2.5 | 0.032 |
| Onion | 2.5 | 0.032 |
| Thyme | 2 | 0.026 |
| Oregano | 2 | 0.026 |
| Cardamom | 1 | 0.013 |

The ingredients in Table 29 provide a total mass of 78 g. The weight per dosage unit is 0.3 g, including about 0.12 g of dried fruiting bodies. This provides 260 dosage units from the ingredients in Table 29. The dried fruiting bodies are about 1.0% psilocybin, providing about 1.2 mg psilocybin per dosage unit.

Table 30 shows the ingredients for Anti-Inflammatory for Bowel formulated as an edible chew dosage form.

TABLE 30
Ingredients for Anti-Inflammatory for
Bowel formulated as edible chews

| Ingredient | Volume | | Weight (g) | Ratio |
|---|---|---|---|---|
| Dark chocolate (70% cacao) | n/a | | 550 | 0.797 |
| Liquid matrix | 80 | ml | 80 | 0.116 |
| Turmeric root | 2 | tbsp | 20 | 0.029 |
| Dried fruiting bodies | n/a | | 15 | 0.022 |
| Cayenne pepper fruit and seeds | 1 | tbsp | 5 | 0.007 |
| Cinnamon stalk | 2 | tsp | 5 | 0.007 |
| Clove fruit, stems and buds | 2 | tsp | 5 | 0.007 |
| Ginger | 1⅔ | tsp | 3 | 0.004 |
| Garlic | 1 | tsp | 2.5 | 0.004 |
| Onion | 1 | tsp | 2.5 | 0.004 |
| Cardamom | 1 | tsp | 2 | 0.003 |
| Oregano oil | 10 | drops | 0.5 | 0.001 |
| Thyme oil | 2 | drops | 1 | <0.001 |

The ingredients in Table 30 provide a total mass of 691 g. The weight per dosage unit is 5 g, including about 0.11 g of dried fruiting bodies. This provides 138 dosage units from the ingredients in Table 30. The dried fruiting bodies are about 1.0% psilocybin, providing about 1.1 mg psilocybin per dosage unit.

### Example Formulation 15—Digestion

Table 31 shows the ingredients for Digestion formulated as material to be included in a capsule dosage form.

TABLE 31
Ingredients for Digestion formulated for capsules

| Ingredient | Weight (g) | Ratio |
|---|---|---|
| Dried fruiting bodies | 25 | 0.37 |
| Turmeric root | 10 | 0.15 |
| Cinnamon stalk | 5 | 0.07 |
| Clove fruit, stems and buds | 5 | 0.07 |
| 70% cacao dark chocolate | 5 | 0.07 |
| Decarboxylated cannabis flower with 1:1 | 5 | 0.07 |
| ratio of THC:CBD and 20% w/w total | | |
| phytocannabinoids | | |
| Peppermint leaves | 5 | 0.07 |
| Ginger | 2.5 | 0.04 |
| Garlic | 2.5 | 0.04 |
| Cardamom | 2 | 0.03 |

The ingredients in Table 31 provide a total mass of 67 g. The weight per dosage unit is 0.3 g, including about 0.11 g of dried fruiting bodies. This provides 223 dosage units from the ingredients in Table 31. The dried fruiting bodies are about 1.0% psilocybin, providing about 1.1 mg psilocybin per dosage unit.

Table 32 shows the ingredients for Digestion formulated as an edible chew dosage form.

TABLE 32
Ingredients for Digestion formulated as edible chews

| Ingredient | Volume | | Weight (g) | Ratio |
|---|---|---|---|---|
| Dark chocolate (70% cacao) | n/a | | 500 | 0.790 |
| Liquid matrix | 80 | ml | 80 | 0.126 |
| Turmeric root | 2 | tbsp | 20 | 0.032 |
| Dried fruiting bodies | n/a | | 15 | 0.024 |
| Cinnamon stalk | 2 | tsp | 5 | 0.008 |
| Clove fruit, stems and buds | 2 | tsp | 5 | 0.008 |
| Garlic | 1 | tsp | 2.5 | 0.004 |
| Ginger | 1 | tsp | 2.5 | 0.004 |
| Cardamom | ¾ | tsp | 2 | 0.003 |
| Peppermint extract | 1 | ml | 1 g | 0.002 |

The ingredients in Table 32 provide a total mass of 633 g. The weight per dosage unit is 5 g, including about 0.11 g of dried fruiting bodies. This provides 126 dosage units from the ingredients in Table 32. The dried fruiting bodies are about 1.0% psilocybin, providing about 1.1 mg psilocybin per dosage unit.

### Example Formulation 16—Analgesia

Table 33 shows the ingredients for Analgesia formulated as material to be included in a capsule dosage form.

TABLE 33
Ingredients for Analgesia formulated for capsules

| Ingredient | Weight (g) | Ratio |
|---|---|---|
| Dried fruiting bodies | 25 | 0.34 |
| Turmeric root | 10 | 0.14 |
| Cayenne pepper fruit and seeds | 5 | 0.07 |
| Cinnamon stalk | 5 | 0.07 |
| Clove fruit, stems and buds | 5 | 0.07 |
| Decarboxylated cannabis flower with 1:4 | 5 | 0.07 |
| ratio or lower of THC:CBD and about | | |
| 20% w/w total phytocannabinoids | | |
| 70% cacao dark chocolate | 5 | 0.07 |
| Peppermint leaves | 5 | 0.07 |
| Peppercorns | 3 | 0.04 |
| Thyme | 2.5 | 0.03 |
| Oregano | 2.5 | 0.03 |

The ingredients in Table 33 provide a total mass of 73 g. The weight per dosage unit is 0.3 g, including about 0.10 g of dried fruiting bodies. This provides 243 dosage units from the ingredients in Table 33. The dried fruiting bodies are about 1.0% psilocybin, providing about 1.0 mg psilocybin per dosage unit.

Table 34 shows the ingredients for Analgesia formulated as an edible chew dosage form.

TABLE 34
Ingredients for Analgesia formulated as edible chews

| Ingredient | Volume | | Weight (g) | Ratio |
|---|---|---|---|---|
| Dark chocolate (70% cacao) | n/a | | 550 | 0.797 |
| Liquid matrix | 80 | ml | 80 | 0.116 |
| Turmeric root | 2 | tbsp | 20 | 0.029 |
| Dried fruiting bodies | n/a | | 15 | 0.022 |
| Cayenne pepper fruit and seeds | 1 | tbsp | 5 | 0.007 |
| Cinnamon stalk | 2 | tsp | 5 | 0.007 |
| Clove fruit, stems and buds | 2 | tsp | 5 | 0.007 |
| Decarboxylated cannabis extract with | n/a | | 5 | 0.007 |
| 1:4 ratio or lower of THC:CBD and | | | | |
| about 60% to 80% w/w total | | | | |
| phytocannabinoids | | | | |
| Peppercorns | n/a | | 3 | 0.004 |
| Oregano oil | 10 | drops | 0.5 | 0.001 |
| Peppermint oil | 10 | drops | 0.5 | 0.001 |
| Thyme oil | 2 | drops | 0.1 | <0.001 |

The ingredients in Table 34 provide a total mass of 689 g. The weight per dosage unit is 5 g, including about 0.11 g of dried fruiting bodies. This provides 137 dosage units from the ingredients in Table 34. The dried fruiting bodies are about 1.0% psilocybin, providing about 1.1 mg psilocybin per dosage unit.

### Example Formulation 17—TBI Treatment

Table 35 shows the ingredients for TBI Treatment formulated as material to be included in a capsule dosage form.

TABLE 35
Ingredients for TBI Treatment formulated for capsules

| Ingredient | Weight (g) | Ratio |
|---|---|---|
| Dried fruiting bodies | 40 | 0.37 |
| Turmeric root | 20 | 0.19 |
| Nutmeg seed | 10 | 0.09 |
| Decarboxylated cannabis flower with 2:1 | 10 | 0.09 |
| ratio or greater of THC:CBD and about | | |
| 20% w/w total phytocannabinoids | | |
| Cayenne pepper fruit and seeds | 5 | 0.05 |
| Cinnamon stalk | 5 | 0.05 |
| Clove fruit, stems and buds | 5 | 0.05 |
| 70% cacao dark chocolate | 5 | 0.05 |
| Ginkgo biloba | 3 | 0.03 |
| Myrrh | 2.5 | 0.02 |
| Frankincense | 2.5 | 0.02 |

The ingredients in Table 35 provide a total mass of 108 g. The weight per dosage unit is 0.3 g, including about 0.11 g of dried fruiting bodies. This provides 360 dosage units from the ingredients in Table 35. The dried fruiting bodies are about 1.0% psilocybin, providing about 1.1 mg psilocybin per dosage unit.

Table 36 shows the ingredients for TBI Treatment formulated as an edible chew dosage form.

TABLE 36
Ingredients for TBI Treatment formulated as edible chews

| Ingredient | Volume | | Weight (g) | Ratio |
|---|---|---|---|---|
| Dark chocolate (70% cacao) | n/a | | 650 | 0.812 |
| Liquid matrix | 80 | ml | 80 | 0.100 |
| Dried fruiting bodies | n/a | | 20 | 0.025 |
| Turmeric root | 2 | tbsp | 20 | 0.025 |
| Nutmeg seed | 1 1/2 | tbsp | 10 | 0.012 |
| Cayenne pepper fruit and seeds | 1 | tbsp | 5 | 0.006 |
| Cinnamon stalk | 2 | tsp | 5 | 0.006 |
| Clove fruit, stems and buds | 2 | tsp | 5 | 0.006 |
| Ginkgo biloba leaves | 2 | tsp | 5 | 0.006 |
| Myrrh extract | 10 | drops | 0.5 | 0.001 |
| Frankincense extract | 2 | drops | 0.1 | <0.001 |

The ingredients in Table 36 provide a total mass of 801 g. The weight per dosage unit is 5 g, including about 0.12 g of dried fruiting bodies. This provides 160 dosage units from the ingredients in Table 36. The dried fruiting bodies are about 1.0% psilocybin, providing about 1.2 mg psilocybin per dosage unit.

### Example Formulation 18—Aphrodisiac

Table 37 shows the ingredients for Aphrodisiac formulated as material to be included in a capsule dosage form.

TABLE 37
Ingredients for Aphrodisiac formulated for capsules

| Ingredient | Weight (g) | Ratio |
|---|---|---|
| Dried fruiting bodies | 30 | 0.28 |
| Turmeric root | 20 | 0.19 |
| Nutmeg seed | 10 | 0.09 |
| Cayenne pepper fruit and seeds | 5 | 0.05 |
| Cinnamon stalk | 5 | 0.05 |
| Clove fruit, stems and buds | 5 | 0.05 |
| Coffee beans | 5 | 0.05 |
| 70% cacao dark chocolate | 5 | 0.05 |
| Peppermint leaves | 5 | 0.05 |
| Kava kava root | 5 | 0.05 |
| Peppercorns | 3 | 0.03 |
| Ginkgo biloba | 3 | 0.03 |
| Frankincense | 2.5 | 0.02 |
| Myrrh | 2.5 | 0.02 |
| Cardamom | 1 | 0.01 |

The ingredients in Table 37 provide a total mass of 107 g. The weight per dosage unit is 0.3 g, including about 0.14 g of dried fruiting bodies. This provides 356 dosage units from the ingredients in Table 37. The dried fruiting bodies are about 1.0% psilocybin, providing about 0.14 mg psilocybin per dosage unit.

Table 38 shows the ingredients for Aphrodisiac formulated as an edible chew dosage form.

TABLE 38
Ingredients for Aphrodisiac formulated as edible chews

| Ingredient | Volume | | Weight (g) | Ratio |
|---|---|---|---|---|
| Dark chocolate (70% cacao) | n/a | | 500 | 0.748 |
| Liquid matrix | 80 | ml | 80 | 0.120 |
| Dried fruiting bodies | n/a | | 25 | 0.037 |
| Turmeric root | 2 | tbsp | 20 | 0.030 |
| Nutmeg seed | 1½ | tbsp | 10 | 0.015 |
| Kava kava root | 2 | tsp | 5 | 0.007 |
| Cayenne pepper fruit and seeds | 1 | tbsp | 5 | 0.007 |
| Cinnamon stalk | 2 | tsp | 5 | 0.007 |
| Clove fruit, stems and buds | 2 | tsp | 5 | 0.007 |
| Coffee beans | 1 | tbsp | 5 | 0.007 |
| Peppercorns | 1½ | tsp | 3 | 0.004 |
| Ginkgo biloba leaves | ¾ | tsp | 2 | 0.003 |
| Cardamom seeds | n/a | | 2 | 0.003 |
| Peppermint extract | 1 | ml | 1 | 0.001 |
| Myrrh | 10 | drops | 0.5 g | 0.001 |
| Frankincense | 2 | drops | 0.1 g | <0.001 |

The ingredients in Table 38 provide a total mass of 668 g. The weight per dosage unit is 5 g, including about 0.18 g of dried fruiting bodies. This provides 133 dosage units from the ingredients in Table 38. The dried fruiting bodies are about 1.0% psilocybin, providing about 1.8 mg psilocybin per dosage unit.

### Example 3

Three example formulations were prepared including some of the examples compositions described in Tables 8 and 9. The Base Complete and Focus formulations were prepared as capsules. The Focus formulation was also prepared as an edible chew. These formulations were prepared with serotonin as the primary 5HT2A agonist, but could be prepared with any suitable 5HT2A agonist. The serotonin was provided as purified and formulated serotonin with 50 mg serotonin in each 400 mg capsule.

Unlike psilocybin, many other tryptamines, many ergolines and many phenethylamines, serotonin is not strongly psychoactive and in many jurisdictions is not a controlled substance. The safety and daily consumption limit have been well studied allowing these formulations to be administered to a broader range of individuals including minors.

### Example Formulation 07—Complete

Table 39 shows the ingredients for Base Complete formulated as material to be included in a capsule dosage form.

TABLE 39
Ingredients for Base Complete formulated for capsules

| Ingredient | Weight (g) | Ratio |
|---|---|---|
| Turmeric root | 20 | 0.26 |
| Compounded serotonin powder | 10 | 0.13 |
| Nutmeg seed | 10 | 0.13 |
| Coffee beans | 10 | 0.13 |
| 70% cacao dark chocolate | 10 | 0.13 |
| Cayenne pepper fruit and seeds | 7 | 0.09 |
| Cinnamon stalk | 5 | 0.06 |
| Clove fruit, stems and buds | 5 | 0.06 |

The ingredients in Table 39 provide a total mass of 77 g. The weight per dosage unit is 0.5 g, including about 0.06 g of compounded serotonin powder. This provides 154 dosage units from the ingredients in Table 39. The compounded serotonin powder is about 12.5% serotonin, providing about 7.5 mg serotonin per dosage unit.

### Example Formulation 12—Focus

Table 40 shows the ingredients for Focus formulated as material to be included in a capsule dosage form.

TABLE 40
Ingredients for Focus formulated for capsules

| Ingredient | Weight (g) | Ratio |
|---|---|---|
| Compounded serotonin powder | 20 | 0.23 |
| Turmeric root | 20 | 0.23 |
| Nutmeg seed | 10 | 0.12 |
| Cayenne pepper fruit and seeds | 5 | 0.06 |
| Cinnamon stalk | 5 | 0.06 |
| Clove fruit, stems and buds | 5 | 0.06 |
| Coffee beans | 5 | 0.06 |
| 70% cacao dark chocolate | 5 | 0.06 |
| Peppermint leaves | 5 | 0.06 |
| Ginkgo biloba leaves | 3 | 0.03 |
| Peppercorns | 3 | 0.03 |

The ingredients in Table 40 provide a total mass of 86 g. The weight per dosage unit is 0.3 g, including about 0.07 g of compounded serotonin powder. This provides 286 dosage units from the ingredients in Table 40. The compounded serotonin powder is about 12.5% serotonin, providing about 8.75 mg serotonin per dosage unit.

Table 41 shows the ingredients for Focus formulated as an edible chew dosage form.

TABLE 41
Ingredients for Focus formulated as edible chews

| Ingredient | Volume | | Weight (g) | Ratio |
|---|---|---|---|---|
| Dark chocolate (70% cacao) | n/a | | 500 | 0.778 |
| Liquid matrix | 80 | ml | 80 | 0.124 |
| Turmeric root | 2 | tbsp | 20 | 0.031 |
| Compounded serotonin powder | n/a | | 15 | 0.016 |
| Nutmeg seed | ¾ | tbsp | 5 | 0.008 |
| Cayenne pepper fruit and seeds | 1 | tbsp | 5 | 0.008 |
| Cinnamon stalk | 2 | tsp | 5 | 0.008 |
| Clove fruit, stems and buds | 2 | tsp | 5 | 0.008 |
| Coffee beans | 1 | tbsp | 5 | 0.008 |
| Peppercorns | 2 | tsp | 5 | 0.008 |
| Ginkgo biloba leaves | n/a | | 2 | 0.003 |
| Peppermint extract | 1 | ml | 1 | 0.002 |

The ingredients in Table 41 provide a total mass of 643 g. The weight per dosage unit is 5 g, including about 0.08 g of compounded serotonin powder. This provides 128 dosage units from the ingredients in Table 41. The compounded serotonin powder is about 12.5% serotonin, providing about 1.0 mg serotonin per dosage unit.

### Example 4

Three example formulations were prepared including some of the examples compositions described in Tables 8 and 9. The Base Complete, formulations were prepared as capsules. These formulations were prepared with ergolines as the 5HT2A agonist, but could be prepared with any suitable 5HT2A agonist. The ergolines were provided either morning glory seeds or Hawaiian baby woodrose seeds that have been crushed and pulverized into a fine powder.

### Example Formulation 07—Base Complete

Table 42 shows the ingredients for Base Complete formulated as material to be included in a capsule dosage form.

TABLE 42
Ingredients for Base Complete formulated for capsules

| Ingredient | Weight (g) | Ratio |
|---|---|---|
| Morning glory seed | 40 | 0.42 |
| Turmeric root | 20 | 0.21 |
| Nutmeg seed | 10 | 0.11 |
| Cayenne pepper fruit and seeds | 5 | 0.05 |
| Cinnamon stalk | 5 | 0.05 |
| Clove fruit, stems and buds | 5 | 0.05 |
| Coffee beans | 5 | 0.05 |
| 70% cacao dark chocolate | 5 | 0.05 |

The ingredients in Table 42 provide a total mass of 95 g. The weight per dosage unit is 0.5 g, including about 0.21 g of morning glory seeds. This provides 253 dosage units from the ingredients in Table 42.

According to one study, morning glory seeds are between 260 μg/g and 300 μg/g LSA and about 0.5 to 1.75 relative abundance of LSH to LSA and ergometrine, providing between 130 μg/g and 525 μg/g LSH in morning glory seeds (Nowak, J., Woźiniakiewicz, M., Klepacki, P., Sowa, A., & Kościelniak, P. (2016). Identification and determination of ergot alkaloids in Morning Glory cultivars. Analytical and bioanalytical chemistry, 408(12), 3093-3102, which is incorporated herein by reference in its entirety). At these concentrations of ergolines, each dosage unit includes between 54 to 63 μg LSA per dosage unit and between 27 and 95 μg LSH per dosage unit.

### Example Formulation 07—Base Complete

Table 43 shows the ingredients for Base Complete formulated as material used in in a tea format. Peppermint tea may be used to mitigate potential stomach cramping, through the addition to menthol providing a 5HT3 antagonist. The tea may also be combined with bergamot, garlic or ginger.

TABLE 43
Ingredients for Base Complete formulated for use in tea

| Ingredient | Weight (g) | Ratio |
|---|---|---|
| Morning glory seed | 42 | 0.42 |
| Turmeric root | 21 | 0.21 |
| Nutmeg seed | 12 | 0.12 |
| Cayenne pepper fruit and seeds | 5 | 0.05 |
| Cinnamon stalk | 5 | 0.05 |
| Clove fruit, stems and buds | 5 | 0.05 |
| Coffee beans | 5 | 0.05 |
| 70% cacao dark chocolate | 5 | 0.05 |

The ingredients in Table 43 provide a total mass of 99 g. One gram of the formulation is used in a cup of tea, which includes including about 0.42 g of morning glory seeds.

According to one study, morning glory seeds are between 260 μg/g and 300 μgig LSA and about 0.5 to 1.75 relative abundance of LSH to LSA and ergometrine, providing between 130 μg/g and 525 μg/g LSH in morning glory seeds (Nowak, 2016). At these concentrations of ergolines, each dosage unit includes between 260 μg and 300 μg LSA per dosage unit and between 130 μg and 525 μg LSH per dosage unit.

### Example Formulation 07—Base Complete

Table 44 shows the ingredients for Base Complete formulated as an edible chew dosage form. This formulation may also include peppercorns or CBD.

TABLE 44
Ingredients for Focus formulated as edible chews

| Ingredient | Volume | | Weight (g) | Ratio |
|---|---|---|---|---|
| Dark chocolate (70% cacao) | n/a | | 600 | 0.780 |
| Liquid matrix | 80 | ml | 80 | 0.104 |
| Morning glory seeds | n/a | | 42 | 0.055 |
| Turmeric root | 2 | tbsp | 20 | 0.027 |
| Nutmeg seed | 2 | tbsp | 12 | 0.016 |
| Cinnamon stalk | 2 | tsp | 5 | 0.006 |
| Clove fruit, stems and buds | 2 | tsp | 5 | 0.006 |
| Cayenne pepper fruit and seeds | ¾ | tbsp | 4 | 0.005 |
| Peppermint oil extract | 0.5 | ml | 0.5 | 0.001 |

The ingredients in Table 44 provide a total mass of 769.5 g. Each dosage unit is 9 g, which includes including about 0.49 g of morning glory seeds. This provides 86 dosage units from the ingredients in Table 44.

According to one study, morning glory seeds are between 260 μg/g and 300 μg/g LSA and about 0.5 to 1.75 relative abundance of LSH to LSA and ergometrine, providing between 130 μg/g and 525 μg/g LSH in morning glory seeds (Nowak, 2016). At these concentrations of ergolines, each dosage unit includes between 130 and 150 μg LSA and between 65 and 260 μg LSH.

### Example Formulation 07—Base Complete

Table 45 shows the ingredients for Base Complete formulated as material to be included in a capsule dosage form with Hawaiian Baby woodrose seeds.

TABLE 45
Ingredients for Base Complete formulated for capsules

| Ingredient | Weight (g) | Ratio |
|---|---|---|
| Turmeric root | 20 | 0.35 |
| Nutmeg seed | 10 | 0.18 |
| 70% cacao dark chocolate | 10 | 0.18 |
| Cayenne pepper fruit and seeds | 7 | 0.12 |
| Cinnamon stalk | 5 | 0.09 |
| Clove fruit, stems and buds | 5 | 0.09 |

The ingredients in Table 45 provide a total mass of 57 g. The weight per dosage unit is 0.3 g. This provides 190 dosage units from the ingredients in Table 45. The capsules may be taken with a separate capsule including between 50 and 300 mg Hawaiian baby woodrose seeds. Due to the number of doses required to achieve efficacy with Morning glory seeds, Hawaiian baby woodrose seeds were pulverized and distributed as a separate pill as a preferred method of consumption. This allows the user to increase either the 5HT2A agonists or the TRP agonists separately.

According to one study, Hawaiian baby woodrose seeds are about 1,400 μgig LSA, 1,800 μg/g iso-LSA, 350 μg/g LSH and 240 μg/g iso-LSH (Chao, 1973). At these concentrations of ergolines, each 50 mg to 300 mg dosage unit of Hawaiian baby woodrose seeds includes between 70 μg and 420 μg LSA, between 90 μg and 540 μg iso-LSA, between 20 μg and 120 μg LSH and between 10 μg and 70 μg iso-LSH.

### Example Formulation 07—Base Complete with Garlic and Onion

Table 46 shows the ingredients for Base Complete with garlic and onion formulated as material to be included in a capsule dosage form.

TABLE 46
Ingredients for Base Complete formulated for capsules

| Ingredient | Weight (g) | Ratio |
|---|---|---|
| Turmeric root | 20 | 0.33 |
| Nutmeg seed | 10 | 0.17 |
| Cayenne pepper fruit and seeds | 5 | 0.08 |
| 70% cacao dark chocolate | 5 | 0.08 |
| Cinnamon stalk | 5 | 0.08 |
| Clove fruit, stems and buds | 5 | 0.08 |
| Ginger | 5 | 0.08 |
| Garlic | 5 | 0.08 |

The ingredients in Table 46 provide a total mass of 60 g. The weight per dosage unit is 0.3 g, This provides 200 dosage units from the ingredients in Table 46. The capsules may be taken with a separate capsule including between 50 and 300 mg Hawaiian baby woodrose seeds.

According to one study, Hawaiian baby woodrose seeds are about 1,400 μg/g LSA, 1,800 μg/g iso-LSA, 350 μg/g LSH and 240 μg/g iso-LSH (Chao, 1973). At these concentrations of ergolines, each 50 mg to 300 mg dosage unit of Hawaiian baby woodrose seeds includes between 70 μg and 420 μg LSA, between 90 μg and 540 μg iso-LSA, between 20 μg and 120 μg LSH and between 10 μg and 70 μg iso-LSH.

### Example Formulation 07—Base Complete

Table 47 shows the ingredients for Base Complete formulated as material to be included in a capsule dosage form.

TABLE 47
Ingredients for Base Complete formulated for capsules

| Ingredient | Weight (g) | Ratio |
|---|---|---|
| Turmeric root | 20 | 0.31 |
| Hawaiian baby woodrose seeds | 10 | 0.16 |
| Nutmeg seed | 10 | 0.16 |
| Cinnamon stalk | 5 | 0.08 |
| Clove fruit, stems and buds | 5 | 0.08 |
| Coffee beans | 5 | 0.08 |
| 70% cacao dark chocolate | 5 | 0.08 |
| Cayenne pepper fruit and seeds | 4 | 0.06 |

The ingredients in Table 47 provide a total mass of 64 g. The weight per dosage unit is 0.5 g, including about 0.16 g of Hawaiian baby woodrose seeds. This provides 128 dosage units from the ingredients in Table 47.

According to one study, Hawaiian baby woodrose seeds are about 1,400 μgig LSA, 1,800 μg/g iso-LSA, 350 μg/g LSH and 240 μg/g iso-LSH (Chao, 1973). At these concentrations of ergolines, each dosage unit includes 220 μg LSA, 290 μg iso-LSA, 60 LSH and 40 μg iso-LSH.

### Example Formulation 19—Enhanced Focus

Table 48 shows the ingredients for an Enhanced Focus LSA/LSH recipe that can be made with either morning glory or Hawaiian baby woodrose.

TABLE 48
Ingredients for Enhanced Focus formulated for capsules

| Ingredient | Weight (g) | Ratio |
|---|---|---|
| Morning Glory citrate powder | 42 | 0.66 |
| Nutmeg seed | 21 | 0.33 |
| Peppermint oil | 0.5 | 0.01 |
| Frankincense oil | 0.25 | 3.92E−05 |

The ingredients in Table 48 provide a total mass of 63.75 g. Each dosage unit is 0.3 g, which includes including about 0. 198 g of morning glory seeds in citrate powder form. This provides 212 dosage units from the ingredients in Table 44.

Reformulation and extraction of LSA and LSH with citric juices or citric acid was found to make the recipe significantly more tolerable and palatable. Seeds were pulverized and soaked in juices of lemon, lime or orange, followed by dehydration in a standard food dehydrator. An alcohol may be added to aid in the process or even extract the active metabolites if desired. The seed mash is soaked in the liquid overnight (12 hours minimum), dried and then reground and used in formulations. The process was also repeated with Citric Acid at pH 3.5 with a similar result being achieved. Conversion of LSA/LSH to a citrate form of the molecule can be confirmed with a UV ‘black’ light as the seed mash fluoresces after this treatment. Though the complete conversions of the molecules cannot be confirmed, it is presumed that at least some of the metabolites are converted. It is suspected this allows them to pass the stomach more easily and bypass some of the interaction with 5HT3 receptors that may cause nausea. According to one study, morning glory seeds are between 260 μg/g and 300 μg/g LSA and about 0.5 to 1.75 relative abundance of LSH to LSA and ergometrine, providing between 130 μg/g and 525 μg/g LSH in morning glory seeds (Nowak, 2016). At these concentrations of ergolines, each dosage unit includes between 130 and 150 μg LSA and between 65 and 260 μg LSH, though at least some of this is suspected to be in a citrate form through chemical reaction processes.

### Example 5

Product development studies were directed to defining MED formulations that would reduce or eliminate any potential negative side effects and also mitigate the psychoactive effects of psilocybin. The specific objectives were fivefold. First, to develop a multimodal a broad spectrum treatment for multi-symptomatic individuals and those who present with more than one persistent medical condition including those related to inflammation, pain and a mental health condition or mood disorder. Second, to prevent any tolerance or resistance to the 5HT2A agonist activity being established with prolonged or consecutive use. Some issues have been reported with taking microdose formulations everyday resulting in a reduction in efficacy, requiring a higher dose to be taken subsequently or a period of abstinence time between doses, for example taken once every three days. Ramping doses of the 5HT2A agonist may complicate stabilization of a condition or of general health. Third, to eliminate any intoxicating effects associated with 5HT2A agonism, facilitating use of formulations during everyday activities without compromising, impairing, perturbing or otherwise altering mental ability as a result of strongly psychoactive effects of the 5HT2A agonist. To mitigate intoxicating effects, potentiator compounds were assessed that would allow for a reduced dose of the 5HT2A agonist while still retaining efficacy of the 5HT2A agonist. Fourth, to achieve a prolonged and sustained effect. Often psilocybin shows a relatively rapid onset with maximum effects at 45 min to 1 hour and then notable reduced effects tapering off from this point. In contrast, some other 5HT2A agonists show sustained activity for sometimes 8 to 12 hours or more. Inclusion of specific ingredients were meant to prolong the effect of the psilocybin or other ingredients to reduce the need for regular dosing and to allow for individuals to gauge the duration of the effects of the compositions. Fifth, to achieve little to no side physical side-effects, including mitigating psychoactive effects, and to mitigate stomach and bowel discomfort, each of which are often reported with use of psilocybin. Some ingredients was specifically to reduce unpleasant physical effects from any of the active ingredients in the formulation.

### Methods

Edible chew and capsule formulations including the Base 07—Complete with coffee beans, the 5HT2A agonist alone with corn starch (positive control) or sugar and placebo formulations including corn starch (negative control) or sugar alone (double negative control) were prepared and provided to small groups of individuals for assessment of the effects of the compositions.

All participants were informed that each dosage unit of active composition or psilocybin control that included, depending on the study, between 0.05 and 0.30 g of dried P. cubensis fruiting bodies, between 0.05 and 0.50 g of morning glory seeds or between 0.05 and 0.50 g of Hawaiian baby woodrose seeds. The formulations also included additional GRAS material. Participants were informed that the data would be used for research and for protection of intellectual property rights. All participants were informed that a survey would follow and that participation in the survey was voluntary. No potential benefits were communicated to participants. All studies with psilocybin were conducted in Jamaica where psilocybin is not scheduled as a controlled substance. All studies with morning glory seeds or Hawaiian baby woodrose seeds were in a jurisdiction where possession and person use of morning glory seeds or Hawaiian baby woodrose seeds is compliant with controlled substances law. Some studies were undertaken in the context of a music festival. Other studies were undertaken at psilocybin retreat events. Numerous individuals also self-administered the formulations in their personal space and observed their daily routines over an extended number of days of using the formulations. Exclusion criteria were applied in a questionnaire to exclude individuals with a schizophrenia diagnosis, individuals with a family history of schizophrenia, pregnant individuals, minors and other high-risk categories for using psilocybin.

Typically, between 1 and 9 dosage units were provided to each participant per day. The studies varied in duration from one day to four weeks. Heavier individuals (<75 kg) were typically given larger dosages. This conservative approach to dosing participants is consistent with the MED approach and with mitigating potential negative results from the study.

Results from Music Festival Surveys

On multiple occasions a standard dose of the chocolate format of the complete recipe was distributed prior to a music festival event in Jamaica. Participants signed waivers and knowingly were consuming a “psilocybin microdose formulation” and a minimal education and safety session was given regarding the dose prior to being distributed. The GRAS material in the formulation was not identified. Some participants completed optional surveys following the experience, and data was gathered from the surveys. Participants were allowed to consume up to 3 chocolates throughout an evening though only one is distributed to start. A minimum delay of one hour was applied for all participants before a second dose or third dose.

Overall, there was an astoundingly positive response. Individuals were happily surprised that they consumed less alcohol or other drugs after taking the formulation. Individuals reported feeling alert and good overall the following day. No hangover of strong intoxication effects was reported. Out of one hundred participants at a music festival, only one participant reported feeling overwhelmed by anxiety related to the effects to the extent of choosing to leave the music festival. A few individuals reported not feeling anything at all. Most reported having an excellent experience with elevation in mood and enhancement of feelings of well-being and energy, as well as lasting positive changes post-experience. Some individuals experienced profound and lasting changes in perspective on life. One individual reported that previous intrusive suicidal thoughts were completely eliminated following one dose of the formulation. All individuals who chose to also consume alcohol reported reduced consumption.

Results from Wellness Retreat Surveys

Participants attended a five-day all inclusive retreat in Jamaica with a complete integrative wellness package. While no health benefits were attributed to psilocybin, participants were aware of optional microdose chocolates or capsules offered up to three times a day. The formulations were of the 07—Base Complete composition. In some cases, the composition was adjusted for guests who may have had specific conditions they were looking to address and formulations of other compositions were provided to these guests. Formulations including Base 2—Mood Support, 11—Relaxation, 12—Focus, 13—Creativity and 18—Aphrodisiac.

Many participants reported a life altering experience with lasting and meaningful change in their lives. Some participants reported that the experience felt like the first vacation they feel truly rested from. Many felt renewed and able to return to their lives and struggles with new skills and tools around health and well being. Both single individuals and couples showed benefits in their lives and relationships.

More specifically, participants reported reduction in negative symptoms, such as, depression, anxiety, suicidal ideation, PTSD symptoms, and negative self-talk. Participants also reported improvements of personal and work relationship communication, sleep health, inspiration to perform self-care tasks, increased creativity and more. No negative side effects or other negative consequences by individuals attending the retreat have been reported.

Formulations of the 07 Base—Complete were provided to multiple individuals who subsequently reported that they had been on the verge of attempting suicide or had recently attempted to commit suicide. In some cases, a single MED drastically improved the individual's mental state. In all cases, the desire to commit suicide was drastically reduced or completely eliminated, facilitating other therapeutic interventions. Most continued daily activities after taking a MED of a formulation of the 07 Base—Complete composition with only intermittent breaks. These individuals have often reported feeling like “themselves again” or “not having felt as good in years”.

Often these individuals were using or addicted to other drugs, whether prescription pharmaceuticals or illicit substances—and have replaced these substances with the formulation, and in some cases reported maintaining societal functions such as working, driving, and other regular activities. Many reported increased interest in daily life activities, a shift in perspective, a presence of positive emotions, increased energy and an increased desire and ability to interact, communicate or feel connected with others.

Results from Unsupervised Regular Home Use.

Many individuals consumed the 07 Base—Complete composition in a capsule or chocolate formulation. In some cases, other recipes were formulated based on the specific condition the individual might have been facing such as comorbidities with depression. Many saw profound changes within a short period of time (hours to days). These changes were often associated with both mental and physical health. Improved mood and increased energy are reported by most individuals consuming the formulation daily. Little to no tolerance was reported with daily use. Some individuals increased dosage as weeks progressed, though most found a single dose effective and were able to perform regular tasks at this dose. Most individuals who used pharmaceutical drugs such as painkillers and antidepressants reported decreased use of these substances, but often required a higher dose (such as 2×) to obtain efficacy. Most individuals who used alcohol reported reduced consumption of alcohol and regular use of other potentially impairing substances after using the formulations.

### Overall Summary of Results

After over three thousand doses distributed in Jamaica, including hundreds of individuals who consumed the compositions for at least three days in a row, there have had almost no reports of negative side effects and zero serious adverse events reported.

In all cases of placebo, no statistical significant improvement was noted and no effects reported. For psilocybin alone, at the point which the individual reported feeling the effects of the psilocybin, they also reported some psychoactive effects and sometimes physical side-effects. With the inclusion of TRP agonists with psilocybin, the amount of psilocybin needed to achieve a notable effect was reduced, resulting in a reduction in psychoactive effects and other side effects. In some cases, TRP agonists alone helped with pain or inflammation issues, but again the addition of both TRP and 5HT2A increases efficacy and reduced the amount of either class of substance required to achieve the effect. In order to achieve a similar effect with Psilocybin, a higher amount (almost triple) was required than when combined with TRP receptor agonists. Formulations of the Base 07—Complete composition produced what many individuals referred to as a “lightness” or “glow” for 4 to 6 hours and a sustained good mood for days or weeks afterwards in some cases. Alterations of the ingredients and amounts of certain ingredients altered the effect of the formulation and allows adjustment to treat specific conditions more effectively, as shown above in Tables 8 and 9. There are many agonists for both the 5HT2A receptor and TRP family receptors. Various molecules and combinations of molecules alter the effects in a subjectively noticeable way

Lack of effect from a single dose was the most common complaint and most these were able to feel the effects of the formulation within three doses. Many individuals who complained of not feeling one dose were over 200 lbs in weight, well above the 150 lb average that was used in calculating the MED Conversely, some smaller individuals, often female, would half the MED dose, suggesting that even 50% of a MED is enough for some people. In individuals who used the formulation for several days, a double dose was common for regular users over 70 kg, but not for those under 70 kg. No significant tolerance effects or buildup have been reported beyond situationally increasing dosage based on increased stress to the system, whether mental or physical.

Many individuals reported minimal to no psychoactive effects from a single MED, but nearly all individuals felt psychoactive effects from three MEDs There is some contraindication of psilocybin with SSRI based on both literature and observation. Blocking serotonin reuptake at the brain may limit the effect of the incoming signal from the gut. Individuals taking SSRIs do not appear to feel the effects of a MED and require an increased dose in order to feel anything at all in most cases. Individuals on low levels of citalopram appeared to show significant results but those on higher doses of fluoxetine or other stronger SSRIs do not often feel the effect from a MED that affects healthy individuals who are not using SSRIs. This trend result was not as pronounced with Wellbutrin, which is a dopamine uptake inhibitor, but still there appeared to be a cross-tolerance or lack of effect to the formulations when SSRIs were present in the system. Some individuals who weened off SSRI medications saw similar results to those not using SSRIs after a few months.

The most commonly noted side effects were digestive issues or stomach cramping. In many cases, stomach cramping was minor and subsided quickly. Two individuals reported severe intestinal pain. One awoke the morning after with sharp pains that were not accompanied by irregular bowel activity. Cramps were sharp and painful but went away within an hour of waking. The second individual develop the symptoms after using the formulation for a significant period of time (over three weeks). Severe cramping would occur not often occur during the dosage, but hours after when the effects had worn off. The second individual then tried psilocybin mushrooms alone with no other additives and the cramping returned, indicating it was not specific to the formulation, but rather psilocybin. The second individual responded well to a formulation lacking psilocybin but containing serotonin as a 5HT2A substitute, demonstrating that psilocybin is not required for efficacy and that serotonin or other agonists may effectively combine with TRP molecules. These cases indicate that a small subset of the population may have some sensitivity to psilocybin or the mushrooms themselves, perhaps as a result of abnormal pain receptor signaling following receptor agonism. It is possible that improper signaling between 5HT2A and TRP Receptors may occur as a result of this stimulated signaling, resulting in the painful sensation. Alternatively, TRP agonists may be flooding the system in large concentrations once metabolized and cause this pain. Regardless, this appears to be sensitization of TRP channels to signal heat and pain.

At the amount of Psilocybin consumed in one MED (>150 mg dried fruiting bodies), there were no reports of intoxication or other psychoactive effects to a degree that influenced motor skills or coordination. The vast majority of individuals reported normal ability to perform simple motor tasks. Some report enhanced positive feelings and none report enhanced negative feelings. Some report this positive effect lasted for weeks after a single MED.

Effects observed in individuals included increased ability to relax, increased ability to concentrate, enhanced cognitive effects, increased energy levels, lower appetite levels, increased disposition to avoid sugar and lowered requirement for prescription medications.

Some individuals reported difficulty sleeping before taking the formulations. Some individuals who reported difficulty sleeping after an active dose were provided with formulations excluding coffee beans or cacao, and thereafter reported less difficulty sleeping.

Most individuals reported persistent effects of the medication six hours after consumption of a MED In contrast most individuals who took equivalent or higher doses of psilocybin alone do not report feeling effects at 6 hours after consumption.

Some individuals reported an absence of suicidal ideation after taking even a single MED, and reported generally higher to feeling amazing and having no desire to kill themselves within a single dose. Amazing results in most individuals following one day of treatment. This is contrary to all available antidepressants on the market.

The compositions may be applied to reduce addiction and pleasure seeking behaviors, finding application in harm reduction approaches. Drug addicts often show reduced 5HT2A expression and lower abundance of 5HT2A receptors. Formulations of the compositions provided herein improved the ability of people to cease use of addictive drugs including alcohol, tobacco (or other nicotine), cocaine and opiates, even when the formulations were consumed at music festivals. Individuals have also reported mitigated addictive behaviors such as gambling.

### Example 6

Variation in the activity of different 5HT2A agonists was confirmed through analysis of compounds known to interact with serotonin receptors, and specifically ergoline class molecules. The Convolvulaceae family, commonly referred to as the bindweed or morning glory family, comprises approximately sixty genera and more than 1,650 species of mostly herbaceous vines. Morning glory seeds have been used to produce psychedelic experiences through ingestion due to the presence of LSA or LSH. A number of species in this family are known to produce hallucinogenic effects when consumed. (Grzegorz, 2013)

Experiments were performed with seeds from three different species, Heavenly Blue morning glory (Ipomoea tricolor), Mexican Morning glory (Ipomoea hederacea), traditionally known as Ololiuqui, and Hawaiian Baby Woodrose (Argyreia nervosa) also known as Elephant creeper. The Heavenly Blue morning glory seemed to be slightly more potent than the Mexican variety at an equivalent weighted dose. The Hawaiian baby woodrose was at least three times as strong as the Heavenly Blue variety of morning glory when considered by mass. This was reflected in the MED50, which was under >300 mg of seeds for Hawaiian baby woodrose and over 1000 mg for Heavenly Blue morning glory. This correlates with literature regarding the amounts of LSA or LSH in the seeds with Hawaiian baby woodrose containing approximately triple the ergoline content compared with the morning glory.

Ingestion of Hawaiian baby woodrose or morning glory seeds alone resulted in significant psychoactive effects at higher doses. For Hawaiian baby woodrose 300 mg of ground seed was required to achieve MED50 and over 500 mg typically resulted in intoxicating effects including visual distortions and mild hallucinations. Similar to psilocybin, the MED for Hawaiian baby woodrose seeds is lowered by the inclusion of TRP agonists and these potentiator compounds influence the effects of the LSA, the LSH and potentially other 5HT2A agonist molecules present in the seeds. With TRP agonists included, even 250 mg was too much for most people to tolerate comfortably and was well above the established MED50 of around 140 mg. The TRP agonists potentiate the combination of LSA, LSH significantly to the point of making it uncomfortable for most individuals even at moderate doses (<200 mg seed with TRPs).

As seen with Psilocybin, the addition of the TRP agonists to species containing LSA, LSH and other ergolines also significantly reduced the MED50, by a similar margin of ⅔. There was a reduction in the MED50 with TRP agonists added to psilocybin by nearly 60% and similar reductions were achieved for both morning glory and Hawaiian baby woodrose seeds (see FIGS. 6 to 8). As such, the TRP agonists make the effects of the 5HT2A agonists about three times more noticeable as when taken alone.

The active ergoline molecules present in Convolvulaceae species are more similar in subjective to LSD than to psilocybin and when consumed on their own, which is consistent with the presence of LSA, LSH and other ergolines. Without the inclusion of the TRP receptor agonists, the effects were significantly different than psilocybin. Even at lower doses, the effects were notably different than previously described formulations with psilocybin. While still producing an improved mood and slight euphoric feeling, there were subjective differences in many areas. The duration was significantly longer with most still feeling the effects 9 hours after a single dose, and in some cases 12 hours after dosing. Unlike psilocybin, the effects do not result in a feeling of connection to others or the environment. While psilocybin often improves openness and promotes communication, LSA, LSH and other ergolines present in morning glory or Hawaiian baby woodrose seeds almost seem to do the opposite. Instead, these appear to be quite introspective. Definitely a desire to be away from groups of people or unknown situations and remain in a comfortable setting. Subjects report a desire to avoid being alone due to the anxiety, and also a desire to avoid strangers or to participate in situations wherein the individual lacks control. Comfort is key to a stress-free or low-stress environment due to the anxiety that may be caused by LSA, LSH and other ergolines present in morning glory or Hawaiian baby woodrose seeds.

While subjects generally appear activities outdoor such as gardening, or in nature such as hiking, the LSA, LSH and other ergolines present in morning glory or Hawaiian baby woodrose seeds appears to be more useful for individual tasks not involving groups or other people. Some individuals described feeling “energized” and compelled to do things after taking formulations including LSA, LSH and other ergolines present in morning glory or Hawaiian baby woodrose seeds. Unfortunately there also appears to be negative effects on the hippocampus, including difficulties with spatial memory. Multiple subjects report forgetting what they were doing or where they had put things down. Participants report it being easy to get lost in thought and also difficult to sit still.

Without the TRP agonists, the effects appeared to overlap somewhat with attention deficit disorder inducing and most individuals did not enjoy the experience. With the presence of TRPs and a reduced dose of LSA, LSH and other ergolines present in morning glory or Hawaiian baby woodrose seeds, therapeutic effects to the formulations may be more accessible, particularly with respect to creativity. Despite a lack of mental focus, there was also an underlying clarity of being, an enhancement of visual and auditory senses, but overpowering or hallucinogenic effects were uncommon. Tunnel-like vision was reported by a few individuals, yet most were still able to perform regular tasks such as riding a bicycle, mowing the lawn, washing dishes, etc. Manty individuals were able to concentrate on specific tasks, but tasks with math, numbers or spreadsheets seemed almost impossible to some individuals. In contrast, music and art are most enjoyable, as either a spectator or creator. Individuals note seeing things they have never seen before and noticing small details they had overlooked. Music appeared to some individuals to have more depth and color.

As indicated by the predicted amounts of LSA, LSH and other ergolines present in morning glory or Hawaiian baby woodrose seeds, the Hawaiian baby woodrose was very strong and felt somewhat “heavy” compared to the morning glory seeds. In some individuals, breathing felt heavy or slowed with Hawaiian baby woodrose and adds to the anxiety effects. This was not as pronounced with morning glory where about 0.5 grams of seeds seems to be an effective and comfortable dose for most people when TRP receptor agonists are present.

Anxiety was reported in almost all users, particularly within the first two hours following ingestion. This is likely due to interaction with 5HT3 serotonin receptor. The 5HT3 serotonin receptor is activated by LSA and LSH. The activation of 5HT3 along with differential activity at the 5HT2A receptor likely accounts for some of the differences between psilocybin and the seeds containing LSA, LSH and other ergolines present in morning glory or Hawaiian baby woodrose seeds. Psilocybin is less likely to activate 5HT3 than are LSA, LSH and other ergolines present in morning glory or Hawaiian baby woodrose seeds. Menthol is an antagonist of 5HT3 (Ashoor, 2013), and inclusion of peppermint in the formulations helped alleviate symptoms of nausea and anxiety. Mixing the ingredients directly into green tea with peppermint was an effective method of administration which likely improved the speed at which the materials exited the stomach and entered the digestive tract.

The 5HT3 receptor is expressed throughout the central and peripheral nervous systems, and mediates a variety of physiological functions. On a cellular level, postsynaptic 5-HT3 receptors mediate fast excitatory synaptic transmission in neocortical interneurons, amygdala, and hippocampus, and in visual cortex. 5HT3 agonism is known to induce nausea through influencing the vomiting center in brain stem. 5HT3 agonism has also been linked to anxiety, seizure propensity, pro-nociception. This may account for some of the unpleasant side effects of morning glory and Hawaiian baby woodrose seed consumption which was mitigated by the inclusion of citric acid as described previously. The conversion of these molecules to a citrate form at lower ph along reduced feelings of uneasiness and anxiety as well as the associated stomach pain or cramping.

While LSA, LSH and other ergolines present in morning glory or Hawaiian baby woodrose seeds resulted in different effects from psilocybin alone or in combination with TRPs, there still appeared to be therapeutic value. The ergolines seemed to result in a greater tendency to lose focus or want to wander. Nature was reported to be the best scenario for consumption of these products. Visual acuity seems enhanced, but with some degree of tunnel vision. Focus and concentration as well as creativity appear to be restored somewhat by the inclusion of Frankincense with LSA/LSH, especially in the citrate form.

One effect of formulations including LSA, LSH and other ergolines present in morning glory or Hawaiian baby woodrose seeds with the TRP agonists that may have therapeutic value is intense polyphagia that may occur within a few hours of consuming the dose. Some participants report hunger that cannot seem to be satiated when using formulations including the TRP agonists and LSA, LSH and other ergolines present in morning glory or Hawaiian baby woodrose seeds as 5HT2A agonists. Polyphagia was unexpected as most 5HT2A agonists are reported to curb appetite. This effect may provide therapeutic benefits for individuals suffering from anorexia and bulimia. Once over the queasiness in the first few hours, appetite stimulation became intense for many individuals and eating is very pleasurable, to the extent that these formulations may have efficacy for reducing nausea. Eating before using the formulations is recommended to mitigate polyphagia. However, eating before using the formulations also appeared to lengthen the onset of the dosage and therefore increase the period of anxiety sometimes reported. Some reported yawning and feeling tired at lower doses. Numerous subjects took a nap while on the dose and reported vivid dreams. This is also contrary to most reported psychoactive 5HT2A agonists, which often prevent sleep while active in the body.

In Vitro Examples 7-49 Showing Effects of 5HT2A Agonists and/or TRP Agonists on Inflammatory Markers on Human Primary Small Intestinal Epithelial Cells (HSIECs)

### Overview of Methods

Cell Culture: Human primary small intestinal epithelial cells (HSIEC), purchased from Cell Biologics were cultured in Epithelial Cell Medium/w Kit. HSIEC cells were incubated at 37° C. in a humidified atmosphere of 5% CO2. HSIEC cells were treated with mycoplasma removal reagent BM-Cyclin (Roche) to ensure mycoplasma negative before treatment.

MTT Cell Viability Assay: The MTT assay is used to measure cellular metabolic activity as an indicator of cell viability, proliferation and cytotoxicity. Cytotoxicity of psilocybin (Psygen), 4-AcO-DMT (ChemLogix), ketanserin (TCI America), capsaicin (Sigma), curcumin (Sigma), and eugenol (Sigma) was measured using MTT assay. Once HSIEC cells grown to 80% confluency, 3×10³ cells/well were replated in 96-well plates. At 24 h after incubation, cells were treated with the indicated concentration of psilocybin, 4-AcO-DMT, ketanserin, capsaicin, curcumin, carvacrol, piperine, cinnemaldehyde, or eugenol, individually or in combination(s), DMSO and/or ethanol served as a control. Assays were performed with 3-(4,5-Dimethylthiazol-2-yl)-2,5-diphenyltetrazolium bromide (MTT) using the Cell Proliferation Kit I (Roche Diagnostics GmbH) in triplicate, as described by the manufacturer. The spectrophotometric absorbance of samples was measured at 595 nm using a microtiter plate reader (FLUOstar Omega, BMG LABTECH). Psilocybin and Eugenol were also analyzed on A172 glioblastoma cell line using a similar methodology as described below. As shown in Table 49 below, most of the compounds tested did not have a significant negative effect on cellular metabolic activity at most of the concentrations tested. Further studies are generally only performed at doses which do no show aberrant cell growth, meaning significantly increased or decreased from the control. High doses of psilocybin and 4AcO-DMT increase growth of cells whereas doses of 40 um and less did not. Most other substances showed some toxicity, reducing cellular growth in a dose dependent fashion. Ketanserin showed an inhibitory effect above 20 μM. All initial doses of capsaicin were found to be inhibitory with very low doses (<10 μM) showing no effect. Cinnamaldehyde also demonstrated significant toxicity over 10 μM concentration. This was similar for piperine, and carvacrol which could only be studied at very low dose (2.5 Eugenol only had a slight inhibitory effect on cell growth but only became toxic at very high concentrations. Combinations of low doses of each 5HT2A agonist with TRP agonists were performed at doses where the individual ingredients did not show significant cellular aberrations. Most substances when combined with either psilocybin or 4AcO-DMT did not significantly reduced growth withing the first 72 hours at concentrations that did not influence cell growth for each individual compound. The dose of Curcumin and Capsaicin specifically had to be reduced farther to combine these ingredients with the 5HT2A agonists and antagonist without causing toxicity.

TABLE 49
Cellular Metabolic Activity in HSIEC Cells
Treated with Compounds of Interest

| | | | | |
|---|---|---|---|---|
| 0 | 24 h | 48 h | 72 h | 96 h |

Single Molecules
Psilocybin

| | | | | | | |
|---|---|---|---|---|---|---|
| Control | Ethanol | 0.040 | 0.097 | 0.196 | 0.463 | 0.660 |
| | 5 μM | 0.047 | 0.098 | 0.186 | 0.380 | 0.621 |
| | 10 μM | 0.038 | 0.107 | 0.198 | 0.388 | 0.624 |
| | 20 μM | 0.038 | 0.150 | 0.238 | 0.415 | 0.635 |
| | 40 μM | 0.041 | 0.182 | 0.259 | 0.467 | 0.685 |
| | 80 μM | 0.042 | 0.345 | 0.371 | 0.579 | 0.805 |
| | 160 μM | 0.042 | 0.523 | 0.604 | 0.808 | 1.099 |

Curcumin

| | | | | | | |
|---|---|---|---|---|---|---|
| Control | Ethanol | 0.040 | 0.097 | 0.196 | 0.463 | 0.660 |
| | 1 μM | 0.040 | 0.086 | 0.184 | 0.375 | 0.611 |
| | 5 μM | 0.036 | 0.079 | 0.148 | 0.299 | 0.595 |
| | 10 μM | 0.036 | 0.070 | 0.115 | 0.215 | 0.466 |
| | 20 μM | 0.040 | 0.059 | 0.057 | 0.060 | 0.038 |
| | 40 μM | 0.039 | 0.038 | 0.026 | 0.014 | 0.00 |
| | 80 μM | 0.041 | 0.042 | 0.019 | 0.018 | 0.005 |

Eugenol

| | | | | | | |
|---|---|---|---|---|---|---|
| Control | Ethanol | 0.040 | 0.099 | 0.190 | 0.384 | 0.837 |
| | 10 μM | 0.047 | 0.064 | 0.168 | 0.346 | 0.632 |
| | 50 μM | 0.038 | 0.079 | 0.145 | 0.337 | 0.647 |
| | 100 μM | 0.038 | 0.075 | 0.136 | 0.346 | 0.662 |
| | 200 μM | 0.041 | 0.064 | 0.111 | 0.296 | 0.652 |
| | 400 μM | 0.042 | 0.059 | 0.128 | 0.242 | 0.549 |
| | 800 μM | 0.042 | 0.053 | 0.091 | 0.146 | 0.268 |

Capsaicin

| | | | | | | |
|---|---|---|---|---|---|---|
| Control | Ethanol | 0.040 | 0.099 | 0.190 | 0.384 | 0.837 |
| | 10 μM | 0.040 | 0.075 | 0.174 | 0.331 | 0.708 |
| | 50 μM | 0.036 | 0.062 | 0.155 | 0.292 | 0.681 |
| | 100 μM | 0.036 | 0.063 | 0.150 | 0.272 | 0.623 |
| | 200 μM | 0.040 | 0.044 | 0.082 | 0.144 | 0.231 |
| | 400 μM | 0.039 | 0.016 | 0.031 | 0.016 | 0.018 |
| | 800 μM | 0.041 | 0 | 0 | 0 | 0 |

Capsaicin (Low Dose repeat)

| | | | | | | |
|---|---|---|---|---|---|---|
| Control | Ethanol | 0.065 | 0.102 | 0.159 | 0.348 | 0.571 |
| | 0.5 μM | 0.068 | 0.088 | 0.154 | 0.288 | 0.469 |
| | 1 μM | 0.066 | 0.084 | 0.150 | 0.326 | 0.502 |
| | 2.5 μM | 0.066 | 0.078 | 0.137 | 0.279 | 0.422 |
| | 5 μM | 0.068 | 0.080 | 0.148 | 0.296 | 0.477 |

4-AcO-DMT

| | | | | | | |
|---|---|---|---|---|---|---|
| Control | DMSO | 0.016 | 0.073 | 0.116 | 0.230 | 0.441 |
| | 5 μM | 0.022 | 0.084 | 0.137 | 0.305 | 0.580 |
| | 10 μM | 0.020 | 0.194 | 0.233 | 0.370 | 0.647 |
| | 20 μM | 0.019 | 0.127 | 0.176 | 0.319 | 0.599 |
| | 40 μM | 0.020 | 0.194 | 0.233 | 0.370 | 0.647 |
| | 80 μM | 0.017 | 0.338 | 0.377 | 0.518 | 0.755 |
| | 160 μM | 0.018 | 0.595 | 0.621 | 0.780 | 0.912 |

Ketanserin

| | | | | | | |
|---|---|---|---|---|---|---|
| Control | DMSO | 0.016 | 0.073 | 0.116 | 0.230 | 0.441 |
| | 1 μM | 0.015 | 0.084 | 0.128 | 0.251 | 0.529 |
| | 5 μM | 0.014 | 0.072 | 0.122 | 0.258 | 0.509 |
| | 10 μM | 0.016 | 0.066 | 0.123 | 0.252 | 0.510 |
| | 20 μM | 0.015 | 0.075 | 0.114 | 0.253 | 0.466 |
| | 40 μM | 0.013 | 0.063 | 0.113 | 0.121 | 0.229 |
| | 80 μM | 0.014 | 0.054 | 0.078 | 0.113 | 0.226 |

Piperine

| | | | | | | |
|---|---|---|---|---|---|---|
| Control | EtOH | 0.026 | 0.068 | 0.163 | 0.261 | 0.559 |
| | 2.5 μM | 0.033 | 0.073 | 0.175 | 0.276 | 0.460 |
| | 5 μM | 0.035 | 0.077 | 0.170 | 0.278 | 0.517 |
| | 10 μM | 0.026 | 0.073 | 0.183 | 0.271 | 0.438 |
| | 20 μM | 0.022 | 0.075 | 0.166 | 0.268 | 0.445 |
| | 40 μM | 0.025 | 0.069 | 0.169 | 0.226 | 0.423 |
| | 80 μM | 0.026 | 0.068 | 0.171 | 0.224 | 0.391 |
| | 100 μM | 0.030 | 0.071 | 0.175 | 0.248 | 0.410 |
| | 200 μM | 0.027 | 0.071 | 0.171 | 0.243 | 0.426 |
| | 400 μM | 0.031 | 0.068 | 0.191 | 0.187 | 0.255 |

Carvacrol

| | | | | | | |
|---|---|---|---|---|---|---|
| Control | EtOH | 0.026 | 0.068 | 0.163 | 0.261 | 0.559 |
| | 2.5 μM | 0.021 | 0.071 | 0.176 | 0.245 | 0.515 |
| | 5 μM | 0.022 | 0.072 | 0.175 | 0.261 | 0.501 |
| | 10 μM | 0.021 | 0.070 | 0.164 | 0.267 | 0.487 |
| | 20 μM | 0.022 | 0.071 | 0.171 | 0.266 | 0.461 |
| | 40 μM | 0.022 | 0.070 | 0.159 | 0.272 | 0.544 |
| | 80 μM | 0.025 | 0.076 | 0.120 | 0.221 | 0.423 |
| | 100 μM | 0.026 | 0.069 | 0.106 | 0.259 | 0.397 |
| | 200 μM | 0.027 | 0.061 | 0.088 | 0.179 | 0.238 |
| | 400 μM | 0.027 | 0.062 | 0.079 | 0.139 | 0.137 |

Cinnamaldehyde

| | | | | | | |
|---|---|---|---|---|---|---|
| Control | EtOH | 0.033 | 0.175 | 0.158 | 0.255 | 0.587 |
| | 2.5 μM | 0.035 | 0.170 | 0.159 | 0.249 | 0.417 |
| | 5 μM | 0.026 | 0.183 | 0.148 | 0.236 | 0.423 |
| | 10 μM | 0.022 | 0.166 | 0.119 | 0.160 | 0.321 |
| | 20 μM | 0.025 | 0.169 | 0.095 | 0.105 | 0.152 |
| | 40 μM | 0.026 | 0.171 | 0.049 | 0.018 | 0.015 |
| | 80 μM | 0.030 | 0.175 | 0.021 | 0 | |
| | 100 μM | 0.027 | 0.171 | 0.016 | | |
| | 200 μM | 0.031 | 0.191 | 0.009 | | |

Molecules in Combination
Psilocybin + Eugenol

| | | | | | | |
|---|---|---|---|---|---|---|
| Control | EtOH | 0.046 | 0.049 | 0.110 | 0.192 | 0.358 |
| | 10 μM PSI + | 0.045 | 0.069 | 0.108 | 0.197 | 0.307 |
| | 25 μM EUG | | | | | |
| | 20 μM PSI + | 0.046 | 0.079 | 0.136 | 0.221 | 0.311 |
| | 25 μM EUG | | | | | |
| | 40 μM PSI + | 0.046 | 0.141 | 0.170 | 0.284 | 0.377 |
| | 25 μM EUG | | | | | |

Psilocybin + Curcumin

| | | | | | | |
|---|---|---|---|---|---|---|
| Control | EtOH | 0.046 | 0.049 | 0.110 | 0.192 | 0.358 |
| | 10 μM PSI + | 0.045 | 0.075 | 0.114 | 0.141 | 0.150 |
| | 25 μM CUR | | | | | |
| | 20 μM PSI + | 0.044 | 0.089 | 0.122 | 0.141 | 0.137 |
| | 25 μM CUR | | | | | |
| | 40 μM PSI + | 0.042 | 0.157 | 0.163 | 0.195 | 0.189 |
| | 25 μM CUR | | | | | |

Psilocybin + Curcumin (0.5 μM CUR)

| | | | | | | |
|---|---|---|---|---|---|---|
| Control | EtOH | 0.094 | 0.116 | 0.185 | 0.494 | 0.759 |
| 0.5 μM Curcumin | 10 μM PSI + | 0.083 | 0.106 | 0.166 | 0.485 | 0.611 |
| | 0.5 μM CUR | | | | | |
| | 20 μM PSI + | 0.090 | 0.135 | 0.169 | 0.421 | 0.595 |
| | 0.5 μM CUR | | | | | |
| | 40 μM PSI + | 0.089 | 0.251 | 0.247 | 0.579 | 0.761 |
| | 0.5 μM CUR | | | | | |

Psilocybin + Curcumin (1 μM CUR)

| | | | | | | |
|---|---|---|---|---|---|---|
| Control | EtOH | 0.094 | 0.116 | 0.185 | 0.494 | 0.759 |
| 1 μM Curcumin | 10 μM PSI + | 0.090 | 0.116 | 0.157 | 0.439 | 0.640 |
| | 1 μM CUR | | | | | |
| | 20 μM PSI + | 0.097 | 0.138 | 0.176 | 0.422 | 0.578 |
| | 1 μM CUR | | | | | |
| | 40 μM PSI + | 0.091 | 0.299 | 0.264 | 0.677 | 0.855 |
| | 1 μM CUR | | | | | |

Psilocybin + Curcumin (2.5 μM CUR)

| | | | | | | |
|---|---|---|---|---|---|---|
| Control | EtOH | 0.094 | 0.116 | 0.185 | 0.494 | 0.759 |
| 2.5 μM Curcumin | 10 μM PSI + | 0.093 | 0.089 | 0.148 | 0.422 | 0.637 |
| | 2.5 μM CUR | | | | | |
| | 20 μM PSI + | 0.092 | 0.131 | 0.171 | 0.480 | 0.661 |
| | 2.5 μM CUR | | | | | |
| | 40 μM PSI + | 0.098 | 0.182 | 0.199 | 0.467 | 0.778 |
| | 2.5 μM CUR | | | | | |

Psilocybin + Capsaicin (0.5 μM CAP)

| | | | | | | |
|---|---|---|---|---|---|---|
| Control | EtOH | 0.065 | 0.102 | 0.159 | 0.348 | 0.571 |
| 0.5 μM Capsaicin | 10 μM PSI + | 0.067 | 0.101 | 0.174 | 0.328 | 0.487 |
| | 0.5 μM CAP | | | | | |
| | 20 μM PSI + | 0.066 | 0.201 | 0.264 | 0.432 | 0.550 |
| | 0.5 μM CAP | | | | | |
| | 40 μM PSI + | 0.061 | 0.185 | 0.208 | 0.390 | 0.599 |
| | 0.5 μM CAP | | | | | |

Psilocybin + Capsaicin (1 μM CAP)

| | | | | | | |
|---|---|---|---|---|---|---|
| Control | EtOH | 0.065 | 0.102 | 0.159 | 0.348 | 0.571 |
| 1 μM Capsaicin | 10 μM PSI + | 0.070 | 0.113 | 0.147 | 0.318 | 0.460 |
| | 1 μM CAP | | | | | |
| | 20 μM PSI + | 0.062 | 0.155 | 0.205 | 0.355 | 0.553 |
| | 1 μM CAP | | | | | |
| | 40 μM PSI + | 0.070 | 0.247 | 0.265 | 0.469 | 0.599 |
| | 1 μM CAP | | | | | |

Psilocybin + Piperine (2.5 μM)

| | | | | | | |
|---|---|---|---|---|---|---|
| Control | EtOH | 0.080 | 0.140 | 0.340 | 0.490 | 0.495 |
| | 10 μM PSI + | 0.076 | 0.177 | 0.355 | 0.506 | 0.328 |
| | 2.5 μM PIP | | | | | |
| | 20 μM PSI + | 0.074 | 0.238 | 0.395 | 0.554 | 0.391 |
| | 2.5 μM PIP | | | | | |
| | 40 μM PSI + | 0.072 | 0.345 | 0.430 | 0.599 | 0.292 |
| | 2.5 μM PIP | | | | | |

Psilocybin + Carvacrol (2.5 μM CAR)

| | | | | | | |
|---|---|---|---|---|---|---|
| Control | EtOH | 0.080 | 0.140 | 0.340 | 0.490 | 0.495 |
| | 10 μM PSI + | 0.075 | 0.181 | 0.376 | 0.529 | 0.378 |
| | 2.5 μM CAR | | | | | |
| | 20 μM PSI + | 0.080 | 0.231 | 0.429 | 0.586 | 0.438 |
| | 2.5 μM CAR | | | | | |
| | 40 μM PSI + | 0.073 | 0.335 | 0.474 | 0.637 | 0.484 |
| | 2.5 μM CAR | | | | | |

Psilocybin + Cinnamaldehyde (0.5 μM CINN)

| | | | | | | |
|---|---|---|---|---|---|---|
| Control | EtOH | 0.080 | 0.140 | 0.340 | 0.490 | 0.495 |
| | 10 μM PSI + | 0.079 | 0.179 | 0.378 | 0.516 | 0.353 |
| | 0.5 μM CINN | | | | | |
| | 20 μM PSI + | 0.078 | 0.250 | 0.410 | 0.563 | 0.348 |
| | 0.5 μM CINN | | | | | |
| | 40 μM PSI + | 0.062 | 0.372 | 0.504 | 0.704 | 0.547 |
| | 0.5 μM CINN | | | | | |

4-AcO-DMT + Eugenol (25 μM)

| | | | | | | |
|---|---|---|---|---|---|---|
| Control | DMSO | 0.047 | 0.046 | 0.103 | 0.191 | 0.342 |
| | 10 μM 4-AcO + | 0.048 | 0.069 | 0.133 | 0.194 | 0.313 |
| | 25 μM EUG | | | | | |
| | 20 μM 4-AcO + | 0.044 | 0.084 | 0.148 | 0.229 | 0.343 |
| | 25 μM EUG | | | | | |
| | 40 μM 4-AcO + | 0.042 | 0.136 | 0.164 | 0.281 | 0.345 |
| | 25 μM EUG | | | | | |

4-AcO-DMT + Curcumin (25 μM)

| | | | | | | |
|---|---|---|---|---|---|---|
| Control | DMSO | 0.047 | 0.046 | 0.103 | 0.191 | 0.342 |
| | 10 μM 4-AcO + | 0.044 | 0.068 | 0.095 | 0.120 | 0.127 |
| | 25 μM CUR | | | | | |
| | 20M 4-AcO + | 0.045 | 0.080 | 0.114 | 0.140 | 0.148 |
| | 25 μM CUR | | | | | |
| | 40 μM 4-AcO + | 0.049 | 0.125 | 0.154 | 0.193 | 0.196 |
| | 25 μM CUR | | | | | |

4-AcO-DMT + Curcumin (0.5 μM )

| | | | | | | |
|---|---|---|---|---|---|---|
| Control | DMSO | 0.089 | 0.091 | 0.162 | 0.458 | 0.753 |
| 0.5 μM Curcumin | 10 μM 4-AcO + | 0.084 | 0.116 | 0.161 | 0.470 | 0.625 |
| | 0.5 μM CUR | | | | | |
| | 20 μM 4-AcO + | 0.086 | 0.130 | 0.173 | 0.484 | 0.676 |
| | 0.5 μM CUR | | | | | |
| | 40 μM 4-AcO + | 0.088 | 0.190 | 0.197 | 0.481 | 0.684 |
| | 0.5 μM CUR | | | | | |

4-AcO-DMT + Curcumin (1 μM)

| | | | | | | |
|---|---|---|---|---|---|---|
| Control | DMSO | 0.089 | 0.091 | 0.162 | 0.458 | 0.753 |
| 1 μM Curcumin | 10 μM 4-AcO + | 0.090 | 0.107 | 0.155 | 0.400 | 0.595 |
| | 1 μM CUR | | | | | |
| | 20 μM 4-AcO + | 0.090 | 0.140 | 0.165 | 0.411 | 0.639 |
| | 1 μM CUR | | | | | |
| | 40 μM 4-AcO + | 0.084 | 0.203 | 0.209 | 0.458 | 0.674 |
| | 1 μM CUR | | | | | |

4-AcO-DMT + Curcumin (2.5 μM)

| | | | | | | |
|---|---|---|---|---|---|---|
| Control | DMSO | 0.089 | 0.091 | 0.162 | 0.458 | 0.753 |
| 2.5 μM Curcumin | 10 μM 4-AcO + | 0.094 | 0.116 | 0.138 | 0.340 | 0.512 |
| | 2.5 μM CUR | | | | | |
| | 20 μM 4-AcO + | 0.086 | 0.137 | 0.153 | 0.399 | 0.661 |
| | 2.5 μM CUR | | | | | |
| | 40 μM 4-AcO + | 0.096 | 0.181 | 0.191 | 0.368 | 0.580 |
| | 2.5 μM CUR | | | | | |

4-AcO-DMT + Capsaicin (0.5 μM)

| | | | | | | |
|---|---|---|---|---|---|---|
| Control | DMSO | 0.066 | 0.067 | 0.154 | 0.262 | 0.491 |
| 0.5 μM Capsaicin | 10 μM 4-AcO + | 0.065 | 0.102 | 0.174 | 0.312 | 0.569 |
| | 0.5 μM CAP | | | | | |
| | 20 μM 4-AcO + | 0.062 | 0.127 | 0.205 | 0.402 | 0.620 |
| | 0.5 μM CAP | | | | | |
| | 40 μM 4-AcO + | 0.063 | 0.197 | 0.242 | 0.456 | 0.647 |
| | 0.5 μM CAP | | | | | |

4-AcO-DMT + Capsaicin (1 μM)

| | | | | | | |
|---|---|---|---|---|---|---|
| Control | DMSO | 0.066 | 0.067 | 0.154 | 0.262 | 0.491 |
| 1 μM Capsaicin | 10 μM 4-AcO + | 0.064 | 0.097 | 0.174 | 0.306 | 0.517 |
| | 1 μM CAP | | | | | |
| | 20 μM 4-AcO + | 0.069 | 0.143 | 0.225 | 0.357 | 0.531 |
| | 1 μM CAP | | | | | |
| | 40 μM 4-AcO + | 0.065 | 0.209 | 0.259 | 0.452 | 0.601 |
| | 1 μM CAP | | | | | |

Ketanserin + Eugenol (25 μM)

| | | | | | | |
|---|---|---|---|---|---|---|
| Control | DMSO | 0.046 | 0.114 | 0.208 | 0.503 | 0.844 |
| | 1 μM KET + | 0.045 | 0.118 | 0.217 | 0.471 | 0.713 |
| | 25 μM EUG | | | | | |
| | 5 μM KET + | 0.046 | 0.114 | 0.217 | 0.516 | 0.740 |
| | 25 μM EUG | | | | | |
| | 10 μM KET + | 0.046 | 0.116 | 0.216 | 0.469 | 0.720 |
| | 25 μM EUG | | | | | |

Ketanserin + Curcumin (5 μM)

| | | | | | | |
|---|---|---|---|---|---|---|
| Control | DMSO | 0.046 | 0.114 | 0.208 | 0.503 | 0.844 |
| | 1 μM KET + | 0.046 | 0.118 | 0.164 | 0.394 | 0.565 |
| | 5 μM CUR | | | | | |
| | 5 μM KET + | 0.045 | 0.096 | 0.157 | 0.340 | 0.585 |
| | 5 μM CUR | | | | | |
| | 10 μM KET + | 0.044 | 0.104 | 0.151 | 0.328 | 0.557 |
| | 5 μM CUR | | | | | |

Ketanserin + Curcumin (0.5 μM )

| | | | | | | |
|---|---|---|---|---|---|---|
| Control | DMSO | 0.064 | 0.048 | 0.108 | 0.256 | 0.476 |
| 0.5 μM Curcumin | 10M KET + | 0.065 | 0.052 | 0.103 | 0.239 | 0.522 |
| | 0.5 μM CUR | | | | | |
| | 5 μM KET + | 0.068 | 0.058 | 0.101 | 0.233 | 0.540 |
| | 0.5 μM CUR | | | | | |
| | 10 μM KET + | 0.066 | 0.045 | 0.099 | 0.223 | 0.472 |
| | 0.5 μM CUR | | | | | |

Ketanserin + Curcumin (1 μM )

| | | | | | | |
|---|---|---|---|---|---|---|
| Control | DMSO | 0.064 | 0.048 | 0.108 | 0.256 | 0.476 |
| 1 μM Curcumin | 1 μM KET + | 0.066 | 0.057 | 0.097 | 0.217 | 0.442 |
| | 1 μM CUR | | | | | |
| | 5 μM KET + | 0.068 | 0.054 | 0.101 | 0.226 | 0.494 |
| | 1 μM CUR | | | | | |
| | 10 μM KET + | 0.067 | 0.047 | 0.101 | 0.215 | 0.468 |
| | 1 μM CUR | | | | | |

Ketanserin + Curcumin (2.5 μM)

| | | | | | | |
|---|---|---|---|---|---|---|
| Control | DMSO | 0.064 | 0.048 | 0.108 | 0.256 | 0.476 |
| 2.5 μM Curcumin | 1 μM KET + | 0.066 | 0.045 | 0.084 | 0.186 | 0.354 |
| | 2.5 μM CUR | | | | | |
| | 5 μM KET + | 0.061 | 0.045 | 0.098 | 0.209 | 0.399 |
| | 2.5 μM CUR | | | | | |
| | 10 μM KET + | 0.070 | 0.044 | 0.086 | 0.158 | 0.298 |
| | 2.5 μM CUR | | | | | |

Ketanserin + Capsaicin (0.5 μM)

| | | | | | | |
|---|---|---|---|---|---|---|
| Control | DMSO | 0.064 | 0.048 | 0.108 | 0.256 | 0.476 |
| 0.5 μM Capsaicin | 10M KET + | 0.066 | 0.049 | 0.109 | 0.252 | 0.507 |
| | 0.5 μM CAP | | | | | |
| | 5 μM KET + | 0.065 | 0.046 | 0.113 | 0.263 | 0.521 |
| | 0.5 μM CAP | | | | | |
| | 10 μM KET + | 0.062 | 0.044 | 0.109 | 0.251 | 0.520 |
| | 0.5 μM CAP | | | | | |

Ketanserin + Capsaicin (1 μM)

| | | | | | | |
|---|---|---|---|---|---|---|
| Control | DMSO | 0.064 | 0.048 | 0.108 | 0.256 | 0.476 |
| 1 μM Capsaicin | 1 μM KET + | 0.063 | 0.054 | 0.103 | 0.240 | 0.464 |
| | 1 μM CAP | | | | | |
| | 5 μM KET + | 0.064 | 0.051 | 0.103 | 0.258 | 0.525 |
| | 1 μM CAP | | | | | |
| | 10 μM KET + | 0.069 | 0.043 | 0.094 | 0.234 | 0.454 |
| | 1 μM CAP | | | | | |

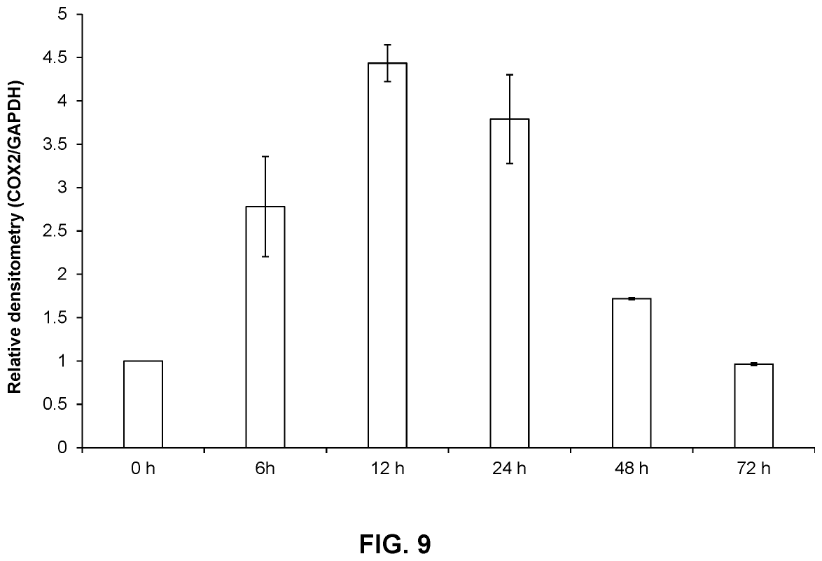

Induction of Inflammation in HSIECs and Treatment with Molecules of Interest: HSIEC cells grown to 80% confluence were treated with 10 ng/ml TNF-α/IFN-γ (Sigma) (Yang et al., 2015), for the time points indicated in FIG. 9, to assess the best time point for determination of anti-inflammatory potential of the molecules and formulations of interest. To do so, COX-2, an enzyme which mediates the bioconversion of arachidonic acid to inflammatory prostaglandins (PGs), was measured via densitometry. GAPDH was used as a reference housekeeping marker for relative densitometry measures. As shown in FIG. 9, the greatest inflammatory effect was observed at 12 hours after treatment with TNF-α/IFN-γ. The anti-inflammatory potential of the molecules and formulations of interest were evaluated at 48 hours post-TNF-α/IFN-γ treatment in the HSIEC assays described below due to reduced variability and ability to assess differences between treatments. Ethanol or DMSO was used, as indicated in the figures, as a vehicle for dilution and as a negative control showing the amount of the inflammatory marker present when inflammation was not induced.

Increasing doses of each molecule of interest, or each combination of molecules of interest, were then distributed to the HSIEC cells treated to induce inflammatory response. Inflammatory markers COX-2 and/or IL-6 were assessed at specific timepoints after induction of inflammation for protein content marker measured via western blot. GAPDH was used as a reference housekeeping marker for relative densitometry measures of protein content.

### Examples 7-9

### Effects of 5HT2A Agonists Psilocybin, and 4-ACO-DMT on COX-2 in In Vitro HSIEC Cell Assays

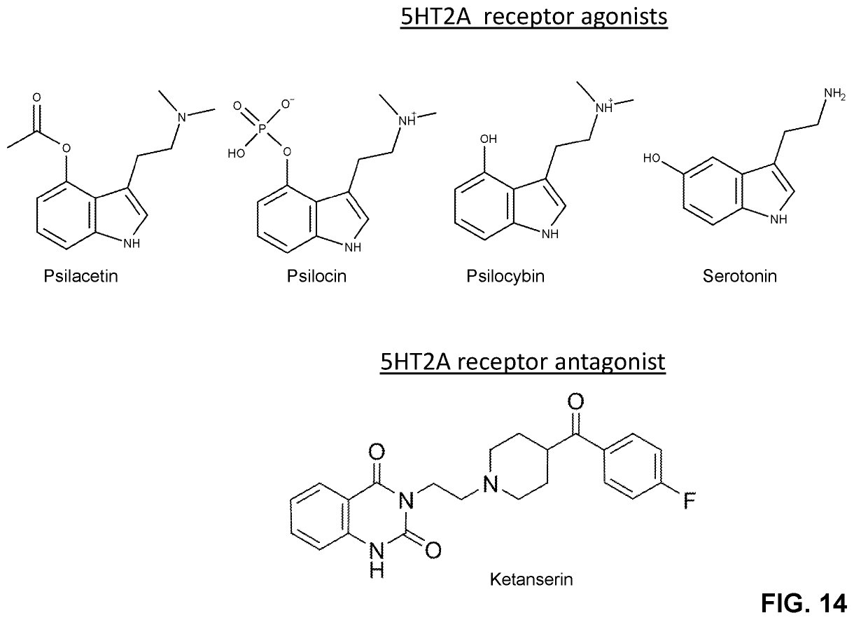

The anti-inflammatory effects of 5HT2A agonists psilocybin and 4-ACO-DMT, as evaluated based on their effects on COX-2, are described below and shown in FIGS. 14-17. FIG. 14 illustrates the structures of 5HT2A agonists psilocybin, 4-ACO-DMT, psilocin, and serotonin. 5HT2A antagonist ketanserin, used as a control in assays 2A-2C, also is shown. Psilocybin is a potent 5HT2A agonist. 4-ACO-DMT (psilacetin) is a synthetic 5HT2A agonist that is similar in structure to psilocybin and psilocin. Ketanserin is a synthetic, high-affinity non-selective antagonist of 5-HT2 receptors in rodents. Based on the mechanism of action of the 5HT2A agonists psilocybin and 4-ACO-DMT, it was hypothesized that both would have anti-inflammatory activity while ketanserin would not.

### Example 7

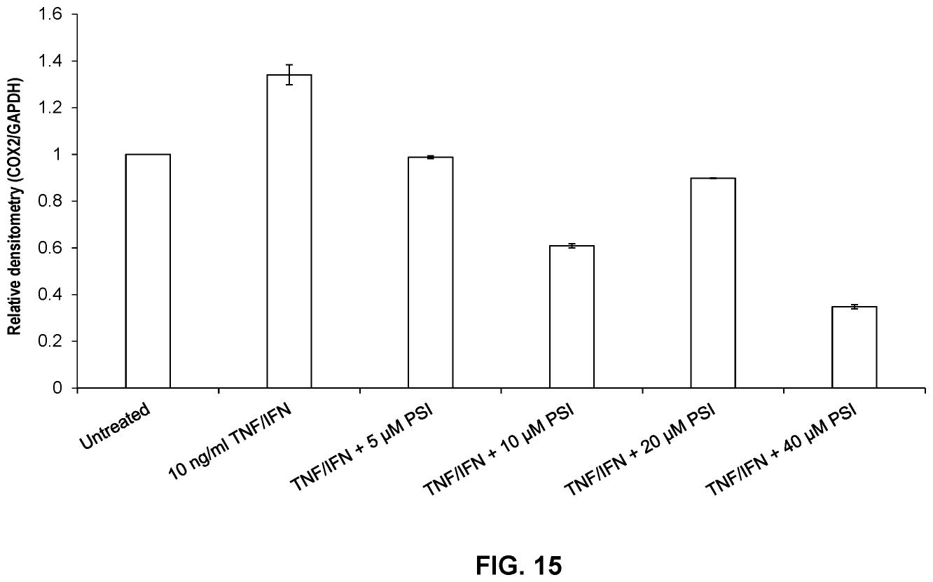

In vitro HSIEC Cell Assay Example 7 is illustrated in FIG. 15, which shows the effects of escalating doses of 5 μM, 10 μM, 20 μM, and 40 μM psilocybin on COX-2 in the HSIEC cells treated with TNF-α/IFN-γ as described above. As can be seen in FIG. all doses of psilocybin resulted in the reduction of inflammatory marker COX-2, with 10 μM and 40 μM psilocybin reducing COX-2 to the greatest extent.

### Example 8

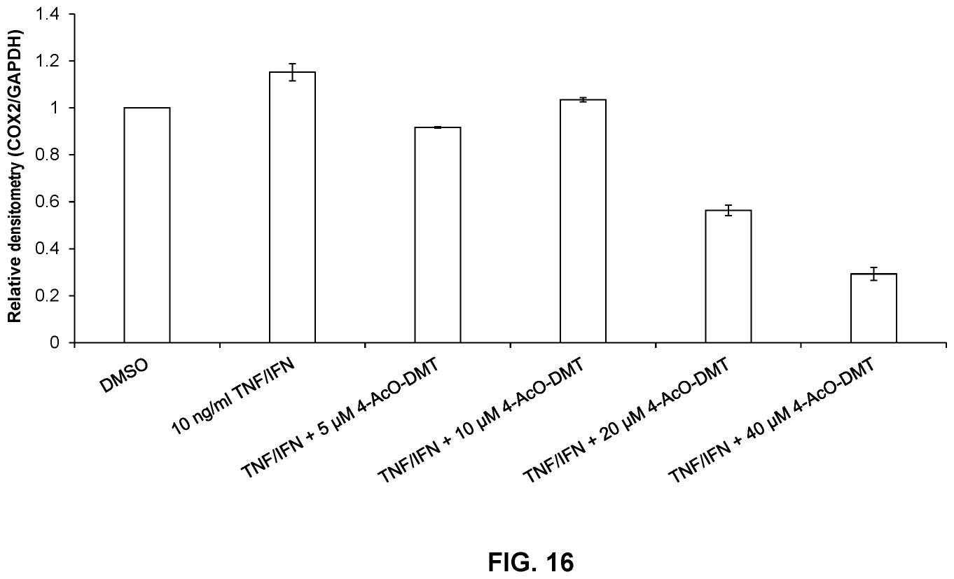

In vitro HSIEC Cell Assay Example 8 is illustrated in FIG. 16, which shows the effects of escalating doses of 5 μM, 10 μM, 20 μM and 40 μM 4-ACO-DMT on COX-2 in the HSIEC cells treated with TNF-α/IFN-γ as described above. As can be seen in FIG. 16, 20 μM, and 40 μM doses resulted in significant reduction of inflammatory marker COX-2, with 40 μM 4-ACO-DMT reducing COX-2 to the greatest extent.

### Example 9

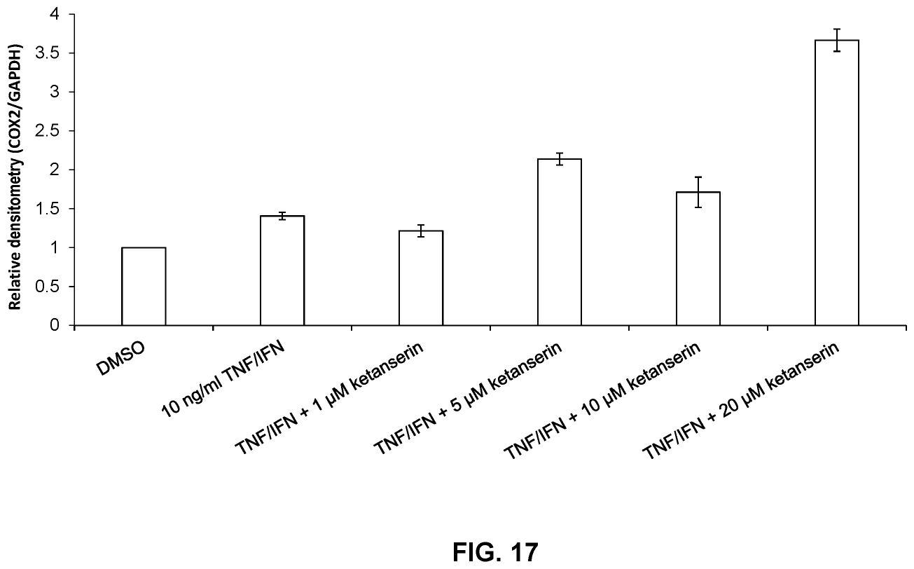

In vitro HSIEC Cell Assay Example 9 is illustrated in FIG. 17, which shows the effects of escalating doses of 1 μM, 5 μM, 10 μM, and 20 μM ketanserin on COX-2 in the HSIEC cells treated with TNF-α/IFN-γ as described above. As expected, none of the doses of ketanserin resulted in a significant reduction in COX-2, while 20 μM ketanserin significantly increased COX-2.

### Examples 10-12

### Effects of TRP Agonists Eugenol, Capsaicin, and Curcumin on COX-2 in In Vitro HSIEC Cell Assays

The anti-inflammatory effects of TRP agonists eugenol, capsaicin, and curcumin,

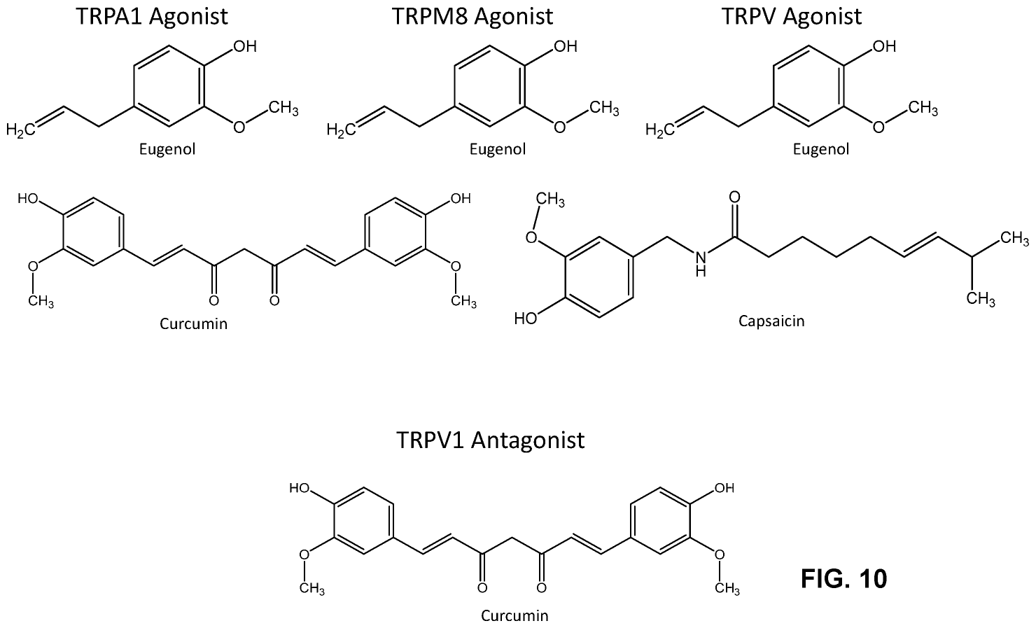

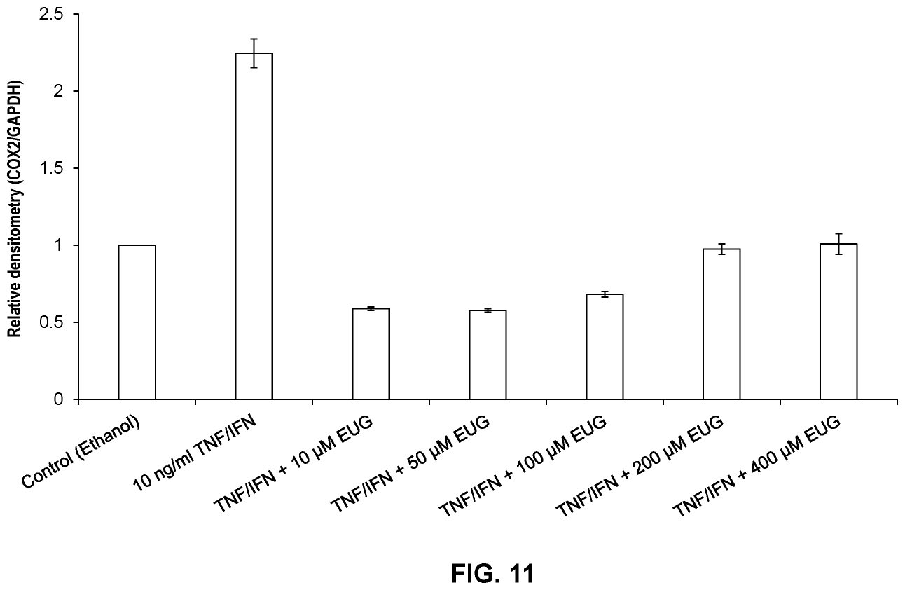

as evaluated based on their effects on COX-2, are described below and shown in FIGS. 11-13. FIG. 10 illustrates the structures of TRP agonists eugenol, capsaicin, and curcumin. Eugenol is a TRPA1, TRPV1, TRPV3 and TRPM8 agonist. Capsaicin is a potent TRPV1 agonist and may have some activity on others TRP receptors such as V3. Curcumin is a TRPV1 antagonist and also predicted to be a TRPA1 agonist. Based on the mechanism of action of these TRP agonists, it was hypothesized that all three would demonstrate some anti-inflammatory activity on their own.

### Example 10

In vitro HSIEC Cell Assay Example 10 is illustrated in FIG. 11, which shows the effects of escalating doses of 10 μM, 50 μM, 100 μM, 200 μM, and 400 μM eugenol on COX-2 in the HSIEC cells treated with TNF-α/IFN-γ as described above. As can be seen in FIG. 11, lower doses of 10 μM-100 μM eugenol resulted in the greatest reduction in inflammatory marker COX-2, while higher doses of 200 μM and 400 μM eugenol reduced COX-2 to a lesser extent.

### Example 11

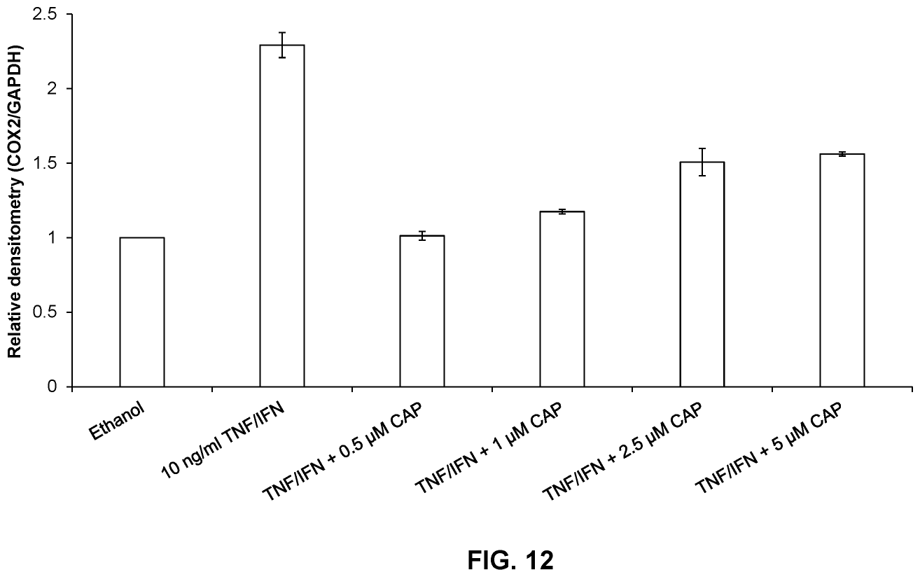

In vitro HSIEC Cell Assay Example 11 is illustrated in FIG. 12, which shows the effects of escalating doses of 0.5 μM, 1 μM, 2.5 μM, and 5 μM capsaicin on COX-2 in the HSIEC cells treated with TNF-α/IFN-γ as described above. As with eugenol, lower doses of 0.5 μM and 1 μM capsaicin resulted in the greatest reduction in inflammatory marker COX-2, while higher doses of 2.5 μM and 5 μM capsaicin reduced COX-2 to a lesser extent.

### Example 12

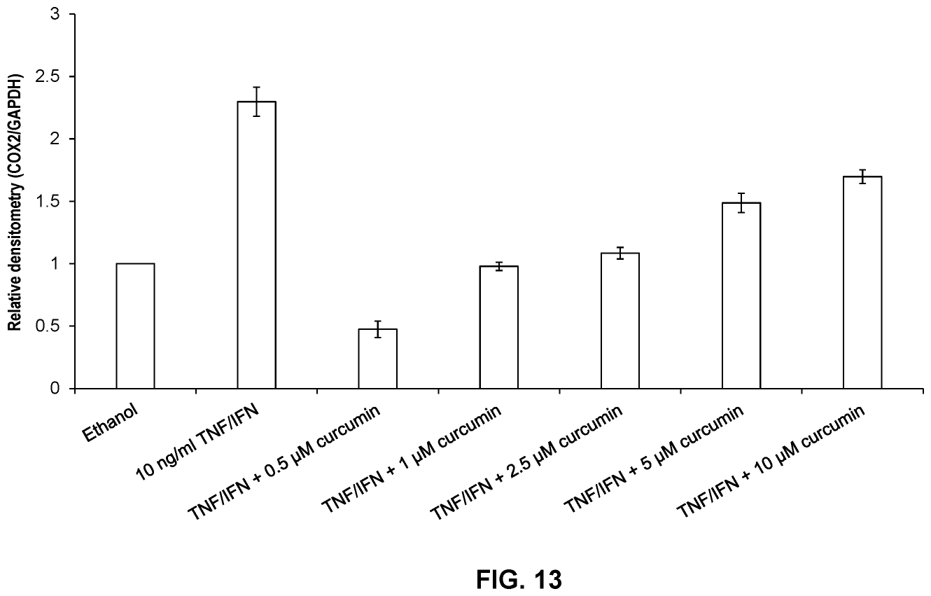

In vitro HSIEC Cell Assay Example 12 is illustrated in FIG. 13, which shows the effects of escalating doses of 0.5 μM, 1 μM, 2.5 μM, and 5 μM curcumin on COX-2 in the HSIEC cells treated with TNF-α/IFN-γ as described above. As with eugenol and capsaicin, lower doses of 0.5 μM and 1 μM curcumin resulted in the greatest reduction in inflammatory marker COX-2, while higher doses of 2.5 μM and 5 μM curcumin reduced COX-2 to a lesser extent.

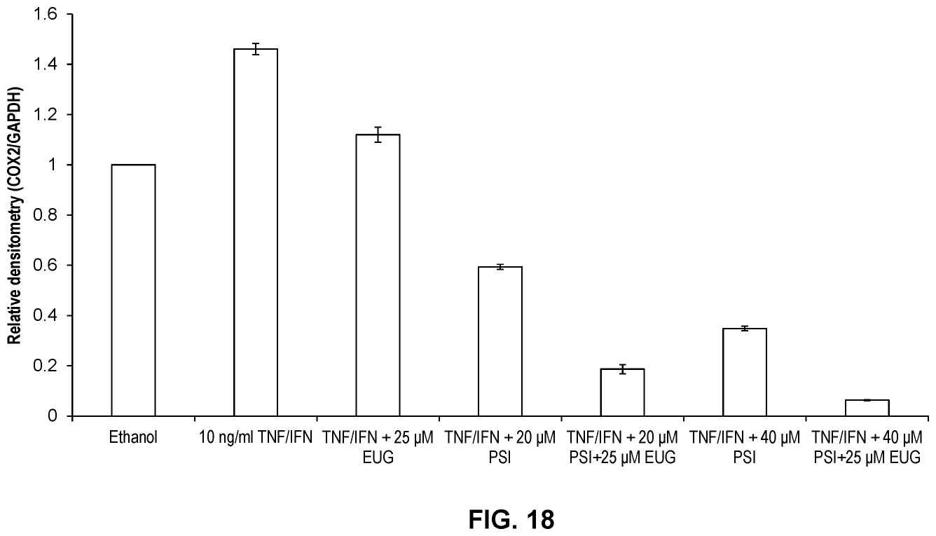

In each of HSIEC Cell Assay Examples 10-12, low doses of the TRP agonist are anti-inflammatory but decrease in efficacy as the concentration increases. This indicates that lower doses may be optimal for reducing inflammatory response. These lower doses were assessed in combination with 5HT2A agonists, as described below with respect to the assays illustrated in FIGS. 18-24 to assess potential synergistic reduction in inflammation of combinations of the 5HT2A with the TRP agonists of interest.

### Examples 13-19

### Effects of Combinations of 5HT2A Agonists Psilocybin and 4-ACO-DMT with Eugenol on COX-2, IL-6, IL-8, and iNOS in In Vitro HSIEC Cell Assays

The anti-inflammatory effects of combinations of 5HT2A agonists psilocybin and 4-ACO-DMT with eugenol, as evaluated based on their effects on COX-2, IL-6, IL-8, and TFN are described below and shown in FIGS. 18-24. Based on the mechanism of action of the 5HT2A agonists psilocybin and 4-ACO-DMT and eugenol, it was hypothesized that combinations of psilocybin and 4-ACO-DMT with eugenol would have synergistic effects on COX-2 and IL-6 while a combination of the 5HT2A antagonist ketanserin with eugenol would not.

### Example 13

In vitro HSIEC Cell Assay Example 13 is illustrated in FIG. 18, which shows the separate and combined effects of psilocybin and eugenol on COX-2 in the HSIEC cells treated with TNF-α/IFN-γ as described above. As can be seen in FIG. 18, combinations of psilocybin and eugenol resulted in the reduction of inflammatory marker COX-2, with the combination of 40 μM psilocybin and 25 μM eugenol reducing COX-2 to the greatest extent, thus indicating synergistic effects of psilocybin and eugenol on COX-2. A combination of 40 μM psilocybin and 25 μM eugenol reduced COX-2 by 83% more than 40 μM psilocybin alone.

### Example 14

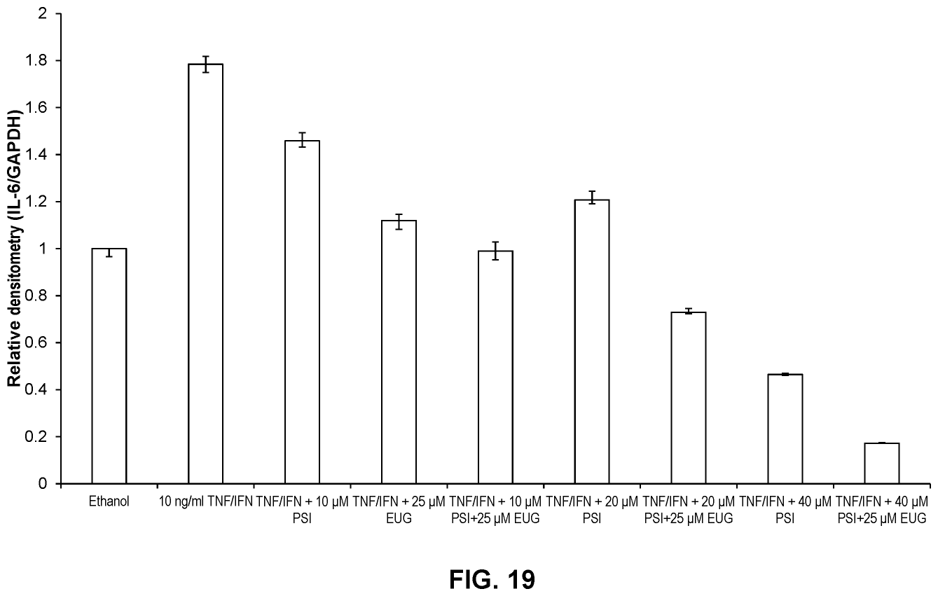

In vitro HSIEC Cell Assay Example 14 is illustrated in FIG. 19, which shows the separate and combined effects of psilocybin and eugenol on IL-6 in the HSIEC cells treated with TNF-α/IFN-γ as described above. As can be seen in FIG. 19, combinations of psilocybin and eugenol resulted in the reduction of inflammatory marker IL-6, with the combination of 40 μM psilocybin and 25 μM eugenol reducing IL-6 to the greatest extent, thus indicating synergistic effects of psilocybin and eugenol on IL-6. A combination of 40 μM psilocybin and 25 μM eugenol reduced IL-6 by 63% more than 40 μM psilocybin alone.

### Example 15

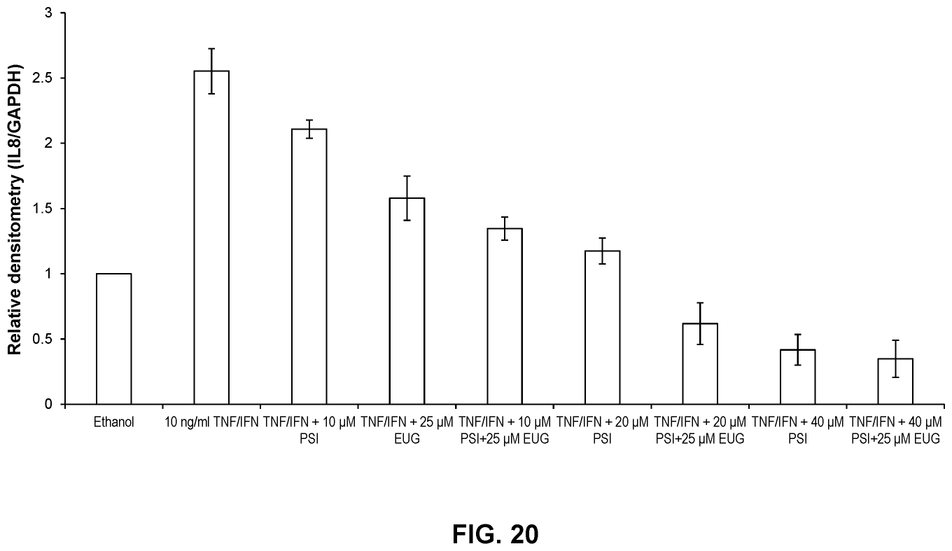

In vitro HSIEC Cell Assay Example 15 is illustrated in FIG. 20, which shows the separate and combined effects of psilocybin and eugenol on IL-8 in the HSIEC cells treated with TNF-α/IFN-γ as described above. As can be seen in FIG. 20, combinations of psilocybin and eugenol resulted in the reduction of inflammatory marker IL-8, with the combination of 40 μM psilocybin and 25 μM eugenol reducing IL-8 to the greatest extent, thus indicating synergistic effects of psilocybin and eugenol on IL-8. A combination of 40 μM psilocybin and 25 μM eugenol reduced IL-8 by 17% more than 40 μM psilocybin alone.

### Example 16

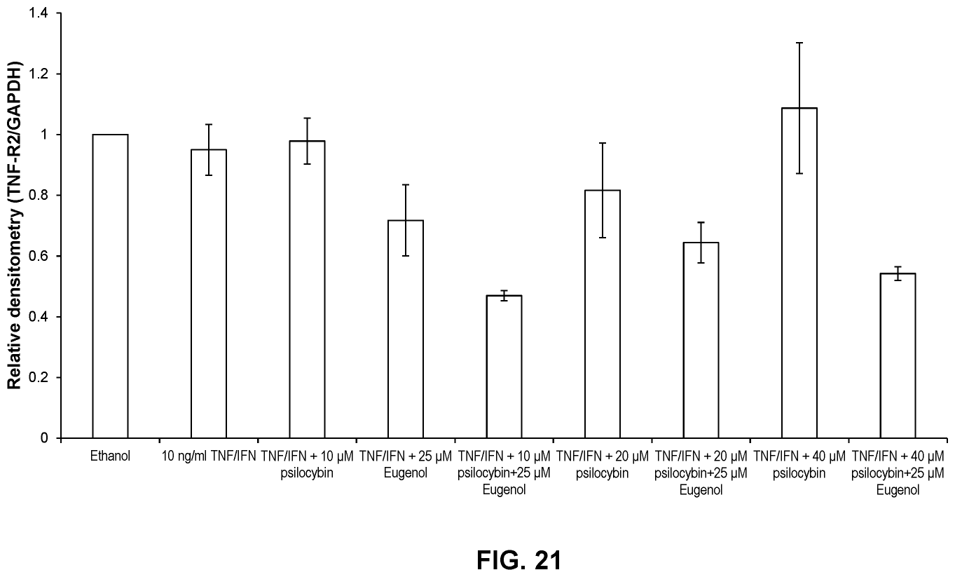

In vitro HSIEC Cell Assay Example 16 is illustrated in FIG. 21, which shows the separate and combined effects of psilocybin and eugenol on TNF receptor 2 (TNF-R2) in the HSIEC cells treated with TNF-α/IFN-γ as described above. As can be seen in FIG. 21, combinations of psilocybin and eugenol resulted in the reduction of inflammatory marker TNF-R2, with the combination of 10 μM psilocybin and 25 μM eugenol reducing TNF-R2 to the greatest extent, thus indicating synergistic effects of psilocybin and eugenol on TNF-R2. A combination of 10 μM psilocybin and 25 μM eugenol reducing TNF by 52% more than 10 μM psilocybin alone.

### Example 17

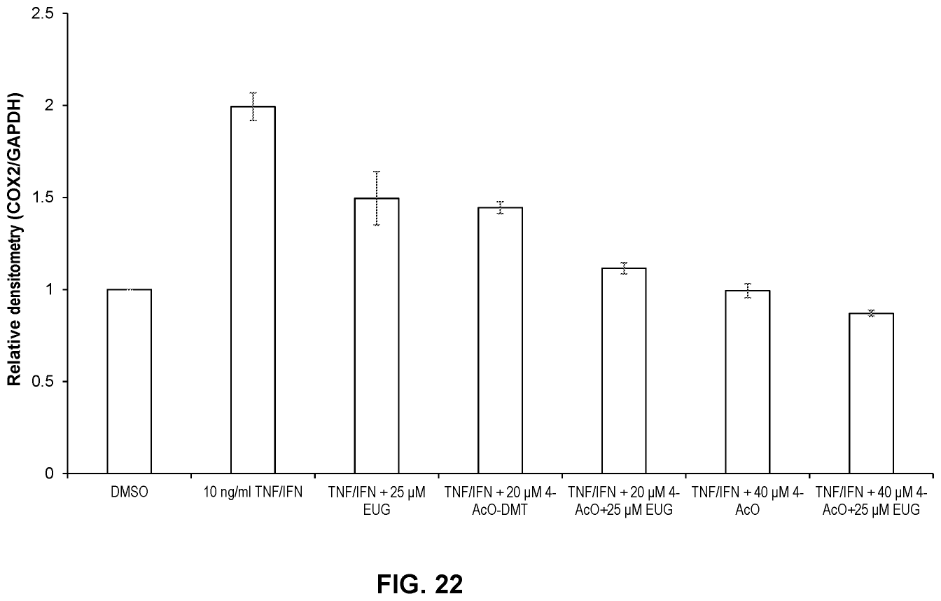

In vitro Assay Example 17 is illustrated in FIG. 22, which shows the separate and combined effects of 4-ACO-DMT and eugenol on COX-2 in the HSIEC cells treated with TNF-α/IFN-γ as described above. As can be seen in FIG. 22, combinations of 4-ACO-DMT and eugenol resulted in the reduction of inflammatory marker COX-2, with 40 μM 4-ACO-DMT and 25 μM eugenol reducing COX-2 to the greatest extent, thus indicating synergistic effects of 4-ACO-DMT and eugenol on COX-2. A 40 μM 4-ACO-DMT and 25 μM eugenol reduced COX-2 by 13% more than 40 μM 4-ACO-DMT alone.

### Example 18

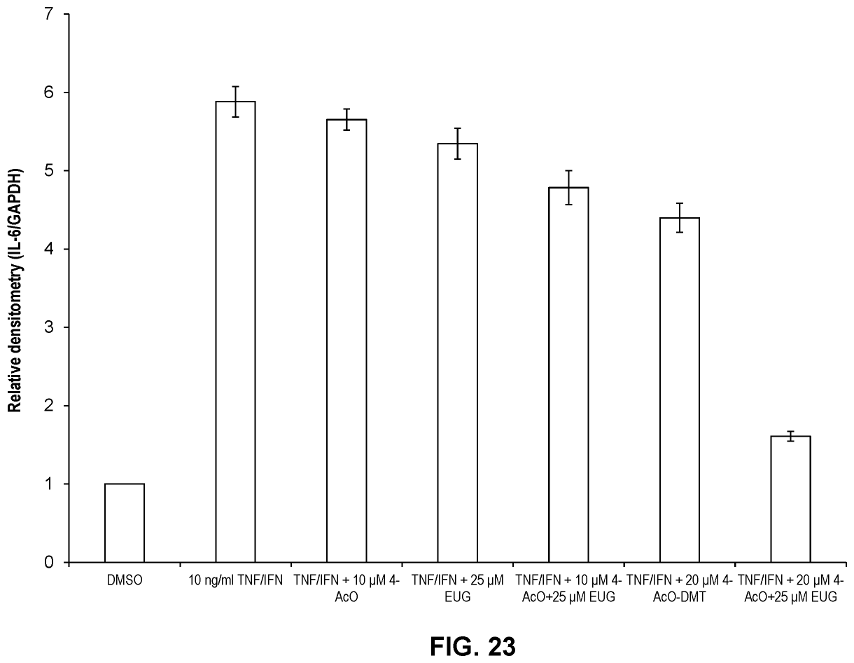

In vitro Assay Example 18 is illustrated in FIG. 23, which shows the separate and combined effects of 4-ACO-DMT and eugenol on IL-6 in the HSIEC cells treated with TNF-α/IFN-γ as described above. As can be seen in FIG. 23, combinations of 4-ACO-DMT and eugenol resulted in the reduction of inflammatory marker IL-6, with the combination of 20 μM 4-ACO-DMT and 25 μM eugenol reducing IL-6 to the greatest extent, thus indicating synergistic effects of 4-ACO-DMT and eugenol on IL-6. A combination of 20 μM 4-ACO-DMT and 25 μM eugenol reduced IL-6 by 63% more than 20 μM 4-ACO-DMT.

### Example 19

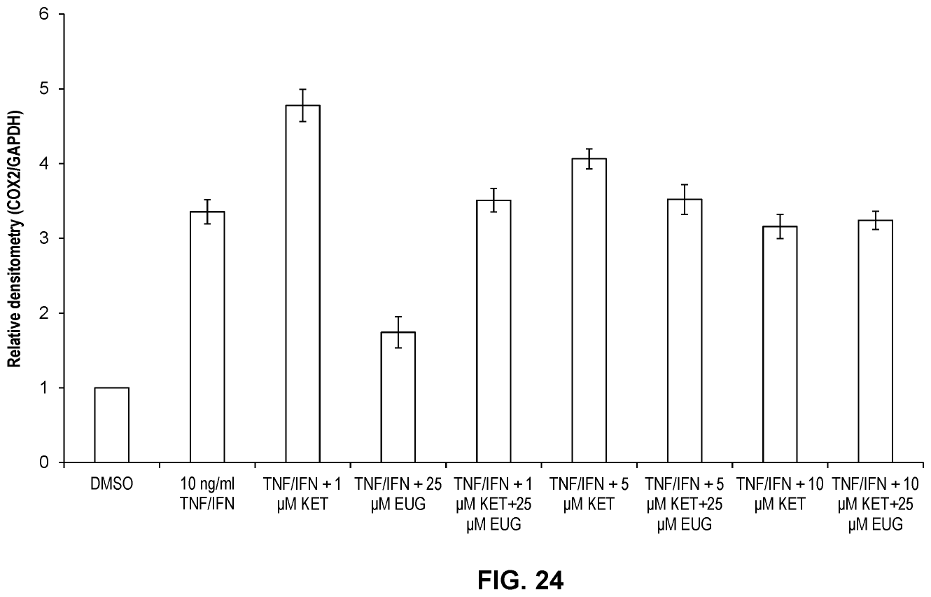

In vitro HSIEC Cell Assay Example 19 is illustrated in FIG. 24, which shows the effects of escalating doses of 1 μM, 5 μM, and 10 μM ketanserin, alone or in combination with 25 μM eugenol, on COX-2 in the HSIEC cells treated with TNF-α/IFN-γ as described above. As expected, none of the doses of ketanserin resulted in a significant reduction in COX-2 when combined with eugenol, while 1 μM ketanserin significantly increased COX-2.

### Example 20

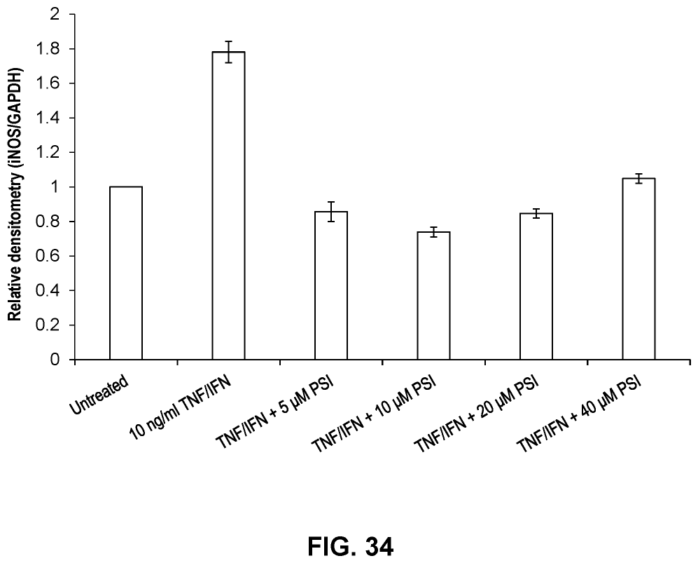

In vitro HSIEC Cell Assay Example 20 is illustrated in FIG. 34, which shows the effects of escalating doses of 5 μM, 10 μM, 20 μM, and 40 μM psilocybin on iNOS in the HSIEC cells treated with TNF-α/IFN-γ as described above. As can be seen in FIG. 34, all doses of psilocybin resulted in the reduction of inflammatory marker iNOS, with 10 μM psilocybin reducing iNOS to the greatest extent.

### Example 21

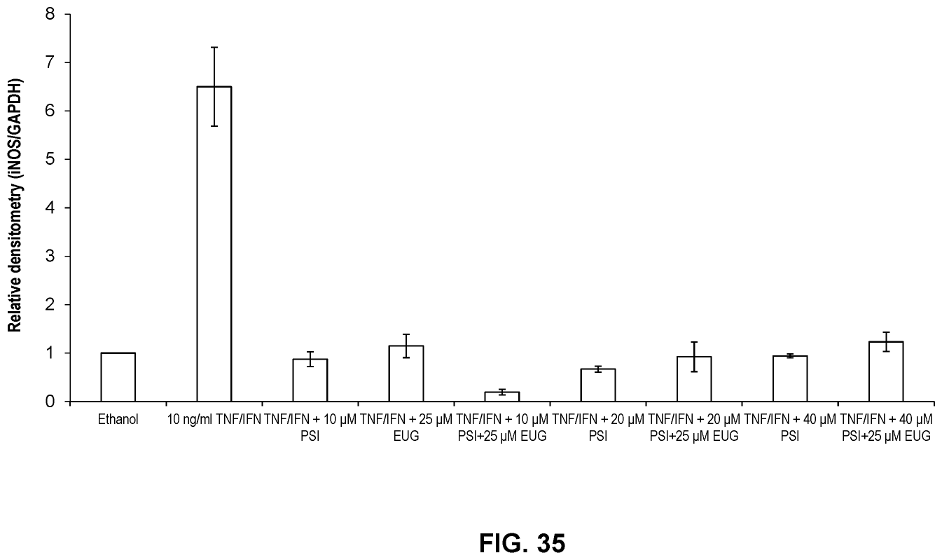

In vitro HSIEC Cell Assay Example 21 is illustrated in FIG. 35, which shows the separate and combined effects of psilocybin and eugenol on iNOS in the HSIEC cells treated with TNF-α/IFN-γ as described above. As can be seen in FIG. 35, the combination of psilocybin with eugenol resulted in an attenuation in the reduction of inflammatory marker iNOS relative to psilocybin alone, with the combinations of 10 μM psilocybin with 25 μM eugenol resulting in the greatest attenuation in the reduction of inflammatory marker iNOS relative to psilocybin alone, thus indicating that eugenol attenuates the reduction in iNOS resulting from psilocybin. A combination of 10 μM psilocybin with 25 μM eugenol reduced iNOS by 77% more than 10 μM psilocybin alone.

### Example 22

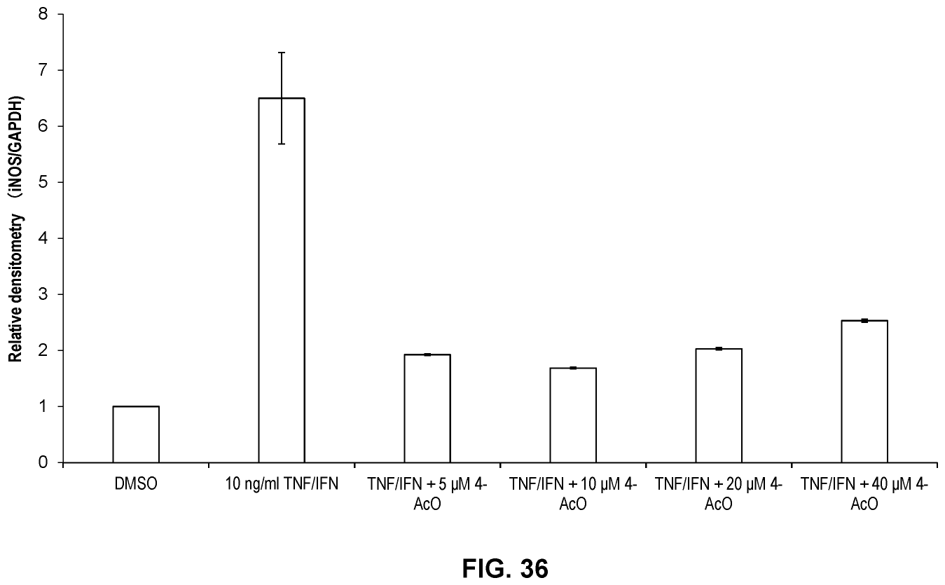

In vitro HSIEC Cell Assay Example 22 is illustrated in FIG. 36, which shows the effects of escalating doses of 5 μM, 10 μM, 20 μM, and 40 μM 4-ACO-DMT on iNOS in the HSIEC cells treated with TNF-α/IFN-γ as described above. As can be seen in FIG. 36, all doses of 4-ACO-DMT resulted in the reduction of inflammatory marker iNOS, with 10 μM 4-ACO-DMT reducing iNOS to the greatest extent.

### Example 23

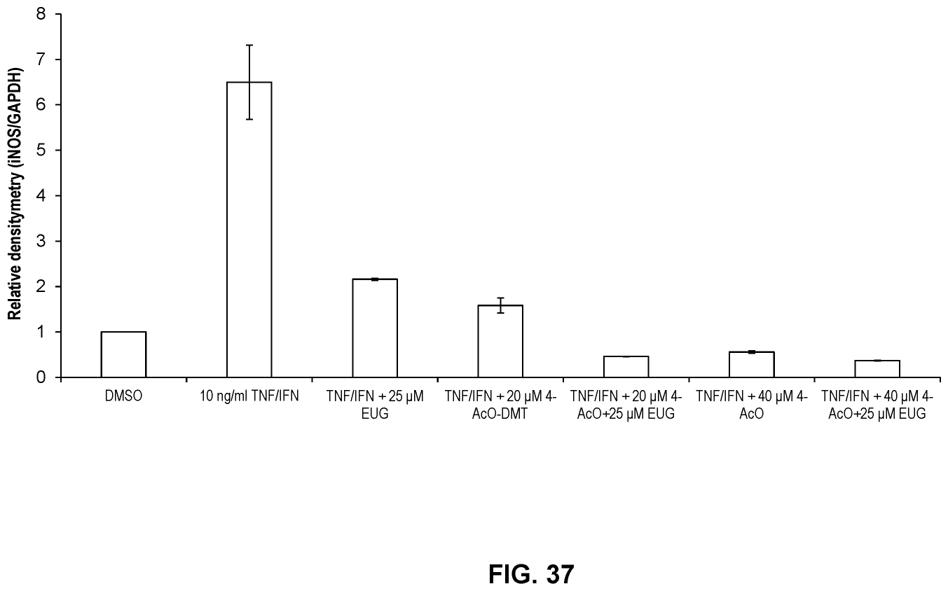

In vitro HSIEC Cell Assay Example 23 is illustrated in FIG. 37, which shows the separate and combined effects of 4-ACO-DMT and eugenol on iNOS in the HSIEC cells treated with TNF-α/IFN-γ as described above. As can be seen in FIG. 37, the combination of 4-ACO-DMT with eugenol resulted in an attenuation in the reduction of inflammatory marker iNOS relative to 4-ACO-DMT alone, with the combinations of 20 μM 4-ACO-DMT with 25 μM eugenol and 40 μM 4-ACO-DMT with 25 μM eugenol resulting in the greatest attenuation in the reduction of inflammatory marker iNOS relative to 4-ACO-DMT alone, thus indicating that eugenol attenuates the reduction in iNOS resulting from 4-ACO-DMT. A combination of 20 μM 4-ACO-DMT with 25 μM eugenol reduced iNOS by 71% more than 20 μM 4-ACO-DMT alone.

### Example 24

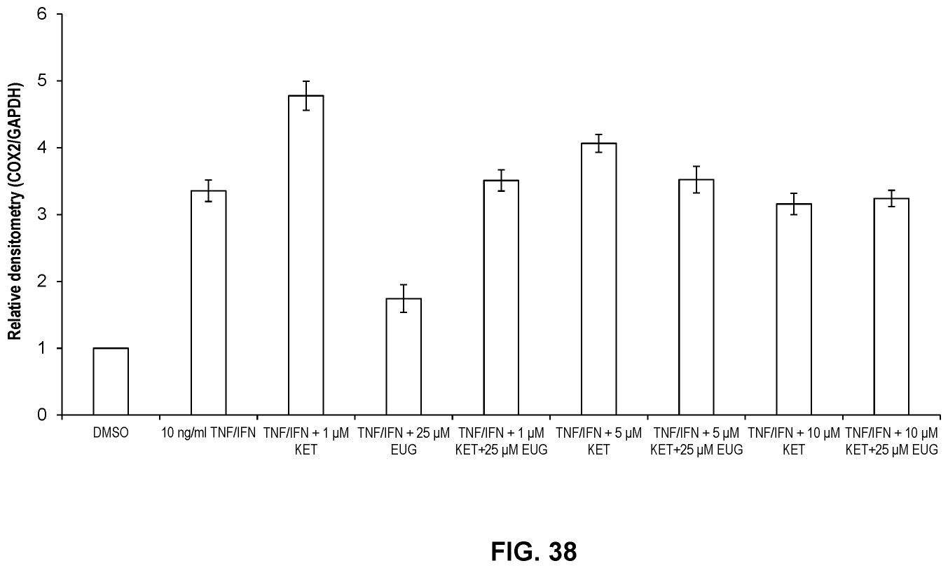

In vitro HSIEC Cell Assay Example 24 is illustrated in FIG. 38, which shows the effects of doses of 1 μM, 5 μM, and 10 μM ketanserin, alone or in combination with 25 μM eugenol, on COX-2 in the HSIEC cells treated with TNF-α/IFN-γ as described above. As expected, none of the doses of ketanserin resulted in a significant reduction in COX-2 when combined with eugenol, while 1 μM and 5 μM ketanserin significantly increased COX-2.

### Examples 25-29

### Effects of Combinations of 5HT2A Agonists Psilocybin and 4-ACO-DMT with Capsaicin on COX-2 and IL-6 in In Vitro HSIEC Cell Assays

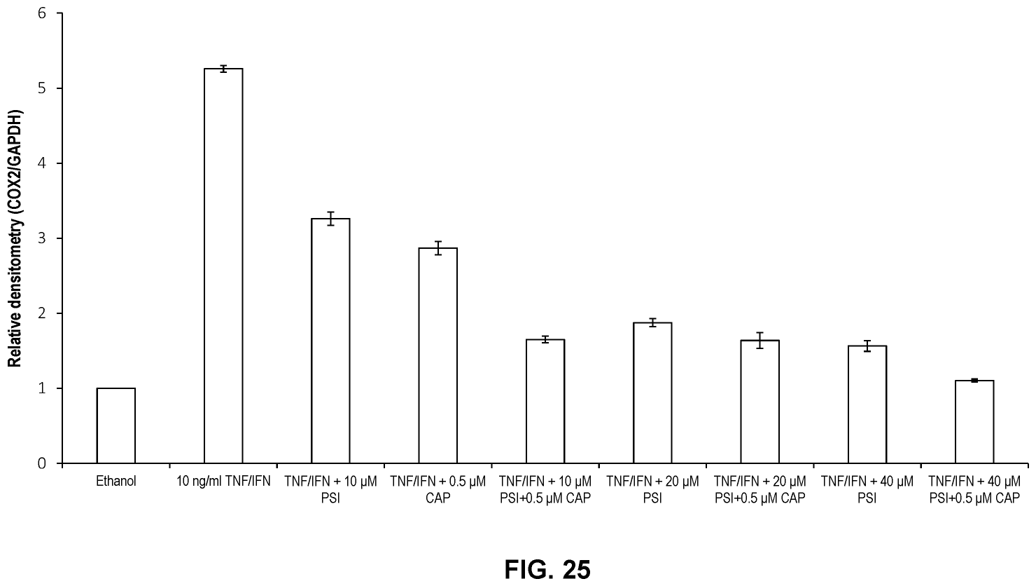

The anti-inflammatory effects of combinations of 5HT2A agonists psilocybin and 4-ACO-DMT with capsaicin, as evaluated based on their effects on COX-2 and IL-6, are described below and shown in FIGS. 25-29. Based on the mechanism of action of the 5HT2A agonists psilocybin and 4-ACO-DMT and capsaicin, it was hypothesized that combinations of psilocybin and 4-ACO-DMT with capsaicin would have synergistic effects on COX-2 and IL-6 while a combination of the 5HT2A antagonist ketanserin with capsaicin would not.

### Example 25

In vitro HSIEC Cell Assay Example 25 is illustrated in FIG. 25, which shows the separate and combined effects of psilocybin and capsaicin on COX-2 in the HSIEC cells treated with TNF-α/IFN-γ as described above. As can be seen in FIG. 25, combinations of psilocybin and capsaicin resulted in the reduction of inflammatory marker COX-2, with the combinations of 10 μM, 20 μM, and 40 μM psilocybin with 0.5 μM capsaicin reducing COX-2 to the greatest extent, thus indicating synergistic effects of psilocybin and capsaicin on COX-2. A combination of 40 μM psilocybin with 0.5 μM capsaicin reduced COX-2 by 33% more than 40 μM psilocybin alone.

### Example 26

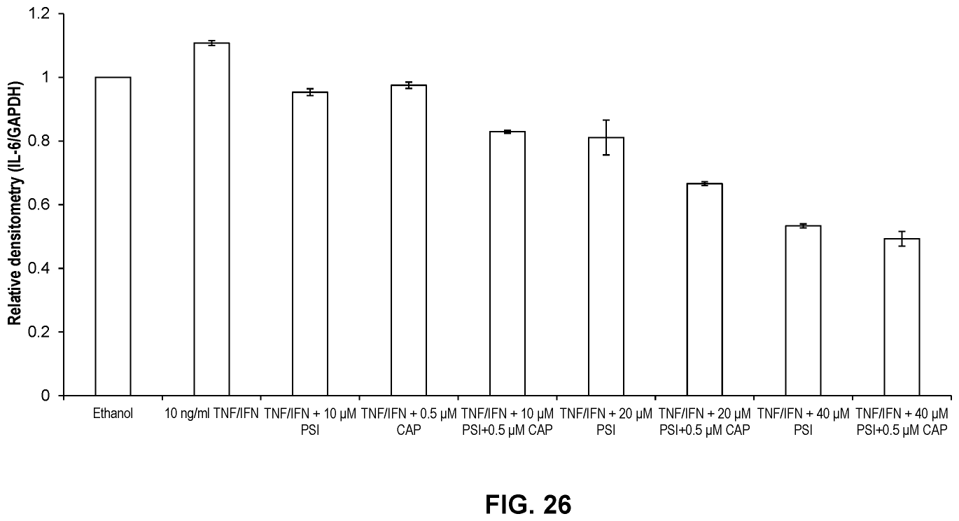

In vitro HSIEC Cell Assay Example 26 is illustrated in FIG. 26, which shows the separate and combined effects of psilocybin and capsaicin on IL-6 in the HSIEC cells treated with TNF-α/IFN-γ as described above. As can be seen in FIG. 26, combinations of psilocybin and capsaicin resulted in the reduction of inflammatory marker IL-6, with the combinations of 10 μM, 20 μM, and 40 μM psilocybin with 0.5 μM capsaicin reducing IL-6 to the greatest extent, thus indicating synergistic effects of psilocybin and capsaicin on IL-6. A combination of 40 μM psilocybin with 0.5 μM capsaicin reduced IL-6 by 8% more than 40 μM psilocybin alone.

### Example 27

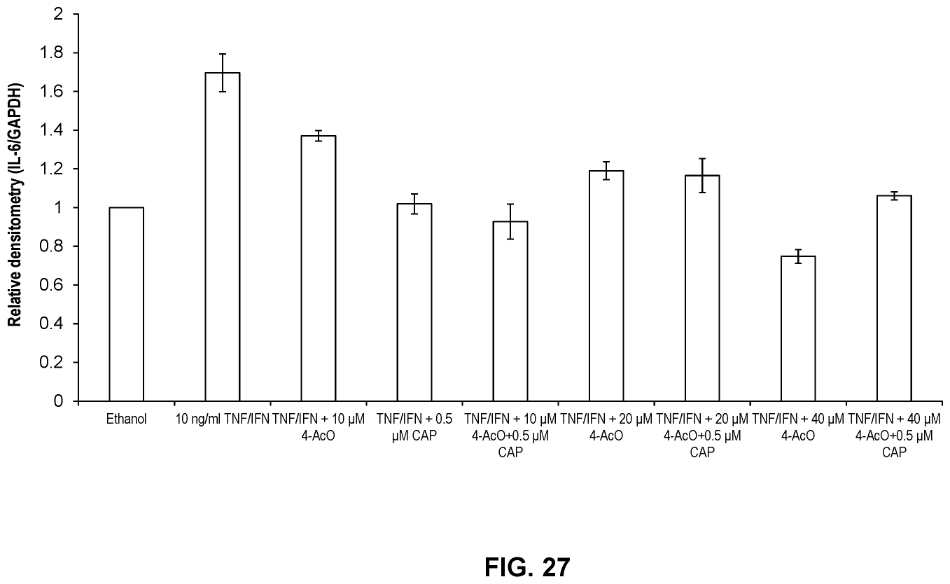

In vitro HSIEC Cell Assay Example 27 is illustrated in FIG. 27, which shows the separate and combined effects of 4-ACO-DMT and capsaicin on IL-6 in the HSIEC cells treated with TNF-α/IFN-γ as described above. As can be seen in FIG. 27, the combinations of 4-ACO-DMT and capsaicin resulted in the reduction of inflammatory marker IL-6, with the combination of 10 μM 4-ACO-DMT with 0.5 μM capsaicin reducing IL-6 to the greatest extent, thus indicating synergistic effects of 4-ACO-DMT and capsaicin on IL-6. A combination of 10 μM 4-ACO-DMT with 0.5 μM capsaicin reduced IL-6 by 32% more than 10 μM 4-ACO-DMT alone.

### Example 28

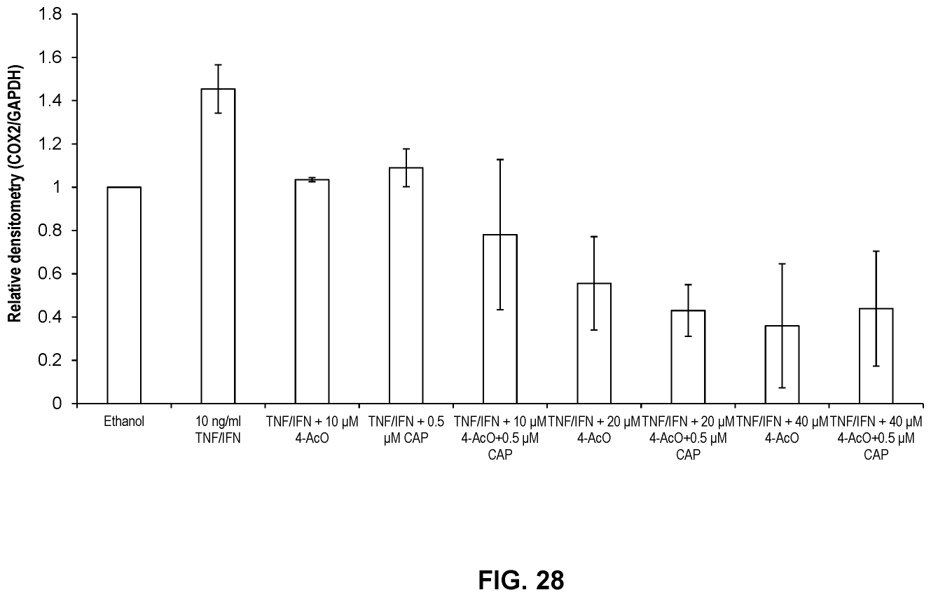

In vitro HSIEC Cell Assay Example 28 is illustrated in FIG. 28, which shows the separate and combined effects of 4-ACO-DMT and capsaicin on COX-2 in the HSIEC cells treated with TNF-α/IFN-γ as described above. As can be seen in FIG. 28, the combinations of psilocybin and capsaicin resulted in the reduction of inflammatory marker COX-2, with the combinations of 10 μM 4-ACO-DMT and 20 μM 4-ACO-DMT with 0.5 μM capsaicin reducing COX-2 to the greatest extent, thus indicating synergistic effects of 4-ACO-DMT and capsaicin on COX-2. A combination of 20 μM 4-ACO-DMT with 0.5 μM capsaicin reduced COX-2 by 23% more than 20 μM 4-ACO-DMT alone.

### Example 29

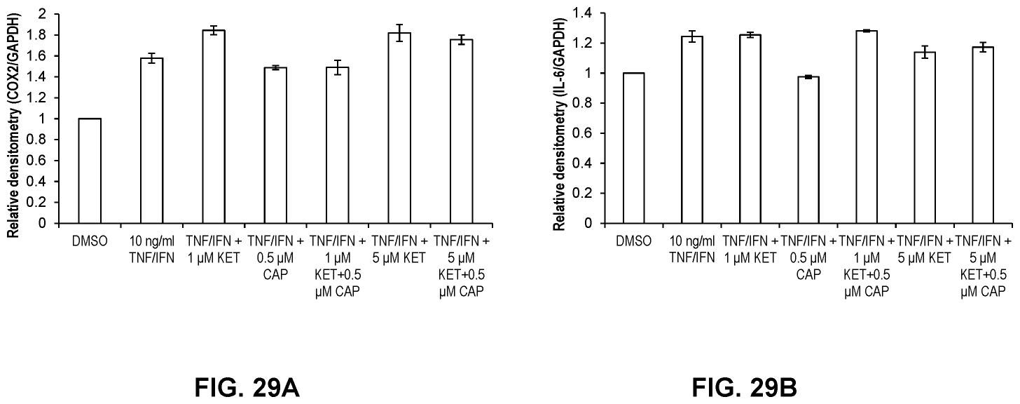

In vitro HSIEC Cell Assay Example 29 is illustrated in FIGS. 29A and 29B,

which show the effects of doses of 1 μM and 5 μM ketanserin, alone or in combination with 0.5 μM capsaicin, on COX-2 and IL-6 in the HSIEC cells treated with TNF-α/IFN-γ as described above. As expected, none of the doses of ketanserin resulted in a significant reduction in COX-2 or IL-6 when combined with capsaicin, while 1 μM ketanserin significantly increased COX-2.

### Examples 30-33

### Effects of Combinations of 5HT2A Agonists Psilocybin and 4-ACO-DMT with Curcumin on COX-2 and IL-6 in In Vitro HSIEC Cell Assays

### Example 30

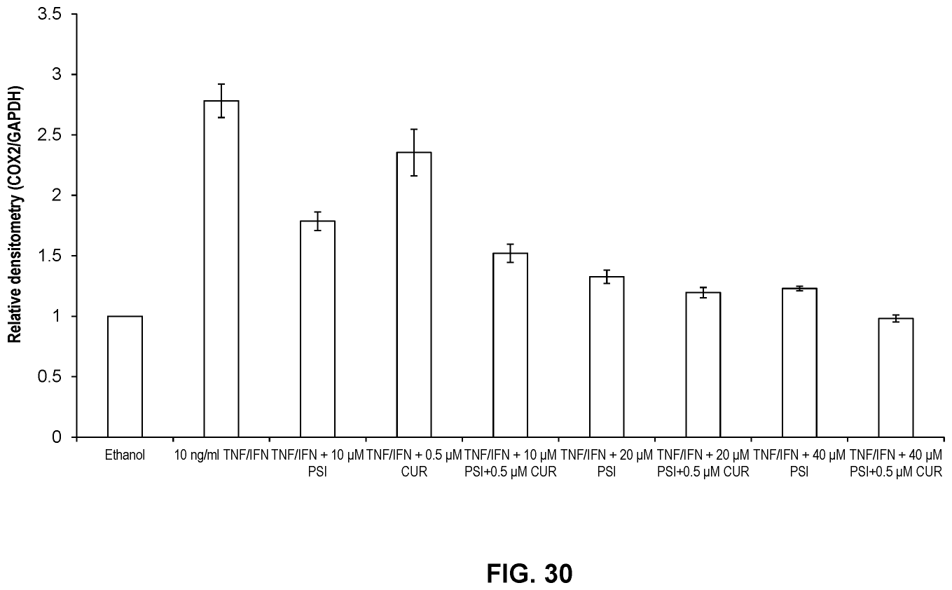

In vitro HSIEC Cell Assay Example 30 is illustrated in FIG. 30, which shows the separate and combined effects of psilocybin and curcumin on COX-2 in the HSIEC cells treated with TNF-α/IFN-γ as described above. As can be seen in FIG. 30, combinations of psilocybin and curcumin resulted in the reduction of inflammatory marker COX-2, with the combinations of 10 μM and 40 μM psilocybin with 0.5 μM curcumin reducing

COX-2 to the greatest extent, thus indicating synergistic effects of psilocybin and curcumin on COX-2. A combination of 40 μM psilocybin with 0.5 μM curcumin reduced COX-2 by 20% more than 40 μM psilocybin alone.

### Example 31

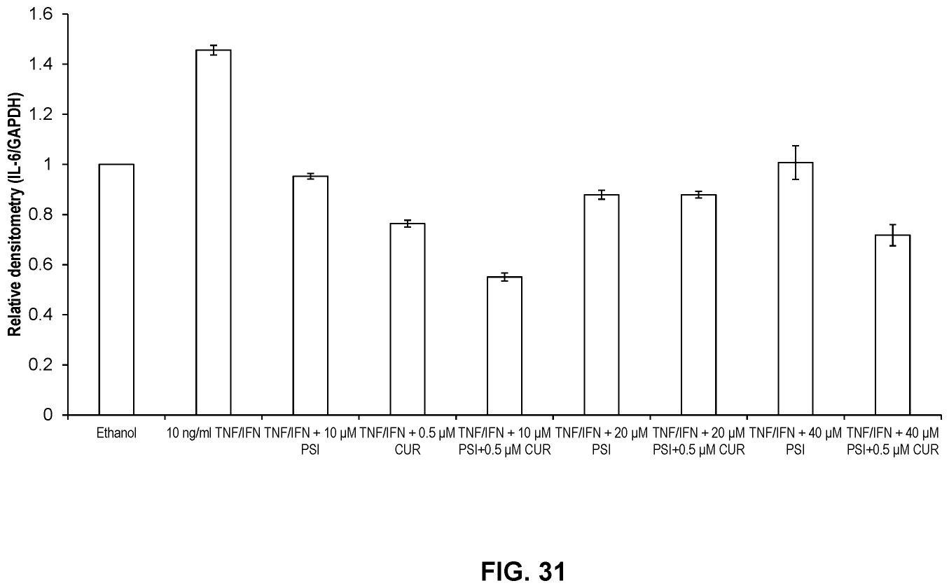

In vitro HSIEC Cell Assay Example 31 is illustrated in FIG. 31, which shows the separate and combined effects of psilocybin and curcumin on IL-6 in the HSIEC cells treated with TNF-α/IFN-γ as described above. As can be seen in FIG. 31, combinations of psilocybin and curcumin resulted in the reduction of inflammatory marker IL-6, with the combinations of 10 μM and 40 μM psilocybin with 0.5 μM curcumin reducing IL-6 to the greatest extent, thus indicating synergistic effects of psilocybin and curcumin on IL-6. A combination of 40 μM psilocybin with 0.5 μM curcumin reduced IL-6 by 28% more than 40 μM psilocybin alone.

### Example 32

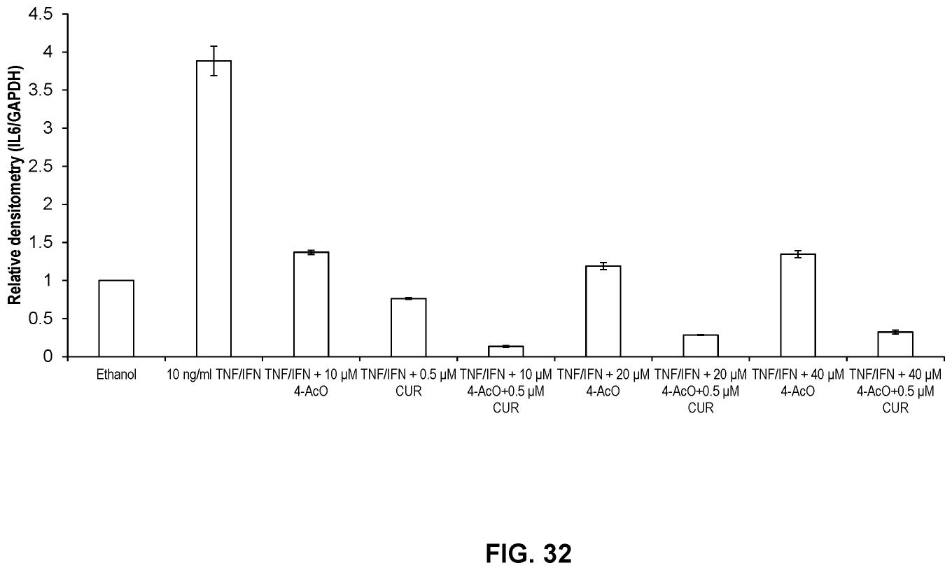

In vitro HSIEC Cell Assay Example 32 is illustrated in FIG. 32, which shows the separate and combined effects of 4-ACO-DMT and curcumin on IL-6 in the HSIEC cells treated with TNF-α/IFN-γ as described above. As can be seen in FIG. 32, the combinations of 4-ACO-DMT and curcumin resulted in the reduction of inflammatory marker IL-6, with the combinations of 10 μM, 20 μM and 40 μM 4-ACO-DMT with 0.5 μM curcumin reducing IL-6 to the greatest extent, thus indicating synergistic effects of 4-ACO-DMT and curcumin on IL-6. A combination of 40 μM 4-ACO-DMT with 0.5 μM curcumin reduced IL-6 by 76% more than 40 μM 4-ACO-DMT alone.

### Example 33

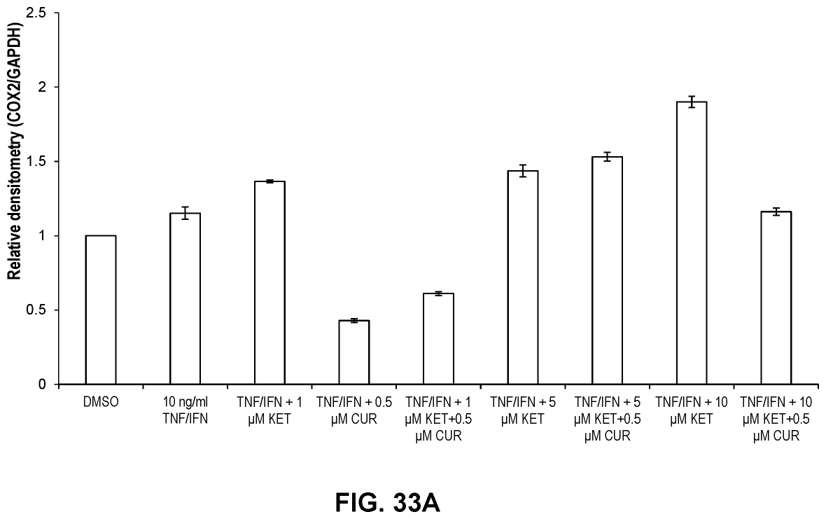

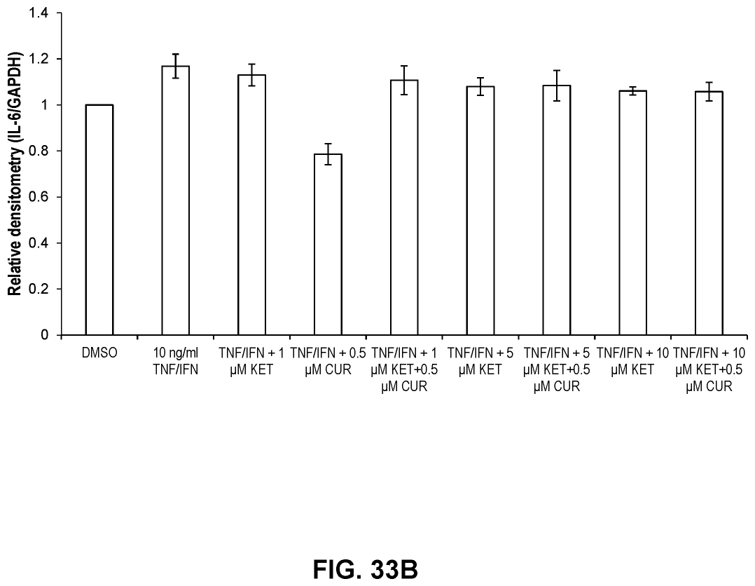

In vitro HSIEC Cell Assay Example 33 is illustrated in FIGS. 33A and 33B, which show the effects of doses of 1 μM, 5 μM, and 10 μM ketanserin, alone or in combination with 0.5 μM curcumin, on COX-2 and IL-6 in the HSIEC cells treated with TNF-α/IFN-γ as described above. As expected, none of the doses of ketanserin resulted in a significant reduction in COX-2 or IL-6 when combined with curcumin, while 1 μM, 5 μM, and 10 μM ketanserin significantly increased COX-2.

### Examples 34-48

### Effects of TRP Agonists Carvacrol, Piperine, and Cinnamaldehyde, Alone or in Combination with 5HT2A Agonist Psilocybin, on COX-2, IL-6, and IL-8 in In Vitro HSIEC Cell Assays

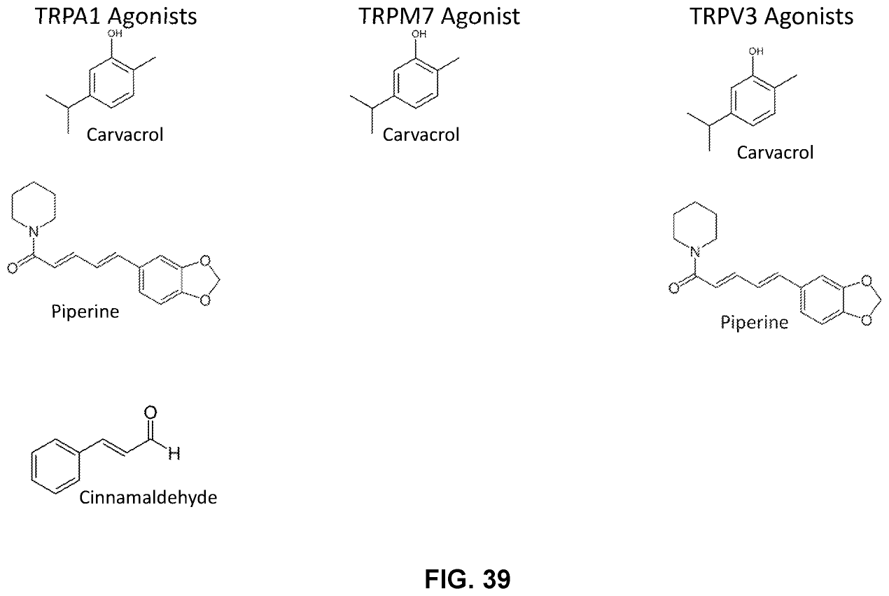

The anti-inflammatory effects of TRP agonists carvacrol, piperine, and cinnamaldehyde, as evaluated based on their effects on COX-2, IL-6, and IL-8 are described below and shown in FIGS. 39-53. FIG. 39 illustrates the structures of TRP agonists carvacrol, piperine, and cinnamaldehyde. Carvacrol is a TRPA1, TRPM7, and TRPV3 agonist. Piperine is a TRPA1 agonist and a TRPV3 agonist. Cinnamaldehyde is a TRPA1 agonist. Based on the mechanism of action of these TRP agonists, it was hypothesized that all three would have some anti-inflammatory activity.

### Example 34

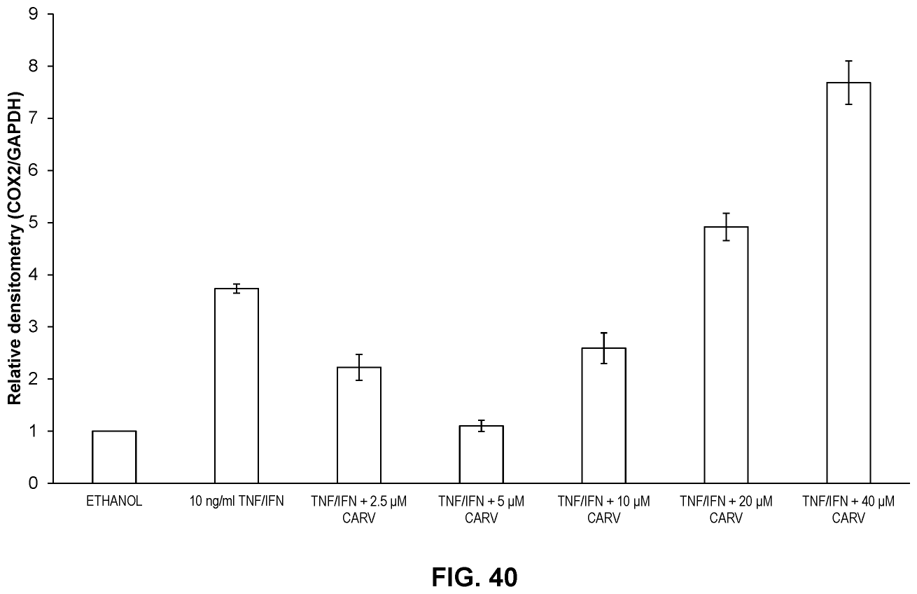

In vitro HSIEC Cell Assay Example 34 is illustrated in FIG. 40, which shows the effects of escalating doses of 2.5 μM, 5 μM, 10 μM, 20 μM and 40 μM carvacrol on COX-2 in the HSIEC cells treated with TNF-α/IFN-γ as described above. As can be seen in FIG. 40, doses of carvacrol of 2.5 μM, 5 μM and 10 μM resulted in the reduction of inflammatory marker COX-2, with 5 μM carvacrol reducing COX-2 to the greatest extent. Doses of carvacrol of 20 μM and 40 μM resulted in the increase of inflammatory marker COX-2.

### Example 35

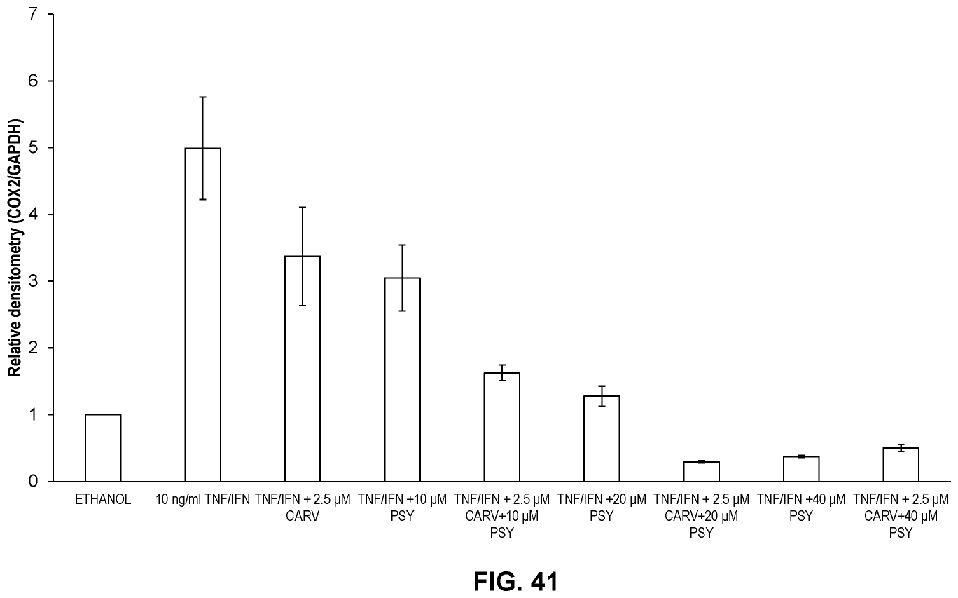

In vitro HSIEC Cell Assay Example 35 is illustrated in FIG. 41, which shows the separate and combined effects of psilocybin and carvacrol on COX-2 in the HSIEC cells treated with TNF-α/IFN-γ as described above. As can be seen in FIG. 41, combinations of psilocybin and carvacrol resulted in the reduction of inflammatory marker COX-2, with the combinations of 20 μM psilocybin with 2.5 μM carvacrol reducing COX-2 to the greatest extent, thus indicating synergistic effects of psilocybin and carvacrol on COX-2. A combination of 20 μM psilocybin with 2.5 μM carvacrol reducing COX-2 by 77% more than 20 μM psilocybin alone.

### Example 36

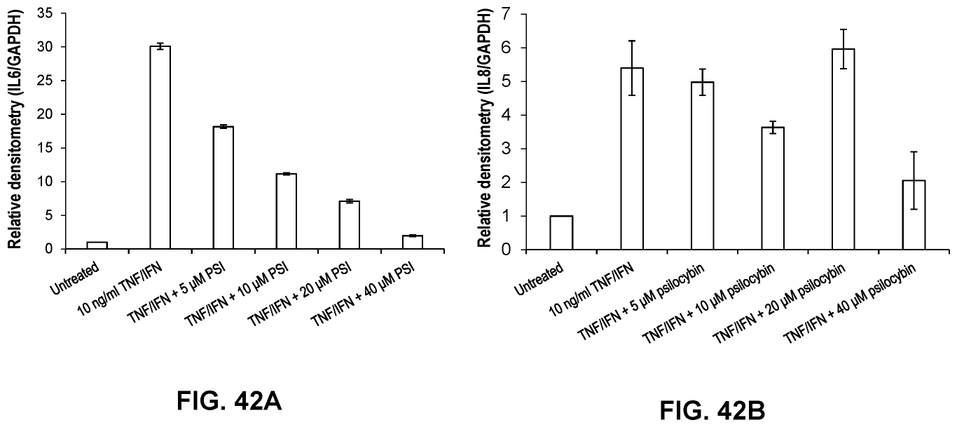

In vitro HSIEC Cell Assay Example 36 is illustrated in FIG. 42A, which shows the effects of psilocybin on IL-6 in the HSIEC cells treated with TNF-α/IFN-γ as described above. As can be seen in FIG. 42A, psilocybin resulted in the reduction of inflammatory marker IL-6, with 20 μM and 40 μM psilocybin reducing IL-6 to the greatest extent.

### Example 37

In vitro HSIEC Cell Assay Example 37 is illustrated in FIG. 42B, which shows the separate and combined effects of psilocybin on IL-8 in the HSIEC cells treated with TNF-α/IFN-γ as described above. As can be seen in FIG. 42B, most dosages of psilocybin resulted in the reduction of inflammatory marker IL-8, with 10 μM and 40 μM psilocybin reducing IL-8 to the greatest extent.

### Example 38

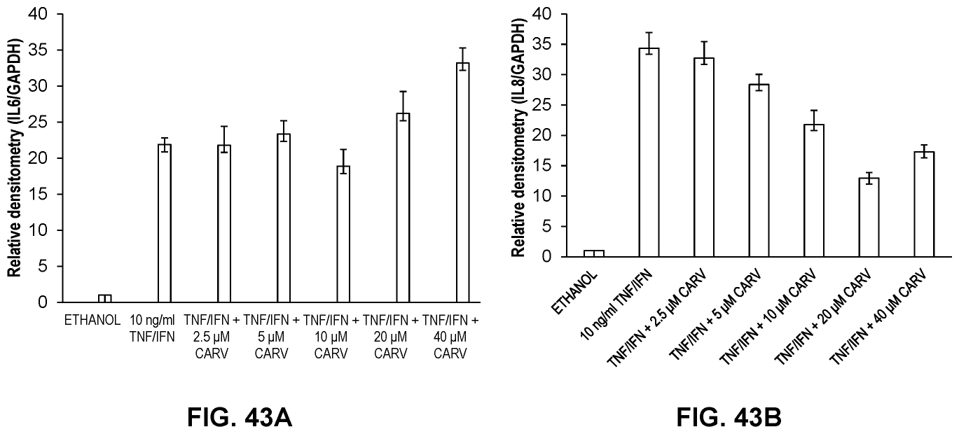

In vitro HSIEC Cell Assay Example 38 is illustrated in FIG. 43A, which shows the effects of escalating doses of 2.5 μM, 5 μM, 10 μM, 20 μM and 40 μM carvacrol on IL-6 in the HSIEC cells treated with TNF-α/IFN-γ as described above. As can be seen in FIG. 43A, doses of carvacrol of 10 μM resulted in the reduction of inflammatory marker IL-6. Doses of 2.5 μM, 5 μM, 20 μM and 40 μM carvacrol resulted in no substantial decrease or in the increase of inflammatory marker IL-6.

### Example 39

In vitro HSIEC Cell Assay Example 39 is illustrated in FIG. 43B, which shows the effects of escalating doses of 2.5 μM, 5 μM, 10 μM, 20 μM and 40 μM carvacrol on IL-8 in the HSIEC cells treated with TNF-α/IFN-γ as described above. As can be seen in FIG. 43B, all doses of carvacrol resulted in the reduction of inflammatory marker IL-8. Doses of 20 and 40 μM carvacrol resulted in the greatest decrease of inflammatory marker IL-8.

### Example 40

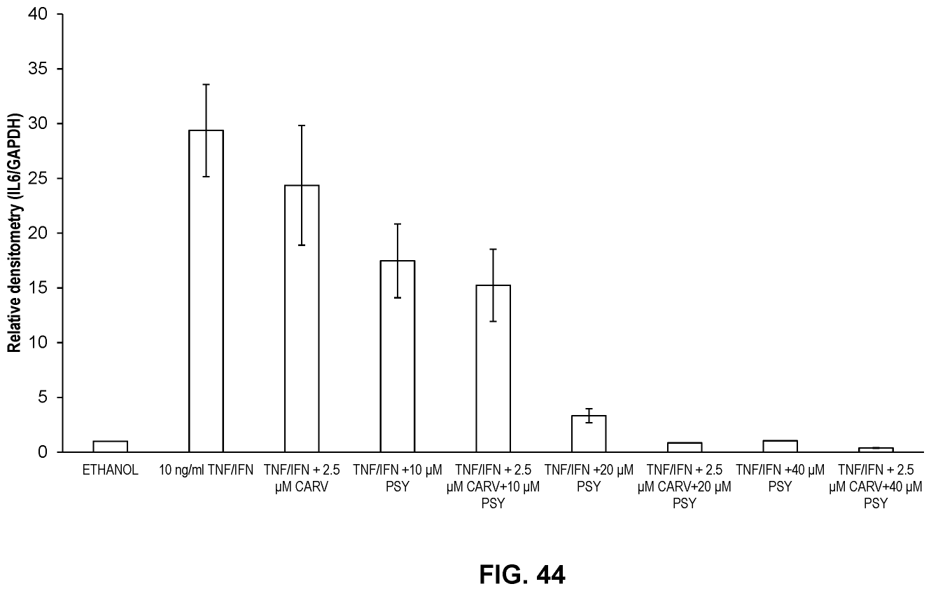

In vitro HSIEC Cell Assay Example 40 is illustrated in FIG. 44, which shows the separate and combined effects of psilocybin and carvacrol on IL-6 in the HSIEC cells treated with TNF-α/IFN-γ as described above. As can be seen in FIG. 44, combinations of psilocybin and carvacrol resulted in the reduction of inflammatory marker IL-6, with the combinations of 40 μM psilocybin with 2.5 μM carvacrol reducing IL-6 to the greatest extent, thus indicating synergistic effects of psilocybin and carvacrol on IL-6. A combination of 40 μM psilocybin with 2.5 μM carvacrol reduced IL-6 by 63% more than IIM psilocybin alone.

### Example 41

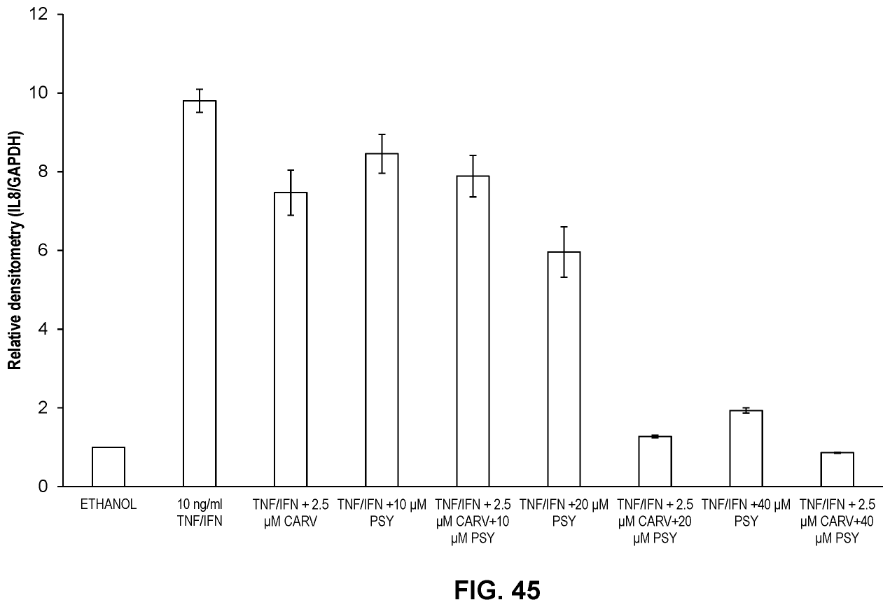

In vitro HSIEC Cell Assay Example 41 is illustrated in FIG. 45, which shows the separate and combined effects of psilocybin and carvacrol on IL-8 in the HSIEC cells treated with TNF-α/IFN-γ as described above. As can be seen in FIG. 45, combinations of psilocybin and carvacrol resulted in the reduction of inflammatory marker IL-8, with the combinations of 40 μM psilocybin with 2.5 μM carvacrol reducing IL-8 to the greatest extent, thus indicating synergistic effects of psilocybin and carvacrol on IL-8. A combination of 40 μM psilocybin with 2.5 μM carvacrol reducing IL-8 by 55% more than IIM psilocybin alone.

### Example 42

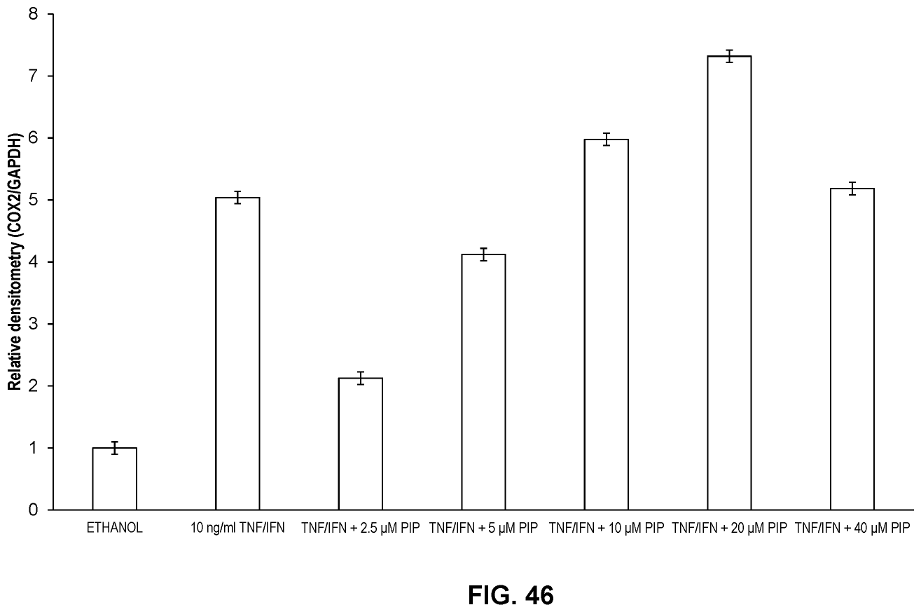

In vitro HSIEC Cell Assay Example 42 is illustrated in FIG. 46, which shows the effects of escalating doses of 2.5 μM, 5 μM, 10 μM, 20 μM, and 40 μM piperine on COX-2 in the HSIEC cells treated with TNF-α/IFN-γ as described above. As can be seen in FIG. 46, doses of piperine of 2.5 μM and 5 μM resulted in the reduction of inflammatory marker COX-2, with 2.5 μM piperine reducing COX-2 to the greatest extent. Doses of piperine of 10 μM, 20 μM, and 40 μM resulted in the increase of inflammatory marker COX-2.

### Example 43

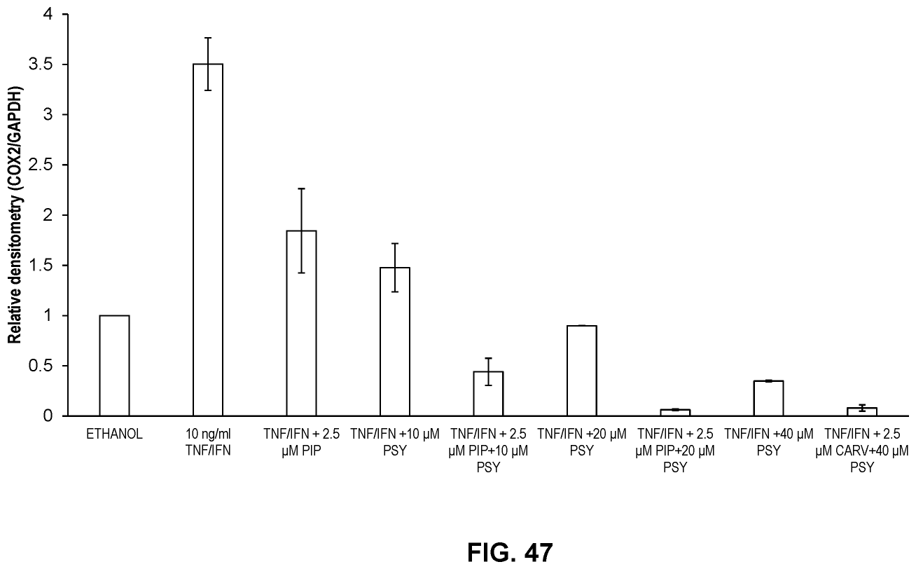

In vitro HSIEC Cell Assay Example 43 is illustrated in FIG. 47, which shows the separate and combined effects of psilocybin and piperine on COX-2 in the HSIEC cells treated with TNF-α/IFN-γ as described above. As can be seen in FIG. 47, combinations of psilocybin and piperine resulted in the reduction of inflammatory marker COX-2, with the combinations of 20 μM psilocybin with 2.5 μM piperine reducing COX-2 to the greatest extent, thus indicating synergistic effects of psilocybin and piperine on COX-2. A combination of 20 μM psilocybin with 2.5 μM piperine reducing COX-2 by 93% more than 20 μM psilocybin alone.

### Example 44

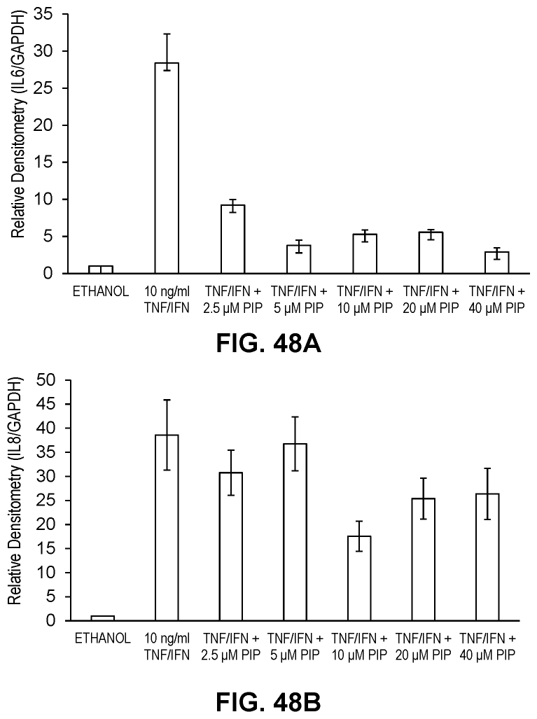

In vitro HSIEC Cell Assay Example 44 is illustrated in FIG. 48A, which shows the effects of piperine on IL-6 in the HSIEC cells treated with TNF-α/IFN-γ as described above. As can be seen in FIG. 48A, piperine resulted in the reduction of inflammatory marker IL-6, with 5 μM and 40 μM piperine reducing IL-6 to the greatest extent.

### Example 45

In vitro HSIEC Cell Assay Example 45 is illustrated in FIG. 48B, which shows the effects of piperine on IL-8 in the HSIEC cells treated with TNF-α/IFN-γ as described above. As can be seen in FIG. 48B, most dosages of piperine resulted in the reduction of inflammatory marker IL-8, with 10 μM, 20 μM, and 40 μM piperine reducing IL-8 to the greatest extent.

### Example 46

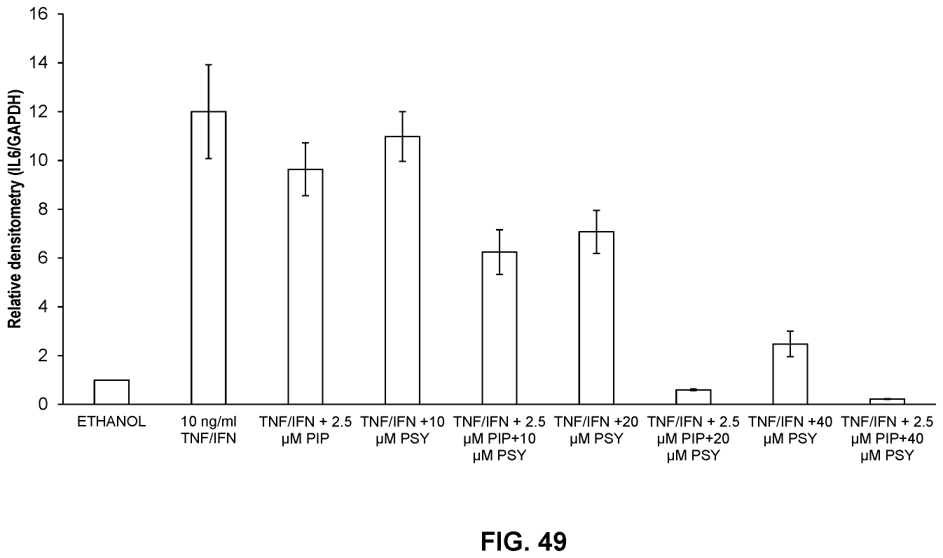

In vitro HSIEC Cell Assay Example 46 is illustrated in FIG. 49, which shows the separate and combined effects of psilocybin and piperine on IL-6 in the HSIEC cells treated with TNF-α/IFN-γ as described above. As can be seen in FIG. 49, combinations of psilocybin and piperine resulted in the reduction of inflammatory marker IL-6, with the combinations of 20 μM or 40 μM psilocybin with 2.5 μM piperine reducing IL-6 to the greatest extent, thus indicating synergistic effects of psilocybin and piperine on IL-6. A combination of 20 μM psilocybin with 2.5 μM piperine reduced IL-6 by 92% more than μM psilocybin, and 40 μM psilocybin with 2.5 μM piperine reduced IL-6 by 91% more than 40 μM psilocybin.

### Example 47

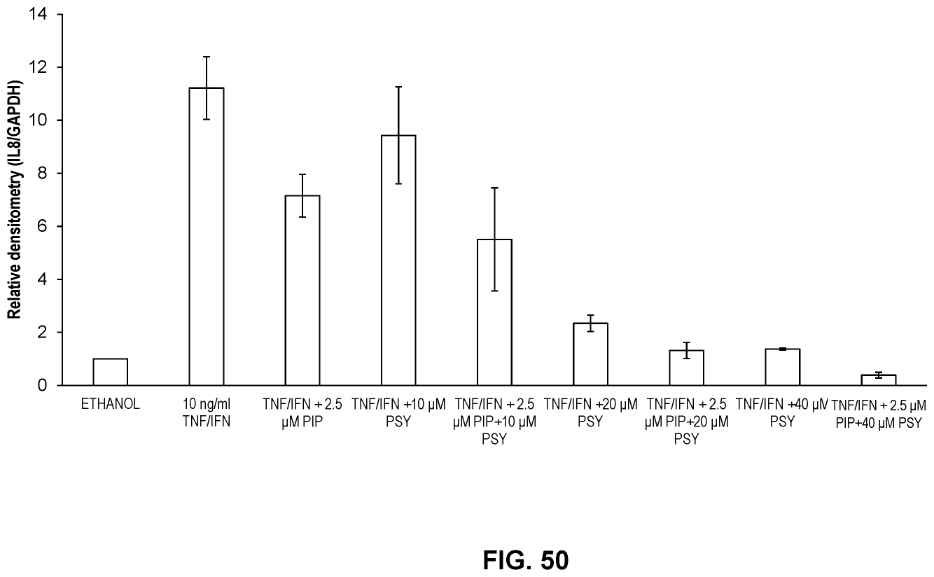

In vitro HSIEC Cell Assay Example 47 is illustrated in FIG. 50, which shows the separate and combined effects of psilocybin and piperine on IL-8 in the HSIEC cells treated with TNF-α/IFN-γ as described above. As can be seen in FIG. 50, combinations of psilocybin and piperine resulted in the reduction of inflammatory marker IL-8, with the combinations of 20 μM or 40 μM psilocybin with 2.5 μM piperine reducing IL-8 to the greatest extent, thus indicating synergistic effects of psilocybin and piperine on IL-8. A combination of 20 μM psilocybin with 2.5 μM piperine reduced IL-8 by 44% more than μM psilocybin, and 40 μM psilocybin with 2.5 μM piperine reduced IL-8 by 72% more than 40 μM psilocybin.

### Example 48

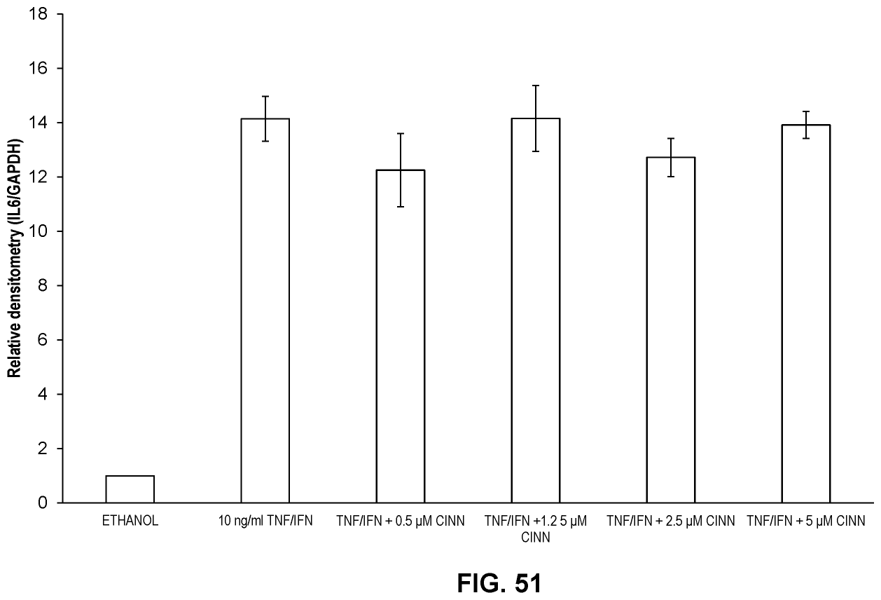

In vitro HSIEC Cell Assay Example 48 is illustrated in FIG. 51, which shows the effects of escalating doses of 0.5 μM, 1.25 μM, 2.5 μM and 5 μM cinnemaldehyde on IL-6 in the HSIEC cells treated with TNF-α/IFN-γ as described above. As can be seen in FIG. 51, doses of cinnemaldehyde of 0.5 μM and 2.5 μM resulted in the reduction of inflammatory marker IL-6. Doses of 1.25 μM and 5 μM cinnemaldehyde resulted in no substantial decrease or in the increase of inflammatory marker IL-6.

### Example 49

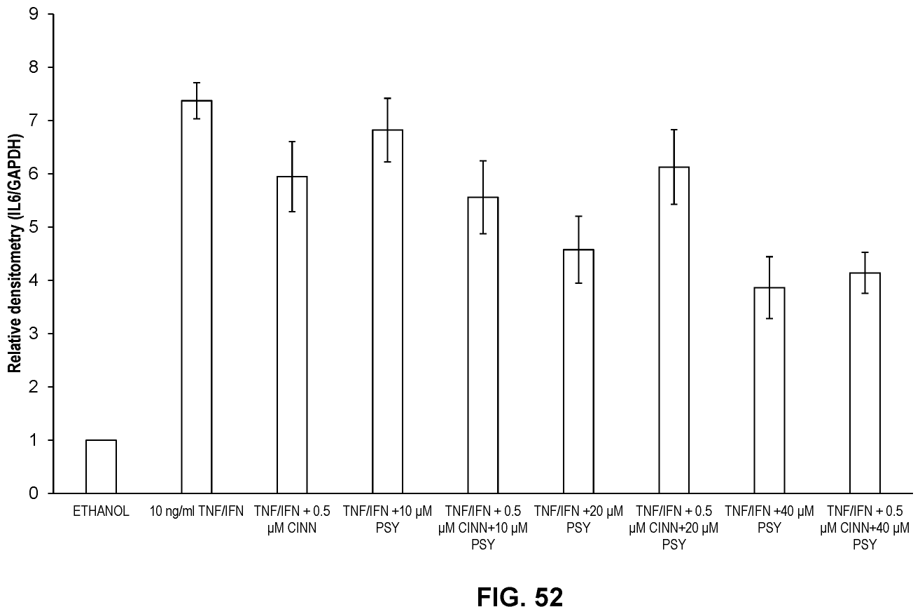

In vitro HSIEC Cell Assay Example 49 is illustrated in FIG. 52, which shows the separate and combined effects of psilocybin and cinnemaldehyde on IL-6 in the HSIEC cells treated with TNF-α/IFN-γ as described above. As can be seen in FIG. 52, combinations of psilocybin and cinnemaldehyde resulted in the reduction of inflammatory marker IL-6, with the combination of 10 μM psilocybin with 0.5 μM cinnemaldehyde showing synergistic effects of psilocybin and cinnemaldehyde on IL-6. A combination of 10 μM psilocybin with 0.5 μM cinnemaldehyde reduced IL-6 by 18% more than 10 μM psilocybin alone.

In Vitro Examples 50-51 Showing Effects of 5HT2A Agonists and/or TRP Agonists on Inflammatory Markers on 3-Dimensional Intestinal Tissue Model

### Overview of Methods

Model: An Epilntestinal tissue model (Mat Tek) was used as inflammation model that exhibits in vivo-like growth and morphological characteristics, in which cells sustain differentiation and metabolic status similar to those of human intestinal epithelium.

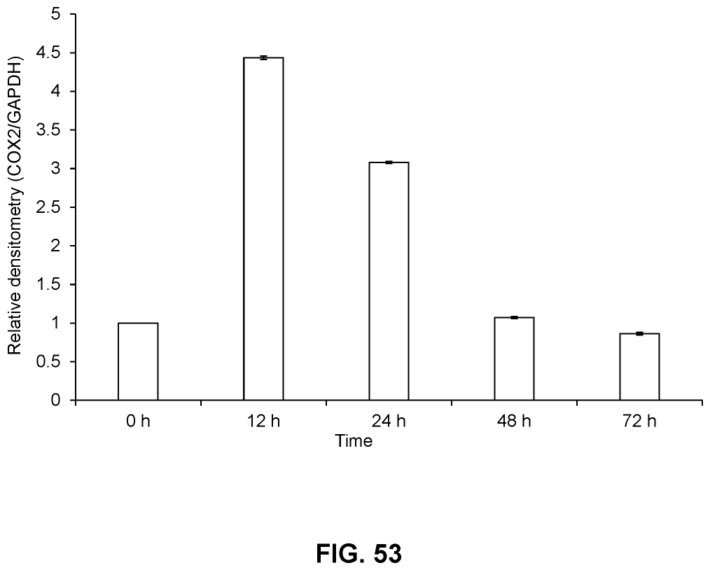

Induction of Inflammation in 3D Tissue Assays: One Mat Tek model was exposed to 10 ng/ml (i.e., the concentration was shown to effectively induce inflammation in the HSIEC assays described above) of TNF/IFN for periods of time ranging from 0-72 hours. GAPDH was used as a reference housekeeping marker for relative densitometry measures. As shown in FIG. 53, the greatest inflammatory effect was observed at 12 hours after treatment with TNF-α/IFN-γ. Thus, the anti-inflammatory potential of the molecules and formulations of interest were evaluated at 12 hours post-TNF-α/IFN-γ treatment in the Mat Tek 3D tissue assays described below. Ethanol was used as a vehicle for dilution and as a negative control showing the amount of the inflammatory marker present when inflammation was not induced.

### Examples 50-51

### Effects of Combinations of 5HT2A Agonist Psilocybin with Capsaicin on COX-2 and IL-6 in In Vitro 3D Tissue Assays

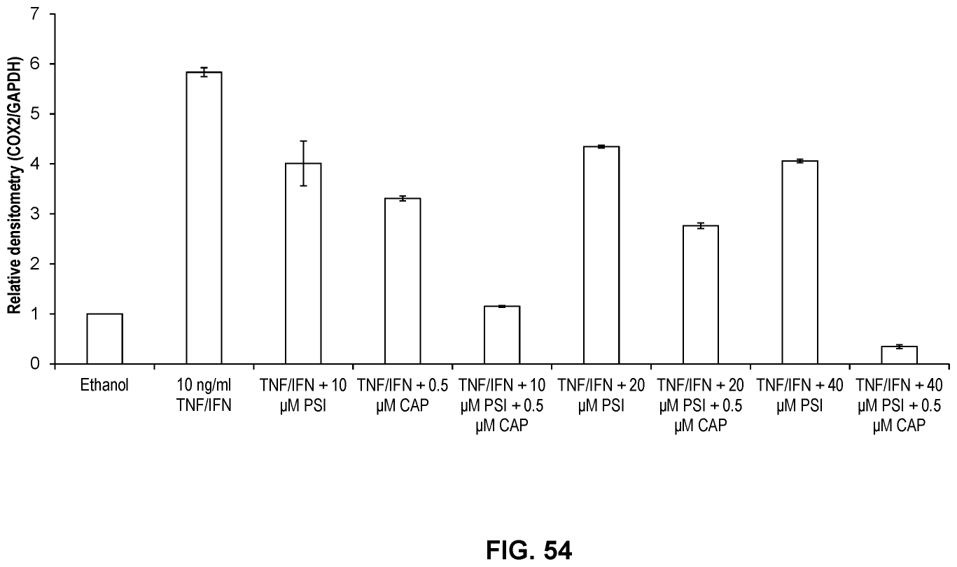

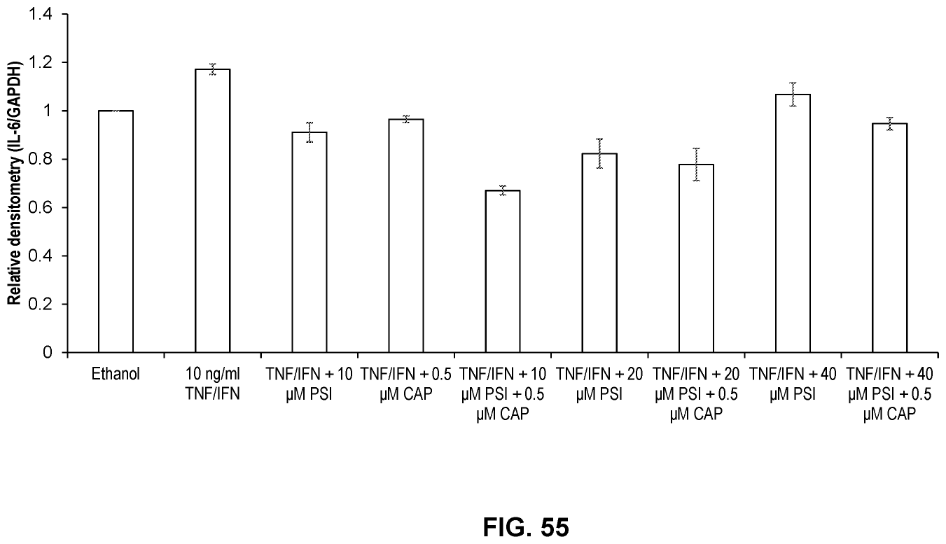

The anti-inflammatory effects of combinations of the 5HT2A agonist psilocybin with TRP agonist capsaicin, as evaluated based on their effects on COX-2 and IL-6, are described below and shown in FIGS. 54-55. Based on the mechanism of action of the agonist psilocybin and capsaicin, it was hypothesized that combinations of psilocybin and 4-ACO-DMT with capsaicin would have synergistic effects on COX-2 and IL-6 in 3D tissue assays.

### Example 50

In vitro 3D Tissue Assay Example 50 is illustrated in FIG. 54, which shows the separate and combined effects of psilocybin and capsaicin on COX-2 in the 3D Mat Tek tissue model treated with TNF-α/IFN-γ as described above. As can be seen in FIG. 54, combinations of psilocybin and capsaicin resulted in the reduction of inflammatory marker COX-2, with the combinations of 10 μM, 20 μM and 40 μM psilocybin with 0.5 μM capsaicin reducing COX-2 to the greatest extent, thus indicating synergistic effects of psilocybin and capsaicin on COX-2. A combination of 40 μM psilocybin with 0.5 μM capsaicin reduced COX-2 by 91% more than 40 μM psilocybin alone.

### Example 51

In vitro Assay Example 51 is illustrated in FIG. 55, which shows the separate and combined effects of psilocybin and capsaicin on IL-6 in the 3D Mat Tek tissue model treated with TNF-α/IFN-γ as described above. As can be seen in FIG. 55, combinations of psilocybin and capsaicin resulted in the reduction of inflammatory marker IL-6, with the combinations of 10 μM, 20 μM and 40 μM psilocybin with 0.5 μM capsaicin reducing IL-6 to the greatest extent, thus indicating synergistic effects of psilocybin and capsaicin on IL-6. A combination of 10 μM psilocybin with 0.5 μM capsaicin reduced IL-6 by 31% more than 10 μM psilocybin.

In Vitro Examples 52-59 Showing Effects of 5HT2A Agonists and/or TRP Agonists on Markers of Depression and Inflammatory Markers in A-172 Glioblastoma Cells

### Overview of Methods

This experiment was designed to explore the synergistic interaction of 5HT2A agonist and a TRP agonists on receptors in the brain that are associated with depression and/or inflammation. A-172 neuronal cells are glioblastoma (cancer) cells. The development of an inflammatory microenvironment is considered important to the initiation and progression of glioblastoma; however, therapeutic approaches to target inflammation have previously been limited. The abnormal function of IL-6 and IL-8 has been predicted to lead to glioblastoma. The effects of psilocybin and eugenol, alone or in combination, on GABA/BDNF (markers related to depression in which patients are often deficient) and IL-6 and IL-8 (markers of inflammation) were analyzed for potential to reduce inflammatory markers and simultaneously increase brain markers associated with depression.

MTT Cell Viability Assay: An MTT assay evaluating the effects of psilocybin and eugenol on cellular metabolic activity was conducted on the A-172 cells in the manner described above with respect to the MTT assay conducted on the HSIEC cells. As shown in Table 50 below, the 80 μM dose of psilocybin resulted in a modest increase in cell growth, while eugenol was observed to have a slight inhibitory effect. Thus, as with the HSIEC cells, relatively low doses of the 5HT2A agonists and the TRP agonists were used with the A-172 cells as described in the Examples below.

TABLE 50
Cellular Metabolic Activity in A-172 Cells
Treated with Compounds of Interest
MTT in A172 Cells

| | | | | |
|---|---|---|---|---|
| 0 | 24 h | 48 h | 72 h | 96 h |

Psilocybin

| | | | | | | |
|---|---|---|---|---|---|---|
| Control | EtOH | 0.092 | 0.149 | 0.236 | 0.341 | 0.638 |
| | 5 μM | 0.090 | 0.155 | 0.253 | 0.374 | 0.530 |
| | 10 μM | 0.090 | 0.181 | 0.269 | 0.363 | 0.577 |
| | 20 μM | 0.082 | 0.201 | 0.303 | 0.370 | 0.636 |
| | 40 μM | 0.086 | 0.287 | 0.369 | 0.457 | 0.703 |
| | 80 μM | 0.084 | 0.469 | 0.528 | 0.586 | 0.871 |

Eugenol

| | | | | | | |
|---|---|---|---|---|---|---|
| Control | EtOH | 0.092 | 0.149 | 0.236 | 0.341 | 0.638 |
| | 10 μM | 0.084 | 0.137 | 0.234 | 0.329 | 0.533 |
| | 25 μM | 0.083 | 0.153 | 0.266 | 0.318 | 0.557 |
| | 50 μM | 0.086 | 0.146 | 0.229 | 0.327 | 0.542 |
| | 100 μM | 0.082 | 0.170 | 0.230 | 0.322 | 0.516 |
| | 200 μM | 0.089 | 0.170 | 0.224 | 0.313 | 0.431 |
| | 400 μM | 0.088 | 0.173 | 0.208 | 0.272 | 0.422 |

Psilocybin + Eugenol

| | | | | | | |
|---|---|---|---|---|---|---|
| Control | EtOH | 0.123 | 0.197 | 0.269 | 0.430 | 0.316 |
| | 25 μM EUG | 0.134 | 0.202 | 0.297 | 0.473 | 0.319 |
| | 5 μM PSI | 0.132 | 0.199 | 0.261 | 0.438 | 0.321 |
| | 5 μM PSI + μm 25 EUG | 0.131 | 0.201 | 0.280 | 0.436 | 0.288 |
| | 10 μM PSI | 0.139 | 0.206 | 0.279 | 0.431 | 0.311 |
| | 10 μM PSI + 25 μM EUG | 0.138 | 0.195 | 0.293 | 0.332 | 0.251 |
| | 20 μM PSI | 0.143 | 0.204 | 0.282 | 0.451 | 0.270 |
| | 20 μM PSI + 25 μM EUG | 0.139 | 0.199 | 0.299 | 0.432 | 0.342 |

The A-172 cells already had elevated content of COX-2 and IL-6 compared to normal cells due to aberrant genetic regulation. Thus, unlike the experiments on HSIEC cells described above, no inflammatory treatment was applied to the A-172 cells. The A-172 cells were grown to −60% confluence on T-25 flasks to allow a 96-hour duration of exposure of the cells to the compounds of interest

### Example 52

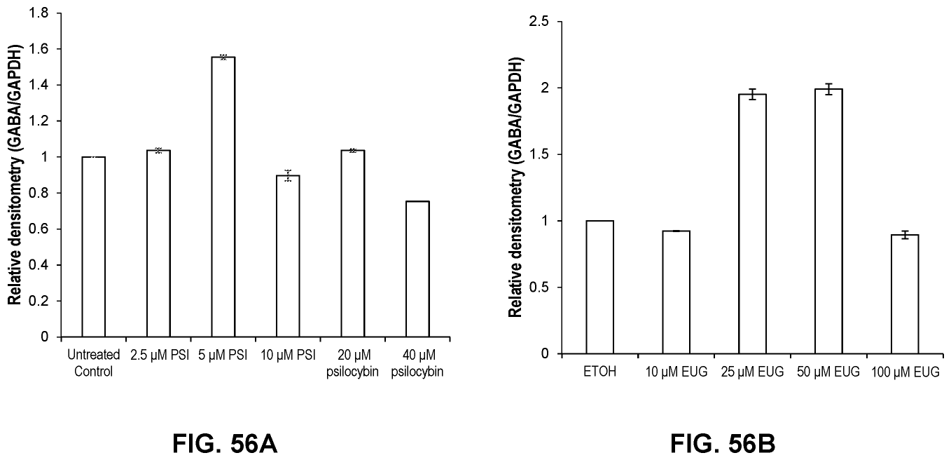

In vitro A-172 Cell Assay Example 52 is illustrated in FIG. 56A, which shows the effects of escalating doses of 2.5 μM, 5 μM, 10 μM, 20 μM, and 40 μM psilocybin on GABA in the A-172 cells described above. As can be seen in FIG. 56A, doses of psilocybin of 2.5 μM, 5 μM, and 20 μM resulted in the increase of depression-related marker GABA, with the 5 μM dose resulting in the greatest increase. Doses of psilocybin of 10 μM and 40 μM resulted in the decrease of GABA.

### Example 53

In vitro A-172 Cell Assay Example 53 is illustrated in FIG. 56B, which shows the effects of escalating doses of 10 μM, 25 μM, 50 μM, and 100 μM eugenol on depression-related marker GABA in the A-172 cells described above. As can be seen in FIG. 56B, doses of eugenol of 25 μM and 50 μM resulted in the increase of GABA. Doses of eugenol of 10 μM and 100 μM had little effect on GABA.

### Example 54

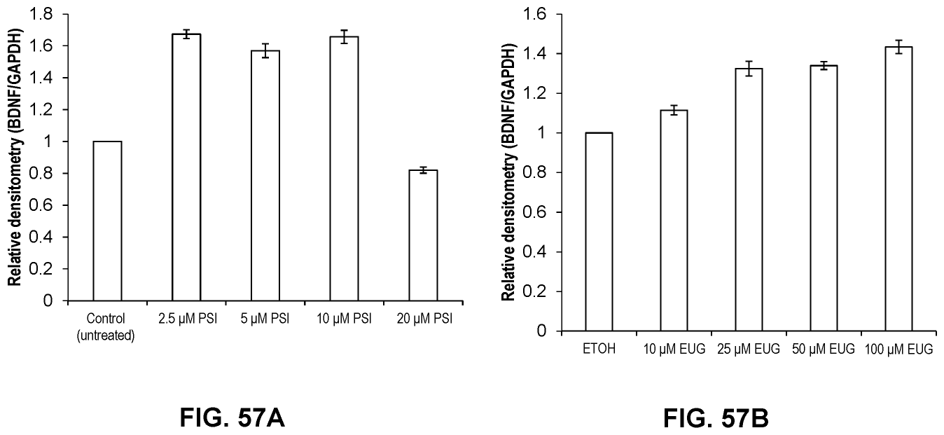

In vitro A-172 Cell Assay Example 54 is illustrated in FIG. 57A, which shows the effects of escalating doses of 2.5 μM, 5 μM, 10 μM, and 20 μM psilocybin on depression-related marker BDNF in the A-172 cells described above. As can be seen in FIG. 57A, doses of psilocybin of 2.5 μM, 5 μM, and 10 μM resulted in the increase of BDNF. Doses of psilocybin of 20 μM resulted in the decrease of BDNF.

### Example 55

In vitro A-172 Cell Assay Example 55 is illustrated in FIG. 57B, which shows the effects of escalating doses of 10 μM, 25 μM, 50 μM, and 100 μM eugenol on depression-related marker BDNF in the A-172 cells described above. As can be seen in FIG. 57B, doses of 10 μM, 25 μM, 50 μM, and 100 μM of eugenol all resulted in the increase of BDNF, with the 100 μM dose resulting in the greatest increase in BDNF.

### Example 56

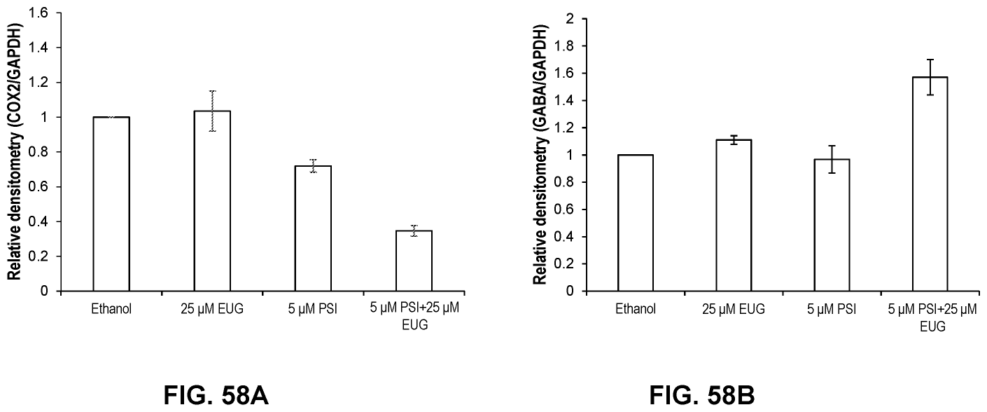

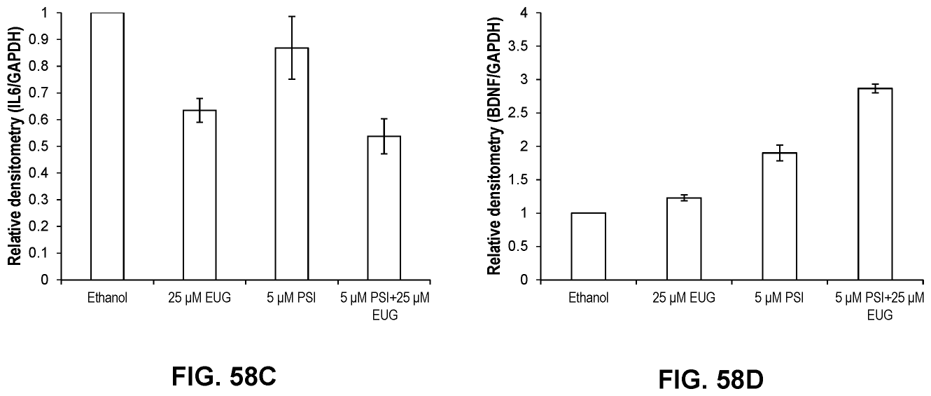

In vitro A-172 Cell Assay Example 56 is illustrated in FIG. 58A, which shows the separate and combined effects of psilocybin and eugenol on COX-2 in the A-172 cells described above. As can be seen in FIG. 58A, the combination of 5 μM psilocybin with 25 μM eugenol resulted in a greater reduction of inflammatory marker COX-2 than 5 μM psilocybin alone, while 25 μM eugenol alone resulted in an increase in COX-2. This indicates the synergistic effects of psilocybin and eugenol on COX-2. A combination of 5 μM psilocybin with 25 μM eugenol reduced COX-2 by 51% more than 5 μM psilocybin alone and by 71% more than 25 μM eugenol alone.

### Example 57

In vitro A-172 Cell Assay Example 57 is illustrated in FIG. 58B, which shows the separate and combined effects of psilocybin and eugenol on GABA in the A-172 cells described above. As can be seen in FIG. 58B, the combination of 5 μM psilocybin with 25 μM eugenol resulted in a greater increase in depression-related marker GABA than 5 μM psilocybin or 25 μM eugenol alone, thus indicating the synergistic effects of psilocybin and eugenol on GABA. A combination of 5 μM psilocybin with 25 μM eugenol increased GABA by 64% more than 5 μM psilocybin alone and by 43% more than 25 μM eugenol alone.

### Example 58

In vitro A-172 Cell Assay Example 58 is illustrated in FIG. 58C, which shows the separate and combined effects of psilocybin and eugenol on IL-6 in the A-172 cells described above. As can be seen in FIG. 58C, the combination of 5 μM psilocybin with 25 μM eugenol resulted in a greater reduction of inflammatory marker IL-6 than 5 psilocybin or 25 μM eugenol alone, thus indicating the synergistic effects of psilocybin and eugenol on IL-6. Indeed, psilocybin and eugenol together resulted in approximately 40% decrease in IL-6 compared to psilocybin alone. A combination of 5 μM psilocybin with 25 μM eugenol reduced IL-6 by 38% more than 5 μM psilocybin alone and by 14% more than 25 μM eugenol alone.

### Example 59

In vitro A-172 Cell Assay Example 59 is illustrated in FIG. 58D, which shows the separate and combined effects of psilocybin and eugenol on BDNF in the A-172 cells described above. As can be seen in FIG. 58D, the combination of 5 μM psilocybin with 25 μM eugenol resulted in a greater increase in depression-related marker BDNF than 5 μM psilocybin or 25 μM eugenol alone, thus indicating the synergistic effects of psilocybin and eugenol on BDNF. A combination of 5 μM psilocybin with 25 eugenol increased BDNF by 51% more than 5 μM psilocybin alone and by 133% more than 25 μM eugenol alone.

### Example 60

Psilocybin combined with Eugenol are selected for further study. Combinations of these two pure compounds demonstrate efficacy as an anti-inflammatory, with increases in brain markers GABA and BDNF. The combination also lacks toxic effects in the MTT assay. The safety and pharmacokinetics of both molecules is well established. Formulations combining these two active molecules are created. Eugenol is a liquid that can be applied directly to a powder as a concentrate or can be diluted in ethanol (or another alcohol) in order to achieve a more consistent mixture at higher volumes. Pure Psilocybin crystal can be added and this mixture can be consumed directly as a tincture to achieve the desired dose (mg) as seen in Table 51.

Alternatively, a eugenol/alcohol solution of desired concentration can be directly applied to the dried fruiting bodies of psilocybin fungi. The ethanol is then allowed to evaporate at low temperature (<35° C.) as it has a significantly lower vapor and boiling point than eugenol, leaving the eugenol infused in the fungal biomass. This can then be homogenized for even distribution during production. Various ratios can be made as seen in Table 51.

Alternatively, both the pure compounds (˜99%) Psilocybin and Eugenol can be formulated to achieve the desired concentrations of both ingredients. In this case, due to the small amounts required, binders, fillers or excipients are likely required to fill out the formulations. The liquid alcohol based solution again can be applied to the powder, dehydrated and homogenized before final productization into a pill or tablet format. Pure psilocybin and pure eugenol can also be applied directly to any number of carriers binders, fillers, excipients or flavoring agents included in the final formulation. Psilocybin may also be first added to the eugenol and/or eugenol ethanol blend and applied to a filler substrate. Formulations outlining the desired concentrations and ratios can be found in Table 51.

TABLE 51
Example Formulations of Psilocybin + Eugenol

| | | | | | Carriers/Fillers/ | | | |
|---|---|---|---|---|---|---|---|---|
| | | Eugenol | | Ratio Range | Binders/Excipients/ | | Final Mass | |
| Product | Psilocybin | (99%+) | | (between) | Flavoring Agents | | or Volume | |
| Mushroom- | 100 mg-300 | 1-3 | μL (mg) | 3:1 to 1:3 | none | | 101-303 | mg |
| based pill | mg @1% | | | | | | | |
| | 100 mg-300 | 10-30 | μL (mg) | 1:10 to 3:10 | 0-190 | mg | 300-330 | mg |
| | mg @1% | | | | | | | |
| | 100 mg @ 1% | 100-300 | μL (mg) | 1:100 to 1:300 | 100 mg-300 mg | | 500 | mg |
| Pure | 1 mg-3 mg | 1-3 | μL (mg) | 3:1 to 1:3 | 294-298 | mg | 300 | mg |
| Pressed | 1 mg-3 mg | 10-30 | μL (mg) | 1:10 to 3:10 | 267-289 | mg | 300 | mg |
| Tablet | 1 mg-3 mg | 100 | μL (mg) | 1:33 to 1:100 | 197-199 | mg | 300 | mg |
| Pure Solid | 1 mg-3 mg | 1-3 | μL (mg) | 3:1 to 1:3 | 294-298 | mg | 300 | mg |
| Capsule | 1 mg-3 mg | 10-30 | μL (mg) | 1:10 to 3:10 | 267-289 | mg | 300 | mg |
| | 1 mg-3 mg | 100 | μL (mg) | 1:100 to 1:300 | 197-199 | mg | 300 | mg |
| Pure | 1 mg-3 mg | 1-3 | μL (mg) | 3:1 to 1:3 | 244-248 | μL | 0.25 | mL |
| Tincture | 1 mg-3 mg | 10-30 | μL (mg) | 1:10 to 3:10 | 217-239 | μL | 0.25 | mL |
| | 1 mg-3 mg | 100 | μL (mg) | 1:33 to 1:100 | 147-149 | μL | 0.25 | mL |

### REFERENCES

Akter, J., Hossain, M. A., Takara, K., Islam, M. Z., & Hou, D.-X. (2019). Antioxidant activity of different species and varieties of turmeric (Curcuma spp): Isolation of active compounds. Comparative Biochemistry and Physiology Part C: Toxicology & Pharmacology, 215, 9-17.
Al Othman, Z. A., Ahmed Y. B. H., Habila, M. A. and Ghafar, A. A. (2011). Determination of Capsaicin and Dihydrocapsaicin in Capsicum Fruit Samples using High Performance Liquid Chromatography. Molecules, 16, 8919-8929; doi:10.3390/molecules16108919
Anderson, R. A., Qin, B., Canini, F., Poulet, L., & Roussel, A. M. (2013). Cinnamon Counteracts the Negative Effects of a High Fat/High Fructose Diet on Behavior, Brain Insulin Signaling and Alzheimer-Associated Changes. PLOS ONE, 8(12), e83243—e83243.
Arun, N., & Nalini, N. (2002). Efficacy of turmeric on blood sugar and polyol pathway in diabetic albino rats. Plant Foods for Human Nutrition, 57(1), 41-52.
Ashoor, Abrar, Jacob C. Nordman, Daniel Veltri, Keun-Hang Susan Yang, Yaroslav Shuba, Lina Al Kury, Bassem Sadek et al. (2013). “Menthol Inhibits 5-HT3 Receptor—Mediated Currents.” Journal of Pharmacology and Experimental Therapeutics, 347(2): 398-409.
Bandell, M., Story, G. M., Hwang, S. W., Viswanath, V., Eid, S. R., Petrus, M. J., Earley, T. J., & Patapoutian, A. (2004). Noxious cold ion channel TRPA1 is activated by pungent compounds and bradykinin. Neuron, 41(6), 849-857.
Bharate, S. S., & Bharate, S. B. (2012). Modulation of thermoreceptor TRPM8 by cooling compounds. ACS Chemical Neuroscience, 3(4), 248-267.
Brouet, I., & Ohshima, H. (1995). Curcumin, an anti-tumor promoter and anti-inflammatory agent, inhibits induction of nitric oxide synthase in activated macrophages. Biochemical and Biophysical Research Communications, 206(2), 533-540.
Can Baser, K. H. (2008). Biological and pharmacological activities of carvacrol and carvacrol bearing essential oils. Current Pharmaceutical Design, 14(29), 3106-3119.
Chao, J-M., & Marderosian, A. H. D. (1973). Ergoline alkaloidal constituents of hawaiian baby wood rose, argyreia nervosa (Burm. f.) bojer. Journal of Pharmaceutical Sciences, 62(4), 588-591.
Chung, G., & Oh, S. B. (2013). Eugenol as local anesthetic. Natural Products, 4001-4015.
Ciardo, M. G., & Ferrer-Montiel, A. (2017). Lipids as central modulators of sensory TRP channels.
Cortés-Rojas, D. F., de Souza, C. R. F., & Oliveira, W. P. (2014). Clove (Syzygium aromaticum): A precious spice. Asian Pacific Journal of Tropical Biomedicine, 4(2), 90-96.
Daniel, A. N., Sartoretto, S. M., Schmidt, G., Caparroz-Assef, S. M., Bersani-Amado, C. A., & Cuman, R. K. N. (2009). Anti-inflammatory and antinociceptive activities A of eugenol essential oil in experimental animal models. Revista Brasileira de Farmacognosia, 19(1B), 212-217.
Jon G. Dean, Tiecheng Liu, Sean Huff, Ben Sheler, Steven A. Barker, Rick J. Strassman, Michael M. Wang, Jimo Borjigin. “Biosynthesis and Extracellular Concentrations of N,N-dimethyltryptamine (DMT) in Mammalian Brain.” Scientific Reports, 2019; 9(1) DOI: 10.1038/s41598-019-45812-w
Della Corte, K. W., Perrar, I., Penczynski, K. J., Schwingshackl, L., Herder, C., & Buyken, A. E. (2018). Effect of dietary sugar intake on biomarkers of subclinical inflammation: A systematic review and meta-analysis of intervention studies. Nutrients, 606-606.
de Sousa Rodrigues, M. E., Bekhbat, M., Houser, M. C., Chang, J., Walker, D. I., Jones, D. P., 0Iler do Nascimento, C. M. P., Barnum, C. J., & Tansey, M. G. (2017). Chronic psychological stress and high-fat high-fructose diet disrupt metabolic and inflammatory gene networks in the brain, liver, and gut and promote behavioral deficits in mice. Brain, Behavior, and Immunity, 59, 158-172.
Dickens, C., McGowan, L., Clark-Carter, D., & Creed, F. (2002). Depression in rheumatoid arthritis: A systematic review of the literature with meta-analysis. Psychosomatic Medicine, 64(1), 52-60.
Doyle, A. A., & Stephens, J. C. (2019). A review of cinnamaldehyde and its derivatives as antibacterial agents. Fitoterapia, 139, 104405-104405.
Feng, L., Li, J., Yu, H.-B., Xue, Q., & Dai, L.-J. (2020). Effects of cinnamaldehyde on anti-respiratory syncytial virus: A protocol of systematic review and meta-analysis. Medicine, 99(20).
Gan, W. Q., Man, S. F. P., Senthilselvan, A., & Sin, D. D. (2004). Association between chronic obstructive pulmonary disease and systemic inflammation: A systematic review and a meta-analysis. Thorax, 59(7), 574-580.
Goldstein, B. I., Kemp, D. E., Soczynska, J. K., & McIntyre, R. S. (2009). Inflammation and the phenomenology, pathophysiology, comorbidity, and treatment of bipolar disorder: A systematic review of the literature. Journal of Clinical Psychiatry, 70(8), 1078-1078.

Grzegorz R. Juszczak & Artur H. Swiergiel (2013) Recreational Use of D-Lysergamide from the Seeds of Argyreia nervosa, Ipomoea tricolor, Ipomoea violacea, and Ipomoea purpurea in Poland. J Psychoactive Drugs 45(1):79-93.
Heim, R, & Wasson, R. G. (1958). Les Champignons hallucinogènes du Mexique, ed. Du Museum national d'Histoire Naturelle.
Hoesel, B., & Schmid, J. A. (2013). The complexity of NF-κB signaling in inflammation and cancer. Molecular Cancer, 12(1), 1-15.
Iwasaki, Y., Tanabe, M., Kobata, K., & Watanabe, T. (2008). TRPA1 agonists—Allyl isothiocyanate and cinnamaldehyde—Induce adrenaline secretion. Bioscience, Biotechnology, and Biochemistry, 72(10), 2608-2614.
Janda, E., Lascala, A., Martino, C., Ragusa, S., Nucera, S., Walker, R., Gratteri, S., & Mollace, V. (2016). Molecular mechanisms of lipid- and glucose-lowering activities of bergamot flavonoids. Pharma Nutrition, 4, S8—S18.
Jiang, J., Emont, M. P., Jun, H., Qiao, X., Liao, J., Kim, D.-I., & Wu, J. (2017). Cinnamaldehyde induces fat cell-autonomous thermogenesis and metabolic reprogramming. Metabolism: Clinical and Experimental, 77, 58-64.
Karlović, D., Serretti, A., Vrkie, N., Martinac, M., & Mareinko, D. (2012). Serum concentrations of CRP, IL-6, TNF-α and cortisol in major depressive disorder with melancholic or atypical features. Psychiatry Research, 198(1), 74-80.
Khalil, A. A., ur Rahman, U., Khan, M. R., Sahar, A., Mehmood, T., & Khan, M. (2017). Essential oil eugenol: Sources, extraction techniques and nutraceutical perspectives. RSC Advances, 7(52), 32669-32681.
Krishnadas, R., & Cavanagh, J. (2012). Depression: An inflammatory illness? Journal of Neurology, Neurosurgery & amp; Psychiatry, 83(5), 495 LP −502.
Kulkarni, S K, Dhir, A., & Akula, K. K. (2009). Potentials of curcumin as an antidepressant. The Scientific World JOURNAL, 9.
Kulkarni, Shrinivas K, Bhutani, M. K., & Bishnoi, M. (2008). Antidepressant activity of curcumin: Involvement of serotonin and dopamine system. Psychopharmacology, 201(3), 435-435.
Lee, C.-H., & Giuliani, F. (2019). The Role of Inflammation in Depression and Fatigue. Frontiers in Immunology, 10, 1696-1696.
Limtrakul, P., Lipigorngoson, S., Namwong, O., Apisariyakul, A., & Dunn, F. W. (1997). Inhibitory effect of dietary curcumin on skin carcinogenesis in mice. Cancer Letters, 116(2), 197-203.
Liu, Y., Ho, R. C.-M., & Mak, A. (2012). Interleukin (IL)-6, tumour necrosis factor alpha (TNF-α) and soluble interleukin-2 receptors (sIL-2R) are elevated in patients with major depressive disorder: A meta-analysis and meta-regression. Journal of Affective Disorders, 139(3), 230-239.
Lopresti A. L. (2017). Curcumin for neuropsychiatric disorders: a review of in vitro, animal and human studies. J Psychopharmacol, 31(3), 287-302. doi: 10.1177/0269881116686883
Lopresti A. L., Hood S. D., Drummond P. D. (2012). Multiple antidepressant potential modes of action of curcumin: A review of its anti-inflammatory, monoaminergic, antioxidant, immune-modulating and neuroprotective effects. J Psychopharmacol, 26(12), 1512-1524. doi: 10.1177/0269881112458732.
Loyd, D. R., Henry, M. A., & Hargreaves, K. M. (2013). Serotonergic neuromodulation of peripheral nociceptors. Seminars in Cell & Developmental Biology, 24(1), 51-57.
Lu, J., Jiao, Z., Yu, Y., Zhang, C., He, X., Li, Q., Xu, D., & Wang, H. (2018). Programming for increased expression of hippocampal GAD67 mediated the hypersensitivity of the hypothalamic-pituitary-adrenal axis in male offspring rats with prenatal ethanol exposure. Cell Death & Disease, 9(6), 659-659.
Ma, K., Zhang, H., & Baloch, Z. (2016). Pathogenetic and therapeutic applications of tumor necrosis factor-α (TNF-α) in major depressive disorder: A systematic review. International Journal of Molecular Sciences, 17(5), 733-733.
McNamara, F. N., Randall, A., & Gunthorpe, M. J. (2005). Effects of piperine, the pungent component of black pepper, at the human vanilloid receptor (TRPV1). British Journal of Pharmacology, 144(6), 781-781.
Mezuk, B., Eaton, W. W., Albrecht, S., & Golden, S. H. (2008). Depression and type 2 diabetes over the lifespan: A meta-analysis. Diabetes Care, 31(12), 2383-2390.
Moussaieff, A., Rimmerman, N., Bregman, T., Straiker, A., Felder, C. C. Shoham, S., Kashman, Y. et al. (2008). Incensole acetate, an incense component, elicits psychoactivity by activating TRPV3 channels in the brain. The FASEB Journal 22(8), 3024-3034.
Nelson, K. M., Dahlin, J. L., Bisson, J., Graham, J., Pauli, G. F., & Walters, M. A. (2017). The Essential Medicinal Chemistry of Curcumin. Journal of Medicinal Chemistry, 60(5), 1620-1637.
Nicolussi, S., Viveros-Paredes, J. M., Gachet, M. S., Rau, M., Flores-Soto, M. E., Blunder, M., & Gertsch, J. (2014). Guineensine is a novel inhibitor of endocannabinoid uptake showing cannabimimetic behavioral effects in BALB/c mice. Pharmacological Research, 80, 52-65.
Nowak, J., Woiniakiewicz, M., Klepacki, P., Sowa, A., & Kościelniak, P. (2016). Identification and determination of ergot alkaloids in Morning Glory cultivars. Analytical and bioanalytical chemistry, 408(12), 3093-3102.
Nutt, D. (2019). Psychedelic drugs-a new era in psychiatry? Dialogues in Clinical Neuroscience, 21(2), 139-147.
Ohta, T., Ikemi, Y., Murakami, M., Imagawa, T., Otsuguro, K., & Ito, S. (2006). Potentiation of transient receptor potential V1 functions by the activation of metabotropic receptors in rat primary sensory neurons. The Journal of Physiology, 576(3), 809-822.
O'Neil, A., Stevenson, C. E., Williams, E. D., Mortimer, D., Oldenburg, B., & Sanderson, K. (2013). The health-related quality of life burden of co-morbid cardiovascular disease and major depressive disorder in Australia: Findings from a population-based, cross-sectional study. Quality of Life Research, 22(1), 37-44.
Oz, M., El Nebrisi, E. G., Yang, K.-H. S., Howarth, F. C., & Al Kury, L. T. (2017). Cellular and Molecular Targets of Menthol Actions. Frontiers in Pharmacology, 8, 472-472.
Palagini, L., Mosca, M., Tani, C., Gemignani, A., Mauri, M., & Bombardieri, S. (2013). Depression and systemic lupus erythematosus: A systematic review. Lupus, 22(5), 409-416.
Patel, A. (2013). The role of inflammation in depression. Psychiatr Danub, 2), S216-23.
Polito, V., & Stevenson, R. J. (2019). A systematic study of microdosing psychedelics. PLOS ONE, 14(2), e0211023—e0211023.
Premkumar, L. S. (2014). Transient receptor potential channels as targets for phytochemicals. ACS chemical neuroscience 5(11), 1117-1130.
Rao, C. V., Rivenson, A., Simi, B., & Reddy, B. S. (1995). Chemoprevention of colon carcinogenesis by dietary curcumin, a naturally occurring plant phenolic compound. Cancer Research, 55(2), 259-266.
Salehi, B., Stojanovie-Radie, Z., Matejie, J., Sharopov, F., Antolak, H., Kregiel, D., Sen, S., Sharifi-Rad, M., Acharya, K., Sharifi-Rad, R., Martorell, M., Sureda, A., Martins, N., & Sharifi-Rad, J. (2018). Plants of Genus Mentha: From Farm to Food Factory. Plants (Basel, Switzerland), 7(3), 70-70.
Sharma, C., Sadek, B Goyal, S. N., Sinha, S., Kamal, M. A., & Ojha, S. (2015). Small Molecules from Nature Targeting G-Protein Coupled Cannabinoid Receptors: Potential Leads for Drug Discovery and Development. Evidence-Based Complementary and Alternative Medicine: ECAM, 2015, 238482-238482.
Sköld, M., Karlberg, A.-T., Matura, M., & Borje, A. (2006). The fragrance chemical β-caryophyllene—Air oxidation and skin sensitization. Food and Chemical Toxicology, 44(4), 538-545.
Slavich, G. M., & Irwin, M. R. (2014). From stress to inflammation and major depressive disorder: A social signal transduction theory of depression. Psychological Bulletin, 140(3), 774-774.
Smalheiser, N. R. (2019). A Neglected Link Between the Psychoactive Effects of Dietary Ingredients and Consciousness-Altering Drugs. Frontiers in Psychiatry, 10, 591-591.
Sommer, C. (2004). Serotonin in pain and analgesia. Molecular Neurobiology, 117-125.
Sreejayan, & Rao, M. N. A. (1994). Curcuminoids as Potent Inhibitors of Lipid Peroxidation. Journal of Pharmacy and Pharmacology, 46(12), 1013-1016.
Stanojević, J. S., Stanojevie, L. P., Cvetkovie, D. J., Danilovie, B. R. (2015). Chemical Composition, Antioxidant and Antimicrobial Activity of the Turmeric Essential Oil (Curcuma longa L.). Advanced technologies, 4(2), 19-25.
Stenvinkel, P., & Alvestrand, A. (2002). Review articles: Inflammation in end-stage renal disease: Sources, consequences, and therapy. Seminars in Dialysis, 15(5), 329-337.
Stiedl, O., Pappa, E., Konradsson-Geuken, Å., & Ögren, S. O. (2015). The role of the serotonin receptor subtypes 5-HT1A and 5-HT7 and its interaction in emotional learning and memory. Frontiers in Pharmacology, 6, 162-162.
Straub, R. H. (2014). TRPV1, TRPA1, and TRPM8 channels in inflammation, energy redirection, and water retention: Role in chronic inflammatory diseases with an evolutionary perspective. Journal of Molecular Medicine, 92(9), 925-937.
Sugivar, T., Bielefeldt, K., & Gebhart, G. F. (2004). TRPV1 function in mouse colon sensory neurons is enhanced by metabotropic 5-hydroxytryptamine receptor activation. Journal of Neuroscience, 24(43), 9521-9530.
Sui, F., Lin, N., Guo, J.-Y., Zhang, C.-B., Du, X.-L., Zhao, B.-S., Liu, H.-B., Yang, N., Li, L.-F., & Guo, S.-Y. (2010). Cinnamaldehyde up-regulates the mRNA expression level of TRPV1 receptor potential ion channel protein and its function in primary rat DRG neurons in vitro. Journal of Asian Natural Products Research, 12(1), 76-87.
Tian, F., Yu, C. T., Ye, W. D., & Wang, Q. (2017). Cinnamaldehyde induces cell apoptosis mediated by a novel circular RNA hsa circ 0043256 in non-small cell lung cancer. Biochemical and Biophysical Research Communications, 493(3), 1260-1266.
Ting, E., Yang, A., & Tsai, S., “Role of Interleukin-6 in Depressive Disorder,” Int. Mol. Sci., 21(6):2194 (March 2020).
Uchida, Kunitoshi, Wuping Sun, Jun Yamazaki, and Makoto Tominaga. “Role of thermo-sensitive transient receptor potential channels in brown adipose tissue.” Biological and Pharmaceutical Bulletin 41, no. 8 (2018): 1135-1144.
Vogt-Eisele, A. K., Weber, K., Sherkheli, M. A., Vielhaber, G., Panten, J., Gisselmann, G., & Hatt, H. (2007). Monoterpenoid agonists of TRPV3. British Journal of Pharmacology, 151(4), 530-540.
Wang, Shaopeng, Caihua Zhang, Guang Yang, and Yanzong Yang. “Biological properties of 6-gingerol: a brief review.” Natural product communications 9, no. 7 (2014): 1934578X1400900736.
Wang, Z.-J., Tabakoff, B., Levinson, S. R., & Heinbockel, T. (2015). Inhibition of Nav1.7 channels by methyl eugenol as a mechanism underlying its antinociceptive and anesthetic actions. Acta Pharmacologica Sinica, 36(7), 791-799.
Woolf, C. J., & Ma, Q. (2007). Nociceptors—Noxious stimulus detectors. Neuron, 55(3), 353-364.
Xu, H., Delling, M., Jun, J. C., & Clapham, D. E. (2006). Oregano, thyme and clove-derived flavors and skin sensitizers activate specific TRP channels. Nature Neuroscience, 9(5), 628-635.
Xu, Y., Ku, B., Tie, L., Yao, H., Jiang, W., Ma, X., & Li, X. (2006). Curcumin reverses the effects of chronic stress on behavior, the HPA axis, BDNF expression and phosphorylation of CREB. Brain Research, 1122(1), 56-64.
Yu, Jing-Jie, Liu-Bao Pei, Yong Zhang, Zi-Yu Wen, and Jian-Li Yang. Chronic supplementation of curcumin enhances the efficacy of antidepressants in major depressive disorder: a randomized, double-blind, placebo-controlled pilot study. Journal of Clinical Psychopharmacology, 35(4), 406-410.
Zhao, X., Wang, C., Zhang, J.-F., Liu, L., Liu, A.-M., Ma, Q., Zhou, W.-H., & Xu, Y. (2014). Chronic curcumin treatment normalizes depression-like behaviors in mice with mononeuropathy: Involvement of supraspinal serotonergic system and GABA A receptor. Psychopharmacology, 231(10), 2171-2187.
Zhi, L., Dong, L., Kong, D., Sun, B., Sun, Q., Grundy, D., Zhang, G. Rong, W. (2013). Curcumin acts via transient receptor potential vanilloid-1 receptors to inhibit gut nociception and reverses visceral hyperalgesia. Neurogastroenterology & Motility, 25(6), e429-e440.

### EXAMPLES ONLY

In the preceding description, for purposes of explanation, numerous details are set forth in order to provide a thorough understanding of the embodiments. However, it will be apparent to one skilled in the art that these specific details are not required. The above-described embodiments are intended to be examples only. Alterations, modifications and variations can be effected to the particular embodiments by those of skill in the art. The scope of the claims should not be limited by the particular embodiments set forth herein, but should be construed in a manner consistent with the specification as a whole.

What is claimed is:

1. A composition comprising a therapeutic combination of a 5HT2A agonist compound and at least one TRP agonist compound,
wherein the therapeutically effective amount of the 5HT2A agonist is between about 1 μg and about 300 mg; and the therapeutically effective amount of the at least one TRP receptor agonist is between about 0.01 mg and about 300 mg.

2. The composition of claim 1, wherein the 5HT2A agonist compound is selected from the group consisting of a tryptamine, an ergoline, a phenethylamine, and a phenylpropanoid.

3. The composition of claim 2, wherein the tryptamine is a 4-substituted tryptamine.

4. The composition of claim 3, wherein the 4-substituted tryptamine is a 4-substituted DMT compound.

5. The composition of claim 4, wherein the 4-substituted DMT compound is selected from the group consisting of 3-[2-(dimethylamino)ethyl]-4-phosphoryloxyindole (psilocybin), 3-[2-(dimethylamino)ethyl]-4-hydroxyindole (psilocin), 3-[2-(dimethylamino)ethyl]-4-acetoxyindole (psilacetin), and any suitable salt of any of the foregoing.

6. The composition of claim 3, wherein the 4-substituted tryptamine is selected from the group consisting of 3-[2(trimethylamino)ethyl]-4-phosphoryloxyindole (aeruginascin), 3-[2-(methylamino)ethyl]-4-phosphoryloxyindole (baeocystin), 3-[2-(methylamino)ethyl]-4-hydroxyindole, 3-[2-(amino)ethyl]-4-hydroxyindole (norpsilocin), 3-[2-(amino)ethyl]-4-phosphoryloxyindole (norbaeocystin), and any suitable salt of any of the foregoing.

7. The composition of any one of claims 3 to 6, wherein the 4-substituted tryptamine is derived from fungi.

8. The composition of claim 7, wherein the fungi is a species of a genus selected from the group consisting of Gymnopilus, Inocybe, Panaeolus, Pholiotina, Pluteus, and Psilocybe.

9. The composition of claim 7 or 8, wherein the fungi is selected from the group consisting of C. cyanopus, C. siligineoides and C. kuehneriana; Copelandia species including C. affinis, C. anomala, C. bispora, C. cambodginiensis, C. chlorocystis, C. cyanescens, C. lentisporus, C. tirunelveliensis, C. tropica, C. tropicalis and C. westii; G. steglichii; G. thiersii, G. aeruginosus, G. braendlei, G. cyanopalmicola, G. intermedius, G. junonius, G. lateritius, G. liquiritiae, G. luteofolius, G. luteoviridis, G. luteus, G. purpuratus, G. subpurpuratus, G. validipes and G. viridans; I. aeruginascens, I. aeruginascens, coelestium, I. corydalina, I. corydalina var. corydalina, I. corydalina var. erinaceomorpha, I. haemacta and I. tricolor; P. cinctulus, P. affinis, P. africanus, P. bisporus, P. cambodginiensis, P. castaneifolius, P. chlorocystis, P. cinctulus, P. cyanescens, P. fimicola, P. lentisporus, P. microsporus, P. moellerianus, P. olivaceus, P. rubricaulis, P. tirunelveliensis, P. tropicalis and P. venezolanus; P. cyanopus and P. smithii; P. americanus, P. albostipitatus, P. americanus, P. cyanopus, P. glaucus, P. glaucotinctus, P. nigroviridis, P. phaeocyanopus, P. salicinus, P. saupei and P. villosus; P. tampanensis, P. acutipilea, P. allenii, P. angustipleurocystidiata, P. antioquiensis, P. atlantis, P. aquamarina, P. armandii, P. aucklandii, P. atlantis, P. aztecorum, P. aztecorum var. aztecorum, P. aztecorum var. bonetii, P. azurescens, P. baeocystis, P. banderillensis, P. bispora, P. brasiliensis, P. brunneocystidiata, P. cubensis, P. caeruleoannulata, P. caerulescens, P. caerulescens var. caerulescens, P. caerulescens var. ombrophila, P. caerulipes, P. callosa, P. carbonaria, P. caribaea, P. chuxiongensis, P. collybioides, P. columbiana, P. cordispora, P. cubensis, P. cyanescens, P. cyanofibrillosa, P. dumontii, P. egonii, P. fagicola, P. fagicola var. fagicola, P. fagicola var. mesocystidiata, P. farinacea, P. fimetaria, P. fuliginosa, P. furtadoana, P. tampanensis, P. galindoi, P. gallaeciae, P. graveolens, P. guatapensis, P. guilartensis, P. heimii Guzman, P. herrerae Guzman, P. hispanica Guzman, P. hoogshagenii, P. hoogshagenii var. hoogshagenii, P. hoogshagenii var. convexa, P. inconspicua, P. indica, P. isabelae, P. jacobsii, P. jaliscana, P. kumaenorum, P. laurae, P. lazoi, P. liniformans, P. liniformans var. liniformans, P. liniformans var. americana, P. mexicana, P. mairei, P. makarorae, P. mammillata, P. medullosa, P. meridensis, P. meridionalis, P. mescaleroensis, P. mexicana, P. moseri, P. muliercula, P. naematoliformis, P. natalensis, P. natarajanii, P. neorhombispora, P. neoxalapensis, P. ovoideocystidiata, P. ovoideocystidiata, P. papuana, P. paulensis, P. pelliculosa, P. pintonii, P. pleurocystidiosa, P. plutonia, P. portoricensis, P. pseudoaztecorum, P. puberula, P. quebecensis, P. ricki, P. rostrata, P. rzedowskii, P. samuiensis, P. schultesii, P. semilanceata, P. septentrionalis, P. serbica, P. sierrae, P. silvatica, P. singeri, P. squamosa, P. strictipes, P. stuntzii, P. subacutipilea, P. subaeruginascens, P. subaeruginosa, P. subbrunneocystidiata, P. subcaerulipes, P. subcubensis, P. subpsilocybioides, P. subtropicalis, P. tampanensis, P. tampanensis, P. thaicordispora, P. thaiaerugineomaculans, P. thaiduplicatocystidiata, P. uruguayensis, P. uxpanapensis, P. venenata, P. villarrealiae, P. weraroa, P. wassoniorum, P. weihi, P. weldenii, P. weraroa, P. wrightii, P. xalapensis, P. yungensis, P. zapotecorum, P. zapotecoantillarum, P. zapotecocaribaea, and P. zapotecorum.

10. The composition of any one of claims 7 to 9, wherein the composition further comprises dried matter of the fungi, wherein the dried matter is selected from the group consisting of fruiting bodies, mycelia, sclerotia, and hyphae, or combinations thereof.

11. The composition of any one of claim 1 or 2, wherein the tryptamine is a 5-substituted tryptamine.

12. The composition of claim 11, wherein the 5-substituted tryptamine is selected from the group consisting of 5-methoxy-DMT (bufotenin), N-acetyl-5-methoxy tryptamine (melatonin), 5-hydroxy tryptamine (serotonin), 5-hydroxy-tryptophan (5-HTP), and any suitable salt of any of the foregoing.

13. The composition of any one of claim 1 or 2, wherein the 5HT2A agonist compound is an ergoline.

14. The composition of claim 13, wherein the ergoline is selected from the group consisting of D-lysergic acid ethylamide (“LAE”),D-lysergic acid beta-propanolamide, D-lysergic acid 2-butyl amide (“LSB”), D-lysergic acid 1-butanolamide, 1-methyl-D-lysergic acid butanolamide, D-lysergic acid 3-pentyl amide (“LSP”), D-N-morpholinyllysergamide (“LSM-775”), D-N-pyrrolidyllysergamide (“LPD-824”), (8(3)-6-methyl-8-(piperidin-1-ylcarbonyl)-9,10-didehydroergoline (“LSD-Pip”), N,N-dimethyllysergamide (“DAM”), D-lysergic acid methylisopropyl amide (“LAMIDE”), D-lysergic acid 2,4-dimethylazetidide (“LSZ”), LSD, D-1-acetyl-lysergic acid diethylamide (“ALD-52”), D-1-propionyl-lysergic acid diethylamide (“1P-LSD”), D-N1-butyryl-lysergic acid diethylamide (“1B-LSD”), D-N1-(cyclopropylmethanoyl)-lysergic acid diethylamide (“1 cP-LSD”), D-N1-methyl-lysergic acid diethylamide (“MLD”), D-6-ethyl-6-nor-lysergic acid diethylamide (“ETH-LAD”), D-1-propionyl-6-ethyl-6-nor-lysergic acid diethyamide (“1P-ETH-LAD”), D-6-allyl-6-nor-lysergic acid diethylamide (“AL-LAD”), D-6-propyl-6-nor-lysergic acid diethylamide (“PRO-LAD”), D-6-isopropyl-6-nor-lysergic acid diethylamide (“IP-LAD”), D-6-propynyl-6-nor-lysergic acid diethylamide (“PARGY-LAD”), D-6-butyl-6-norlysergic acid diethylamide (“BU-LAD”), N,N-diallyllysergamide (“DAL”) and D-N-ethyl-N-cyclopropyllysergamide (“ECPLA”).

15. The composition of any one of claim 13 or 14, wherein the ergoline is derived from fungi or a plant.

16. The composition of claim 15, wherein the fungi or plant is a species selected from the group consisting of Claviceps purpurea, Rivea corymbosa, Ipomoea violacea, I. tricolor, I. purpurae, I. alba, Argeyreia nervosa, and a Periglandula species.

17. The composition of any one of claim 1 or 2, wherein the 5HT2A agonist compound is a phenethylamine.

18. The composition of claim 17, wherein the phenethylamine is selected from the group consisting of 3,4,5-trimethoxyphenethylamine (mescaline), trimethoxyamphetamine (“TMA”), 4-bromo-2,5-dimethoxybenzeneethanamine (“2C-B”), 4-bromo-2,5-dimethoxyamphetamine (“DOB”), 4-methyl-2,5-dimethoxyamphetamine (“DOM”), 4-methyl-2,5-dimethoxyb enzeneethanamine (“2C-D”), 3,4-methylenedi oxyamphetamine (“MDA”), N-methyl-3,4-methylenedioxyamphetamine (“MDMA”).

19. The composition of any one of claim 17 or 18, wherein the phenethylamine is plant-derived.

20. The composition of claim 19, wherein the plant includes a species selected from the group consisting of Lophophora wilhamsii, Trichocereus pachanoi, Echinopsis pachanoi, Trichocereus peruvianus, Echinopsis peruviana, Trichocereus bridgesii, Echinopsis lageniformis, and Trichocereus/Echinopsis scopulicola.

21. The composition of any one of claim 1 or 2, wherein the 5HT2A agonist compound is a phenylpropanoid.

22. The composition of claim 21, wherein the phenylpropanoid is 1,2,3-timethoxy-5-(prop-2-en-1-yl)benzene (elemicin).

23. The composition of any one of claim 21 or 22, wherein the phenylpropanoid is plant-derived.

24. The composition of claim 23, wherein the plant is a species in the Myristicaceae family.

25. The composition of any one of claims 1 to 24, wherein the TRP agonist compound is selected from the group consisting of a capsiate, eugenol, elemicin, myrcene, piperine and gingerol.

26. The composition of claim 25, where the capsiate is capsaicin.

27. The composition of any one of claims 1 to 26, wherein the TRP agonist compound is plant-derived.

28. The composition of claim 27, wherein the plant includes one or more species selected from the group consisting of cayenne pepper, turmeric, clove, cinnamon, nutmeg, pepper, cannabis, bergamot and ginger.

29. The composition of any one of claims 1 to 28, wherein the TRP agonist compound is selected from the group consisting of a curcuminoid, cinnamaldehyde, alpha terpineol, thymol, piperine and allicin.

30. The composition of claim 29, wherein the curcuminoid is curcumin.

31. The composition of any one of claim 29 or 30, wherein the TRP agonist compound is plant-derived.

32. The composition of claim 31, wherein the plant includes one or more species selected from the group consisting of curcumin, cinnamon, turmeric, nutmeg, cannabis, thyme, pepper, garlic, and onion.

33. The composition of any one of claims 1 to 32, wherein the TRP agonist compound is selected from the group consisting of eugenol, cinnamaldehyde, carvacrol, thymol, menthol, and 1-8 cineole.

34. The composition of claim 33, wherein the TRP agonist compound is plant-derived.

35. The composition of claim 34, wherein the plant includes one or more species selected from the group consisting of turmeric, clove, cinnamon, pepper, nutmeg, cannabis, bergamot, oregano, thyme, cardamom, peppermint, and eucalyptus.

36. The composition of any one of claims 1 to 35, wherein the TRP agonist compound is selected from the group consisting of eugenol, β-caryophyllene, (−)-epicatechin, CBD, CBDA, CBGA, CBGV, THCV, THCVA, eriodictyol, cinnamaldehyde, incensole, boswellic acid, eucalyptol, and thymol.

37. The composition of claim 36, wherein the TRP agonist compound is plant-derived.

38. The composition of claim 37, wherein the plant includes one or more species selected from the group consisting of turmeric, clove, cinnamon, pepper, nutmeg, cannabis, bergamot, oregano, thyme, cardamom, peppermint, and eucalyptus.

39. The composition of any one of claims 1 to 5, 7 to 10, and 25 to 38, wherein the 5HT2A agonist compound is psilocybin, and wherein the therapeutically effective amount of psilocybin is between about 100 mg and about 300 mg.

40. The composition of any one of claims 1 to 5, 7 to 10, and 25 to 39, wherein the 5HT2A agonist is psilocybin, and wherein the therapeutically effective amount of psilocybin is between about 110 mg and about 290 mg, about 120 mg and about 280 mg, about 130 mg and about 270 mg, about 140 mg and about 260 mg, about 150 mg and about 250 mg, about 160 mg and about 240 mg, about 170 mg and about 230 mg, about 180 mg and about 220 mg, about 190 mg and about 210 mg, or about 195 mg and about 205 mg.

41. The composition of any one of claims 1 to 40, wherein the at least one TRP agonist compound is capsaicin in an amount of about 0.1 mg and about 1 mg, about 0.2 mg and bout 0.9 mg, about 0.3 mg and about 0.8 mg, about 0.4 and about 0.7 mg, or about 0.5 mg and about 0.6 mg.

42. The composition of any one of claims 1 to 40, wherein the at least one TRP agonist compound is capsaicin, and wherein the composition comprises a ratio (w/w) of between about 22:1 and about 270,000:1 of the 5HT2A agonist to capsaicin, about 50:1 and about 200,000:1 of the 5HT2A agonist to capsaicin, about 100:1 and about 150,000:1 of the agonist to capsaicin, about 500:1 and about 100,000:1 of the 5HT2A agonist to capsaicin, about 1,000:1 and about 50,000:1 of the 5HT2A agonist to capsaicin, about and about 40,000:1 of the 5HT2A agonist to capsaicin, about 10,000:1 and about of the 5HT2A agonist to capsaicin, or about 15,000:1 and about 25,000:1 of the agonist to capsaicin.

43. The composition of any one of claims 1 to 40, wherein the at least one TRP agonist compound is eugenol in an amount of about 1 mg and about 300 mg, about 5 mg and about 290 mg, about 10 mg and about 280 mg, about 15 mg and about 270 mg, about 20 mg and about 260 mg, about 25 mg and about 250 mg, about 30 mg and about 240 mg, about 35 mg and about 230 mg, about 40 mg and about 220 mg, about 40 mg and about 210 mg, about 50 mg and about 210 mg, about 55 mg and about 200 mg, about 60 mg and about 190 mg, about 65 mg and about 180 mg, about 70 mg and about 170 mg, about 75 mg and about 160 mg, about 80 mg and about 150 mg, about 85 mg and about 140 mg, about 90 mg and about 130 mg, about 95 mg and about 120 mg, or about 100 mg and about 110 mg.

44. The composition of any one of claims 1 to 40, wherein the at least one TRP agonist compound is eugenol, and wherein the composition comprises a ratio (w/w) of between about 0.6:1 and about 270,000:1 of the 5HT2A agonist to eugenol, about 1:1 and about 250,000:1 of the 5HT2A agonist to eugenol, about 5:1 and about 225,000:1 of the 5HT2A agonist to eugenol, about 10:1 and about 200,000:1 of the 5HT2A agonist to eugenol, about 50:1 and about 175,000:1 of the 5HT2A agonist to eugenol, about 100:1 and about 150,000:1 of the 5HT2A agonist to eugenol, about 150:1 and about 125,000:1 of the agonist to eugenol, about 300:1 and about 100,000:1 of the 5HT2A agonist to eugenol, about 500:1 and about 75,000:1 of the 5HT2A agonist to eugenol, about 1,000:1 and about 50,000:1 of the 5HT2A agonist to eugenol, about 5,000:1 and about 45,000:1 of the 5HT2A agonist to eugenol, about 10,000:1 and about 40,000:1 of the 5HT2A agonist to eugenol, about 15,000:1 and about 35,000:1 of the 5HT2A agonist to eugenol, or about 20,000:1 and about 30,000:1 of the 5HT2A agonist to eugenol.

45. The composition of any one of claims 1 to 40, wherein the at least one TRP agonist compound is curcumin in an amount of about 0.1 mg to about 10 mg, about 0.5 mg to about 9 mg, about 1 mg to about 8 mg, about 2 mg to about 7 mg, about 3 mg to about 6 mg, or about 4 mg to about 5 mg.

46. The composition of any one of claims 1 to 40, wherein the at least one TRP agonist compound is curcumin, and wherein the composition comprises a ratio (w/w) of between about 0.04:1 and about 10:1 of the 5HT2A agonist to curcumin, about 0.1:1 and about 9.5:1 of the 5HT2A agonist to curcumin, about 0.5:1 and about 9:1 of the 5HT2A agonist to curcumin, about 1:1 and about 8.5:1 of the 5HT2A agonist to curcumin, about 1.5:1 and about 8:1 of the 5HT2A agonist to curcumin, about 2:1 and about 7.5:1 of the 5HT2A agonist to curcumin, about 2.5:1 and about 7:1 of the 5HT2A agonist to curcumin, about 3:1 and about 6.5:1 of the 5HT2A agonist to curcumin, about 3.5:1 and about 6:1 of the agonist to curcumin, about 4:1 and about 5.5:1 of the 5HT2A agonist to curcumin, or about 4.5:1 and about 5:1 of the 5HT2A agonist to curcumin.

47. The composition of any one of claims 1 to 40, wherein the at least one TRP agonist compound is β-caryophyllene, and wherein the composition comprises a ratio (w/w) of between about 0.33:1 and about 36:1 of the 5HT2A agonist to β-caryophyllene, about 1:1 and about 33:1 of the 5HT2A agonist to β-caryophyllene, about 3:1 and about 30:1 of the agonist to β-caryophyllene, about 5:1 and about 27:1 of the 5HT2A agonist to β-caryophyllene, about 7:1 and about 25:1 of the 5HT2A agonist to β-caryophyllene, about and about 22:1 of the 5HT2A agonist to β-caryophyllene, about 15:1 and about 20:1 of the 5HT2A agonist to β-caryophyllene, or about 17:1 and about 18:1 of the 5HT2A agonist to β-caryophyllene.

48. The composition of any one of claims 1 to 40, wherein the at least one TRP agonist compound is cinnamaldehyde in an amount of between about 0.1 mg and about 10 mg, about 0.5 mg and about 9.5 mg, about 1 mg and about 9 mg, about 1.5 mg and about 8.5 mg, about 2 mg and about 8 mg, about 2.5 mg and about 7.5 mg, about 3 mg and about 7 mg, about 3.5 mg and about 6.5 mg, about 4 mg and about 6 mg, or about 4.5 mg and about 5.5 mg.

49. The composition of any one of claims 1 to 40, wherein the at least one TRP agonist compound is cinnamaldehyde, and wherein the composition comprises a ratio (w/w) of between about 0.5:1 and about 36:1 of the 5HT2A agonist to cinnamaldehyde, about 1:1 and about 33:1 of the 5HT2A agonist to cinnamaldehyde, about 3:1 and about 30:1 of the agonist to cinnamaldehyde, about 5:1 and about 27:1 of the 5HT2A agonist to cinnamaldehyde, about 7:1 and about 25:1 of the 5HT2A agonist to cinnamaldehyde, about 10:1 and about 22:1 of the 5HT2A agonist to cinnamaldehyde, about 15:1 and about 20:1 of the 5HT2A agonist to cinnamaldehyde, or about 17:1 and about 18:1 of the agonist to cinnamaldehyde.

50. The composition of any one of claims 1 to 49, wherein the composition is formulated for oral administration.

51. The composition of claim 50, further comprising at least one pharmaceutically acceptable excipient, diluent, or filler.

52. The composition of any one of claim 50 or 51, wherein the composition is selected from the group consisting of a tablet, capsule, sachets, granules, sublingual film, buccal film, and a suspension.

53. A method for reducing inflammation in a subject, comprising administering the composition of any one of claims 1 to 52 to the subject.

54. The method of claim 53, wherein the inflammation is acute or chronic.

55. The method of any one of claim 53 or 54, comprising administering the composition 1, 2, 3, 4, 5, 6, 7, 8, 9, or 10 times per day.

56. The method of any one of claims 53 to 55, whereby the reduction in inflammation is measured by an 50% reduction of at least one biomarker selected from the group consisting of COX-2, interferon-γ, interleukin 1, interleukin-2, interleukin-6, interleukin-8, interleukin-10, tumor necrosis factor (TNF), and reactive oxygen species (ROS) when measured via densitometry.

57. The method of claim 56, wherein the ROS is inducible nitric oxide synthase (iNOS).

58. The method of any one of claims 53 to 57, wherein the subject is suffering from a condition selected from the group consisting of cancer, neurological disorder, diabetic complications, mental health disorder (MHD), bone, muscular and skeletal disease, metabolic disorder, chronic inflammatory disorder and cardiovascular disease.

59. The method of claim 58, wherein the MHD is selected from depression, anxiety, post-traumatic stress disorder, schizophrenia, bipolar disorder, ADD, ADHD, borderline personality disorder, seasonal affective disorder, and premenstrual dysphoric disorder.

60. The method of claim 58, wherein the MHD is depression, and wherein the reduction in inflammation is accompanied by a reduction in at least one symptom of depression.

61. The method of claim 58, wherein the MHD is anxiety, and wherein the reduction in inflammation is accompanied by a reduction in at least one symptom of anxiety.

62. A method for reducing at least one biomarker in a mammalian cell, wherein the biomarker is selected from the group consisting of COX-2, interferon-γ, interleukin 1, interleukin-2, interleukin-6, interleukin-8, interleukin-10, tumor necrosis factor (TNF), and reactive oxygen species (ROS), comprising administering the composition of any one of claims 1 to 52 to a subject,
wherein administering the composition reduces the biomarker in the mammalian cell between about 10% and about 90%.

63. The method of claim 62, wherein the 5HT2A agonist is psilocybin in an amount of about 100 mg to about 300 mg, and wherein the TRP agonist is eugenol in an amount of about 100 mg to about 300 mg.

64. The method of claim 62 or 63, wherein the 5HT2A agonist is psilocybin in an amount of about 100 mg to about 300 mg, about 110 mg to about 290 mg, about 120 mg to about 280 mg, about 130 mg to about 270 mg, about 140 mg to about 260 mg, about 150 mg to about 250 mg, about 160 mg to about 240 mg, about 170 mg to about 230 mg, about 180 mg to about 220 mg, about 190 mg to about 210 mg, or about 195 mg to about 205 mg, and wherein TRP agonist is eugenol in an amount of about 100 mg to about 300 mg, about 110 mg to about 290 mg, about 120 mg to about 280 mg, about 130 mg to about 270 mg, about 140 mg to about 260 mg, about 150 mg to about 250 mg, about 160 mg to about 240 mg, about 170 mg to about 230 mg, about 180 mg to about 220 mg, about 190 mg to about 210 mg, or about 195 mg to about 205 mg.

65. The method of any one of claims 62-64, wherein administering the composition reduces IL-6 in the mammalian cell by about an additional 20% relative to administering the therapeutically effective amount of psilocybin alone.

66. The method of claim 65, wherein administering the composition reduces IL-6 in the mammalian cell by about an additional 25% relative to administering the therapeutically effective amount of psilocybin alone.

67. The composition of any one of claims 1-38, wherein the therapeutically effective amount of the 5HT2A agonist is between about 10 μg and about 195 mg, about 50 μg and about 190 mg, about 100 μg and about 185 mg, about 200 μg and about 180 mg, about 300 μg and about 175 mg, about 400 μg and about 170 mg, about 500 μg and about 165 mg, about 600 μg and about 160 mg, about 700 μg and about 155 mg, about 800 μg and about 150 mg, about 900 μg and about 145 mg, about 1 mg and about 140 mg, about 5 mg and about 135 mg, about 10 mg and about 130 mg, about 15 mg and about 125 mg, about 20 mg and about 120 mg, about 25 mg and about 115 mg, about 30 mg and about 110 mg, about 35 mg and about 105 mg, about 40 mg and about 100 mg, about 45 mg and about mg, about 50 mg and about 90 mg, about 55 mg and about 85 mg, about 60 mg and about 80 mg, or about 65 mg and about 75 mg.

68. The composition of any one of claims 1-38, wherein the therapeutically effective amount of the at least one TRP receptor agonist is between about 0.1 mg and about 24 mg, about mg and about 23 mg, about 1 mg and about 22 mg, about 2 mg and about 21 mg, about 3 mg and about 20 mg, about 4 mg and about 19 mg, about 5 mg and about 18 mg, about 6 mg and about 17 mg, about 7 mg and about 16 mg, about 8 mg and about 15 mg, about 9 mg and about 14 mg, about 10 mg and about 13 mg, or about 11 mg and about 12 mg.

69. The composition of any one of claims 1-38, wherein the therapeutically effective amount of the 5HT2A agonist is between about 10 μg and about 195 mg, about 50 μg and about 190 mg, about 100 μg and about 185 mg, about 200 μg and about 180 mg, about 300 μg and about 175 mg, about 400 μg and about 170 mg, about 500 μg and about 165 mg, about 600 μg and about 160 mg, about 700 μg and about 155 mg, about 800 μg and about 150 mg, about 900 μg and about 145 mg, about 1 mg and about 140 mg, about 5 mg and about 135 mg, about 10 mg and about 130 mg, about 15 mg and about 125 mg, about 20 mg and about 120 mg, about 25 mg and about 115 mg, about 30 mg and about 110 mg, about 35 mg and about 105 mg, about 40 mg and about 100 mg, about 45 mg and about mg, about 50 mg and about 90 mg, about 55 mg and about 85 mg, about 60 mg and about 80 mg, or about 65 mg and about 75 mg, and wherein the therapeutically effective amount of the at least one TRP receptor agonist is between about 0.1 mg and about 24 mg, about 0.5 mg and about 23 mg, about 1 mg and about 22 mg, about 2 mg and about 21 mg, about 3 mg and about 20 mg, about 4 mg and about 19 mg, about 5 mg and about 18 mg, about 6 mg and about 17 mg, about 7 mg and about 16 mg, about 8 mg and about mg, about 9 mg and about 14 mg, about 10 mg and about 13 mg, or about 11 mg and about 12 mg.
